(19) United States
(12) Patent Application Publication
(10) Pub. No.: US 2026/0347495 A1
(43) Pub. Date: Nov. 5, 2026

(54) Antibodies that bind CD228

(71) Applicant: Seagen Inc., Bothell, WA (US)

(72) Inventors: Ryan Heiser, Bothell, WA (US); Sharsti Sandall, Bothell, WA (US); Erin Scherer, Bothell, WA (US)

(73) Assignee: Seagen Inc., Bothell, WA (US)

(21) Appl. No.: 19/111,642

(22) PCT Filed: Sep. 20, 2023
(86) PCT No.: PCT/US2023/074630

§ 371 Date: Mar. 13, 2025

Related U.S. Application Data
(60) Provisional application No. 63/408,605, filed on Sep. 21, 2022.

(51) Int. Cl. C07K 16/28; A61K 39/00; A61K 47/68; A61P 35/00

(52) U.S. Cl. CPC C07K 16/2896; A61K 47/68031; A61K 47/6849; A61P 35/00; A61K 2039/505; C07K 2317/24; C07K 2317/565; C07K 2317/622; C07K 2317/92

(57) ABSTRACT

The disclosure provides antibodies or antigen-binding fragments thereof specific for CD228. Such antibodies or antigen-binding fragments can be used in many pharmaceutical applications, for example, as anti-cancer agents and/or immune modulators. The present disclosure also concerns methods of making the antibodies or antigen-binding fragments described herein as well as compositions comprising such antibodies or antigen-binding fragments. The present disclosure further relates to nucleic acid molecules encoding such antibodies or antigen-binding fragments. In addition, the application discloses therapeutic and/or diagnostic uses of such antibodies or antigen-binding fragments.

### I. CROSS-REFERENCE TO RELATED APPLICATIONS

This application claims the benefit of priority of U.S. Provisional Application No. 63/408,605, filed Sep. 21, 2022, which is incorporated by reference herein in its entirety for any purpose.

### II. INTRODUCTION AND SUMMARY

Cluster of differentiation 228 or CD228 (also known as melanotransferrin, MELTF, p97 and MFI2) is a glycosylphosphatidylinositol (GPI)-anchored glycoprotein that belongs to the transferrin family of iron-binding proteins and was first described as an oncofetal protein highly expressed on malignant melanoma cells (Rose et al., Proc Natl Acad Sci USA, 1986).

CD228 is expressed in a variety of cancers, including melanoma, mesothelioma, thyroid cancer, lung cancer, liver cancer, pancreatic cancer, head and neck cancer, stomach cancer, colorectal cancer, urothelial cancer, breast cancer, and cervical cancer. Melanoma, also known as malignant melanoma, is a type of cancer that develops from melanocytes, which are pigment-containing cells. Melanoma is the most dangerous type of skin cancer. In 2015, were 3.1 million people with active disease and melanoma resulted in 59,800 deaths. Surgery can be effective for early-stage melanoma but may not be a treatment option for disease that has metastasized to distant organs. Melanomas that spread often do so to the lymph nodes in the area before spreading elsewhere. Attempts to improve survival by removing lymph nodes surgically were associated with many complications but no overall survival benefit. Immunotherapy, chemotherapy, and radiation therapy have all been used, but are often not curative, particularly for late-stage melanoma. When there is distant metastasis, the cancer is generally considered incurable. The five-year survival rate of stage IV disease is 15-20%.

The present disclosure provides, among other things, novel antibodies and antigen-binding fragments thereof that bind CD228.

### III. DEFINITIONS

The following list defines terms, phrases, and abbreviations used throughout the instant specification. All terms listed and defined herein are intended to encompass all grammatical forms.

As used herein, unless otherwise specified, “CD137” means human CD137 (huCD137). Human CD137 means a full-length protein defined by UniProt Q07011, a fragment thereof, or a variant thereof. CD137 is also known as 4-1BB, tumor necrosis factor receptor superfamily member 9 (TNFRSF9), and induced by lymphocyte activation (ILA). In some particular embodiments, CD137 of non-human species, e.g., cynomolgus CD137 or mouse CD137, is used.

As used herein, unless otherwise specified, “CD228” means human CD228. Human CD228 means a full-length protein defined by UniProt P08582, a mature form thereof, an isoform thereof, a fragment thereof, or a variant thereof. Human CD228 is encoded by the MELTF gene. CD228 is also known as melanotransferrin, MELTF, p97 and MFI2, which terms may be used interchangeably herein. In some particular embodiments, CD228 of non-human species, e.g., cynomolgus CD228 or mouse CD228, is used.

As used herein, “binding affinity” describes the ability of a biomolecule (e.g., a polypeptide or a protein) of the disclosure (e.g., an antibody or antigen-binding fragment thereof, a fusion protein, or any other peptide or protein) to bind a selected target (and form a complex). Binding affinity is measured by a number of methods known to those skilled in the art including, but not limited to, fluorescence titration, enzyme-linked immunosorbent assay (ELISA)-based assays, including direct and competitive ELISA, calorimetric methods, such as isothermal titration calorimetry (ITC), and surface plasmon resonance (SPR). These methods are well-established in the art and some examples of such methods are further described herein. Binding affinity is thereby reported as a value of the dissociation constant (K_{D}), half maximal effective concentration (EC₅₀), or half maximal inhibitory concentration (IC₅₀) measured using such methods. A lower K_{D}, EC₅₀, or IC₅₀ value reflects better (higher) binding ability (affinity). Accordingly, the binding affinities of two biomolecules toward a selected target can be measured and compared. When comparing the binding affinities of two biomolecules toward the selected target, the term “comparable to”, “about the same,” “substantially the same” or “substantially similar” means one biomolecule has a binding affinity reported as a K_{D}, an EC₅₀, or an IC₅₀ value that is identical or similar to that of another molecule within the experimental variability of the binding affinity measurement. Preferably, “comparable to”, “about the same,” “substantially the same” or “substantially similar” relate to a value that is within 50% deviation to a given reference value, more preferably within 20% deviation, most preferably within 10% deviation. The experimental variability of the binding affinity measurement is dependent upon the specific method used and is known to those skilled in the art.

As used herein, the term “substantially” may also refer to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “detect”, “detection”, “detectable”, or “detecting” is understood both on a quantitative and a qualitative level, as well as a combination thereof. It thus includes quantitative, semi-quantitative, and qualitative measurements performed on a biomolecule of the disclosure.

As used herein, “detectable affinity” generally means the binding ability between a biomolecule and its target, reported by a K_{D}, EC₅₀, or IC₅₀ value, is at most about 10⁻⁵ M or lower. A binding affinity, reported by a K_{D}, EC₅₀, or IC₅₀ value, higher than 10⁻⁵ M is generally no longer measurable with common methods such as ELISA and SPR and is therefore of secondary importance. Thus, “detectable affinity” may refer to a K_{D} value of about 10⁻⁵ M or lower as determined by ELISA or SPR, preferably SPR.

As used herein, “specific for”, “specific binding”, “specifically bind”, or “binding specificity” relates to the ability of a biomolecule to discriminate between the desired target (for example, CD228) and one or more reference targets. It is understood that such specificity is not an absolute but a relative property and can be determined, for example, by means of SPR, western blots, ELISA, fluorescence activated cell sorting (FACS), radioimmunoassay (RIA), electrochemiluminescence (ECL), immunoradiometric assay (IRMA), ImmunoHistoChemistry (IHC), and peptide scans.

When used herein in the context of a biomolecule, such as an antibody, an antigen-binding fragment thereof, or a fusion protein, of the present disclosure that binds to CD228, the term “specific for”, “specific binding”, “specifically bind”, or “binding specificity” means that the biomolecule binds to, reacts with, or is directed against CD228, as described herein, but does not substantially bind another protein. The term “another protein” includes any proteins that are not CD228 nor proteins closely related to or being homologous to CD228. However, CD228 from species other than human and fragments and/or variants of CD228 are not excluded by the term “another protein.” The term “does not substantially bind” means that a biomolecule of the present disclosure binds another protein with lower binding affinity than CD228, i.e., shows a cross-reactivity of less than 30%, preferably 20%, more preferably 10%, particularly preferably less than 9, 8, 7, 6, or 5%. Whether the biomolecule specifically reacts as defined herein above can easily be tested, inter alia, by comparing the reaction of a biomolecule of the present disclosure with CD228 and the reaction of said biomolecule with (an) other protein(s).

As used herein, the term “lipocalin” refers to a monomeric protein of approximately 18-20 kDa in weight, having a cylindrical β-pleated sheet supersecondary structural region comprising a plurality of β-strands (preferably eight β-strands designated A to H) connected pair-wise by a plurality of (preferably four) loops at one end to thereby comprise a ligand-binding pocket and define the entrance to the ligand-binding pocket. Preferably, the loops comprising the ligand-binding pocket used in the present disclosure are loops connecting the open ends of β-strands A and B, C and D, E and F, and G and H, and are designated loops AB, CD, EF, and GH. It is well-established that the diversity of said loops in the otherwise rigid lipocalin scaffold gives rise to a variety of different binding modes among the lipocalin family members, each capable of accommodating targets of different sizes, shape, and chemical character (reviewed, e.g., in Skerra, Biochim Biophys Acta, 2000; Flower et al., Biochim Biophys Acta, 2000; Flower, Biochem J, 1996). It is understood that the lipocalin family of proteins has naturally evolved to bind a wide spectrum of ligands, sharing unusually low levels of overall sequence conservation (often with sequence identities of less than 20%) yet retaining a highly conserved overall folding pattern. The correspondence between positions in various lipocalins is also well-known to one of skill in the art (see, e.g., U.S. Pat. No. 7,250,297). Proteins falling in the definition of “lipocalin” as used herein include, but are not limited to, human lipocalins including tear lipocalin (Tlc, Lcn1), Lipocalin-2 (Lcn2) or neutrophil gelatinase-associated lipocalin (NGAL), apolipoprotein D (ApoD), apolipoprotein M, α₁-acid glycoprotein 1, α₁-acid glycoprotein 2, α₁-microglobulin, complement component 8γ, retinol-binding protein (RBP), the epididymal retinoic acid-binding protein, glycodelin, odorant-binding protein IIa, odorant-binding protein IIb, lipocalin-15 (Lcn15), and prostaglandin D synthase.

As used herein, a “mutein”, a “mutated” entity (whether protein or nucleic acid), or “mutant” refers to the exchange, deletion, or insertion of one or more amino acids or nucleotides, compared to the naturally occurring (wild-type) protein or nucleic acid. Said term also includes fragments of a mutein as described herein. In some embodiments, a lipocalin mutein (also referred to as an Anticalin® protein), as described herein, has a cylindrical β-pleated sheet supersecondary structural region comprising eight β-strands connected pair-wise by four loops at one end to thereby comprise a ligand-binding pocket and define the entrance of the ligand-binding pocket, wherein at least one amino acid located within said four loops has been mutated as compared to the native sequence lipocalin.

As used herein, the term “variant” relates to derivatives of a protein or polypeptide that include mutations, for example by substitutions, deletions, insertions, and/or chemical modifications of an amino acid sequence or nucleotide sequence. In some embodiments, such mutations and/or chemical modifications do not reduce the functionality of the protein or peptide. Such substitutions may be conservative, i.e., an amino acid residue is replaced with a chemically similar amino acid residue. Examples of conservative substitutions are the replacements among the members of the following groups: 1) alanine, serine, threonine, and valine; 2) aspartic acid, glutamic acid, glutamine, asparagine, and histidine; 3) arginine, lysine, glutamine, asparagine, and histidine; 4) isoleucine, leucine, methionine, valine, alanine, phenylalanine, threonine, and proline; and 5) isoleucine, leucine, methionine, phenylalanine, tyrosine, and tryptophan. Such variants include proteins or polypeptides, wherein one or more amino acids have been substituted by their respective D-stereoisomers or by amino acids other than the naturally occurring 20 amino acids, such as, for example, ornithine, hydroxyproline, citrulline, homoserine, hydroxylysine, norvaline. Such variants also include, for instance, proteins or polypeptides in which one or more amino acid residues are added or deleted at the N- and/or C-terminus. Generally, a variant has at least about 50%, 60%, 70%, 75%, 80%, 85%, 90%, 92%, 95% or at least about 98% amino acid sequence identity with the native sequence protein or polypeptide. A variant preferably retains the biological activity, e.g., binding the same target, of the protein or polypeptide it is derived from.

The term “variant”, as used herein with respect to an antibody or an antigen-binding fragment thereof relates to an antibody or an antigen-binding fragment thereof of the disclosure, wherein the sequence has mutations, including substitutions, deletions, insertions, and/or chemical modifications. A variant of an antibody or an antigen-binding fragment thereof as described herein retains the biological activity, e.g., binding to CD228, of the antibody or antigen-binding fragment thereof from which it is derived. Generally, a variant of an antibody or an antigen-binding fragment thereof has at least about 50%, 60%, 70%, 75%, 80%, 85%, 90%, 92%, 95%, or 98% amino acid sequence identity with the antibody or antigen-binding fragment thereof from which it is derived.

As used herein, the term “sequence identity” or “identity” denotes a property of sequences that measures their similarity or relationship. The term “sequence identity” or “identity” as used in the present disclosure means the percentage of pair-wise identical residues—following (homologous) alignment of a sequence of a protein or polypeptide of the disclosure with a sequence in question—with respect to the number of residues in the longer of these two sequences. Sequence identity is measured by dividing the number of identical amino acid residues by the total number of residues and multiplying the product by 100.

As used herein, the term “sequence homology” or “homology” has its usual meaning, and a homologous amino acid includes identical amino acids as well as amino acids which are regarded to be conservative substitutions at equivalent positions in the linear amino acid sequence of a protein or polypeptide of the disclosure.

A skilled artisan will recognize available computer programs, for example BLAST (Altschul et al., Nucleic Acids Res, 1997), BLAST2 (Altschul et al., J Mol Biol, 1990), and Smith-Waterman (Smith and Waterman, J Mol Biol, 1981), for determining sequence homology or sequence identity using standard parameters. The percentage of sequence homology or sequence identity can, for example, be determined herein using the program BLASTP, version 2.2.5 (Nov. 16, 2002; (Altschul et al., Nucleic Acids Res, 1997). In some embodiments, the percentage of homology is based on the alignment of the entire protein or polypeptide sequences (matrix: BLOSUM 62; gap costs: 11.1; cutoff value set to 10⁻³) including the propeptide sequences, preferably using the wild-type protein scaffold as reference in a pairwise comparison. It is calculated as the percentage of numbers of “positives” (homologous amino acids) indicated as result in the BLASTP program output divided by the total number of amino acids selected by the program for the alignment.

“Gaps” are spaces in an alignment that are the result of additions or deletions of amino acids. Thus, two copies of exactly the same sequence have 100% identity, but sequences that are less highly conserved, and have deletions, additions, or replacements, may have a lower degree of sequence identity.

As used interchangeably herein, the terms “conjugate”, “conjugation”, “fuse”, “fusion”, or “linked” refer to the joining together of two or more subunits, through all forms of covalent or non-covalent linkage, by means including, but not limited to, genetic fusion, chemical conjugation, coupling through a linker or a cross-linking agent, and non-covalent association.

The term “fusion polypeptide” or “fusion protein” as used herein refers to a polypeptide or protein comprising two or more subunits. In some embodiments, a fusion protein as described herein comprises two or more subunits, at least one of these subunits being capable of specifically binding to CD228. Within the fusion protein, these subunits may be linked by covalent or non-covalent linkage. Preferably, the fusion protein is a translational fusion between the two or more subunits. The translational fusion may be generated by genetically engineering the coding sequence for one subunit in a reading frame with the coding sequence of a further subunit. Both subunits may be interspersed by a nucleotide sequence encoding a linker. However, the subunits of a fusion protein of the present disclosure may also be linked through chemical conjugation. The subunits forming the fusion protein are typically linked to each other as follows: C-terminus of one subunit to N-terminus of another subunit, or C-terminus of one subunit to C-terminus of another subunit, or N-terminus of one subunit to N-terminus of another subunit, or N-terminus of one subunit to C-terminus of another subunit. The subunits of the fusion protein can be linked in any order and may include more than one of any of the constituent subunits. If one or more of the subunits are part of a protein (complex) that consists of more than one polypeptide chain, the term “fusion protein” may also refer to the protein comprising the fused sequences and all other polypeptide chain(s) of the protein (complex). As an illustrative example, where a full-length immunoglobulin/antibody is fused to a lipocalin mutein via a heavy or light chain of the immunoglobulin/antibody, the term “fusion protein” may refer to the single polypeptide chain comprising the lipocalin mutein and the heavy or light chain of the immunoglobulin/antibody. The term “fusion protein” may also refer to the entire immunoglobulin/antibody (both light and heavy chains) and the lipocalin mutein fused to one or both of its heavy and/or light chains.

As used herein, the term “subunit” of a fusion protein disclosed herein refers to a single protein or a separate polypeptide chain, which can form a stable folded structure by itself and may define a unique function of providing a binding motif towards a target. In some embodiments, a preferred subunit of the disclosure is an antibody, such as a full-length antibody, or an antigen-binding domain/fragment thereof.

A “linker” that may be comprised by a fusion protein of the present disclosure joins together two or more subunits of a fusion protein as described herein.

As used herein, the term “albumin” includes all mammalian albumins, such as human serum albumin or bovine serum albumin or rat serum albumin.

As used herein, the term “organic molecule” or “small organic molecule” denotes an organic molecule comprising at least two carbon atoms, but preferably not more than 7 or 12 rotatable carbon bonds, having a molecular weight in the range between 100 and 2,000 daltons, preferably between 100 and 1,000 daltons, and optionally including one or two metal atoms.

A “sample” is defined as a biological sample taken from any subject. Biological samples include, but are not limited to, blood, serum, urine, feces, semen, or tissue, including tumor tissue.

A “subject” is a vertebrate, preferably a mammal, more preferably a human. The term “mammal” is used herein to refer to any animal classified as a mammal, including, without limitation, humans, domestic and farm animals, and zoo, sports, or pet animals, such as sheep, dogs, horses, cats, cows, rats, pigs, apes such as cynomolgus monkeys, to name only a few illustrative examples. Preferably, the “mammal” used herein is human.

An “effective amount” is an amount sufficient to yield beneficial or desired results. An effective amount can be administered in one or more individual administrations or doses.

As used herein, “antibody” includes whole antibodies or any antigen-binding fragment (i.e., “antigen-binding portion” or “antigen-binding domain”) or single chain thereof. The terms “antibody” and “immunoglobulin” can be and are used interchangeably herein. A whole antibody refers to a glycoprotein comprising at least two heavy chains (HCs) and two light chains (LCs) inter-connected by disulfide bonds. Each heavy chain is comprised of a heavy chain variable domain/region (V_{H} or HCVR) and a heavy chain constant region (C_{H}). The heavy chain constant region is comprised of three domains, C_{H1}, C_{H2} and C_{H3}. Each light chain is comprised of a light chain variable domain/region (V_{L} or LCVR) and a light chain constant region (C_{L}). The light chain constant region is comprised of one domain, C_{L}. The VH and VL regions can be further subdivided into regions of hypervariability, termed complementarity determining regions (CDRs), interspersed with regions that are more conserved, termed framework regions (FRs). Each V_{H} and VL is composed of three CDRs and four FRs, arranged in the following order from the amino-terminus to the carboxy-terminus: FR1, CDR1, FR2, CDR2, FR3, CDR3, FR4. The variable regions of the heavy and light chains contain a binding domain that interacts with an antigen (for example, CD228). The constant regions of the antibodies may optionally mediate the binding of the immunoglobulin to host tissues or factors, including various cells of the immune system (e.g., effector cells) and the first component (C1q) of the classical complement system.

As used herein, “antigen-binding fragment” (also referred to as “antigen-binding domain”) of an antibody refers to one or more fragments of an antibody that retain the ability to specifically bind to an antigen (e.g., CD228). It has been shown that the antigen-binding function of an antibody can be performed by fragments of a full-length antibody. Examples of binding fragments encompassed within the term “antigen-binding fragment” of an antibody include (i) a Fab fragment consisting of the V_{H}, V_{L}, C_{L} and C_{H} domains; (ii) a F(ab′)₂ fragment comprising two Fab fragments linked by a disulfide bridge at the hinge region; (iii) a Fab′ fragment consisting of the V_{H}, V_{L}, C_{L} and C_{H1} domains and the region between the C_{H1} and C_{H2} domains; (iv) an Fd fragment consisting of the V_{H} and C_{H1} domains; (v) a single-chain Fv fragment consisting of the V_{H} and V_{L} domains of a single arm of an antibody, (vi) a dAb fragment (Ward et al., Nature, 1989) consisting of a V_{H} domain; (vii) an isolated complementarity determining region (CDR) or a combination of two or more isolated CDRs which may optionally be joined by a synthetic linker; (viii) a “diabody” comprising the VH and VL connected in the same polypeptide chain using a short linker (see, e.g., patent documents EP 404,097; WO 93/11161; and Holliger et al., Proc Natl Acad Sci USA, 1993); and (ix) a “domain antibody fragment” containing only the V_{H} or V_{L}, where in some instances two or more VH regions are covalently joined.

Antibodies may be polyclonal or monoclonal; xenogeneic, allogeneic, or syngeneic; or modified forms thereof (e.g., humanized, chimeric, or multispecific). Antibodies may also be fully human.

As used herein, “framework” or “FR” refers to the variable domain residues other than the hypervariable region (CDR) residues.

“Fragment crystallizable region” or “Fc region” refers to the C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof numbering according to EU index of Kabat (Johnson and Wu, Nucleic Acids Res, 2000). The C-terminal lysine (residue 447 according to EU index of Kabat) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the disclosure include human IgG1, IgG2 (IgG2A, IgG2B), IgG3, and IgG4.

“Fc receptor” or “FcR” refers to a receptor that binds to the Fc region of an antibody.

As used herein, “isolated antibody” refers to an antibody that is substantially free of its natural environment. For instance, an isolated antibody is substantially free of cellular material and other proteins from the cell or tissue source from which it is derived. An “isolated antibody” further refers to an antibody that is substantially free of other antibodies having different antigenic specificities. In the present case, an isolated antibody that binds specifically CD228 is substantially free of antibodies that specifically bind antigens other than CD228. However, an isolated antibody that specifically binds CD228 may have cross-reactivity to other antigens, such as CD228 molecules from other species.

As used herein, “monoclonal antibody” refers to a preparation of antibody molecules of single molecular composition. A monoclonal antibody composition displays a single binding specificity and affinity for a particular epitope.

As used herein, “humanized antibody” refers to an antibody that consists of the CDRs of antibodies derived from mammals other than human, and the FR region and the constant region of a human antibody or derived from a human antibody. A humanized antibody may comprise a variable domain that has a variable region amino acid sequence which, analyzed as a whole, is closer to human than to other species as assessed using the Immunogenetics Information System (IMGT) DomainGapAlign tool, as described by Ehrenmann et al. (2010). A humanized antibody may be useful as an effective component in a therapeutic agent due to the reduced antigenicity. The term “therapeutic agent” or “therapeutically active agent”, as used herein, refers to an agent which is therapeutically useful. A therapeutic agent may be any agent for the prevention, amelioration, or treatment of a disease, a physiological condition, a symptom, or for the evaluation or diagnosis thereof.

As used herein, “human antibody” includes antibodies having variable regions in which both the framework and CDR regions are derived from human germline immunoglobulin sequences. Furthermore, if the antibody contains a constant region, the constant region is also derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

As used herein, “antibody clone X” may also be referred to as “OMTX.” For example, “antibody clone 30” may also be referred to as “OMT30.” Antibody clone 8 may also be referred to as “EE03”; antibody clone 24 may also be referred to as “EB03”; antibody clone 30 may also be referred to as “OC04”; antibody clone 35 may also be referred to as “OB02”; and antibody clone 36 may also be referred to as “OE12.”

As used herein, “AAFX” refers to a fusion protein containing antibody clone X and the lipocalin mutein having the amino acid sequence of SEQ ID NO: 40 (Lipocalin Mutein I). For example, “AAF30” refers to a fusion protein containing antibody clone 30 and Lipocalin Mutein I. The notation “HC” in the name of a fusion protein refers to the lipocalin mutein therein being conjugated to the antibody via the antibody's heavy chain, and the notation “LC” in the name of a fusion protein refers to the lipocalin mutein therein being conjugated to the antibody via the antibody's light chain. A fusion protein name that contains a clone number followed by “HC” or “LC” refers to a fusion protein containing that antibody clone and Lipocalin Mutein I, conjugated via the antibody's heavy chain or light chain, respectively. For example, “30HC” refers to a fusion protein containing antibody clone 30 and Lipocalin Mutein I conjugated thereto via the antibody's heavy chain.

An antibody or fusion protein may also be denoted by its heavy and light chain sequences (e.g., the fusion protein having the sequences of SEQ ID NOs: 80 and 76), one of which in the case of a fusion protein (e.g., SEQ ID NO: 80) contains the sequence of a lipocalin mutein.

### IV. DESCRIPTIONS OF FIGURES

FIG. 1 provides a schematic showing CD137 clustering by bridging CD137-positive T cells with CD228-expressing tumor cells using the fusion proteins provided herein.

FIGS. 2A and 2B show CDR sequences of certain antibodies provided herein

FIG. 3 shows results of an ELISA, in which anti-CD228 antibodies bind recombinant human CD228.

FIGS. 4A-4C show binding of anti-CD228 antibodies to CD228-expressing cells, as measured by fluorescence intensity. FIG. 4D shows the EC₅₀ value of each antibody for each cell line.

FIGS. 5A-5E show results of biolayer inferometry (BLI) assays for binding kinetics and affinity of anti-CD228 antibodies to CD228.

FIGS. 6A-6C show results of binding assays of anti-CD228 antibodies to CD228 and certain other transferrin family members.

FIGS. 7A-7B and 8A-8C show results of binding assays of anti-CD228 antibodies to human, murine, and cynomolgus CD228.

FIG. 9 shows results of a cross-competition assay between various anti-CD228 antibodies.

FIG. 10 shows results of an antibody internalization assay using CD228+ tumor cell lines.

FIGS. 11A-11E show results of antigen recall assays in which fusion proteins co-stimulate innate and adaptive immune cytokines from PBMC in response to viral peptides.

FIGS. 12, 13, 14, and 15 show results of antigen recall assays in which fusion proteins co-stimulate T and NK cell responses in response to viral peptides.

FIGS. 16A-16E show results of antigen recall assays in which fusion proteins co-stimulate cytotoxic effector molecules and cytokines from PBMC in response to viral peptides.

FIGS. 17A-17B show the ability of representative fusion proteins to co-stimulate T cell activation in a CD228-target-dependent manner.

FIGS. 18A-18C show production of T cell cytokines in cocultures with an anti-CD3 scFv engineered CD228-expressing tumor cell line.

FIG. 19 shows CD8 T cell proliferation in cocultures with an anti-CD3 scFv engineered CD228-expressing tumor cell line, and FIG. 20 shows tumor cell killing in the coculture.

FIG. 21 shows pharmacokinetics of fusion proteins in cynomolgus monkeys.

FIGS. 22A-22D, 23A-23C, and 24A-24B show in vivo activity of fusion proteins in humanized xenograft models.

### V. DETAILED DESCRIPTION OF THE DISCLOSURE

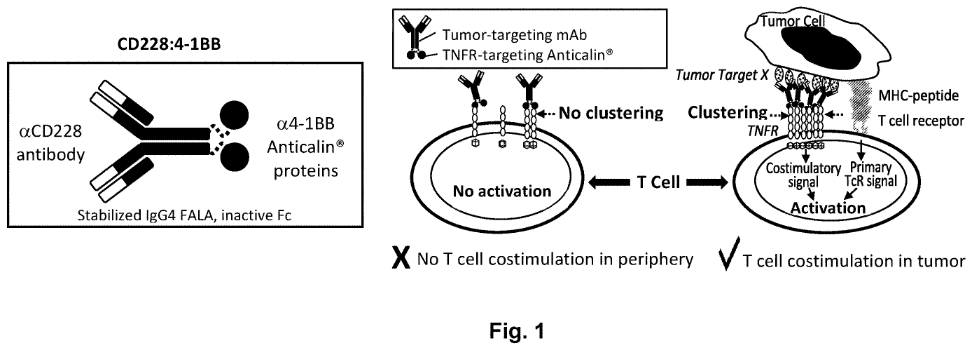

As is described herein, in one aspect, the present disclosure provides antibodies or antigen-binding fragments thereof that bind CD228. Such anti-CD228 antibodies or antigen-binding fragments thereof can be used by themselves as antibody therapeutics, conjugated to a therapeutic agent to generate an antibody-drug conjugate, included as part of a bispecific or multispecific antibody, or included as part of a fusion molecule. In certain embodiments, the antibody or antigen-binding fragment is incorporated into a fusion molecule that includes a binding domain that binds a different target than CD228. For example, as described in greater detail below, in some embodiments, the antibody or antigen-binding fragment is incorporated into a fusion molecule that can bind both CD228 and CD137. The use of the anti-CD228 antibodies or antigen-binding fragments as provided herein in fusion proteins, for example those that target both CD228 and CD137 can be utilized to promote CD137 clustering by bridging CD137-positive T cells with CD228-expressing tumor cells located in the tumor microenvironment, as shown in exemplary FIG. 1. The anti-CD228 antibodies and antigen-binding fragments provided herein can be used to generate fusion proteins directed to other targets to achieve similar effects. In other aspects, the present disclosure provides methods and useful applications of the anti-CD28 antibodies and antigen binding fragments thereof that are provided herein. The disclosure also provides methods of making CD228-binding antibodies or antigen-binding fragments thereof described herein as well as compositions comprising such proteins. CD228-binding antibodies or antigen-binding fragments thereof of the disclosure as well as compositions thereof may be used in methods of detecting CD228 in a sample, or in methods of binding of CD228 in a subject. No such antibodies or antigen-binding fragments thereof having these features attendant to the uses provided by present disclosure have been previously described.

### A. Exemplary Antibodies or Antigen-Binding Fragments Thereof Specific for CD228

In some embodiments, an antibody or an antigen-binding fragment thereof that binds CD228 provided herein comprises:
i) a heavy chain variable domain (VH) comprising (a) CDR-H1 comprising the amino acid sequence of SEQ ID NO: 110, (b) CDR-H2 comprising the amino acid sequence of SEQ ID NO: 111, and (c) CDR-H3 comprising the amino acid sequence of SEQ ID NO: 112, and a light chain variable domain (VL) comprising (d) CDR-L1 comprising the amino acid sequence of SEQ ID NO: 116, (e) CDR-L2 comprising the amino acid sequence of SEQ ID NO: 117, and (f) CDR-L3 comprising the amino acid sequence of SEQ ID NO: 118;
ii) a VH comprising (a) CDR-H1 comprising the amino acid sequence of SEQ ID NO: 113, (b) CDR-H2 comprising the amino acid sequence of SEQ ID NO: 114, and (c) CDR-H3 comprising the amino acid sequence of SEQ ID NO: 115, and a VL comprising (d) CDR-L1 comprising the amino acid sequence of SEQ ID NO: 119, (e) CDR-L2 comprising the amino acid sequence of SEQ ID NO: 120, and (f) CDR-L3 comprising the amino acid sequence of SEQ ID NO: 121;
iii) a VH comprising (a) CDR-H1 comprising the amino acid sequence of SEQ ID NO: 130, (b) CDR-H2 comprising the amino acid sequence of SEQ ID NO: 131, and (c) CDR-H3 comprising the amino acid sequence of SEQ ID NO: 132, and a VL comprising (d) CDR-L1 comprising the amino acid sequence of SEQ ID NO: 136, (e) CDR-L2 comprising the amino acid sequence of SEQ ID NO: 137, and (f) CDR-L3 comprising the amino acid sequence of SEQ ID NO: 138;
iv) a VH comprising (a) CDR-H1 comprising the amino acid sequence of SEQ ID NO: 133, (b) CDR-H2 comprising the amino acid sequence of SEQ ID NO: 134, and (c) CDR-H3 comprising the amino acid sequence of SEQ ID NO: 135, and a VL comprising (d) CDR-L1 comprising the amino acid sequence of SEQ ID NO: 139, (e) CDR-L2 comprising the amino acid sequence of SEQ ID NO: 140, and (f) CDR-L3 comprising the amino acid sequence of SEQ ID NO: 141;
v) a VH comprising (a) CDR-H1 comprising the amino acid sequence of SEQ ID NO: 150, (b) CDR-H2 comprising the amino acid sequence of SEQ ID NO: 151, and (c) CDR-H3 comprising the amino acid sequence of SEQ ID NO: 152, and a VL comprising (d) CDR-L1 comprising the amino acid sequence of SEQ ID NO: 156, (e) CDR-L2 comprising the amino acid sequence of SEQ ID NO: 157, and (f) CDR-L3 comprising the amino acid sequence of SEQ ID NO: 158;
vi) a VH comprising (a) CDR-H1 comprising the amino acid sequence of SEQ ID NO: 153, (b) CDR-H2 comprising the amino acid sequence of SEQ ID NO: 154, and (c) CDR-H3 comprising the amino acid sequence of SEQ ID NO: 155, and a VL comprising (d) CDR-L1 comprising the amino acid sequence of SEQ ID NO: 159, (e) CDR-L2 comprising the amino acid sequence of SEQ ID NO: 160, and (f) CDR-L3 comprising the amino acid sequence of SEQ ID NO: 161;
vii) a VH comprising (a) CDR-H1 comprising the amino acid sequence of SEQ ID NO: 170, (b) CDR-H2 comprising the amino acid sequence of SEQ ID NO: 171, and (c) CDR-H3 comprising the amino acid sequence of SEQ ID NO: 172, and a VL comprising (d) CDR-L1 comprising the amino acid sequence of SEQ ID NO: 176, (e) CDR-L2 comprising the amino acid sequence of SEQ ID NO: 177, and (f) CDR-L3 comprising the amino acid sequence of SEQ ID NO: 178;
viii) a VH comprising (a) CDR-H1 comprising the amino acid sequence of SEQ ID NO: 173, (b) CDR-H2 comprising the amino acid sequence of SEQ ID NO: 174, and (c) CDR-H3 comprising the amino acid sequence of SEQ ID NO: 175, and a VL comprising (d) CDR-L1 comprising the amino acid sequence of SEQ ID NO: 179, (e) CDR-L2 comprising the amino acid sequence of SEQ ID NO: 180, and (f) CDR-L3 comprising the amino acid sequence of SEQ ID NO: 181;
ix) a VH comprising (a) CDR-H1 comprising the amino acid sequence of SEQ ID NO: 190, (b) CDR-H2 comprising the amino acid sequence of SEQ ID NO: 191, and (c) CDR-H3 comprising the amino acid sequence of SEQ ID NO: 192, and a VL comprising (d) CDR-L1 comprising the amino acid sequence of SEQ ID NO: 196, (e) CDR-L2 comprising the amino acid sequence of SEQ ID NO: 197, and (f) CDR-L3 comprising the amino acid sequence of SEQ ID NO: 198;
x) a VH comprising (a) CDR-H1 comprising the amino acid sequence of SEQ ID NO: 193, (b) CDR-H2 comprising the amino acid sequence of SEQ ID NO: 194, and (c) CDR-H3 comprising the amino acid sequence of SEQ ID NO: 195, and a VL comprising (d) CDR-L1 comprising the amino acid sequence of SEQ ID NO: 199, (e) CDR-L2 comprising the amino acid sequence of SEQ ID NO: 200, and (f) CDR-L3 comprising the amino acid sequence of SEQ ID NO: 201;
xi) a VH comprising (a) CDR-H1 comprising the amino acid sequence of SEQ ID NO: 210, (b) CDR-H2 comprising the amino acid sequence of SEQ ID NO: 211, and (c) CDR-H3 comprising the amino acid sequence of SEQ ID NO: 212, and a VL comprising (d) CDR-L1 comprising the amino acid sequence of SEQ ID NO: 216, (e) CDR-L2 comprising the amino acid sequence of SEQ ID NO: 217, and (f) CDR-L3 comprising the amino acid sequence of SEQ ID NO: 218;
xii) a VH comprising (a) CDR-H1 comprising the amino acid sequence of SEQ ID NO: 213, (b) CDR-H2 comprising the amino acid sequence of SEQ ID NO: 214, and (c) CDR-H3 comprising the amino acid sequence of SEQ ID NO: 215, and a VL comprising (d) CDR-L1 comprising the amino acid sequence of SEQ ID NO: 219, (e) CDR-L2 comprising the amino acid sequence of SEQ ID NO: 220, and (f) CDR-L3 comprising the amino acid sequence of SEQ ID NO: 221;
xiii) a VH comprising (a) CDR-H1 comprising the amino acid sequence of SEQ ID NO: 230, (b) CDR-H2 comprising the amino acid sequence of SEQ ID NO: 231, and (c) CDR-H3 comprising the amino acid sequence of SEQ ID NO: 232, and a VL comprising (d) CDR-L1 comprising the amino acid sequence of SEQ ID NO: 236, (e) CDR-L2 comprising the amino acid sequence of SEQ ID NO: 237, and (f) CDR-L3 comprising the amino acid sequence of SEQ ID NO: 238;
xiv) a VH comprising (a) CDR-H1 comprising the amino acid sequence of SEQ ID NO: 233, (b) CDR-H2 comprising the amino acid sequence of SEQ ID NO: 234, and (c) CDR-H3 comprising the amino acid sequence of SEQ ID NO: 235, and a VL comprising (d) CDR-L1 comprising the amino acid sequence of SEQ ID NO: 239, (e) CDR-L2 comprising the amino acid sequence of SEQ ID NO: 240, and (f) CDR-L3 comprising the amino acid sequence of SEQ ID NO: 241;
xv) a VH comprising (a) CDR-H1 comprising the amino acid sequence of SEQ ID NO: 250, (b) CDR-H2 comprising the amino acid sequence of SEQ ID NO: 251, and (c) CDR-H3 comprising the amino acid sequence of SEQ ID NO: 252, and a VL comprising (d) CDR-L1 comprising the amino acid sequence of SEQ ID NO: 256, (e) CDR-L2 comprising the amino acid sequence of SEQ ID NO: 257, and (f) CDR-L3 comprising the amino acid sequence of SEQ ID NO: 258;
xvi) a VH comprising (a) CDR-H1 comprising the amino acid sequence of SEQ ID NO: 253, (b) CDR-H2 comprising the amino acid sequence of SEQ ID NO: 254, and (c) CDR-H3 comprising the amino acid sequence of SEQ ID NO: 255, and a VL comprising (d) CDR-L1 comprising the amino acid sequence of SEQ ID NO: 259, (e) CDR-L2 comprising the amino acid sequence of SEQ ID NO: 260, and (f) CDR-L3 comprising the amino acid sequence of SEQ ID NO: 261;
xvii) a VH comprising (a) CDR-H1 comprising the amino acid sequence of SEQ ID NO: 270, (b) CDR-H2 comprising the amino acid sequence of SEQ ID NO: 271, and (c) CDR-H3 comprising the amino acid sequence of SEQ ID NO: 272, and a VL comprising (d) CDR-L1 comprising the amino acid sequence of SEQ ID NO: 276, (e) CDR-L2 comprising the amino acid sequence of SEQ ID NO: 277, and (f) CDR-L3 comprising the amino acid sequence of SEQ ID NO: 278; or
xviii) a VH comprising (a) CDR-H1 comprising the amino acid sequence of SEQ ID NO: 273, (b) CDR-H2 comprising the amino acid sequence of SEQ ID NO: 274, and (c) CDR-H3 comprising the amino acid sequence of SEQ ID NO: 275, and a VL comprising (d) CDR-L1 comprising the amino acid sequence of SEQ ID NO: 279, (e) CDR-L2 comprising the amino acid sequence of SEQ ID NO: 280, and (f) CDR-L3 comprising the amino acid sequence of SEQ ID NO: 281.

In some embodiments, the antibody or the antigen-binding fragment thereof that binds CD228 provided herein comprises:
i) a VH comprising (a) CDR-H1 comprising the amino acid sequence of SEQ ID NO: 210, (b) CDR-H2 comprising the amino acid sequence of SEQ ID NO: 211, and (c) CDR-H3 comprising the amino acid sequence of SEQ ID NO: 212, and a VL comprising (d) CDR-L1 comprising the amino acid sequence of SEQ ID NO: 216, (e) CDR-L2 comprising the amino acid sequence of SEQ ID NO: 217, and (f) CDR-L3 comprising the amino acid sequence of SEQ ID NO: 218;
ii) a VH comprising (a) CDR-H1 comprising the amino acid sequence of SEQ ID NO: 213, (b) CDR-H2 comprising the amino acid sequence of SEQ ID NO: 214, and (c) CDR-H3 comprising the amino acid sequence of SEQ ID NO: 215, and a VL comprising (d) CDR-L1 comprising the amino acid sequence of SEQ ID NO: 219, (e) CDR-L2 comprising the amino acid sequence of SEQ ID NO: 220, and (f) CDR-L3 comprising the amino acid sequence of SEQ ID NO: 221;
iii) a VH comprising (a) CDR-H1 comprising the amino acid sequence of SEQ ID NO: 250, (b) CDR-H2 comprising the amino acid sequence of SEQ ID NO: 251, and (c) CDR-H3 comprising the amino acid sequence of SEQ ID NO: 252, and a VL comprising (d) CDR-L1 comprising the amino acid sequence of SEQ ID NO: 256, (e) CDR-L2 comprising the amino acid sequence of SEQ ID NO: 257, and (f) CDR-L3 comprising the amino acid sequence of SEQ ID NO: 258; or
iv) a VH comprising (a) CDR-H1 comprising the amino acid sequence of SEQ ID NO: 253, (b) CDR-H2 comprising the amino acid sequence of SEQ ID NO: 254, and (c) CDR-H3 comprising the amino acid sequence of SEQ ID NO: 255, and a VL comprising (d) CDR-L1 comprising the amino acid sequence of SEQ ID NO: 259, (e) CDR-L2 comprising the amino acid sequence of SEQ ID NO: 260, and (f) CDR-L3 comprising the amino acid sequence of SEQ ID NO: 261.

In some embodiments, the antibody or the antigen-binding fragment thereof that binds CD228 provided herein comprises: a VH comprising (a) CDR-H1 comprising the amino acid sequence of SEQ ID NO: 210, (b) CDR-H2 comprising the amino acid sequence of SEQ ID NO: 211, and (c) CDR-H3 comprising the amino acid sequence of SEQ ID NO: 212, and a VL comprising (d) CDR-L1 comprising the amino acid sequence of SEQ ID NO: 216, (e) CDR-L2 comprising the amino acid sequence of SEQ ID NO: 217, and (f) CDR-L3 comprising the amino acid sequence of SEQ ID NO: 218; or a VH comprising (a) CDR-H1 comprising the amino acid sequence of SEQ ID NO: 213, (b) CDR-H2 comprising the amino acid sequence of SEQ ID NO: 214, and (c) CDR-H3 comprising the amino acid sequence of SEQ ID NO: 215, and a VL comprising (d) CDR-L1 comprising the amino acid sequence of SEQ ID NO: 219, (e) CDR-L2 comprising the amino acid sequence of SEQ ID NO: 220, and (f) CDR-L3 comprising the amino acid sequence of SEQ ID NO: 221.

In some embodiments, the antibody or the antigen-binding fragment thereof that binds CD228 provided herein comprises:
i) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 122, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 124;
ii) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 142, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 144;
iii) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 162, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 164;
iv) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 182, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 184;
v) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 202, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 204;
vi) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 222, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 224;
vii) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 242, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 244;
viii) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 262, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 264; or
ix) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 282, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 284.

In some embodiments, the antibody or the antigen-binding fragment thereof that binds CD228 provided herein comprises: a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 222, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 224; or a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 262, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 264.

In some embodiments, the antibody or the antigen-binding fragment thereof that binds CD228 provided herein comprises: a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 222, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 224.

In some embodiments, the antibody or the antigen-binding fragment thereof that binds CD228 provided herein comprises:
i) a VH comprising the amino acid sequence of SEQ ID NO: 122, and a VL comprising the amino acid sequence of SEQ ID NO: 124;
ii) a VH comprising the amino acid sequence of SEQ ID NO: 142, and a VL comprising the amino acid sequence of SEQ ID NO: 144;
iii) a VH comprising the amino acid sequence of SEQ ID NO: 162, and a VL comprising the amino acid sequence of SEQ ID NO: 164;
iv) a VH comprising the amino acid sequence of SEQ ID NO: 182, and a VL comprising the amino acid sequence of SEQ ID NO: 184;
v) a VH comprising the amino acid sequence of SEQ ID NO: 202, and a VL comprising the amino acid sequence of SEQ ID NO: 204;
vi) a VH comprising the amino acid sequence of SEQ ID NO: 222, and a VL comprising the amino acid sequence of SEQ ID NO: 224;
vii) a VH comprising the amino acid sequence of SEQ ID NO: 242, and a VL comprising the amino acid sequence of SEQ ID NO: 244;
viii) a VH comprising the amino acid sequence of SEQ ID NO: 262, and a VL comprising the amino acid sequence of SEQ ID NO: 264; or
ix) a VH comprising the amino acid sequence of SEQ ID NO: 282, and a VL comprising the amino acid sequence of SEQ ID NO: 284.

In some embodiments, the antibody or the antigen-binding fragment thereof that binds CD228 provided herein comprises: a VH comprising the amino acid sequence of SEQ ID NO: 222, and a VL comprising the amino acid sequence of SEQ ID NO: 224; or a VH comprising the amino acid sequence of SEQ ID NO: 262, and a VL comprising the amino acid sequence of SEQ ID NO: 264.

In some embodiments, the antibody or the antigen-binding fragment thereof that binds CD228 provided herein comprises: a VH comprising the amino acid sequence of SEQ ID NO: 222, and a VL comprising the amino acid sequence of SEQ ID NO: 224.

In some embodiments, the antibody or the antigen-binding fragment thereof that binds CD228 provided herein comprises:
i) a heavy chain (HC) comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 126, and a light chain (LC) comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 128;
ii) a HC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 146, and a LC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 148;
iii) a HC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 166, and a LC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 168;
iv) a HC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 186, and a LC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 188;
v) a HC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 206, and a LC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 208;
vi) a HC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 226, and a LC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 228;
vii) a HC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 246, and a LC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 248;
viii) a HC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 266, and a LC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 268; or
ix) a HC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 286, and a LC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 288.

In some embodiments, the antibody or the antigen-binding fragment thereof that binds CD228 provided herein comprises: a HC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 226, and a LC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 228; or a HC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 266, and a LC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 268.

In some embodiments, the antibody or the antigen-binding fragment thereof that binds CD228 provided herein comprises: a HC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 226, and a LC comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 228.

In some embodiments, the antibody or the antigen-binding fragment thereof that binds CD228 provided herein comprises:
i) a HC comprising the amino acid sequence of SEQ ID NO: 126, and a LC comprising the amino acid sequence of SEQ ID NO: 128;
ii) a HC comprising the amino acid sequence of SEQ ID NO: 146, and a LC comprising the amino acid sequence of SEQ ID NO: 148;
iii) a HC comprising the amino acid sequence of SEQ ID NO: 166, and a LC comprising the amino acid sequence of SEQ ID NO: 168;
iv) a HC comprising the amino acid sequence of SEQ ID NO: 186, and a LC comprising the amino acid sequence of SEQ ID NO: 188;
v) a HC comprising the amino acid sequence of SEQ ID NO: 206, and a LC comprising the amino acid sequence of SEQ ID NO: 208;
vi) a HC comprising the amino acid sequence of SEQ ID NO: 226, and a LC comprising the amino acid sequence of SEQ ID NO: 228;
vii) a HC comprising the amino acid sequence of SEQ ID NO: 246, and a LC comprising the amino acid sequence of SEQ ID NO: 248;
viii) a HC comprising the amino acid sequence of SEQ ID NO: 266, and a LC comprising the amino acid sequence of SEQ ID NO: 268; or
ix) a HC comprising the amino acid sequence of SEQ ID NO: 286, and a LC comprising the amino acid sequence of SEQ ID NO: 288.

In some embodiments, the antibody or the antigen-binding fragment thereof that binds CD228 provided herein comprises: a HC comprising the amino acid sequence of SEQ ID NO: 226, and a LC comprising the amino acid sequence of SEQ ID NO: 228; or a HC comprising the amino acid sequence of SEQ ID NO: 266, and a LC comprising the amino acid sequence of SEQ ID NO: 268.

In some embodiments, the antibody or the antigen-binding fragment thereof that binds CD228 provided herein comprises: a HC comprising the amino acid sequence of SEQ ID NO: 226, and a LC comprising the amino acid sequence of SEQ ID NO: 228.

In some embodiments, the antibody that binds CD228 provided herein is a monoclonal antibody. In some embodiments, the antibody that binds CD228 provided herein is a humanized or chimeric antibody. In some embodiments, the antibody that binds CD228 provided herein is an IgG1, IgG2, IgG3, or IgG4 antibody.

In some embodiments, the antibody or the antigen-binding fragment thereof that binds CD228 provided herein binds CD228 with a K_{D} value of 175 nM or less. In some embodiments, the antibody or the antigen-binding fragment thereof that binds CD228 provided herein binds CD228 with a K_{D} value of 150 nM or less. In some embodiments, the antibody or the antigen-binding fragment thereof that binds CD228 provided herein binds CD228 with a K_{D} value of 100 nM or less. In some embodiments, the antibody or the antigen-binding fragment thereof that binds CD228 provided herein binds CD228 with a K_{D} value of 50 nM or less. In some embodiments, the antibody or the antigen-binding fragment thereof that binds CD228 provided herein binds CD228 with a K_{D} value of 75 nM or less. In some embodiments, the antibody or the antigen-binding fragment thereof that binds CD228 provided herein binds CD228 with a K_{D} value of 25 nM or less. In some embodiments, the antibody or the antigen-binding fragment thereof that binds CD228 provided herein binds CD228 with a K_{D} value of 20 nM or less. In some embodiments, the antibody or the antigen-binding fragment thereof that binds CD228 provided herein binds CD228 with a K_{D} value of 15 nM or less. In some embodiments, the antibody or the antigen-binding fragment thereof that binds CD228 provided herein binds CD228 with a K_{D} value of 10 nM or less. In some embodiments, the antibody or the antigen-binding fragment thereof that binds CD228 provided herein binds CD228 with a K_{D} value of 5 nM or less. In some embodiments, the antibody or the antigen-binding fragment thereof that binds CD228 provided herein binds CD228 with a K_{D} value of 4 nM or less. In some embodiments, the antibody or the antigen-binding fragment thereof that binds CD228 provided herein binds CD228 with a K_{D} value of 3 nM or less. In some embodiments, the antibody or the antigen-binding fragment thereof that binds CD228 provided herein binds CD228 with a K_{D} value of 2.5 nM or less. In some embodiments, the antibody or the antigen-binding fragment thereof that binds CD228 provided herein binds CD228 with a K_{D} value of 2 nM or less. In some embodiments, the antibody or the antigen-binding fragment thereof that binds CD228 provided herein binds CD228 with a K_{D} value of 1.5 nM or less. In some embodiments, the antibody or the antigen-binding fragment thereof that binds CD228 provided herein binds CD228 with a K_{D} value of 1 nM or less.

In some embodiments, the antibody or the antigen-binding fragment thereof that binds CD228 provided herein binds cynomolgus monkey CD228. In some such embodiments, the antibody or the antigen-binding fragment thereof that binds CD228 provided herein comprises:
i) a VH comprising (a) CDR-H1 comprising the amino acid sequence of SEQ ID NO: 170, (b) CDR-H2 comprising the amino acid sequence of SEQ ID NO: 171, and (c) CDR-H3 comprising the amino acid sequence of SEQ ID NO: 172, and a VL comprising (d) CDR-L1 comprising the amino acid sequence of SEQ ID NO: 176, (e) CDR-L2 comprising the amino acid sequence of SEQ ID NO: 177, and (f) CDR-L3 comprising the amino acid sequence of SEQ ID NO: 178;
ii) a VH comprising (a) CDR-H1 comprising the amino acid sequence of SEQ ID NO: 173, (b) CDR-H2 comprising the amino acid sequence of SEQ ID NO: 174, and (c) CDR-H3 comprising the amino acid sequence of SEQ ID NO: 175, and a VL comprising (d) CDR-L1 comprising the amino acid sequence of SEQ ID NO: 179, (e) CDR-L2 comprising the amino acid sequence of SEQ ID NO: 180, and (f) CDR-L3 comprising the amino acid sequence of SEQ ID NO: 181;
iii) a VH comprising (a) CDR-H1 comprising the amino acid sequence of SEQ ID NO: 210, (b) CDR-H2 comprising the amino acid sequence of SEQ ID NO: 211, and (c) CDR-H3 comprising the amino acid sequence of SEQ ID NO: 212, and a VL comprising (d) CDR-L1 comprising the amino acid sequence of SEQ ID NO: 216, (e) CDR-L2 comprising the amino acid sequence of SEQ ID NO: 217, and (f) CDR-L3 comprising the amino acid sequence of SEQ ID NO: 218;
iv) a VH comprising (a) CDR-H1 comprising the amino acid sequence of SEQ ID NO: 213, (b) CDR-H2 comprising the amino acid sequence of SEQ ID NO: 214, and (c) CDR-H3 comprising the amino acid sequence of SEQ ID NO: 215, and a VL comprising (d) CDR-L1 comprising the amino acid sequence of SEQ ID NO: 219, (e) CDR-L2 comprising the amino acid sequence of SEQ ID NO: 220, and (f) CDR-L3 comprising the amino acid sequence of SEQ ID NO: 221;
v) a VH comprising (a) CDR-H1 comprising the amino acid sequence of SEQ ID NO: 250, (b) CDR-H2 comprising the amino acid sequence of SEQ ID NO: 251, and (c) CDR-H3 comprising the amino acid sequence of SEQ ID NO: 252, and a VL comprising (d) CDR-L1 comprising the amino acid sequence of SEQ ID NO: 256, (e) CDR-L2 comprising the amino acid sequence of SEQ ID NO: 257, and (f) CDR-L3 comprising the amino acid sequence of SEQ ID NO: 258; or
vi) a VH comprising (a) CDR-H1 comprising the amino acid sequence of SEQ ID NO: 253, (b) CDR-H2 comprising the amino acid sequence of SEQ ID NO: 254, and (c) CDR-H3 comprising the amino acid sequence of SEQ ID NO: 255, and a VL comprising (d) CDR-L1 comprising the amino acid sequence of SEQ ID NO: 259, (e) CDR-L2 comprising the amino acid sequence of SEQ ID NO: 260, and (f) CDR-L3 comprising the amino acid sequence of SEQ ID NO: 261.

In some such embodiments, the antibody or the antigen-binding fragment thereof that binds CD228 provided herein comprises a set of CDRs as set forth above and:
i) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 182, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 184;
ii) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 222, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 224; or
iii) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 262, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 264.

In some such embodiments, the antibody or the antigen-binding fragment thereof that binds CD228 provided herein comprises: a VH comprising the amino acid sequence of SEQ ID NO: 182, and a VL comprising the amino acid sequence of SEQ ID NO: 184; a VH comprising the amino acid sequence of SEQ ID NO: 222, and a VL comprising the amino acid sequence of SEQ ID NO: 224; or a VH comprising the amino acid sequence of SEQ ID NO: 262, and a VL comprising the amino acid sequence of SEQ ID NO: 264.

In some embodiments, the antibody or the antigen-binding fragment thereof that binds CD228 provided herein does not bind murine CD228, or binds murine CD228 with at least 100-fold reduced affinity compared to human CD228. In some embodiments, the antibody or the antigen-binding fragment thereof that binds CD228 provided herein does not bind transferrin or lactotransferrin. In some such embodiments, the antibody or the antigen-binding fragment thereof that binds CD228 provided herein comprises:
i) a VH comprising (a) CDR-H1 comprising the amino acid sequence of SEQ ID NO: 170, (b) CDR-H2 comprising the amino acid sequence of SEQ ID NO: 171, and (c) CDR-H3 comprising the amino acid sequence of SEQ ID NO: 172, and a VL comprising (d) CDR-L1 comprising the amino acid sequence of SEQ ID NO: 176, (e) CDR-L2 comprising the amino acid sequence of SEQ ID NO: 177, and (f) CDR-L3 comprising the amino acid sequence of SEQ ID NO: 178;
ii) a VH comprising (a) CDR-H1 comprising the amino acid sequence of SEQ ID NO: 173, (b) CDR-H2 comprising the amino acid sequence of SEQ ID NO: 174, and (c) CDR-H3 comprising the amino acid sequence of SEQ ID NO: 175, and a VL comprising (d) CDR-L1 comprising the amino acid sequence of SEQ ID NO: 179, (e) CDR-L2 comprising the amino acid sequence of SEQ ID NO: 180, and (f) CDR-L3 comprising the amino acid sequence of SEQ ID NO: 181;
iii) a VH comprising (a) CDR-H1 comprising the amino acid sequence of SEQ ID NO: 190, (b) CDR-H2 comprising the amino acid sequence of SEQ ID NO: 191, and (c) CDR-H3 comprising the amino acid sequence of SEQ ID NO: 192, and a VL comprising (d) CDR-L1 comprising the amino acid sequence of SEQ ID NO: 196, (e) CDR-L2 comprising the amino acid sequence of SEQ ID NO: 197, and (f) CDR-L3 comprising the amino acid sequence of SEQ ID NO: 198;
iv) a VH comprising (a) CDR-H1 comprising the amino acid sequence of SEQ ID NO: 193, (b) CDR-H2 comprising the amino acid sequence of SEQ ID NO: 194, and (c) CDR-H3 comprising the amino acid sequence of SEQ ID NO: 195, and a VL comprising (d) CDR-L1 comprising the amino acid sequence of SEQ ID NO: 199, (e) CDR-L2 comprising the amino acid sequence of SEQ ID NO: 200, and (f) CDR-L3 comprising the amino acid sequence of SEQ ID NO: 201;
v) a VH comprising (a) CDR-H1 comprising the amino acid sequence of SEQ ID NO: 210, (b) CDR-H2 comprising the amino acid sequence of SEQ ID NO: 211, and (c) CDR-H3 comprising the amino acid sequence of SEQ ID NO: 212, and a VL comprising (d) CDR-L1 comprising the amino acid sequence of SEQ ID NO: 216, (e) CDR-L2 comprising the amino acid sequence of SEQ ID NO: 217, and (f) CDR-L3 comprising the amino acid sequence of SEQ ID NO: 218;
vi) a VH comprising (a) CDR-H1 comprising the amino acid sequence of SEQ ID NO: 213, (b) CDR-H2 comprising the amino acid sequence of SEQ ID NO: 214, and (c) CDR-H3 comprising the amino acid sequence of SEQ ID NO: 215, and a VL comprising (d) CDR-L1 comprising the amino acid sequence of SEQ ID NO: 219, (e) CDR-L2 comprising the amino acid sequence of SEQ ID NO: 220, and (f) CDR-L3 comprising the amino acid sequence of SEQ ID NO: 221;
vii) a VH comprising (a) CDR-H1 comprising the amino acid sequence of SEQ ID NO: 230, (b) CDR-H2 comprising the amino acid sequence of SEQ ID NO: 231, and (c) CDR-H3 comprising the amino acid sequence of SEQ ID NO: 232, and a VL comprising (d) CDR-L1 comprising the amino acid sequence of SEQ ID NO: 236, (e) CDR-L2 comprising the amino acid sequence of SEQ ID NO: 237, and (f) CDR-L3 comprising the amino acid sequence of SEQ ID NO: 238;
viii) a VH comprising (a) CDR-H1 comprising the amino acid sequence of SEQ ID NO: 233, (b) CDR-H2 comprising the amino acid sequence of SEQ ID NO: 234, and (c) CDR-H3 comprising the amino acid sequence of SEQ ID NO: 235, and a VL comprising (d) CDR-L1 comprising the amino acid sequence of SEQ ID NO: 239, (e) CDR-L2 comprising the amino acid sequence of SEQ ID NO: 240, and (f) CDR-L3 comprising the amino acid sequence of SEQ ID NO: 241;
ix) a VH comprising (a) CDR-H1 comprising the amino acid sequence of SEQ ID NO: 250, (b) CDR-H2 comprising the amino acid sequence of SEQ ID NO: 251, and (c) CDR-H3 comprising the amino acid sequence of SEQ ID NO: 252, and a VL comprising (d) CDR-L1 comprising the amino acid sequence of SEQ ID NO: 256, (e) CDR-L2 comprising the amino acid sequence of SEQ ID NO: 257, and (f) CDR-L3 comprising the amino acid sequence of SEQ ID NO: 258;
x) a VH comprising (a) CDR-H1 comprising the amino acid sequence of SEQ ID NO: 253, (b) CDR-H2 comprising the amino acid sequence of SEQ ID NO: 254, and (c) CDR-H3 comprising the amino acid sequence of SEQ ID NO: 255, and a VL comprising (d) CDR-L1 comprising the amino acid sequence of SEQ ID NO: 259, (e) CDR-L2 comprising the amino acid sequence of SEQ ID NO: 260, and (f) CDR-L3 comprising the amino acid sequence of SEQ ID NO: 261;
xi) a VH comprising (a) CDR-H1 comprising the amino acid sequence of SEQ ID NO: 270, (b) CDR-H2 comprising the amino acid sequence of SEQ ID NO: 271, and (c) CDR-H3 comprising the amino acid sequence of SEQ ID NO: 272, and a VL comprising (d) CDR-L1 comprising the amino acid sequence of SEQ ID NO: 276, (e) CDR-L2 comprising the amino acid sequence of SEQ ID NO: 277, and (f) CDR-L3 comprising the amino acid sequence of SEQ ID NO: 278; or
xii) a VH comprising (a) CDR-H1 comprising the amino acid sequence of SEQ ID NO: 273, (b) CDR-H2 comprising the amino acid sequence of SEQ ID NO: 274, and (c) CDR-H3 comprising the amino acid sequence of SEQ ID NO: 275, and a VL comprising (d) CDR-L1 comprising the amino acid sequence of SEQ ID NO: 279, (e) CDR-L2 comprising the amino acid sequence of SEQ ID NO: 280, and (f) CDR-L3 comprising the amino acid sequence of SEQ ID NO: 281.

In some such embodiments, the antibody or the antigen-binding fragment thereof that binds CD228 provided herein comprises a set of CDRs as set forth above and:
i) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 182, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 184;
ii) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 202, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 204;
iii) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 222, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 224;
iv) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 242, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 244; or
v) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 262, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 264; or
vi) a VH comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 282, and a VL comprising an amino acid sequence having at least 80%, at least 85%, at least 90%, at least 91%, at least 92%, at least 93%, at least 94%, at least 95%, at least 96%, at least 97%, at least 98%, or at least 99% sequence identity to SEQ ID NO: 284.

In some such embodiments, the antibody or the antigen-binding fragment thereof that binds CD228 provided herein comprises: a VH comprising the amino acid sequence of SEQ ID NO: 182, and a VL comprising the amino acid sequence of SEQ ID NO: 184; a VH comprising the amino acid sequence of SEQ ID NO: 202, and a VL comprising the amino acid sequence of SEQ ID NO: 204; a VH comprising the amino acid sequence of SEQ ID NO: 222, and a VL comprising the amino acid sequence of SEQ ID NO: 224; a VH comprising the amino acid sequence of SEQ ID NO: 242, and a VL comprising the amino acid sequence of SEQ ID NO: 244; a VH comprising the amino acid sequence of SEQ ID NO: 262, and a VL comprising the amino acid sequence of SEQ ID NO: 264; or a VH comprising the amino acid sequence of SEQ ID NO: 282, and a VL comprising the amino acid sequence of SEQ ID NO: 284.

In some embodiments, an antibody or an antigen-binding fragment thereof that binds CD228 is provided, wherein the antibody or the antigen-binding fragment thereof competes for binding with CD228 with any one of the antibodies or antigen-binding fragments thereof disclosed herein. In some such embodiments, the antibody is a monoclonal antibody. In some such embodiments, the antibody is a humanized or chimeric antibody. In some such embodiments, the antibody is an IgG1, IgG2, IgG3, or IgG4 antibody. In some such embodiments, the antibody or the antigen-binding fragment thereof binds CD228 with a K_{D} value of 175 nM or less. In some such embodiments, the antibody or the antigen-binding fragment thereof binds cynomolgus monkey CD228. In some such embodiments, the antibody or the antigen-binding fragment thereof does not bind murine CD228, or binds murine CD228 with at least 100-fold reduced affinity compared to human CD228. In some such embodiments, the antibody or the antigen-binding fragment thereof does not bind transferrin or lactotransferrin. In some such embodiments, the antibody or antigen-binding fragment thereof competes for binding with CD228 with an antibody or antigen-binding fragment thereof comprising: a VH comprising the amino acid sequence of SEQ ID NO: 122, and a VL comprising the amino acid sequence of SEQ ID NO: 124; a VH comprising the amino acid sequence of SEQ ID NO: 142, and a VL comprising the amino acid sequence of SEQ ID NO: 144; a VH comprising the amino acid sequence of SEQ ID NO: 162, and a VL comprising the amino acid sequence of SEQ ID NO: 164; a VH comprising the amino acid sequence of SEQ ID NO: 182, and a VL comprising the amino acid sequence of SEQ ID NO: 184; a VH comprising the amino acid sequence of SEQ ID NO: 202, and a VL comprising the amino acid sequence of SEQ ID NO: 204; a VH comprising the amino acid sequence of SEQ ID NO: 222, and a VL comprising the amino acid sequence of SEQ ID NO: 224; a VH comprising the amino acid sequence of SEQ ID NO: 242, and a VL comprising the amino acid sequence of SEQ ID NO: 244; a VH comprising the amino acid sequence of SEQ ID NO: 262, and a VL comprising the amino acid sequence of SEQ ID NO: 264; or a VH comprising the amino acid sequence of SEQ ID NO: 282, and a VL comprising the amino acid sequence of SEQ ID NO: 284. In some such embodiments, the antibody or antigen-binding fragment thereof competes for binding with CD228 with an antibody or antigen-binding fragment thereof comprising: a VH comprising the amino acid sequence of SEQ ID NO: 202, and a VL comprising the amino acid sequence of SEQ ID NO: 204; a VH comprising the amino acid sequence of SEQ ID NO: 242, and a VL comprising the amino acid sequence of SEQ ID NO: 244; or a VH comprising the amino acid sequence of SEQ ID NO: 262, and a VL comprising the amino acid sequence of SEQ ID NO: 264. In some such embodiments, the antibody or antigen-binding fragment thereof competes for binding with CD228 with an antibody or antigen-binding fragment thereof comprising: a VH comprising the amino acid sequence of SEQ ID NO: 202, and a VL comprising the amino acid sequence of SEQ ID NO: 204; or a VH comprising the amino acid sequence of SEQ ID NO: 242, and a VL comprising the amino acid sequence of SEQ ID NO: 244. In some such embodiments, competition for binding with CD228 is measured by flow cytometry using at least one labeled antibody or antigen-binding fragment thereof.

In some embodiments, CDR sequences disclosed herein are defined according to the Kabat numbering scheme as described in Kabat et al. (1991), “Sequences of Proteins of Immunological Interest,” 5th Ed. Public Health Service, National Institutes of Health, Bethesda, MD. In some embodiments, CDR sequences disclosed herein are defined according to the IMGT method as described in Lefranc, M.-P., The Immunologist, 7, 132-136 (1999).

Antibodies specifically binding to CD228 as included in fusion proteins of the disclosure may comprise an Fc part which allows for extending the in vivo half-life of the bispecific binding molecule of the disclosure. In some embodiments, such Fc part is preferably from human origin, more preferably a human Fc part of an IgG1 or IgG4 antibody, even more preferably an engineered human Fc part of an IgG1 or IgG4 with activating or silencing effector functions. In some embodiments, silencing effector functions may be preferred over activating effector functions. In some embodiments, such an Fc part is an engineered to silence effector functions with mutation(s) at positions 234 and/or 235, numbering according to EU index of Kabat (Johnson and Wu, Nucleic Acids Res, 2000). In some embodiments, mutations in positions F234 and L235 of a provided anti-CD228 antibody may be introduced to silence effector functions. In other embodiments, mutations in positions D265 and P329 of a provided anti-CD228 antibody may be introduced to silence effector function. Numbering for both sets of these potential mutations is according to the EU index of Kabat (Shields et al., J Biol Chem, 2001). In some embodiments, the provided CD228 antibody has an engineered IgG4 backbone with the mutations S228P, F234A and L235A.

Various techniques for the production of antibodies and antigen-binding fragments thereof are well known in the art and described, e.g., in Altshuler et al. (2010). Thus, for example, polyclonal antibodies can be obtained from the blood of an animal following immunization with an antigen in mixture with additives and adjuvants and monoclonal antibodies can be produced by any technique which provides antibodies produced by continuous cell line cultures. Examples of such techniques are described, e.g., Harlow and Lane (1999), (1988), and include the hybridoma technique originally described by Kohler and Milstein, 1975, the trioma technique, the human B cell hybridoma technique (see e.g., Li et al., Proc Natl Acad Sci USA, 2006; Kozbor and Roder, Immunol Today, 1983) and the EBV-hybridoma technique to produce human monoclonal antibodies (Cole et al., Cancer Res, 1984). Furthermore, recombinant antibodies may be obtained from monoclonal antibodies or can be prepared de novo using various display methods such as phage, ribosomal, mRNA, or cell display. In some embodiments, a suitable system for the expression of the recombinant (humanized) antibodies or fragments thereof may be selected from, for example, bacteria, yeast, insects, mammalian cell lines or transgenic animals or plants (see, e.g., U.S. Pat. No. 6,080,560; Holliger and Hudson, Nat Biotechnol, 2005). Further, techniques described for the production of single chain antibodies (see, inter alia, U.S. Pat. No. 4,946,778) can be adapted to produce single chain antibodies specific for the target of this invention. Surface plasmon resonance as employed in the BIAcore system can be used to increase the efficiency of phage antibodies.

### B. Immunoconjugates of Antibodies or Antigen-Binding Fragments Thereof

The antibodies or antigen-binding fragments thereof provided herein can be conjugated to cytotoxic or cytostatic moieties (including pharmaceutically compatible salts thereof) to form immunoconjugates, such as an antibody drug conjugate (ADC). Particularly suitable moieties for conjugation to the antibodies or antigen-binding fragments thereof are cytotoxic agents (e.g., chemotherapeutic agents), prodrug converting enzymes, radioactive isotopes or compounds, or toxins (these moieties being collectively referred to as a therapeutic agent). For example, an antibody or antigen-binding fragment thereof can be conjugated to a cytotoxic agent such as a chemotherapeutic agent, or a toxin (e.g., a cytostatic or cytocidal agent such as, for example, abrin, ricin A, pseudomonas exotoxin, or diphtheria toxin). Examples of useful classes of cytotoxic agents include, for example, DNA minor groove binders, DNA alkylating agents, and tubulin inhibitors. Exemplary cytotoxic agents include, for example, auristatins, camptothecins, calicheamicins, duocarmycins, etoposides, maytansinoids (e.g., DM1, DM2, DM3, DM4), taxanes, benzodiazepines (e.g., pyrrolo[1,4]benzodiazepines, indolinobenzodiazepines, and oxazolidinobenzodiazepines) and vinca alkaloids.

In one embodiment, an antibody or antigen-binding fragment thereof is conjugated to a pro-drug converting enzyme. The pro-drug converting enzyme can be recombinantly fused to the antibody or antigen-binding fragment thereof or chemically conjugated thereto using known methods. Exemplary pro-drug converting enzymes are carboxypeptidase G2, β-glucuronidase, penicillin-V-amidase, penicillin-G-amidase, β-lactamase, β-glucosidase, nitroreductase and carboxypeptidase A.

Techniques for conjugating therapeutic agents to antibodies or antigen-binding fragments thereof are well-known. (See, e.g., Alley et al., Current Opinion in Chemical Biology 2010 14:1-9; Senter, Cancer J., 2008, 14(3): 154-169.) The therapeutic agent can be conjugated in a manner that reduces its activity unless it is cleaved off the antibody or antigen-binding fragment thereof (e.g., by hydrolysis, by proteolytic degradation, or by a cleaving agent). In some aspects, the therapeutic agent is attached to the antibody or antigen-binding fragment thereof with a cleavable linker that is sensitive to cleavage in the intracellular environment of the CD228-expressing cancer cell but is not substantially sensitive to the extracellular environment, such that the immunoconjugate is cleaved from the antibody or antigen-binding fragment thereof when it is internalized by the CD228-expressing cancer cell (e.g., in the endosomal or, for example by virtue of pH sensitivity or protease sensitivity, in the lysosomal environment or in the caveolear environment). In some aspects, the therapeutic agent can also be attached to the antibody or antigen-binding fragment thereof with a non-cleavable linker.

Typically, the immunoconjugate comprises a linker region between the therapeutic agent and the antibody or antigen-binding fragment thereof. The linker generally is cleavable under intracellular conditions, such that cleavage of the linker releases the therapeutic agent from the antibody or antigen-binding fragment thereof in the intracellular environment (e.g., within a lysosome or endosome or caveolea). The linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in CD228-expressing cells. For example, a peptidyl linker that is cleavable by the thiol-dependent protease cathepsin-B, which is highly expressed in cancerous tissue, can be used (e.g., a linker comprising a Phe-Leu or a Val-Cit peptide).

The cleavable linker can be pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, Pharm. Therapeutics 83:67-123, 1999; Neville et al., Biol. Chem. 264:14653-14661, 1989.) Such linkers are relatively stable under neutral pH conditions, such as those in the blood, but are unstable at below pH 5.5 or 5.0, the approximate pH of the lysosome.

Other linkers are cleavable under reducing conditions (e.g., a disulfide linker). Disulfide linkers include those that can be formed using SATA (N-succinimidyl-S-acetylthioacetate), SPDP (N-succinimidyl-3-(2-pyridyldithio) propionate), SPDB (N-succinimidyl-3-(2-pyridyldithio) butyrate) and SMPT (N-succinimidyl-oxycarbonyl-alpha-methyl-alpha-(2-pyridyl-dithio) toluene), SPDB and SMPT. (See, e.g., Thorpe et al., Cancer Res. 47:5924-5931, 1987; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

The linker can also be a malonate linker (Johnson et al., Anticancer Res. 15:1387-93, 1995), a maleimidobenzoyl linker (Lau et al., Bioorg-Med-Chem. 3:1299-1304, 1995), or a 3′-N-amide analog (Lau et al., Bioorg-Med-Chem. 3:1305-12, 1995).

In other embodiments, the linker is a non-cleavable linker, such as an maleimido-alkylene- or maleimide-aryl linker that is directly attached to the therapeutic agent and released by proteolytic degradation of the antibody or antigen-binding fragment thereof.

Typically, the linker is not substantially sensitive to the extracellular environment, meaning that no more than about 20%, typically no more than about 15%, more typically no more than about 10%, and even more typically no more than about 5%, no more than about 3%, or no more than about 1% of the linkers in a sample of the immunoconjugate is cleaved when the immunoconjugate is present in an extracellular environment (e.g., in plasma). Whether a linker is not substantially sensitive to the extracellular environment can be determined, for example, by incubating independently with plasma both (a) the immunoconjugate (the “immunoconjugate sample”) and (b) an equal molar amount of unconjugated antibody or antigen-binding fragment thereof or therapeutic agent (the “control sample”) for a predetermined time period (e.g., 2, 4, 8, 16, or 24 hours) and then comparing the amount of unconjugated antibody or antigen-binding fragment thereof or therapeutic agent present in the immunoconjugate sample with that present in control sample, as measured, for example, by high performance liquid chromatography.

The linker can also promote cellular internalization. The linker can promote cellular internalization when conjugated to the therapeutic agent (i.e., in the milieu of the linker-therapeutic agent moiety of the immunoconjugate or immunoconjugate derivate as described herein). Alternatively, the linker can promote cellular internalization when conjugated to both the therapeutic agent and the antibody or antigen-binding fragment thereof (i.e., in the milieu of the immunoconjugate as described herein).

Exemplary immunoconjugates include auristatin-based conjugates, meaning that the drug component is an auristatin drug. Auristatins bind tubulin, have been shown to interfere with microtubule dynamics and nuclear and cellular division, and have anticancer activity. Typically, the auristatin based antibody-drug conjugate comprises a linker between the auristatin drug and the antibody or antigen-binding fragment thereof. The linker can be, for example, a cleavable linker (e.g., a peptidyl linker) or a non-cleavable linker (e.g., linker released by degradation of the antibody). The auristatin can be auristatin E or a derivative thereof. The auristatin can be, for example, an ester formed between auristatin E and a keto acid. For example, auristatin E can be reacted with paraacetyl benzoic acid or benzoylvaleric acid to produce auristatin EB (AEB) and auristatin EVB (AEVB), respectively. Other typical auristatins include MMAF, and MMAE. The synthesis and structure of exemplary auristatins are described in U.S. Pat. Nos. 7,659,241, 7,498,298, 2009-0111756, 2009-0018086, and U.S. Pat. No. 7,968,687, each of which is incorporated herein by reference in its entirety and for all purposes.

Exemplary auristatin based conjugates include vcMMAE, vcMMAF and mcMMAF immunoconjugates as shown below, wherein Ab is an anti-CD228 antibody or antigen-binding fragment thereof as described herein and val-cit represents the valine-citrulline dipeptide. The drug loading is represented by p, the number of drug-linker molecules per antibody or antigen-binding fragment thereof. Depending on the context, p can represent the average number of drug-linker molecules per antibody or antigen-binding fragment thereof in a composition of antibodies or antigen-binding fragments thereof, also referred to the average drug loading. In some embodiments, p ranges from 1 to 20 and is preferably from 1 to 8. In some embodiments, when p represents the average drug loading, p ranges from about 2 to about 5. In some embodiments, p is about 2, about 3, about 4, or about 5. The average number of drugs per antibody in a preparation may be characterized by conventional means such as mass spectroscopy, HIC, ELISA assay, and HPLC. In some aspects, the anti-CD228 antibody or antigen-binding fragment thereof is attached to the drug-linker through a cysteine residue of the antibody or antigen-binding fragment thereof. In some embodiments, the cysteine residue is one that is engineered into the antibody or antigen-binding fragment thereof. In other aspects, the cysteine residue is an interchain disulfide cysteine residue.

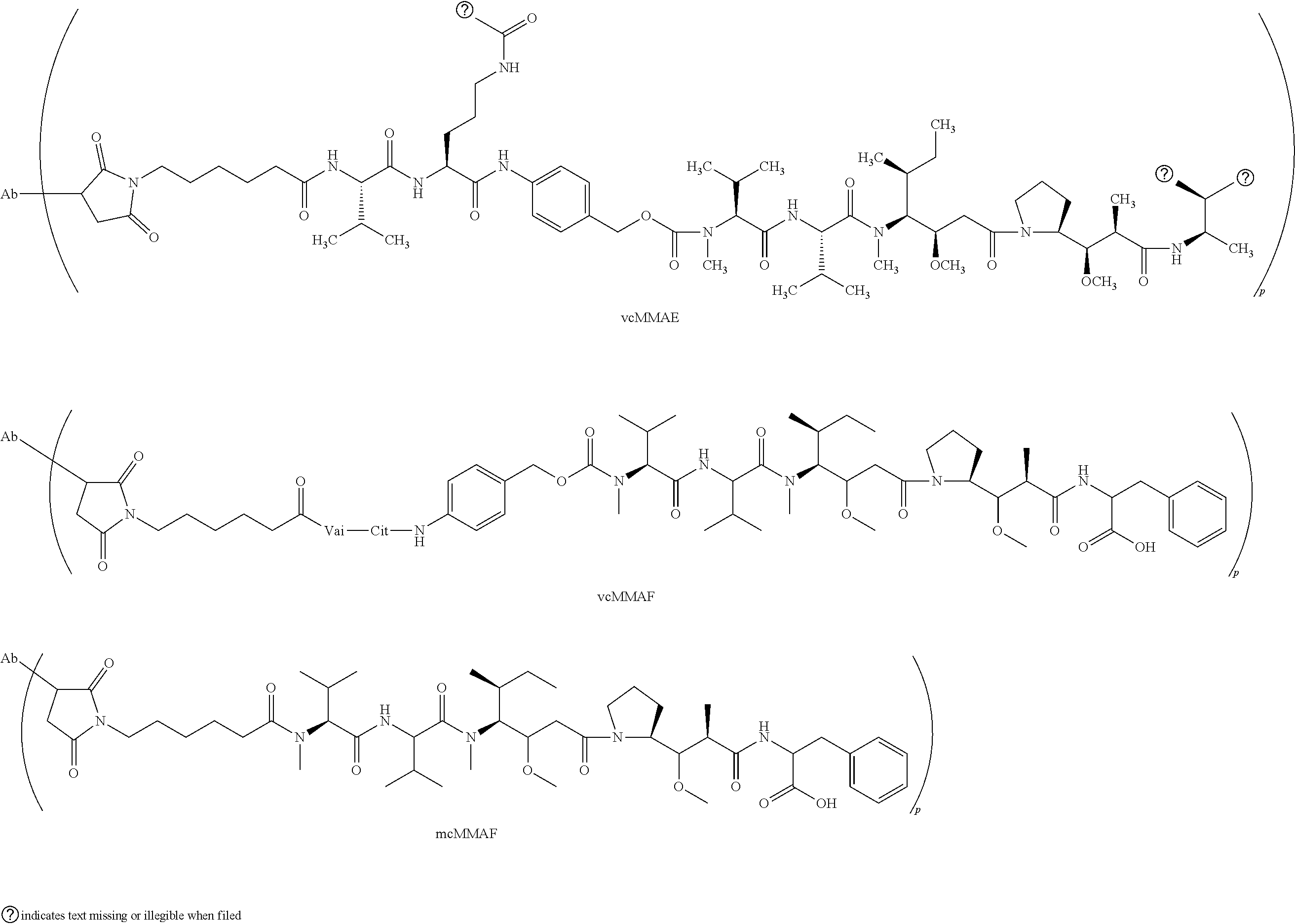

### C. Exemplary Uses and Applications of Antibodies or Antigen-Binding Fragments Thereof Specific for CD228, or of Immunoconjugates of Antibodies or Antigen-Binding Fragments Thereof.

In some aspects, the present disclosure provides diagnostic and/or analytical kits comprising one or more antibodies, antigen-binding fragments thereof, or immunoconjugates of antibodies or antigen-binding fragments thereof according to the disclosure.

In addition to their use in diagnostics, in yet another aspect, the disclosure contemplates pharmaceutical compositions comprising one or more antibodies, antigen-binding fragments thereof, or immunoconjugates of antibodies or antigen-binding fragments thereof of the disclosure and a pharmaceutically acceptable excipient.

Furthermore, in some embodiments, provided antibodies, antigen-binding fragments thereof, or immunoconjugates of antibodies or antigen-binding fragments thereof may be used in therapy, e.g., as anti-tumor and/or anti-infection agents and/or immune modulators. In some embodiments, provided antibodies, antigen-binding fragments thereof, or immunoconjugates of antibodies or antigen-binding fragments thereof may be used in the manufacture of a medicament, e.g., a medicament for the treatment of cancer, including CD228-positive cancer. In some embodiments, antibodies, antigen-binding fragments thereof, or immunoconjugates of antibodies or antigen-binding fragments thereof of the present disclosure are envisaged to be used in a method of prevention, amelioration, or treatment of human diseases, such as a cancer, including CD228-positive cancer. Accordingly, also provided are methods of preventing, ameliorating, or treating human diseases, such as cancer, including CD228-positive cancer, in a subject in need thereof, comprising administering to said subject a therapeutically effective amount of one or more antibodies, antigen-binding fragments thereof, or immunoconjugates of antibodies or antigen-binding fragments thereof of the disclosure or one or more compositions comprising such antibodies, antigen-binding fragments thereof, or immunoconjugates of antibodies or antigen-binding fragments thereof. In some embodiments, the cancer is CD228-positive cancer.

Examples of cancers that may be treated using the antibodies, antigen-binding fragments thereof, or immunoconjugates of antibodies or antigen-binding fragments thereof of the disclosure include lung cancer and melanoma, e.g., cutaneous or intraocular malignant melanoma, pancreatic cancer, mesothelioma, colorectal cancer, thyroid cancer, breast cancer, cholangiocarcinoma, esophageal cancer, and head and neck cancer. In some embodiments, cancer includes metastatic cancers.

In some embodiments, fusion proteins of the disclosure may simultaneously target tumor cells where CD228 is expressed and activate lymphocytes of the host immune system adjacent to such tumor cells. In some embodiments, fusion proteins of the disclosure may increase targeted anti-tumor T cell activity, enhance anti-tumor immunity and, and/or have a direct inhibiting effect on tumor growth, thereby producing synergistic anti-tumor results. In some embodiments, fusion proteins of the disclosure may activate immune responses in a tumor microenvironment. In some embodiments, fusion proteins of the disclosure may reduce side effects of effector lymphocytes towards healthy cells, i.e., off-target toxicity, for example, via locally inhibiting oncogene activity and/or inducing lymphocyte activation.

In some embodiments, the present disclosure encompasses the use of a fusion protein of the disclosure, or a composition comprising a provided fusion protein, for inducing a localized lymphocyte response in the vicinity of CD228-positive tumor cells. Accordingly, in some embodiments, the present disclosure provides methods of inducing a localized lymphocyte response in the vicinity of CD228-positive tumor cells, comprising applying one or more fusion proteins of the disclosure or of one or more compositions comprising such fusion proteins.

### D. Production of Exemplary Provided Antibodies or Antigen-Binding Fragments Thereof Specific for CD228

In some embodiments, the present disclosure provides nucleic acid molecules (e.g., DNA or RNA) that include nucleotide sequences encoding provided antibodies or antigen-binding fragments thereof. In some embodiments, the disclosure encompasses a vector containing a provided nucleic acid molecule. In some embodiments, the disclosure encompasses a host cell containing a provided nucleic acid molecule or vector. Since the degeneracy of the genetic code permits substitutions of certain codons by other codons specifying the same amino acid, the disclosure is not limited to a specific nucleic acid molecule encoding an antibody or antigen-binding fragment thereof as described herein, rather, encompassing all nucleic acid molecules that include nucleotide sequences encoding a functional antibody or antigen-binding fragment thereof. In this regard, the present disclosure also relates to nucleotide sequences encoding provided antibodies or antigen-binding fragments thereof.

A nucleic acid molecule, such as DNA, is referred to as “capable of expressing a nucleic acid molecule” or “able to allow expression of a nucleotide sequence” if it includes sequence elements that contain information regarding to transcriptional and/or translational regulation, and such sequences are “operably linked” to the nucleotide sequence encoding the protein. An operable linkage is a linkage in which the regulatory sequence elements and the sequence to be expressed are connected in a way that enables gene expression. The precise nature of the regulatory regions necessary for gene expression may vary among species, but in general these regions include a promoter, which, in prokaryotes, contains both the promoter per se, i.e., DNA elements directing the initiation of transcription, as well as DNA elements which, when transcribed into RNA, will signal the initiation of translation. Such promoter regions normally include 5′ non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native protein to a specific compartment of a host cell.

In addition, 3′ non-coding sequences may contain regulatory elements involved in transcriptional termination, polyadenylation or the like. If, however, these termination sequences are not satisfactorily functional in a particular host cell, then they may be substituted with signals functional in that cell.

Therefore, a nucleic acid molecule of the disclosure may be “operably linked” to one or more regulatory sequences, such as a promoter sequence, to allow expression of this nucleic acid molecule. In some embodiments, a nucleic acid molecule of the disclosure includes a promoter sequence and a transcriptional termination sequence. Suitable prokaryotic promoters are, for example, the tet promoter, the lacUV5 promoter or the T7 promoter. Examples of promoters useful for expression in eukaryotic cells are the SV40 promoter or the CMV promoter.

In some embodiments, provided nucleic acid molecules can also be part of a vector or any other kind of cloning vehicle, such as a plasmid, a phagemid, a phage, a baculovirus, a cosmid or an artificial chromosome.

In some embodiments, a provided nucleic acid molecule may be included in a phagemid. As used in this context, a phagemid vector denotes a vector encoding the intergenic region of a temperate phage, such as M13 or f1, or a functional part thereof fused to the cDNA of interest. For example, in some embodiments, after superinfection of bacterial host cells with such a provided phagemid vector and an appropriate helper phage (e.g., M13K07, VCS-M13 or R408) intact phage particles are produced, thereby enabling physical coupling of the encoded heterologous cDNA to its corresponding polypeptide displayed on the phage surface (Lowman, Annu Rev Biophys Biomol Struct, 1997, Rodi and Makowski, Curr Opin Biotechnol, 1999).

In accordance with various embodiments, cloning vehicles can include, aside from the regulatory sequences described above and a nucleic acid sequence encoding an antibody or antigen-binding fragment thereof as described herein, replication and control sequences derived from a species compatible with the host cell that is used for expression as well as selection markers conferring a selectable phenotype on transformed or transfected cells. Large numbers of suitable cloning vectors are known in the art and are commercially available.

The disclosure also relates, in some embodiments, to methods for the production of antibodies or antigen-binding fragments thereof of the disclosure starting from a nucleic acid coding for an antibody or antigen-binding fragment thereof or any subunit(s) therein using genetic engineering methods. In some embodiments, a provided method can be carried out in vivo, wherein a provided antibody or antigen-binding fragment thereof can, for example, be produced in a bacterial or eukaryotic host organism, and then isolated from this host organism or its culture. It is also possible to produce an antibody or antigen-binding fragment thereof of the disclosure in vitro, for example, using an in vitro translation system.

When producing an antibody or antigen-binding fragment thereof in vivo, a nucleic acid encoding such antibody or antigen-binding fragment thereof may be introduced into a suitable bacterial or eukaryotic host organism using recombinant DNA technology well known in the art. In some embodiments, a DNA molecule encoding an antibody or antigen-binding fragment thereof as described herein, and in particular a cloning vector containing the coding sequence of such an antibody or antigen-binding fragment thereof can be transformed into a host cell capable of expressing the gene. Transformation can be performed using standard techniques. Thus, the disclosure is also directed to host cells containing a nucleic acid molecule as disclosed herein.

In some embodiments, transformed host cells may be cultured under conditions suitable for expression of the nucleotide sequence encoding an antibody or antigen-binding fragment thereof of the disclosure. In some embodiments, host cells can be prokaryotic, such as Escherichia coli (E. coli) or Bacillus subtilis, or eukaryotic, such as Saccharomyces cerevisiae, Pichia pastoris, SF9 or High5 insect cells, immortalized mammalian cell lines (e.g., Hela cells or CHO cells) or primary mammalian cells.

In some embodiments, it is also possible to produce an antibody or antigen-binding fragment thereof of the disclosure in the cytosol of a host cell, preferably E. coli. In this case, a provided antibody or antigen-binding fragment thereof can either be directly obtained in a soluble and folded state or recovered in the form of inclusion bodies, followed by renaturation in vitro. A further option is the use of specific host strains having an oxidizing intracellular milieu, which may thus allow the formation of disulfide bonds in the cytosol (Venturi et al., J Mol Biol, 2002).

In some embodiments, an antibody or antigen-binding fragment thereof of the disclosure as described herein may be not necessarily generated or produced, in whole or in part, via use of genetic engineering. Rather, such protein can also be obtained by any of the many conventional and well-known techniques such as plain organic synthesis strategies, solid phase-assisted synthesis techniques, commercially available automated synthesizers, or by in vitro transcription and translation. It is, for example, possible that promising antibodies or antigen-binding fragments thereof are identified using molecular modeling, synthesized in vitro, and investigated for the binding activity for the target(s) of interest. Methods for the solid phase and/or solution phase synthesis of proteins are well known in the art (see, e.g., Bruckdorfer et al., Curr Pharm Biotechnol, 2004).

In some embodiments, an antibody or antigen-binding fragment thereof of the disclosure may be produced by in vitro transcription/translation employing well-established methods known to those skilled in the art.

In some further embodiments, antibodies or antigen-binding fragments thereof as described herein may also be prepared by conventional recombinant techniques alone or in combination with conventional synthetic techniques.

The skilled worker will appreciate methods useful to prepare antibodies or antigen-binding fragments thereof contemplated by the present disclosure but whose protein or nucleic acid sequences are not explicitly disclosed herein. As an overview, such modifications of the amino acid sequence include, e.g., directed mutagenesis of single amino acid positions to simplify sub-cloning of a protein gene or its parts by incorporating cleavage sites for certain restriction enzymes. Also, these mutations can be incorporated to further improve the affinity of an antibody or antigen-binding fragment thereof for its target(s) (e.g., CD228). Furthermore, mutations can be introduced to modulate one or more characteristics of the protein such as to improve folding stability, serum stability, protein resistance or water solubility or to reduce aggregation tendency, if necessary.

Additional objects, advantages, and features of this disclosure will become apparent to those skilled in the art upon examination of the following Examples and the attached Figures, which are not intended to be limiting. Thus, it should be understood that although the present disclosure is specifically disclosed by exemplary embodiments and optional features, modification and variation of the disclosures embodied therein herein disclosed may be resorted to by those skilled in the art and that such modifications and variations are considered to be within the scope of this disclosure.

### VI. EXAMPLES

### Example 1: Generation of Anti-CD228 Antibodies

A human immunoglobulin transgenic rat strain (OmniRat®; OMT, Inc.) was used to develop monoclonal antibody expressing hybridoma cells. The OmniRat® contains a chimeric human/rat IgH locus (comprising 22 human VHs, all human D and JH segments in natural configuration linked to the rat CH locus) together with fully human IgL loci (12 VKs linked to JK-CK and 16 VAs linked to JA-CA). See Osborn, et al. (2013) J Immunol 190 (4): 1481-1490); WO 2014/093908. In response to immunization, the introduced human heavy and light chain transgenes undergo class switching and somatic mutation to generate high affinity IgG monoclonal antibodies.

Transgenic rats were immunized with recombinant human CD228 protein. A subcutaneous injection included 48.2 μg of recombinant protein in Complete Freund's adjuvant on day 1 and 48 μg of recombinant protein in Incomplete Freund's adjuvant on days 33, 81, and 127.

The presence of antibodies directed against human CD228 in sera of transgenic rats was monitored by flow cytometry on days 33, 81, and 127 with RPMI-7951 cells engineered to stably express human CD228. Transgenic rats with detectable immune responses were boosted five and seven days before harvesting spleen and lymph nodes. The boost prior to organ harvest included a 90 μg intravascular injection and a 18 μg intraperitoneal injection of recombinant human CD228 protein suspended in Phosphate Buffer Solution (PBS).

Splenocytes and lymphocytes demonstrating B cell lineage and specificity to fluorescently labeled recombinant human CD228 were sorted as single cells by flow activated cell sorting (FACS) into cell lysis buffer. RNA from single B cells was reverse transcribed to cDNA and amplified from primer sets to known human variable regions. Amplicons underwent DNA purification for Sanger sequencing with appropriate sequencing primers, after which heavy and light chain sequences were identified and annotated by IgBLAST.

Productive heavy and light chain sequences of human variable regions identified from single B cell cloning and Sanger sequencing were selected for gene synthesis and cloned into expression vectors using traditional cloning techniques. The expression vectors encode human IgG1, human kappa, or human lambda constant regions that are in frame and downstream of the human variable region. Sanger sequencing verified the antibody expression constructs.

To generate full length antibodies, heavy and light chain expression vectors were co-transfected into ExpiCHO cells according to the manufacturer's protocols (Life Technologies). Cell culture supernatants were collected by centrifugation 9 days after transfection. Filtered culture supernatants were spiked with 10% Triton-X (final concentration 0.1%) and left overnight at 4 °C on a shake platform. Supernatants were then loaded onto a 5 mL HiTrap MabSelectSuRe column at 2.5 mL/min using an AKTA Avant 25 chromatography system. The column was then washed with: 5 column volumes (CV) of endo wash buffer (1×PBS, 0.1% Triton-X), 5 CV of high salt buffer (1×PBS, 0.5 M NaCl), and 7.5 CV standard 1×PBS, pH 7.4. Protein was eluted with 1 CV of 20 mM citrate, pH 3, and immediately buffer exchanged into 1×PBS, pH 7.4 using a HiPrep Sephadex G-25 Desalting column. Fractionation was determined via A280 UV watch parameters, allowing for optimal collection of purified protein.

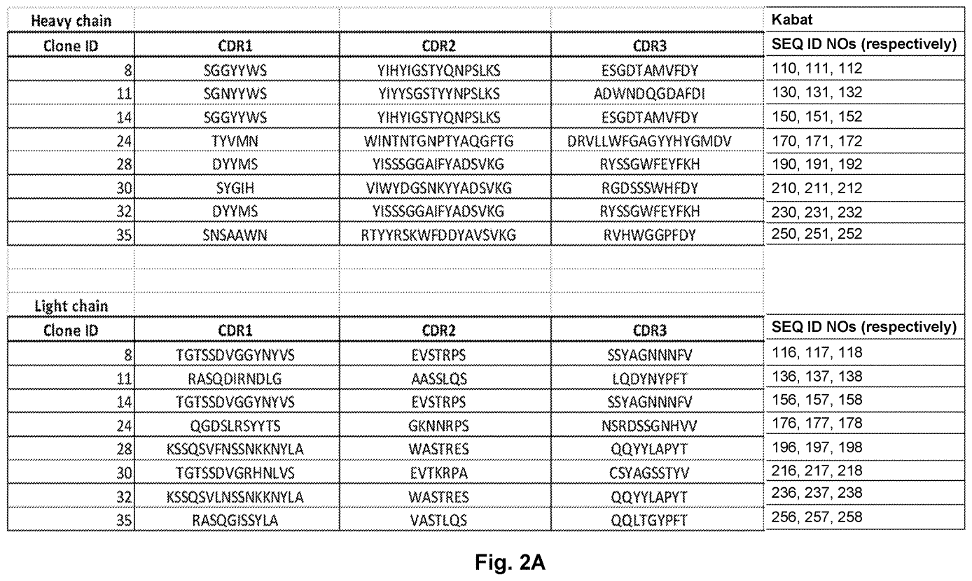

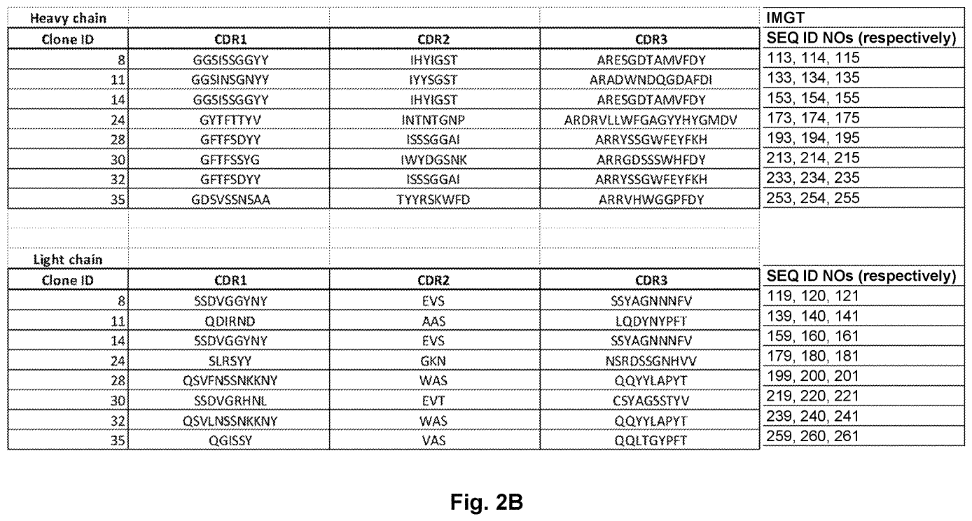

FIGS. 2A and 2B show the CDR sequences of the antibodies, as defined by Kabat (FIG. 2A) and IMGT (FIG. 2B).

### Example 2: Binding of Anti-CD228 Antibodies to Recombinant Human CD228

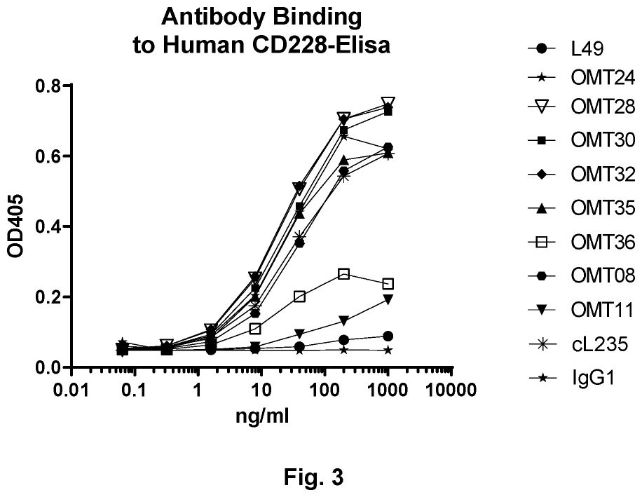

Humanized antibodies specific for human melanotransferrin (CD228) were evaluated for binding to recombinant human CD228 (R & D Systems) by ELISA. Antibodies were titrated on plates coated with 1 μg/ml recombinant human CD228, detected with an HRP-labeled goat anti-human IgG secondary reagent (Thermo), and developed with TMB substrate (Thermo). OD450 values were read on a plate reader with SoftMax Pro software. OD450 values for respective experiments were transferred to GraphPad Prism 8 for plotting and analysis. The results are shown in FIG. 3.

### Example 3: Binding of Anti-CD228 Antibodies to Cells

Humanized antibodies specific for human CD228 were evaluated for relative binding to CD228-expressing cancer cell lines. Cells were incubated with a titration of Alexa-647 labeled humanized antibody clones, washed, and evaluated for fluorescence intensity by flow cytometry on an Attune NXT flow cytometer. MFI values were transferred to GraphPad Prism 8 for plotting and analysis. EC50 values were determined by non-linear regression. The numbers next to the names of the cell lines reflect estimated surface CD228 copy number as estimated using a QIFIKIT quantitative analysis kit (Agilent).

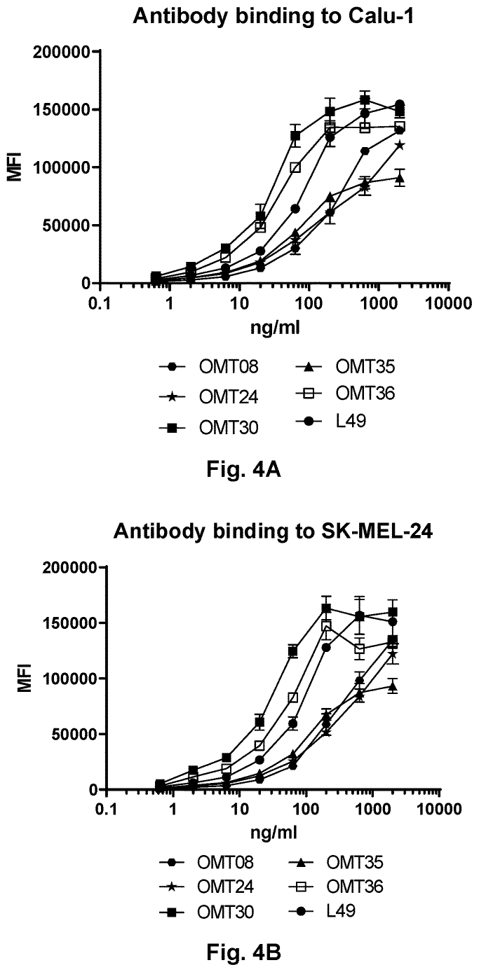

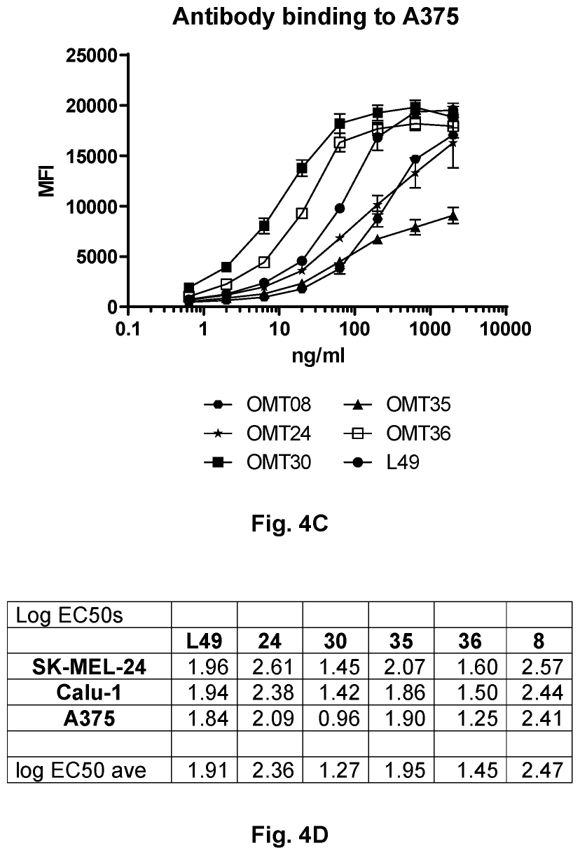

Results are shown in FIGS. 4A-4C as raw Mean Fluorescent Intensity (MFI) values. FIG. 4D shows the EC₅₀ value of each antibody for each cell line.

### Example 4: Binding Profiles of Anti-CD228 Antibodies

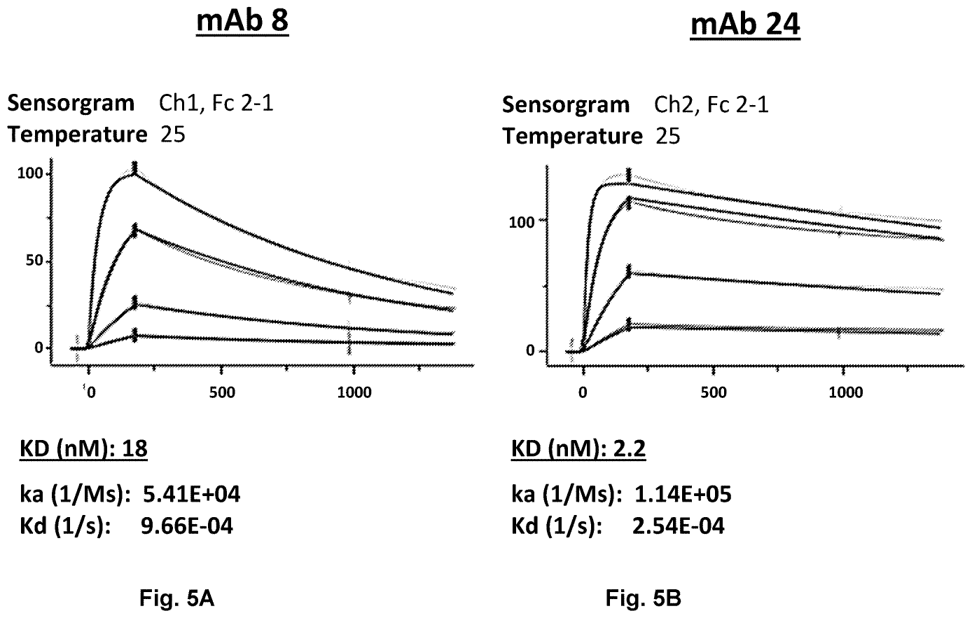

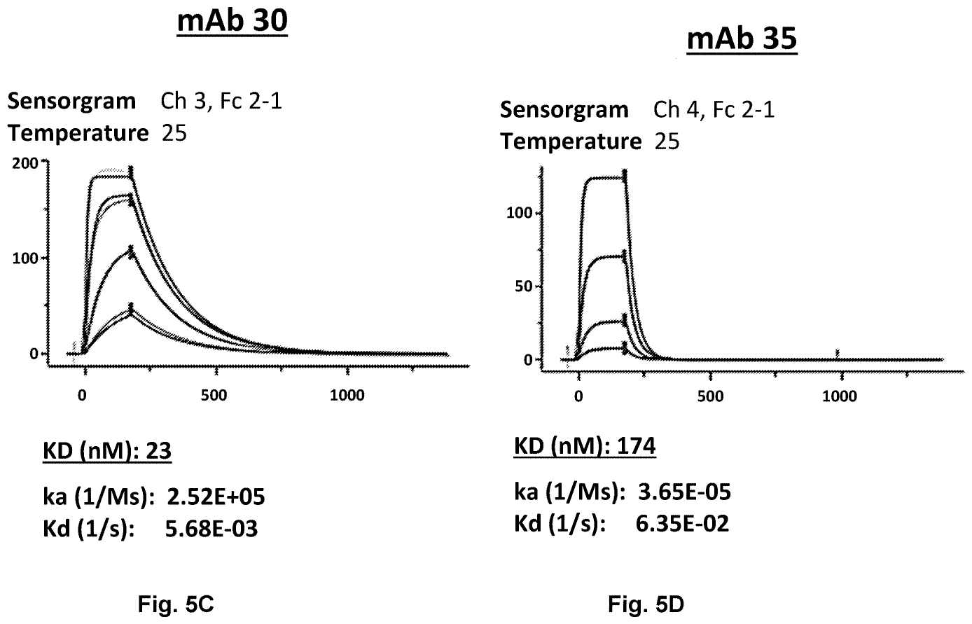

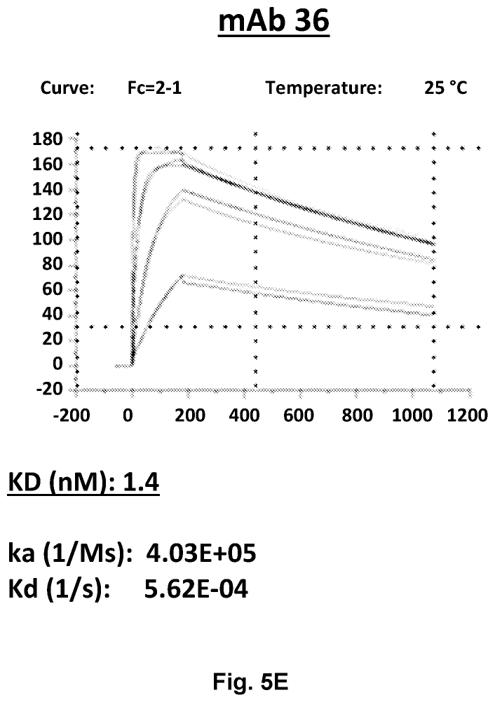

The binding kinetics and affinity of human CD228 with a C-terminal polyhistidine tag (R&D Systems) were determined by biolayer inferometry (BLI) using an Octet® RED384 system (Sartorius). Anti-human antibody capture AHC (GE Healthcare) biosensors were used for the analysis. Subsequently, anti-CD228 antibodies OMT8, OMT24, OMT30, OMT35, and OMT36 (IgG) at 0.5 g/mL in HBS-EP+ buffer were captured by the anti-human IgG-Fc antibody at the chip surface for 180 seconds. After each capture step, the biosensors were washed in HBS-EB+ blank. For affinity determination, dilutions of recombinant huCD228 (100 nM, 40 nM, 16 nM, 6.4 nM, 2.6 nM, and 1.0 nM) or blanks were prepared in HBS-EP+ buffer and applied to the biosensor. The binding assay was carried out with a contact time of 300 seconds and a dissociation time of 1,200 seconds. All measurements were performed at 25 °C. Fresh AHC biosensors were used for each analysis. Data were evaluated with Satorius Octet® Data Analysis Software (v12.0), and results are shown in FIGS. 5A-5E. Single referencing was used, and the 1:1 binding model was used to fit the raw data.

### Example 5: Lack of Cross-Reactivity of Anti-CD228 Antibodies to Transferrin and Lactotransferrin

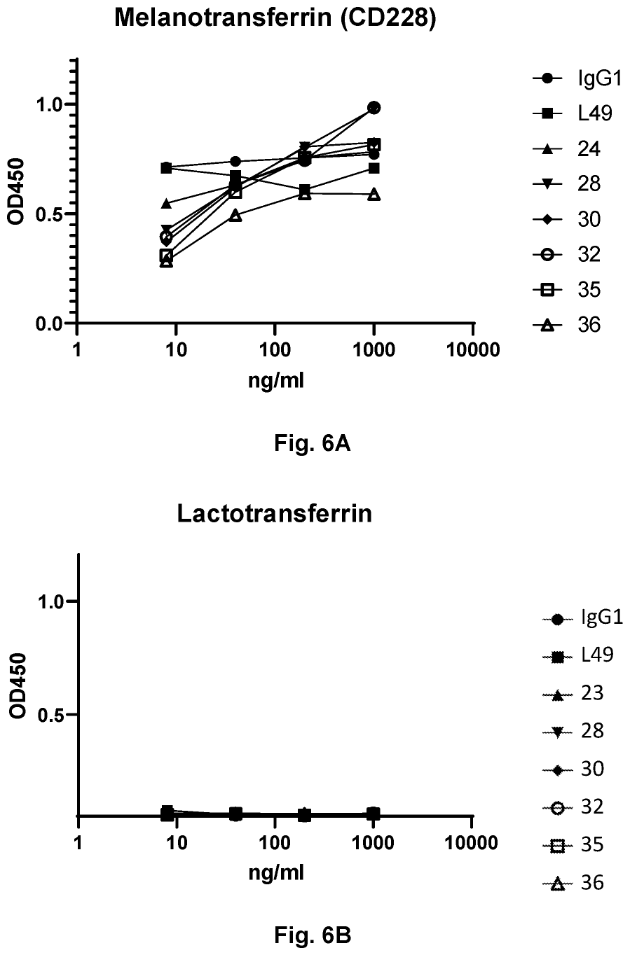

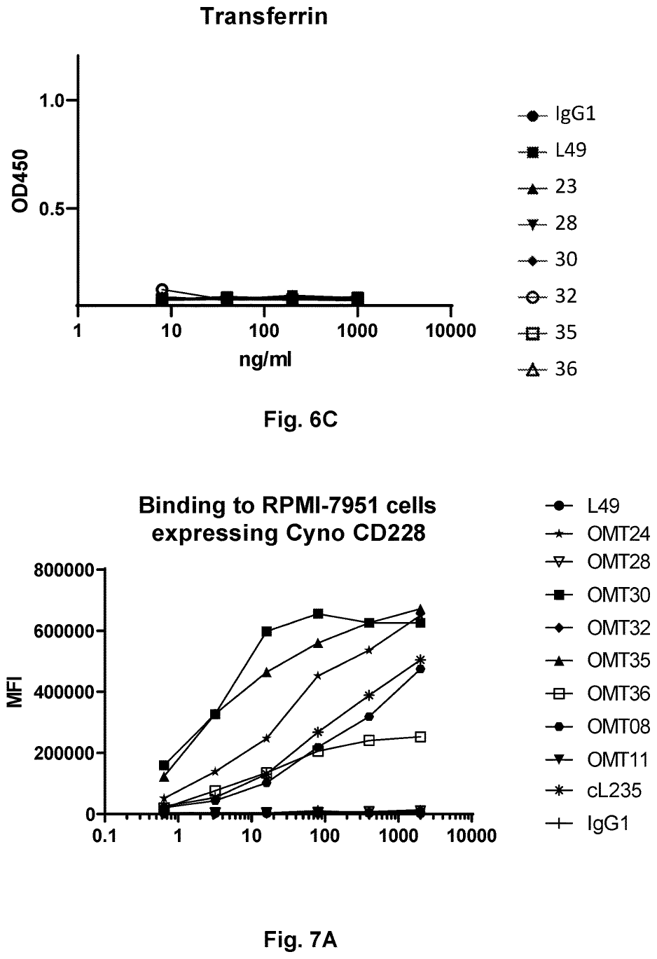

Humanized antibodies specific for human melanotransferrin (CD228) were evaluated for binding to related transferrin family members, lactotransferrin and transferrin, by ELISA. Antibodies were titrated on plates coated with 1 μg/mL of recombinant human melanotransferrin (CD228), lactotransferrin, and transferrin and detected with an HRP-labeled goat anti-human IgG secondary reagent (Thermo) developed with TMB substrate (Thermo), and OD450 values were read on a plate reader with SoftMax Pro software. OD450 values for respective experiments were transferred to GraphPad Prism 8 for plotting and analysis. These results, as shown in FIGS. 6A-6C, indicate the absence of cross-reactive binding of the anti-CD228 antibodies to other transferrin family members.

### Example 6: Cross-Reactivity of Anti-CD228 Antibodies to Non-Human CD228

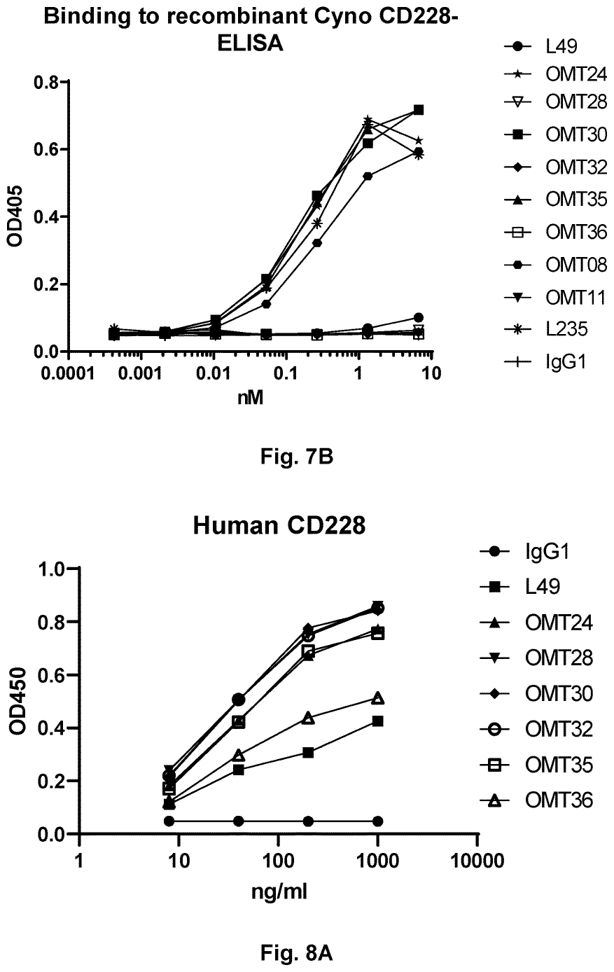

The CD228-negative human melanoma cell line RPMI-7951 was engineered to express cynomolgus CD228 to test binding of anti-CD228 antibody clones. 50,000 RPMI-7951 cells were incubated with a titration of humanized antibody clones, washed with staining buffer to remove excess antibodies, and incubated with 250 ng/ml fluorochrome-labeled monoclonal antibodies against human IgG1 (Thermo) to detect bound antibodies. Results are shown in FIG. 7A as Mean Fluorescent Intensity (MFI) as determined by measurement on an Attune NXT flow cytometer (Thermo).

Reactivity of CD228 antibody clones was tested against recombinant his-tagged cynomolgus CD228 by ELISA. Plates were coated with 1 μg/ml of recombinant his-cynomolgus CD228 in phosphate buffered saline, plates were blocked with 250 μL of Superblock Blocking Buffer (Thermo), antibodies were titrated across the plate, bound antibodies were detected with HRP-labeled goat anti-human IgG (Sigma), developed with TMB substrate (Thermo), and OD450 values were read on a plate reader with SoftmaxPro software. Results are shown in FIG. 7B.

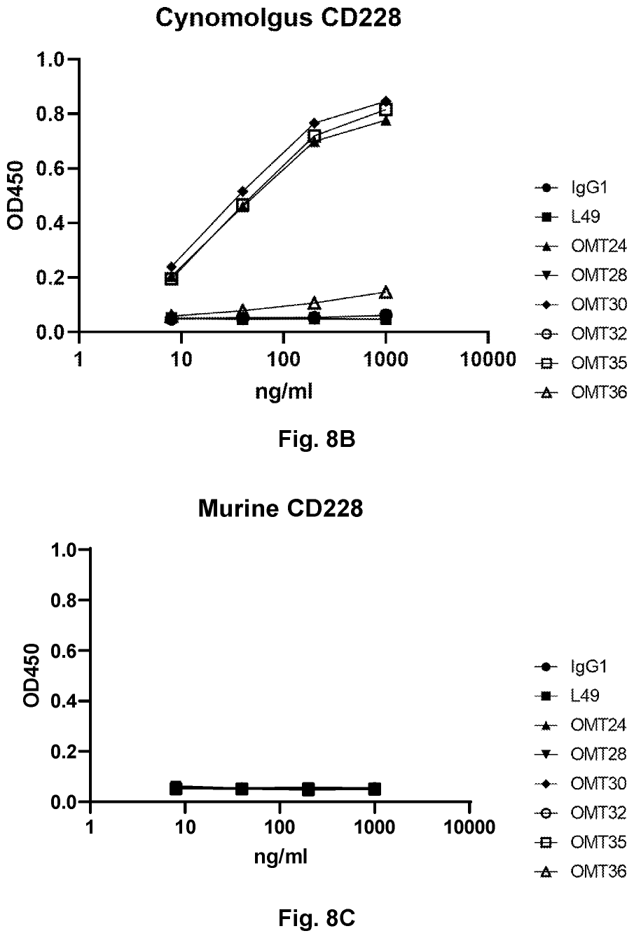

Humanized antibodies specific for human CD228 were evaluated for binding to recombinant cynomolgus and murine CD228 by ELISA. Antibodies were titrated on plates coated with 1 μg/mL recombinant human, cynomolgus, and murine CD228 and detected with an HRP labeled goat anti-human IgG secondary reagent (Thermo) developed with TMB substrate (Thermo), and OD450 values were read on a plate reader with SoftMax Pro software. OD450 values for respective experiments are shown in FIGS. 8A-8C.

These results indicate that a subset of antibody clones (OMT30, OMT35, OMT24, OMT8, OMT36, and L235) are cross-reactive to cynomolgus CD228 with minimal cross reactivity to murine CD228.

### Example 7: Anti-CD228 Antibody Cross-Competition Assay

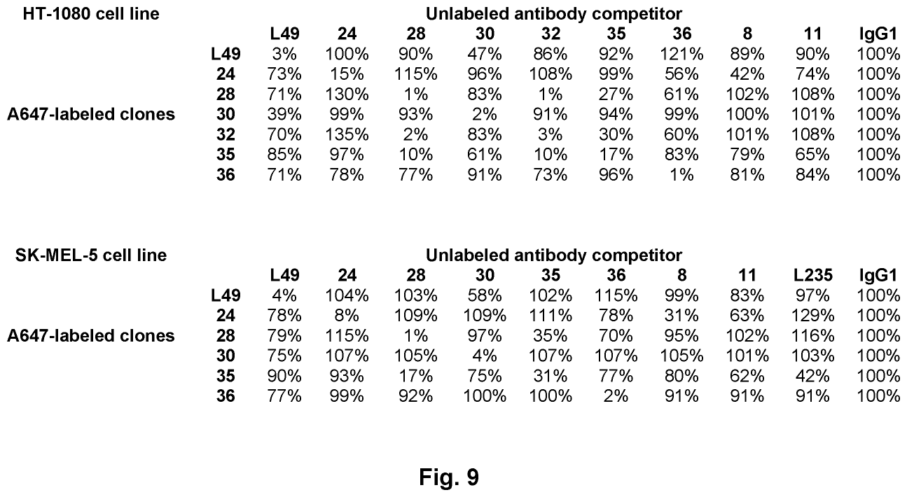

Fluorochrome-labeled humanized monoclonal antibodies specific for human CD228 were evaluated for binding to the HT-1080 tumor cell line and the SK-MEL-5 tumor cell line in competition with unlabeled humanized monoclonal antibodies to identify potential shared epitopes. HT-1080 cells and SK-MEL-5 cells were preincubated with 1 μg/ml of unlabeled humanized antibodies in staining buffer for 30 minutes at 4 °C, washed in staining buffer, and incubated with 200 ng/ml of A647-labeled versions. Bound fluorochome-labeled antibody was determined by flow cytometry on an Attune NXT flow cytometer. Results are shown in FIG. 9 as the percent of Mean Fluorescent Intensity compared to preincubation with a control non-binding human IgG1 (Sigma). Clones with direct epitope or steric competition show low staining percentage values. These results indicate antibody clones 28, 32, and 35 may share a closely related epitope, and clones 8, 11, 24, 30, and 36 likely bind unique CD228 epitopes.

### Example 8: Internalization of Antibody Clones on CD228+ Tumor Cell Lines

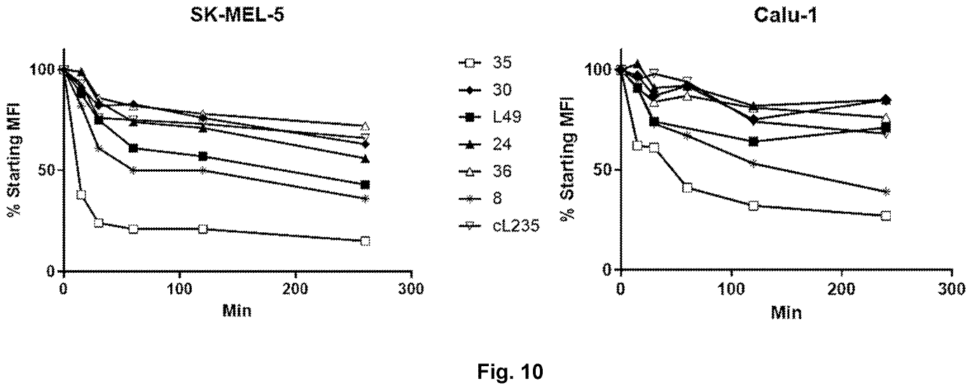

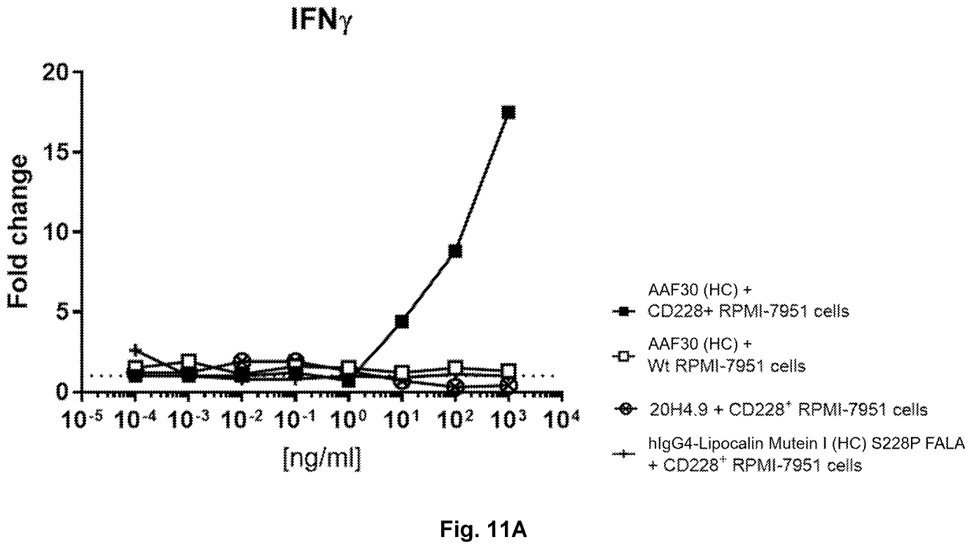

Humanized antibodies specific for human CD228 were evaluated for relative internalization on the CD228-expressing melanoma and lung cancer cell lines, SK-MEL-5 and Calu-1, respectively. Cells were incubated with 2 μg/ml of anti-CD228 antibodies, washed three times to remove free antibody, and incubated at 37 °C in 5% CO₂ for the times shown. At each timepoint, cells were fixed (BD Cytofix) and stained for bound human IgG1 with a fluorochome-labeled anti-human IgG1 antibody (Invitrogen). At the end of the time-course, cells were evaluated by FACS for the mean fluorescence intensity of surface bound antibody on an Attune NXT flow cytometer. Internalization results are shown in FIG. 10 as the % of MFI relative to the starting MFI at timepoint 0. These results demonstrate a range of internalization rates among antibody clones, with antibody clone 35 internalizing at the fastest rate.

### Example 9: Expression and Analysis of Representative Fusion Proteins

Representative antibody-lipocalin mutein fusion proteins were generated by fusing together CD228-specific antibodies and CD137-specific lipocalin muteins, such as the lipocalin mutein of SEQ ID NO: 40, via a linker, such as the unstructured (G4S) 3 linker of SEQ ID NO: 13, to engage CD228 and CD137 at the same time. Two different exemplary CD228-specific antibodies were used. A first CD228-specific antibody had the heavy chain provided by SEQ ID NO: 75 (or comprised a heavy chain variable domain of SEQ ID NO: 70, or comprised the heavy chain CDRs (HCDR1, HCDR2, HCDR3) of SEQ ID NOs: 58-60) and had the light chain provided by SEQ ID NO: 76 (or comprised a light chain variable domain of SEQ ID NO: 71, or comprised the light chain CDRs (LCDR1, LCDR2, LCDR3) of SEQ ID NOs: 61-63). A second CD228-specific antibody had the heavy chain provided by SEQ ID NO: 78 (or comprised a heavy chain variable domain of SEQ ID NO: 72, or comprised the heavy chain CDRs (HCDR1, HCDR2, HCDR3) of SEQ ID NOs: 64-66) and had the light chain provided by SEQ ID NO: 79 (or comprised a light chain variable domain of SEQ ID NO: 73, or comprised the light chain CDRs (LCDR1, LCDR2, LCDR3) of SEQ ID NOs: 67-69). The exemplary fusion proteins of SEQ ID NOs: 80 and 76, SEQ ID NOs: 82 and 79, SEQ ID NOs: 75 and 81, and SEQ ID NOs: 78 and 83 were bivalent to CD137 with a CD137-specific lipocalin mutein being fused to the C-terminus of each heavy chain or each light chain.

The CD228-specific antibodies as well as all antibody lipocalin mutein fusion proteins described in this Example had an engineered IgG4 backbone, which contained a S228P mutation to minimize IgG4 half-antibody exchange in-vitro and in-vivo (Silva et al., J Biol Chem, 2015). Additional mutations in the IgG4 backbones may also exist in all antibodies described here, including any one or more of mutations F234A, L235A, M428L, N434S, M252Y, S254T, and T256E. F234A and L235A mutations may be introduced to decrease ADCC and ADCP (Glaesner et al., Diabetes Metab Res Rev, 2010). M428L and N434S mutations or M252Y, S254T, and T256E mutations may be introduced for extended serum half-life (Dall'Acqua et al., J Biol Chem, 2006; Zalevsky et al., Nat Biotechnol, 2010). All antibodies were expressed without the carboxy-terminal lysine to avoid heterogeneity.

The constructs of exemplary fusion proteins were generated by gene synthesis and cloned into a mammalian expression vector. They were then transiently expressed in suspension-adapted CHO-K1 cells.

### Example 10: Assessment of PBMC Response to Viral Peptides in the Presence of Tumor Cells with High or No Expression of CD228

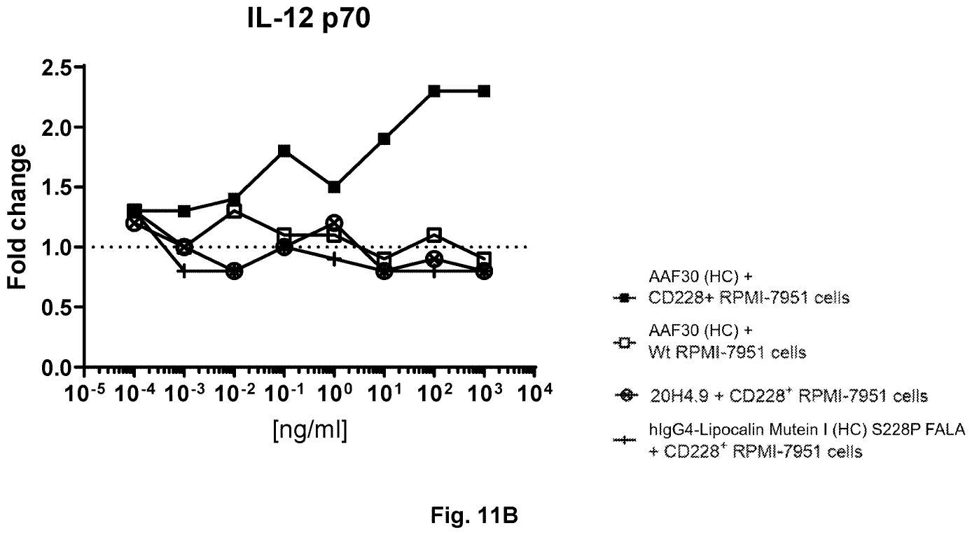

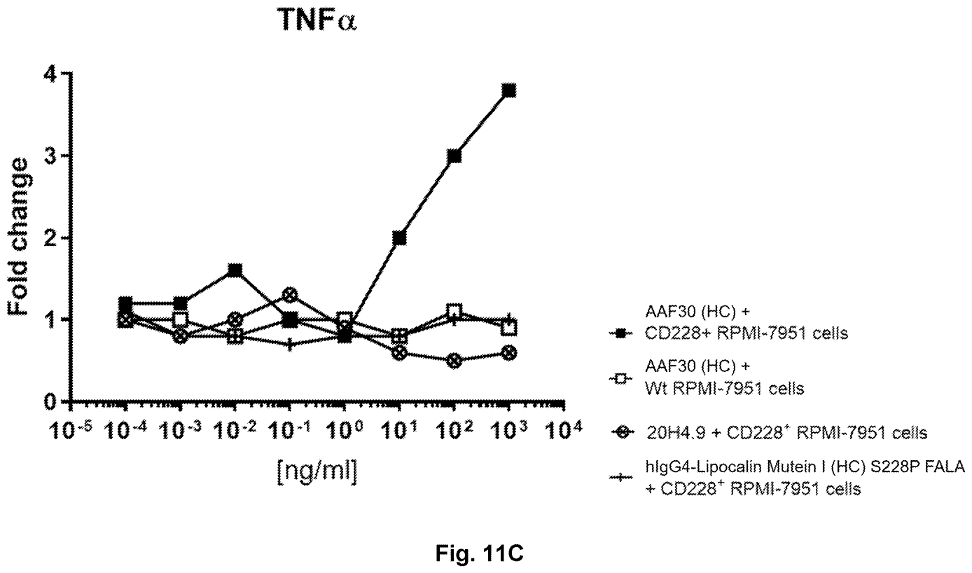

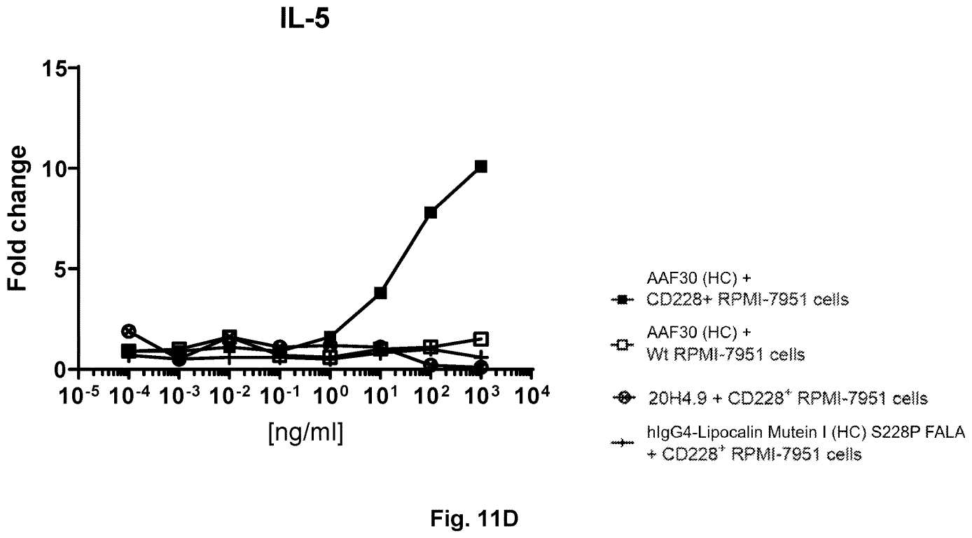

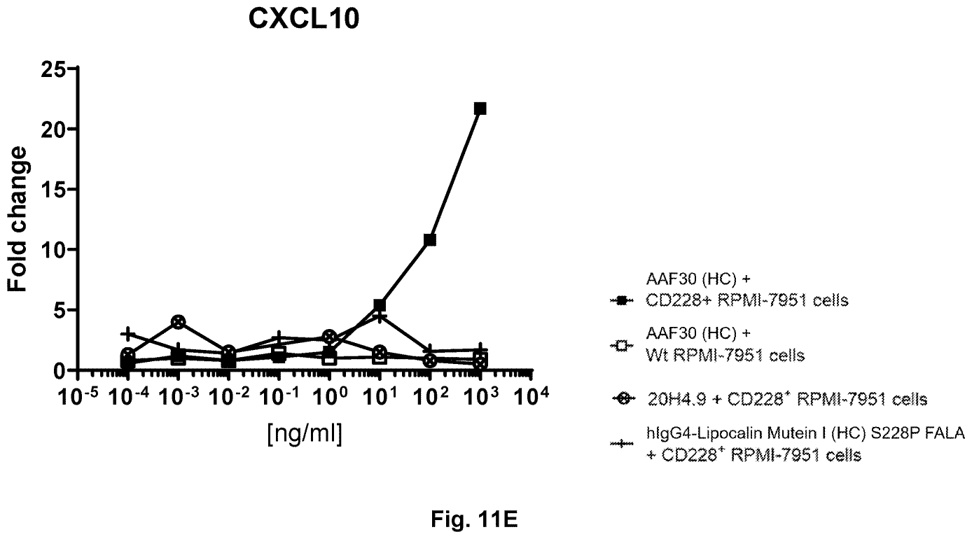

An assay was conducted to assess the ability of the fusion proteins to co-stimulate innate and adaptive immune cytokines from PBMC in response to viral peptides in a CD228 target dependent manner. PBMC isolated from healthy donors were co-incubated with engineered CD228+ or wild type (CD228−) RPMI-7951 tumor cell lines 10:1 in RPMI with 10% FCS, and were provided viral peptides from CMV, EBV, and flu viruses (CEF peptides). Bispecific fusion proteins and controls were titrated into the assay and changes in IFN-γ, TNF-α, IL-5, IL-12 (p70), and CXCL10 (IP-10) were measured at the end of 4-day stimulation at 37 °C in 5% CO₂. Supernatants were evaluated by Luminex® multiplex cytokine array for T cell (IFN-γ and TNF-α) and myeloid cell (IL-12 and CXCL10) responses. Data are shown in FIGS. 11A-11E as the fold change of cytokines relative to the untreated control wells. Samples were pooled from triplicate test article treatments prior to cytokine measurement. These results show that the fusion proteins (e.g., AAF30 (HC)), compared to antibodies, co-stimulate various innate and adaptive immune cytokines from PBMC in response to viral peptides in a CD228 target dependent manner and a dose dependent manner.

### Example 11: Assessment of PBMC Cellular Response to Viral Peptides in the Presence of CD228+ Tumor Cells

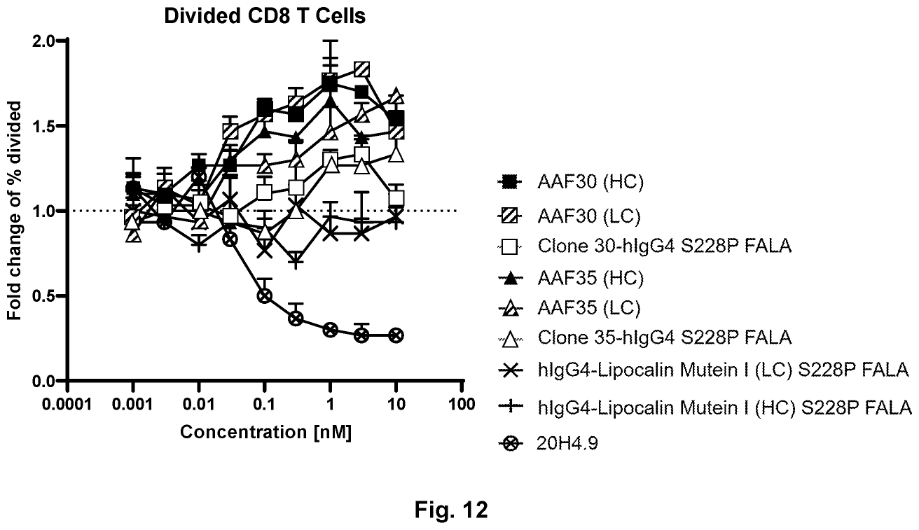

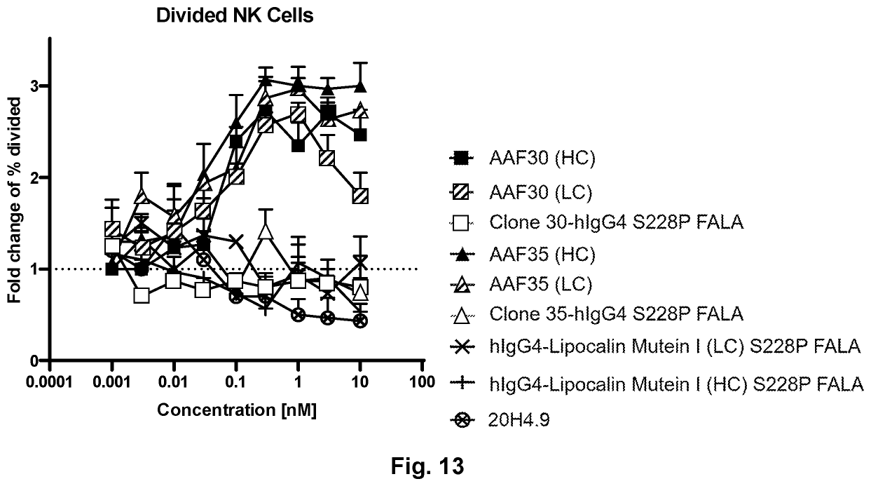

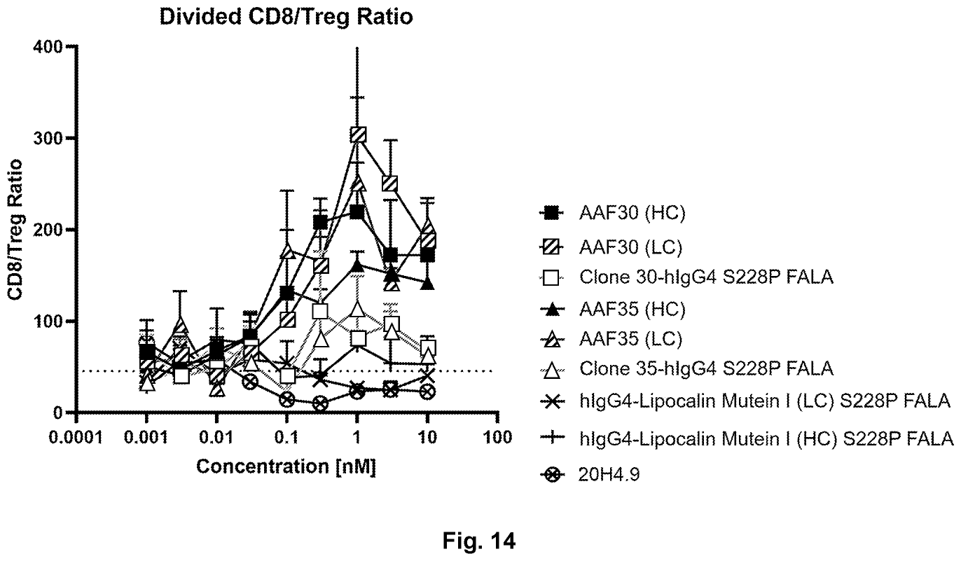

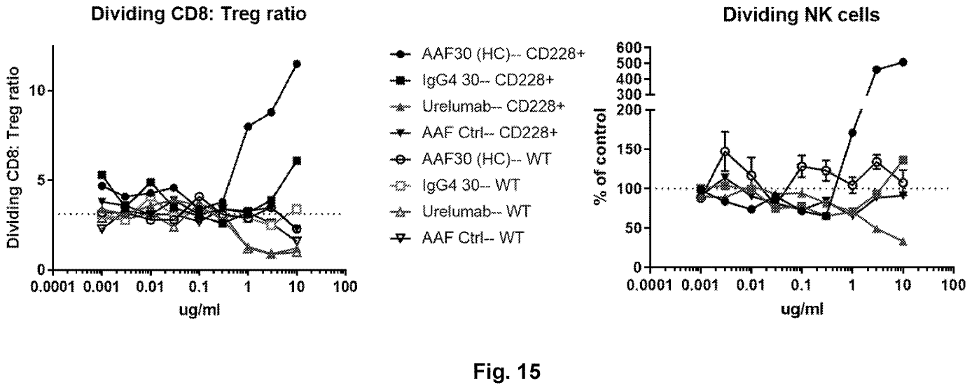

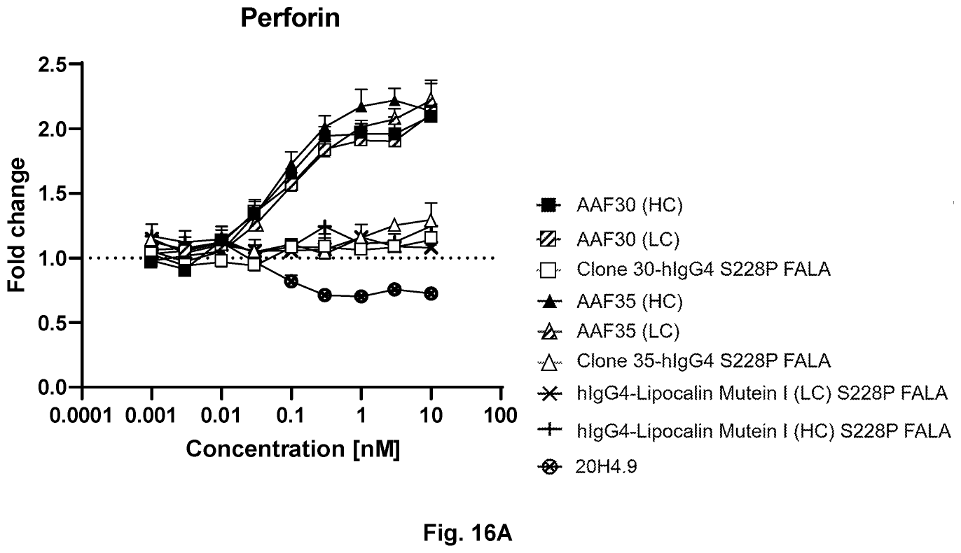

To evaluate the activity of bispecific fusion proteins in an antigen recall assay, healthy donor cryopreserved PBMC (Bloodworks Northwest) were thawed in prewarmed RPMI 10% FCS, washed, and labeled with 1.5 mL of 10 nM CFSE PBS 3% FCS at room temperature. To quench the labeling reaction, cells were washed 2× with 12 mL of RPMI 10% FCS. Cells were counted and mixed 10:1 with CD228-expressing cell line RPMI-7951^{CD228} (ATCC) (engineered to express human CD228) in RPMI-complete (10% FCS, 1×Glutamax, 1×MEM NEAA, 1× sodium pyruvate, 1× penicillin/streptomycin (Gibco™)) and distributed in non-adherent 96-well round bottom plates (Sbio), 1.5×10⁵ cells/well. CEF peptides were added to a final concentration of 100 ng/ml, and fusion protein bispecifics and controls were added at equimolar titrations in triplicate. The assay was allowed to incubate for 5 days at 37 °C 5% CO₂. FIG. 12, FIG. 13, and FIG. 14 respectively show representative examples of CD8 T cell, NK cell and CD8 T cell/Treg ratio calculations from antigen recall assays in coculture with CD228-engineered RPMI-7951 cell line. Similarly, FIG. 15 shows representative examples of NK cell and CD8 T cell/Treg ratio calculations from antigen recall assays. These results show that the fusion proteins (e.g., AAF30 (HC)), compared to antibodies, co-stimulate proliferation/division of CD8+ T cells and NK cells in response to viral peptides in a CD228 target dependent manner and a dose dependent manner.

### Example 12: Assessment of PBMC Cytokine Response to Viral Peptides in the Presence of CD228+ Tumor Cells

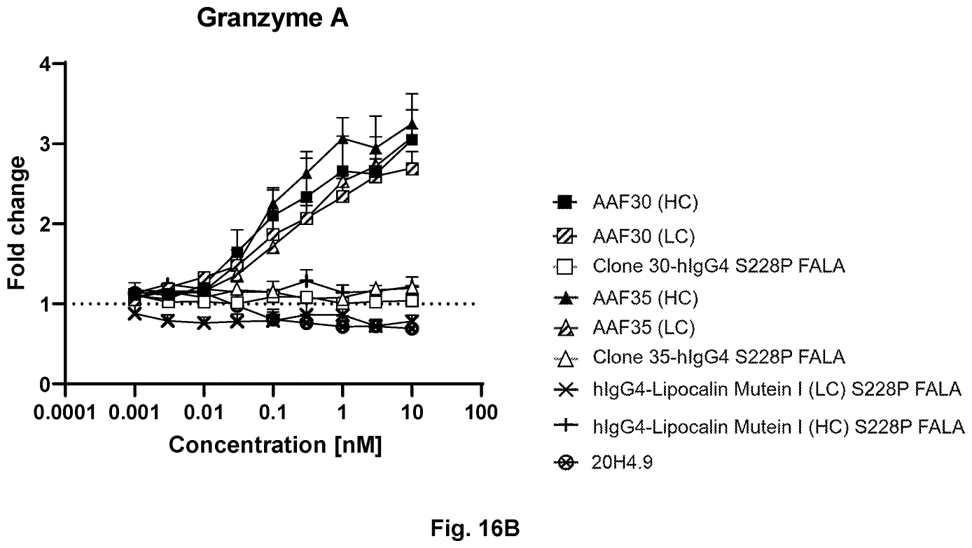

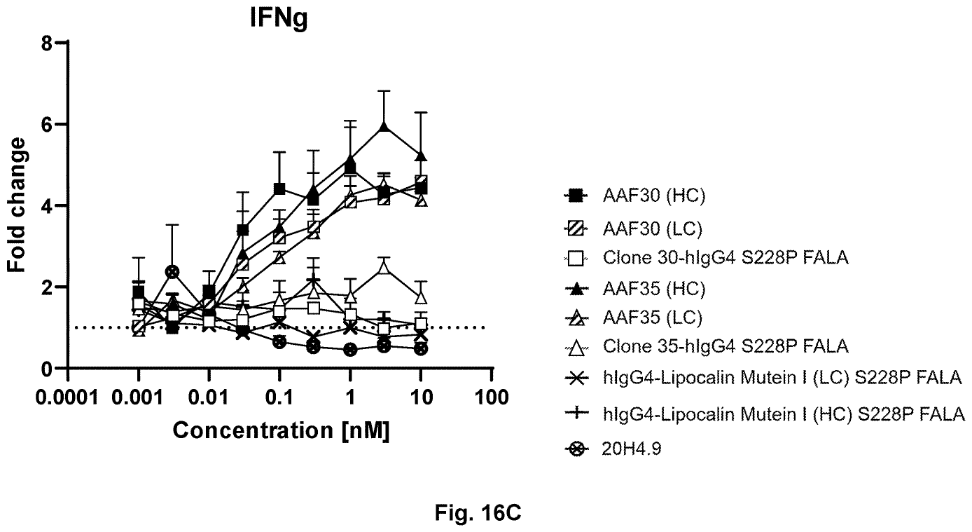

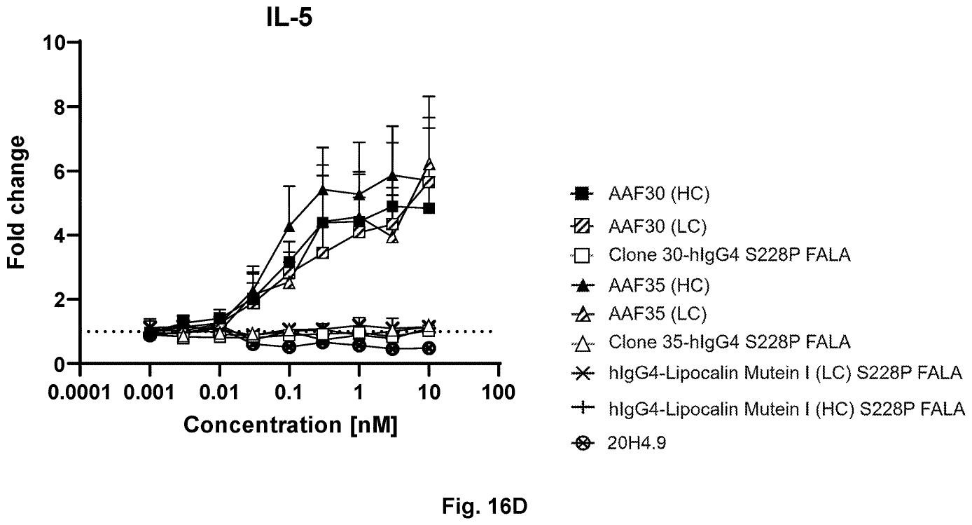

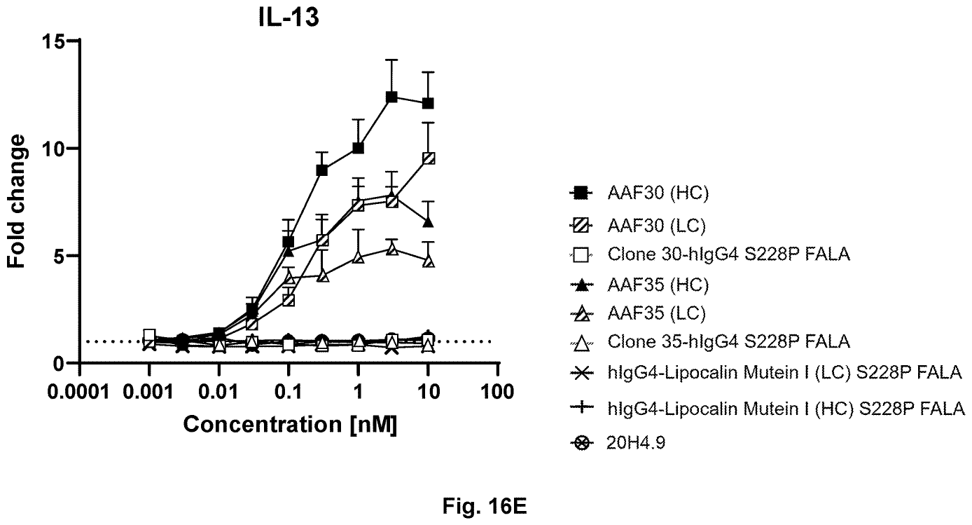

To evaluate the activity of bispecific fusion proteins in an antigen recall assay, healthy donor cryopreserved PBMC (Bloodworks Northwest) were thawed in prewarmed RPMI 10% FCS, washed, and labeled with 1.5 mL of 10 nM CFSE PBS 3% FCS at room temperature. To quench the labeling reaction, cells were washed 2× with 12 mL of RPMI 10% FCS. Cells were counted and mixed 10:1 with CD228-expressing cell line RPMI-7951^{CD228} (ATCC) (engineered to express human CD228), CALU-1 cells (ATCC), or H3677 cells (Seagen) in RPMI-complete (10% FCS, 1× Glutamax, 1×MEM NEAA, 1× sodium pyruvate, 1× penicillin/streptomycin (Gibco™)) and distributed in non-adherent 96-well round bottom plates (Sbio), 1.5×10⁵ cells/well. CEF peptides were added to a final concentration of 100 ng/ml, and fusion protein bispecifics and controls were added at equimolar titrations in triplicate. The assay was allowed to incubate for 5 days at 37 °C 5% CO₂. At the end of the assay, plates were spun down, and supernatants were collected for cytokine evaluation. Cytokines were measured using the MILLIPLEX® MAP Human CD8+ T Cell Magnetic Bead Panel Premixed 17 Plex and read out on a Luminex® MAGPIX® system. FIGS. 16A-16E show representative fold-change averages of cytokines across three CD228 expressing cell lines. Raw Luminex® data were exported and analyzed in Microsoft Excel. These data highlight consistent changes in cytotoxic effector molecules and diverse cytokines in antigen recall in the presence of bispecific fusion proteins.

### Example 13: Assessment of T Cell Activation in Presence of Tumor Cells with High or No Expression of CD228

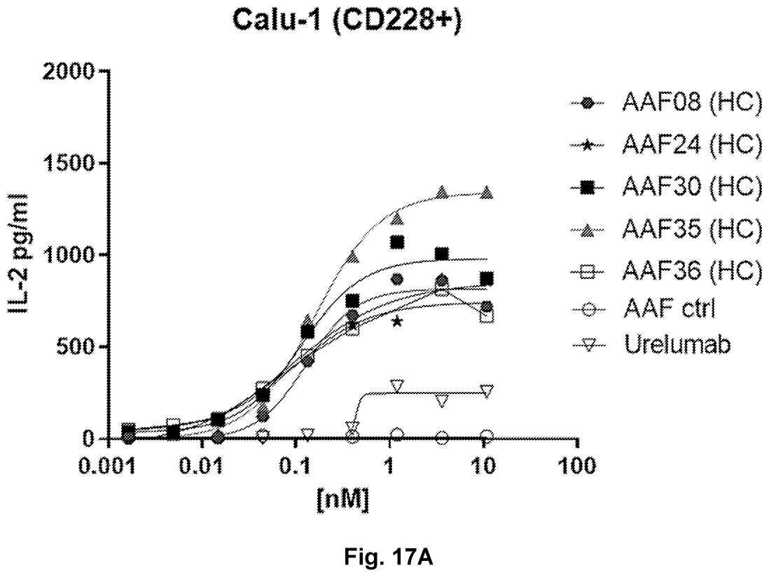

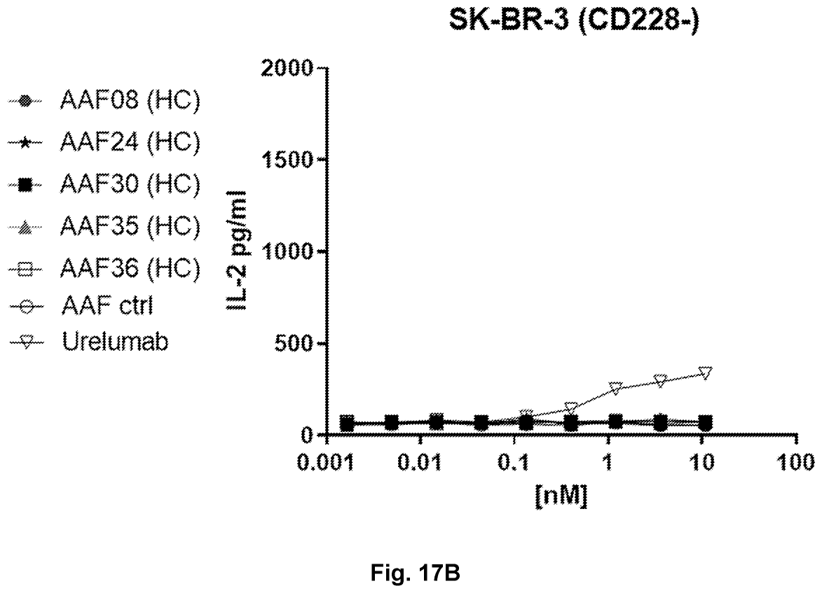

An assay was conducted to assess the requirement for CD228 expression by cancer cells for primary T cell costimulation with fusion proteins. Healthy donor primary T cells were incubated in anti-CD3 coated plates (0.25 μg/ml in 50 μL PBS, overnight) to drive T cell receptor stimulation, and CD228-positive Calu-1 cells or CD228-negative SK-BR-3 cells were added to cultures with a titration of fusion proteins. T cells were mixed with tumor cells at a final ratio of 10:1 in RPMI with 10% FCS. At the end of a three-day stimulation at 37 °C in 5% CO₂, supernatants were collected and measured for the cytokine IL-2. FIGS. 17A-17B show IL-2 secretion in response to costimulation by the fusion proteins and urelumab. These results show that the fusion proteins (e.g., AAF35 (HC)) co-stimulate T cell activation in a CD228 target-dependent manner and in a dose-dependent manner.

### Example 14: Assessment of T Cell Cytokines Produced in Cocultures with Anti-CD3 scFv Engineered CD228-Expressing Tumor Cell Line

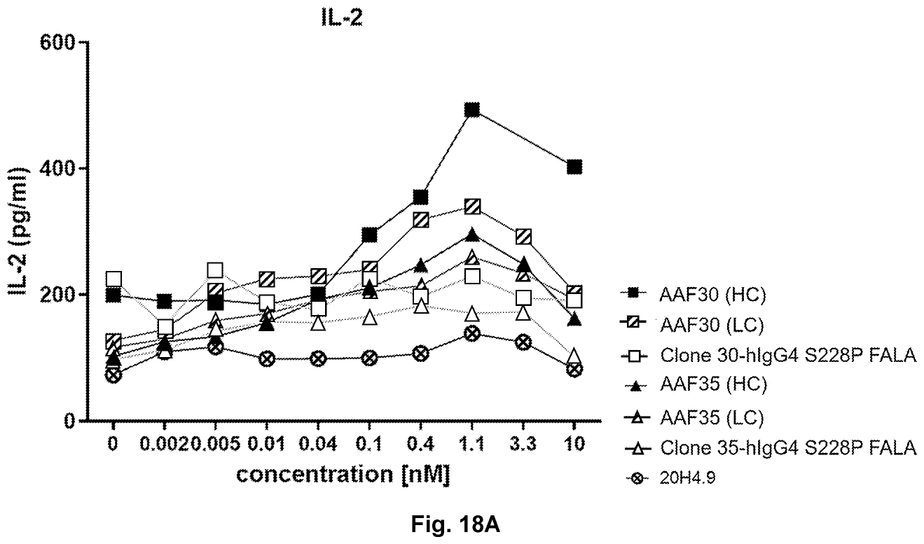

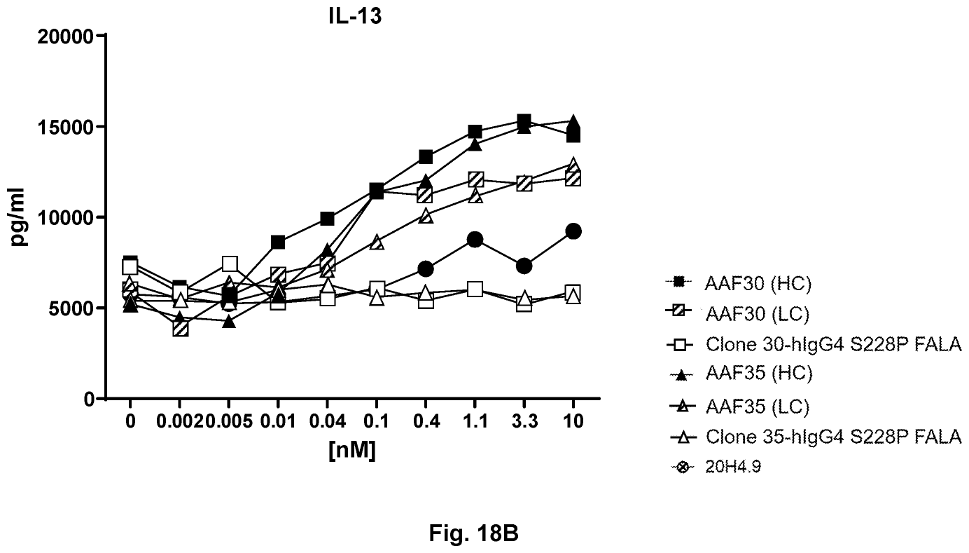

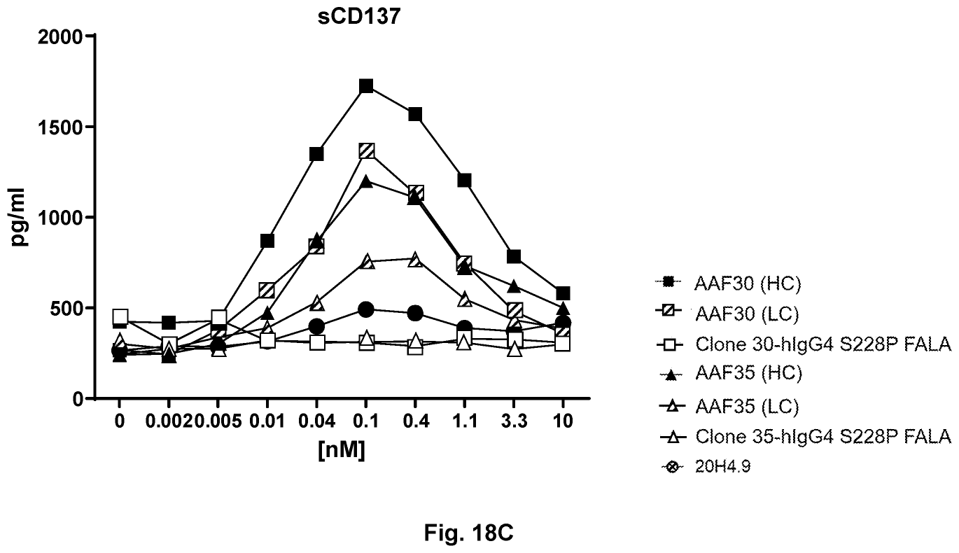

Experiments were conducted to assess the impact of fusion proteins on cytokine and soluble 4-1BB (sCD137) production from T cells receiving direct T cell receptor stimulation from CD228-expressing tumor cells. Healthy donor PBMC were co-cultured with CD228+ CALU-1 tumor cells (10:1, in RPMI 10% FCS) engineered to express surface anti-CD3 scFv to elicit direct T cell receptor engagement by tumor cells. A titration of fusion proteins or controls were added in triplicate in a 96-well round bottom plate. At the end of 3-day stimulation at 37 °C in 5% CO₂, supernatants were collected and measured for soluble cytokines IL-2, IL-13, and sCD137 by Luminex® multiplex array. Samples were pooled from triplicate test article treatments prior to cytokine measurement. The results are shown in FIGS. 18A-18C, which demonstrate that the fusion proteins augment cytokines from direct T cell tumor cell interactions.

### Example 15: Assessment of CD8 T Cell Proliferation in Cocultures with an Anti-CD3 scFv Engineered CD228-Expressing Tumor Cell Line

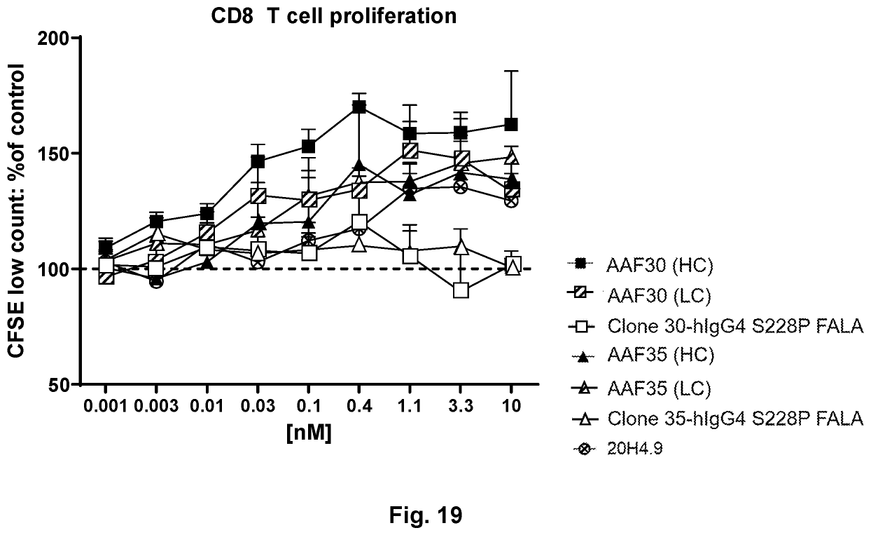

Experiments were conducted to assess the impact of fusion protein bispecifics on cytotoxic T cells receiving direct T cell receptor stimulation from CD228-expressing tumor cells. Healthy donor PBMCs were CFSE-labeled and co-cultured with CD228+ CALU-1 tumor cells (10:1) engineered to express surface anti-CD3 scFv to elicit T cell receptor engagement. A titration of bispecific fusion proteins or controls were added in triplicate in a 96-well round bottom plate. After 72 hours of incubation at 37 °C in 5% CO₂, plates were washed, stained with a live dead viability dye and antibodies specific for CD3, CD4, and CD8, and CD228. Plates were evaluated by Attune™ NXT flow cytometer for live CD8 T cell proliferation and CD228+ tumor cells. FIG. 19 shows the proportion of CD8 T cells with diluted CFSE relative to wells without treatment added. FIG. 20 shows the percent of viable tumor cells remaining relative to wells without treatment added, reflecting tumor cell killing. These results show that in the presence of CD228, the fusion proteins (e.g., AAF30 (HC)), compared to antibodies, co-stimulate CD8+ T cell proliferation and killing of tumor cells in a dose dependent manner.

### Example 16: Pharmacokinetics of Fusion Proteins in Cynomolgus Monkeys

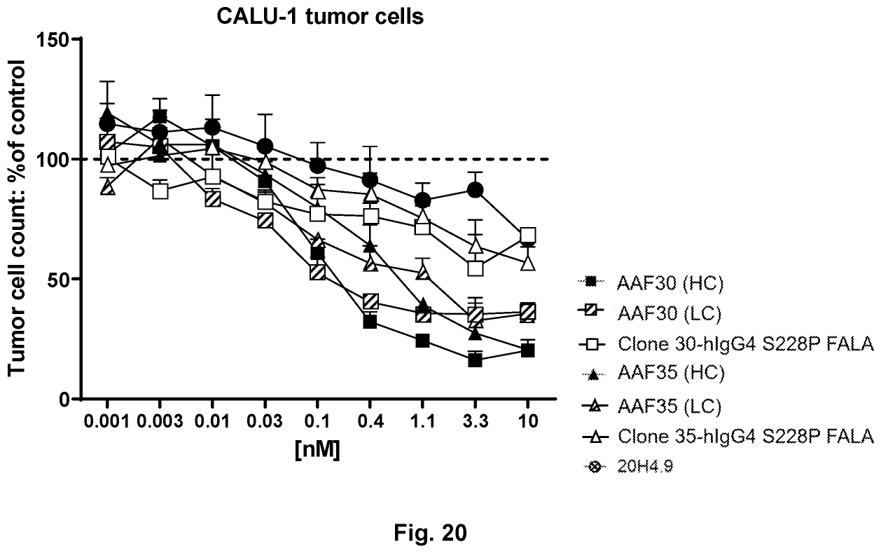

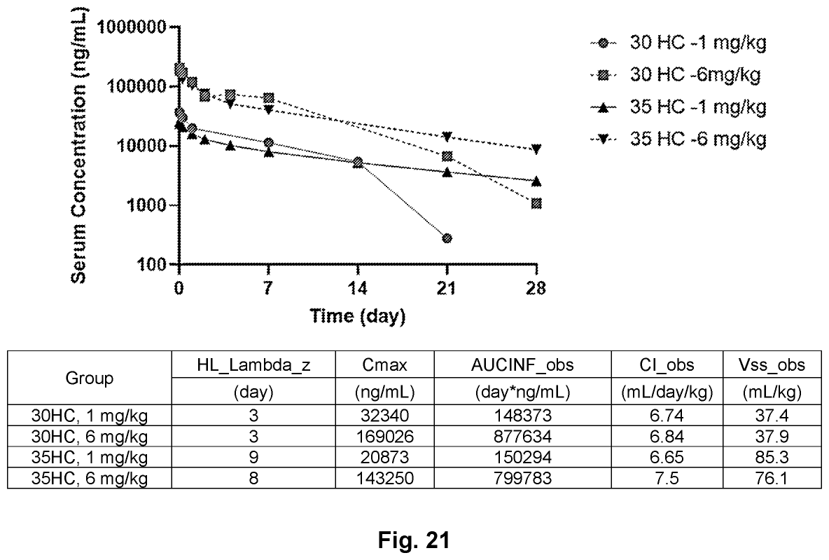

Heavy chain fusion protein bispecifics 30HC and 35HC (SEQ ID NOs: 80 and 76 and SEQ ID NOs: 82 and 79, respectively) were compared for pharmacokinetic differences in cynomolgus monkeys. Animals received a single dose of fusion bispecifics at a 1 mg/kg or 6 mg/kg doses. As shown in FIG. 21, bispecific fusion proteins exhibited a similar pharmacokinetic profile with slight differences after day 14, which may be due to an anti-drug antibody response.

### Example 17: Assessment of In Vivo Activity of Fusion Proteins in a Humanized Xenograft Model

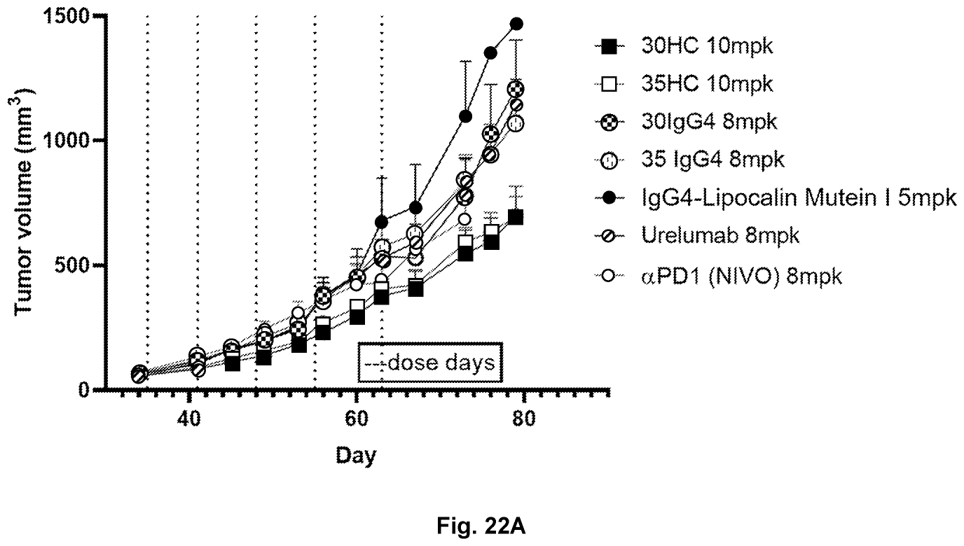

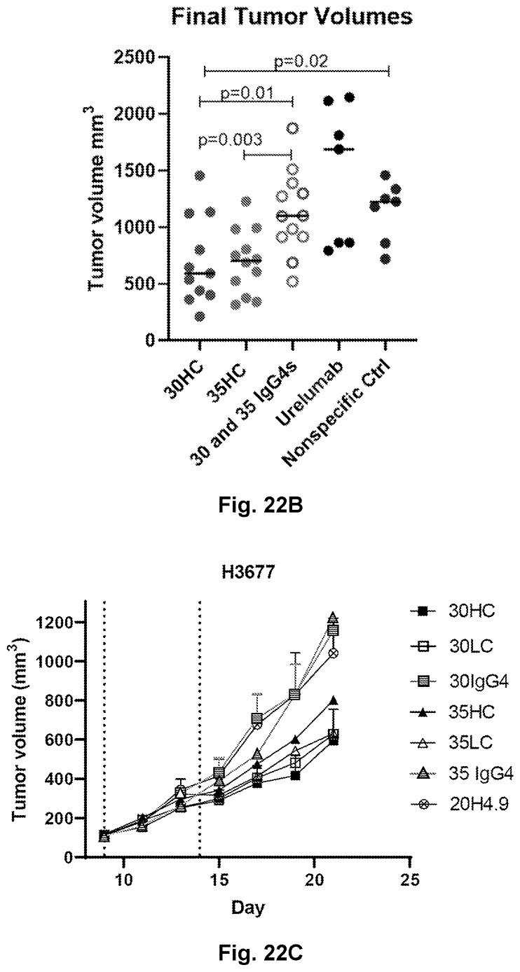

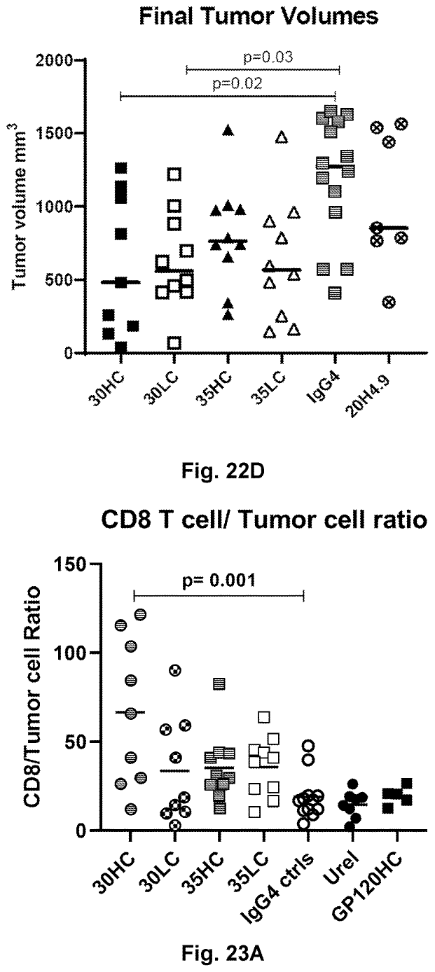

To assess the activity of bispecific fusion proteins in vivo, humanized xenograft models using CD228+ melanoma cell lines CALU-1 (ATCC) and H3677 (SGEN, internal) were performed. Tumor cells were implanted into immune-deficient NSG mice in 25% Matrigel® (Corning) and monitored until tumors reached an average volume of 100 mm³, at which point healthy donor peripheral blood mononuclear cells (PBMC) were adoptively transferred via tail vein injection to provide a source of human T cells. Mice were subsequently dosed at 5-day intervals with equimolar amounts of bispecific fusion proteins (10 mg/kg), antibodies (non-bispecific antibodies, anti-PD-1, and/or anti-4-1BB), or controls (8 mg/kg) and monitored for tumor growth. Growth curves across the Calu-1 study are shown in FIG. 22A, and final tumor volumes at the end study, Day 80, are shown in FIG. 22B. Growth curves across the H3677 study are shown in FIG. 22C, and final tumor volumes at the end study, Day 22, are shown in FIG. 22D. Statistics provided are the result of Tukey's analysis. These results show that the fusion proteins (e.g., AAF30 (HC)), compared to antibodies, reduce CD228+ tumor cell growth in vivo.

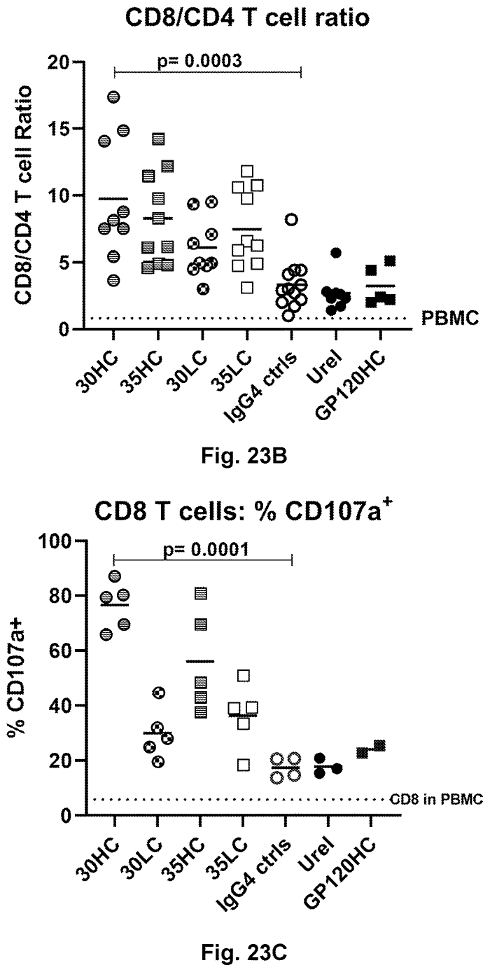

To evaluate intratumoral PD effects from bispecific-fusion-treated animals in the humanized CD228+ Calu-1 xenograft model, tumors were collected and processed into single cell suspensions for staining of tumor and immune cell populations and analysis by flow cytometry. FIG. 23A shows the CD8+ T cell to tumor cell ratio as determined by counts of CD8+ T cells divided by counts of CD228+ non-immune cells. FIG. 23B shows changes in CD8+/CD4+ T cell ratio. FIG. 23C shows the proportion of CD8+ T cells in active degranulation, as determined by surface expression of CD107a, at the end of the study. Dashed lines in figures indicate the CD8+/CD4+ T cell ratio (FIG. 23B) or the percentage of CD107a-expressing CD8 T cells (FIG. 23C) in resting PBMC (i.e., non-tumor model) samples from the same donor. These data demonstrate enhanced expansion and activation of cytotoxic CD8+ T cells upon treatment with bispecific fusion proteins that are superior to controls. Statistics provided are the result of Tukey's analysis.

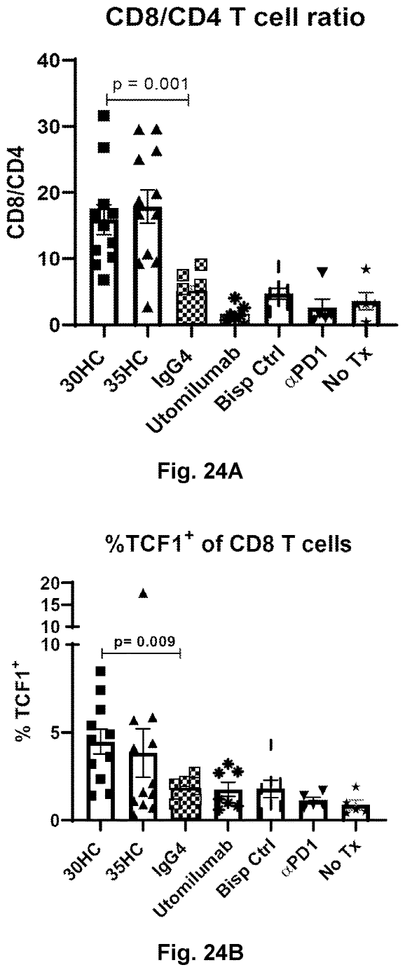

To evaluate intratumoral PD effects from bispecific-fusion-treated animals in a separate humanized CD228+ Calu-1 xenograft model, tumors were collected and processed into single cell suspensions for staining of tumor and immune cell populations and analysis by flow cytometry. FIG. 24A shows the CD8+ T cell to CD4+ T cell ratio. FIG. 24B shows changes in intracellular TCF1 expression. These data demonstrate enhanced expansion and differentiation of cytotoxic CD8+ T cells into cells with more stem-like properties, indicative of greater anti-tumor potential, as identified by expression of the transcription factor TCF1 following treatment with bispecific fusion proteins targeting 4-1BB. Statistics provided are the result of Tukey's analysis.

Embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, equivalents, modifications and variations thereof may be resorted to by those skilled in the art, and that such equivalents, modifications and variations are considered to be within the scope of this invention. All patents, patent applications, textbooks and (peer-reviewed) publications described herein are hereby incorporated by reference in their entirety to the same extent as if each individual publication, patent or patent application was specifically and individually indicated to be incorporated herein by reference. Furthermore, where a definition or use of a term in a reference, which is incorporated by reference herein is inconsistent or contrary to the definition of that term provided herein, the definition of that term provided herein applies and the definition of that term in the reference does not apply. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group. Further embodiments will become apparent from the following claims.

### VII. TABLE OF SEQUENCES

### Table of Exemplary Sequences

| SEQ ID | | |
|---|---|---|
| NO | Description | Sequence |
| 1 | Mature wt hTlc (without | HHLLASDEEIQDVSGTWYLKAMTVDREFPEMNLESVTPMTL |
| | signal sequence) | TTLEGGNLEAKVTMLISGRCQEVKAVLEKTDEPGKYTADGG |
| | | KHVAYIIRSHVKDHYIFYCEGELHGKPVRGVKLVGRDPKNN |
| | | LEALEDFEKAAGARGLSTESILIPRQSETCSPGSD |
| 2 | Mature wt hNGAL | QDSTSDLIPAPPLSKVPLQQNFQDNQFQGKWYVVGLAGNAI |
| | (without signal | LREDKDPQKMYATIYELKEDKSYNVTSVLERKKKCDYWIRT |
| | sequence) | FVPGCQPGEFTLGNIKSYPGLTSYLVRVVSTNYNQHAMVFF |
| | | KKVSQNREYFKITLYGRTKELTSELKENFIRFSKSLGLPEN |
| | | HIVFPVPIDQCIDG |
| 3 | Not Used | |
| 4 | Not Used | |
| 5 | Not Used | |
| 6 | human CD228 full | GMEVRWCATSDPEQHKCGNMSEAFREAGIQPSLLCVRGTSA |
| | length (as used in | DHCVQLIAAQEADAITLDGGAIYEAGKEHGLKPVVGEVYDQ |
| | examples) | EVGTSYYAVAVVRRSSHVTIDTLKGVKSCHTGINRTVGWNV |
| | | PVGYLVESGRLSVMGCDVLKAVSDYFGGSCVPGAGETSYSE |
| | | SLCRLCRGDSSGEGVCDKSPLERYYDYSGAFRCLAEGAGDV |
| | | AFVKHSTVLENTDGKTLPSWGQALLSQDFELLCRDGSRADV |
| | | TEWRQCHLARVPAHAVVVRADTDGGLIFRLLNEGQRLFSHE |
| | | GSSFQMFSSEAYGQKDLLFKDSTSELVPIATQTYEAWLGHE |
| | | YLHAMKGLLCDPNRLPPYLRWCVLSTPEIQKCGDMAVAFRR |
| | | QRLKPEIQCVSAKSPQHCMERIQAEQVDAVTLSGEDIYTAG |
| | | KTYGLVPAAGEHYAPEDSSNSYYVVAVVRRDSSHAFTLDEL |
| | | RGKRSCHAGFGSPAGWDVPVGALIQRGFIRPKDCDVLTAVS |
| | | EFFNASCVPVNNPKNYPSSLCALCVGDEQGRNKCVGNSQER |
| | | YYGYRGAFRCLVENAGDVAFVRHTTVEDNINGHNSEPWAAE |
| | | LRSEDYELLCPNGARAEVSQFAACNLAQIPPHAVMVRPDTN |
| | | IFTVYGLLDKAQDLFGDDHNKNGFKMFDSSNYHGQDLLFKD |
| | | ATVRAVPVGEKTTYRGWLGLDYVAALEGMSSQQCSGHHHHH |
| | | H |
| 7 | human CD137 full | LQDPCSNCPAGTFCDNNRNQICSPCPPNSESSAGGQRTCDI |
| | length (as used in | CRQCKGVERTRKECSSTSNAECDCTPGFHCLGAGCSMCEQD |
| | examples) | CKQGQELTKKGCKDCCFGTENDQKRGICRPWTNCSLDGKSV |
| | | LVNGTKERDVVCGPSPADLSPGASSVTPPAPAREPGHSPQI |
| | | ISFFLALTSTALLFLLFELTLRFSVVKRGRKKLLYIFKQPF |
| | | MRPVQTTQEEDGCSCRFPEEEEGGCEL |
| 8 | cyno CD228 (as used | GMEVRWCVTSDPEQQKCSNMSTAFREAGIQPSLLCVQGTSP |
| | in examples) | DHCIQLIAAQEADAITLDGGAIYEAGKEHGLKPVVGEVYDQ |
| | | EVGTSYYAVAVVKRSSQVTINTLKGVKSCHTGINRTVGWNV |
| | | PVGYLVESGRLSVMGCDVLKAVSDYEGGSCVPGAGETRYSE |
| | | SLCRLCRGDSSGEGVCDKSPLERYYDYSGAFRCLAEGAGDV |
| | | AFVKHSTVLENTDGKTLPSWGQALLSQDFELLCRDGSRADV |
| | | TEWRQCHLARVPAHAVVVRADTDGGLIFRLLNEGQRLFSHE |
| | | GSSFQMFSSKAYGQKDLLFKDSTSELVPIATQTYEAWLGQE |
| | | YLHAMKGLLCDPNRLPPYLRWCVLSTPEIQKCGDMAVAFGR |
| | | QQLKPEIQCVSAKSPQHCMEQIQAGQIDTVTLSGEDIYTAG |
| | | KTYGLAPAAGEGYASEDSSNSYFVVAVVRRDSSHAFTLDEL |
| | | RGKRSCHAGFGSPAGWDIPVGALIRRGFIRPKDCDVLTAVS |
| | | EFFNASCVPVNNPKNYPSSLCALCVGDEQGRNKCVGNSQER |
| | | YYGNSGAFRCLVENAGDVAFVRHTTVEDNINGHNSEPWAAE |
| | | LRSEDYELLCPNGARAEVSQFAACNLAQMPPHAVMVRPDTN |
| | | IFTVYGLLDKAQDLFGDDHNKNGFKMEDSSNYHGQDLLFKD |
| | | ATVRAVPVGEKTTYRDWLGLDYVAALEGMLSEQCSGGHHHH |
| | | HHHHHH |
| 9 | cyno CD137 (as used | LQDLCSNCPAGTFCDNNRSQICSPCPPNSESSAGGQRTCDI |
| | in examples) | CRQCKGVFKTRKECSSTSNAECDCISGYHCLGAECSMCEQD |
| | | CKQGQELTKKGCKDCCFGTENDQKRGICRPWTNCSLDGKSV |
| | | LVNGTKERDVVCGPSPADLSPGASSATPPAPAREPGHSPQI |
| | | IFFLALTSTVVLFLLFFLVLRESVVKRSRKKLLYIFKQPEM |
| | | RPVQTTQEEDGCSCRFPEEEEGGCEL |
| 10 | Not Used | |
| 11 | Not Used | |
| 12 | Not Used | |
| 13 | Not Used | |
| 14 | Not Used | |
| 15 | Not Used | |
| 16 | Not Used | |
| 17 | Not Used | |
| 18 | Not Used | |
| 19 | Not Used | |
| 20 | Not Used | |
| 21 | Not Used | |
| 22 | Not Used | |
| 23 | Not Used | |
| 24 | hIgG4 HC | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPSCPAPEFLGGPSVFLF |
| | | PPKPKDTLMISRTPEVTCVVVDVSQEDPEVQFNWYVDGVEV |
| | | HNAKTKPREEQFNSTYRVVSVLTVLHQDWLNGKEYKCKVSN |
| | | KGLPSSIEKTISKAKGQPREPQVYTLPPSQEEMTKNQVSLT |
| | | CLVKGFYPSDIAVEWESNGQPENNYKTTPPVLDSDGSFFLY |
| | | SRLTVDKSRWQEGNVESCSVMHEALHNHYTQKSLSLSLGK |
| 25 | hIgG4 LC | EIVLTQSPATLSLSPGERATLSCRASQSVSSYLAWYQQKPG |
| | | QAPRLLIYDASNRATGIPARFSGSGSGTDFTLTISSLEPED |
| | | FAVYYCQQSSNWPRTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 26 | Urelumab (20H4.9) HC | QVQLQQWGAGLLKPSETLSLTCAVYGGSFSGYYWSWIRQSP |
| | (as used in examples) | EKGLEWIGEINHGGYVTYNPSLESRVTISVDTSKNQFSLKL |
| | | SSVTAADTAVYYCARDYGPGNYDWYFDLWGRGTLVTVSSAS |
| | | TKGPSVFPLAPCSRSTSESTAALGCLVKDYFPEPVTVSWNS |
| | | GALTSGVHTFPAVLQSSGLYSLSSVVTVPSSSLGTKTYTCN |
| | | VDHKPSNTKVDKRVESKYGPPCPPCPAPEFLGGPSVFLFPP |
| | | KPKDTLMISRTPEVTCVVVDVSQEDPEVQFNWYVDGVEVHN |
| | | AKTKPREEQFNSTYRVVSVLTVLHQDWLNGKEYKCKVSNKG |
| | | LPSSIEKTISKAKGQPREPQVYTLPPSQEEMTKNQVSLTCL |
| | | VKGFYPSDIAVEWESNGQPENNYKTTPPVLDSDGSFFLYSR |
| | | LTVDKSRWQEGNVFSCSVMHEALHNHYTQKSLSLSLGK |
| 27 | Urelumab (20H4.9) LC | EIVLTQSPATLSLSPGERATLSCRASQSVSSYLAWYQQKPG |
| | (as used in examples) | QAPRLLIYDASNRATGIPARFSGSGSGTDFTLTISSLEPED |
| | | FAVYYCQQRSNWPPALTFGGGTKVEIKRTVAAPSVFIFPPS |
| | | DEQLKSGTASVVCLLNNFYPREAKVQWKVDNALQSGNSQES |
| | | VTEQDSKDSTYSLSSTLTLSKADYEKHKVYACEVTHQGLSS |
| | | PVTKSFNRGEC |
| 28 | Fc, IgG4v1 (used in | ESKYGPPCPPCPAPEAAGGPSVFLFPPKPKDTLMISRTPEV |
| | fusion proteins) | TCVVVDVSQEDPEVQFNWYVDGVEVHNAKTKPREEQENSTY |
| | | RVVSVLTVLHQDWLNGKEYKCKVSNKGLPSSIEKTISKAKG |
| | | QPREPQVYTLPPSQEEMTKNQVSLTCLVKGFYPSDIAVEWE |
| | | SNGQPENNYKTTPPVLDSDGSFFLYSRLTVDKSRWQEGNVE |
| | | SCSVMHEALHNHYTQKSLSLSLG |
| 29 | Fc, IgG4v2 | ESKYGPPCPPCPAPEFLGGPSVFLFPPKPKDTLMISRTPEV |
| | | TCVVVDVSQEDPEVQFNWYVDGVEVHNAKTKPREEQENSTY |
| | | RVVSVLTVLHQDWLNGKEYKCKVSNKGLPSSIEKTISKAKG |
| | | QPREPQVYTLPPSQEEMTKNQVSLTCLVKGFYPSDIAVEWE |
| | | SNGQPENNYKTTPPVLDSDGSFFLYSRLTVDKSRWQEGNVE |
| | | SCSVMHEALHNHYTQKSLSLSLG |
| 30 | Fc, IgG4v3 | ESKYGPPCPPCPAPEFLGGPSVFLFPPKPKDTLMISRTPEV |
| | | TCVVVDVSQEDPEVQFNWYVDGVEVHNAKTKPREEQENSTY |
| | | RVVSVLTVLHQDWLNGKEYKCKVSNKGLPSSIEKTISKAKG |
| | | QPREPQVYTLPPSQEEMTKNQVSLTCLVKGFYPSDIAVEWE |
| | | SNGQPENNYKTTPPVLDSDGSFFLYSRLTVDKSRWQEGNVE |
| | | SCSVLHEALHSHYTQKSLSLSLG |
| 31 | Fc, IgG4v4 | ESKYGPPCPPCPAPEFLGGPSVFLFPPKPKDTLYITREPEV |
| | | TCVVVDVSQEDPEVQFNWYVDGVEVHNAKTKPREEQENSTY |
| | | RVVSVLTVLHQDWLNGKEYKCKVSNKGLPSSIEKTISKAKG |
| | | QPREPQVYTLPPSQEEMTKNQVSLTCLVKGFYPSDIAVEWE |
| | | SNGQPENNYKTTPPVLDSDGSFFLYSRLTVDKSRWQEGNVE |
| | | SCSVMHEALHNHYTQKSLSLSLG |
| 32 | Not Used | |
| 33 | Not Used | |
| 34 | Not Used | |
| 35 | Not Used | |
| 36 | Not Used | |
| 37 | Not Used | |
| 38 | Not Used | |
| 39 | Not Used | |
| 40 | Lipocalin Mutein I | QDSTSDLIPAPPLSKVPLQQNFQDNQFHGKWYVVGQAGNIR |
| | | LREDKDPIKMMATIYELKEDKSYDVTMVKFDDKKCMYDIWT |
| | | FVPGSQPGEFTLGKIKSFPGHTSSLVRVVSTNYNQHAMVFF |
| | | KFVFQNREEFYITLYGRTKELTSELKENFIRFSKSLGLPEN |
| | | HIVEPVPIDQCIDG |
| 41 | Not Used | |
| 42 | Not Used | |
| 43 | Not Used | |
| 44 | Not Used | |
| 45 | Not Used | |
| 46 | Not Used | |
| 47 | Not Used | |
| 48 | Not Used | |
| 49 | Not Used | |
| 50 | Not Used | |
| 51 | Not Used | |
| 52 | Not Used | |
| 53 | Not Used | |
| 54 | Not Used | |
| 55 | Not Used | |
| 56 | Not Used | |
| 57 | Not Used | |
| 58 | OC04 HCDR1 | Same as SEQ ID NO: 213 |
| 59 | OC04 HCDR2 | Same as SEQ ID NO: 214 |
| 60 | OC04 HCDR3 | Same as SEQ ID NO: 215 |
| 61 | OC04 LCDR1 | Same as SEQ ID NO: 219 |
| 62 | OC04 LCDR2 | Same as SEQ ID NO: 220 |
| 63 | OC04 LCDR3 | Same as SEQ ID NO: 221 |
| 64 | OB02 HCDR1 | Same as SEQ ID NO: 253 |
| 65 | OB02 HCDR2 | Same as SEQ ID NO: 254 |
| 66 | OB02 HCDR3 | Same as SEQ ID NO: 255 |
| 67 | OB02 LCDR1 | Same as SEQ ID NO: 259 |
| 68 | OB02 LCDR2 | Same as SEQ ID NO: 260 |
| 69 | OB02 LCDR3 | Same as SEQ ID NO: 261 |
| 70 | VH OC04 | Same as SEQ ID NO: 222 |
| 71 | VL OC04 | Same as SEQ ID NO: 224 |
| 72 | VH OB02 | Same as SEQ ID NO: 262 |
| 73 | VL OB02 | Same as SEQ ID NO: 264 |
| 74 | OC04-hIgG1-HC | Same as SEQ ID NO: 226 |
| 75 | OC04-hIgG4v1-HC | QVQLVESGGGVVQPGRSLRLSCAASGFTFSSYGIHWVRQAP |
| | | GKGLEWVAVIWYDGSNKYYADSVKGRFTISRDNSKNTLYLQ |
| | | MNSLRAEDTAVYYCARRGDSSSWHFDYWGQGTLVTVSSAST |
| | | KGPSVFPLAPCSRSTSESTAALGCLVKDYFPEPVTVSWNSG |
| | | ALTSGVHTFPAVLQSSGLYSLSSVVTVPSSSLGTKTYTCNV |
| | | DHKPSNTKVDKRVESKYGPPCPPCPAPEAAGGPSVFLFPPK |
| | | PKDTLMISRTPEVTCVVVDVSQEDPEVQFNWYVDGVEVHNA |
| | | KTKPREEQFNSTYRVVSVLTVLHQDWLNGKEYKCKVSNKGL |
| | | PSSIEKTISKAKGQPREPQVYTLPPSQEEMTKNQVSLTCLV |
| | | KGFYPSDIAVEWESNGQPENNYKTTPPVLDSDGSFFLYSRL |
| | | TVDKSRWQEGNVESCSVMHEALHNHYTQKSLSLSLG |
| 76 | OC04-LC-lambda | QSALTQPASVSGSPGQSITISCTGTSSDVGRHNLVSWYQQH |
| | | PGKVPKVMIYEVTKRPAGVSNRFSGSKSGNTASLTISGLQA |
| | | EDEADYYCCSYAGSSTYVFGTGTRVTVLGQPKAAPSVTLFP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS |
| 77 | OB02-hIgG1-HC | Same as SEQ ID NO: 266 |
| 78 | OB02-hIgG4v1-HC | QVQLQQSGPGLVKPSQTLSLTCAISGDSVSSNSAAWNWIRQ |
| | | SPSSGLEWLGRTYYRSKWFDDYAVSVKGRITINPDTSKNQF |
| | | SLQLKEVIPEDTAVYYCARRVHWGGPFDYWGQGTLVTVSSA |
| | | STKGPSVFPLAPCSRSTSESTAALGCLVKDYFPEPVTVSWN |
| | | SGALTSGVHTFPAVLQSSGLYSLSSVVTVPSSSLGTKTYTC |
| | | NVDHKPSNTKVDKRVESKYGPPCPPCPAPEAAGGPSVFLFP |
| | | PKPKDTLMISRTPEVTCVVVDVSQEDPEVQFNWYVDGVEVH |
| | | NAKTKPREEQFNSTYRVVSVLTVLHQDWLNGKEYKCKVSNK |
| | | GLPSSIEKTISKAKGQPREPQVYTLPPSQEEMTKNQVSLTC |
| | | LVKGFYPSDIAVEWESNGQPENNYKTTPPVLDSDGSFFLYS |
| | | RLTVDKSRWQEGNVFSCSVMHEALHNHYTQKSLSLSLG |
| 79 | OB02-LC-kappa | DIQLTQSPSFLSASVGDRVTITCRASQGISSYLAWYQQRPG |
| | | KAPKVLISVASTLQSGVPSRFSGSGSGTDFTLTISSLQPED |
| | | FATYYCQQLTGYPFTFGPGTKVDIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 80 | OC04-hIgG4v1-HC-L1- | QVQLVESGGGVVQPGRSLRLSCAASGFTFSSYGIHWVRQAP |
| | Lipocalin Mutein I | GKGLEWVAVIWYDGSNKYYADSVKGRFTISRDNSKNTLYLQ |
| | | MNSLRAEDTAVYYCARRGDSSSWHFDYWGQGTLVTVSSAST |
| | | KGPSVFPLAPCSRSTSESTAALGCLVKDYFPEPVTVSWNSG |
| | | ALTSGVHTFPAVLQSSGLYSLSSVVTVPSSSLGTKTYTCNV |
| | | DHKPSNTKVDKRVESKYGPPCPPCPAPEAAGGPSVFLFPPK |
| | | PKDTLMISRTPEVTCVVVDVSQEDPEVQFNWYVDGVEVHNA |
| | | KTKPREEQFNSTYRVVSVLTVLHQDWLNGKEYKCKVSNKGL |
| | | PSSIEKTISKAKGQPREPQVYTLPPSQEEMTKNQVSLTCLV |
| | | KGFYPSDIAVEWESNGQPENNYKTTPPVLDSDGSFFLYSRL |
| | | TVDKSRWQEGNVFSCSVMHEALHNHYTQKSLSLSLGGGGGS |
| | | GGGGSGGGGSQDSTSDLIPAPPLSKVPLQQNFQDNQFHGKW |
| | | YVVGQAGNIRLREDKDPIKMMATIYELKEDKSYDVTMVKED |
| | | DKKCMYDIWTFVPGSQPGEFTLGKIKSFPGHTSSLVRVVST |
| | | NYNQHAMVFFKFVFQNREEFYITLYGRTKELTSELKENFIR |
| | | FSKSLGLPENHIVFPVPIDQCIDG |
| 81 | OC04-LC-lambda-L1- | QSALTQPASVSGSPGQSITISCTGTSSDVGRHNLVSWYQQH |
| | Lipocalin Mutein I | PGKVPKVMIYEVTKRPAGVSNRFSGSKSGNTASLTISGLQA |
| | | EDEADYYCCSYAGSSTYVFGTGTRVTVLGQPKAAPSVTLEP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECSGGGGSGGGGSGGGGSQDSTSDLIPAPPLSK |
| | | VPLQQNFQDNQFHGKWYVVGQAGNIRLREDKDPIKMMATIY |
| | | ELKEDKSYDVTMVKEDDKKCMYDIWTFVPGSQPGEFTLGKI |
| | | KSFPGHTSSLVRVVSTNYNQHAMVFFKFVFQNREEFYITLY |
| | | GRTKELTSELKENFIRFSKSLGLPENHIVFPVPIDQCIDG |
| 82 | OB02-hIgG4v1-HC-L1- | QVQLQQSGPGLVKPSQTLSLTCAISGDSVSSNSAAWNWIRQ |
| | Lipocalin Mutein I | SPSSGLEWLGRTYYRSKWFDDYAVSVKGRITINPDTSKNQF |
| | | SLQLKFVIPEDTAVYYCARRVHWGGPFDYWGQGTLVTVSSA |
| | | STKGPSVFPLAPCSRSTSESTAALGCLVKDYFPEPVTVSWN |
| | | SGALTSGVHTFPAVLQSSGLYSLSSVVTVPSSSLGTKTYTC |
| | | NVDHKPSNTKVDKRVESKYGPPCPPCPAPEAAGGPSVFLFP |
| | | PKPKDTLMISRTPEVTCVVVDVSQEDPEVQFNWYVDGVEVH |
| | | NAKTKPREEQFNSTYRVVSVLTVLHQDWLNGKEYKCKVSNK |
| | | GLPSSIEKTISKAKGQPREPQVYTLPPSQEEMTKNQVSLTC |
| | | LVKGFYPSDIAVEWESNGQPENNYKTTPPVLDSDGSFFLYS |
| | | RLTVDKSRWQEGNVFSCSVMHEALHNHYTQKSLSLSLGGGG |
| | | GSGGGGSGGGGSQDSTSDLIPAPPLSKVPLQQNFQDNQFHG |
| | | KWYVVGQAGNIRLREDKDPIKMMATIYELKEDKSYDVTMVK |
| | | FDDKKCMYDIWTFVPGSQPGEFTLGKIKSFPGHTSSLVRVV |
| | | STNYNQHAMVFFKFVFQNREEFYITLYGRTKELTSELKENF |
| | | IRFSKSLGLPENHIVFPVPIDQCIDG |
| 83 | OB02-LC-kappa-L1- | DIQLTQSPSELSASVGDRVTITCRASQGISSYLAWYQQRPG |
| | Lipocalin Mutein I | KAPKVLISVASTLQSGVPSRFSGSGSGTDFTLTISSLQPED |
| | | FATYYCQQLTGYPFTFGPGTKVDIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGECGGGGsGGGGSGGGGSQDSTSDLIPAPPLSKVP |
| | | LQQNFQDNQFHGKWYVVGQAGNIRLREDKDPIKMMATIYEL |
| | | KEDKSYDVTMVKEDDKKCMYDIWTFVPGSQPGEFTLGKIKS |
| | | FPGHTSSLVRVVSTNYNQHAMVFFKFVFQNREEFYITLYGR |
| | | TKELTSELKENFIRFSKSLGLPENHIVFPVPIDQCIDG |
| 84 | IgG4-Lipocalin Mutein I | ESKYGPPCPPCPAPEAAGGPSVELFPPKPKDTLMISRTPEV |
| | S228P FALA | TCVVVDVSQEDPEVQFNWYVDGVEVHNAKTKPREEQENSTY |
| | | RVVSVLTVLHQDWLNGKEYKCKVSNKGLPSSIEKTISKAKG |
| | | QPREPQVYTLPPSQEEMTKNQVSLTCLVKGFYPSDIAVEWE |
| | | SNGQPENNYKTTPPVLDSDGSFFLYSRLTVDKSRWQEGNVE |
| | | SCSVMHEALHNHYTQKSLSLSLGKGGGGSGGGGSGGGGSQD |
| | | STSDLIPAPPLSKVPLQQNFQDNQFHGKWYVVGQAGNIRLR |
| | | EDKDPIKMMATIYELKEDKSYDVTMVKEDDKKCMYDIWTFV |
| | | PGSQPGEFTLGKIKSFPGHTSSLVRVVSTNYNQHAMVFFKE |
| | | VFQNREEFYITLYGRTKELTSELKENFIRFSKSLGLPENHI |
| | | VFPVPIDQCIDG |
| 85 | Nivolumab HC (as | QVQLVESGGGVVQPGRSLRLDCKASGITFSNSGMHWVRQAP |
| | used in examples) | GKGLEWVAVIWYDGSKRYYADSVKGRFTISRDNSKNTLFLQ |
| | | MNSLRAEDTAVYYCATNDDYWGQGTLVTVSSASTKGPSVFP |
| | | LAPCSRSTSESTAALGCLVKDYFPEPVTVSWNSGALTSGVH |
| | | TFPAVLQSSGLYSLSSVVTVPSSSLGTKTYTCNVDHKPSNT |
| | | KVDKRVESKYGPPCPPCPAPEFLGGPSVFLFPPKPKDTLMI |
| | | SRTPEVTCVVVDVSQEDPEVQFNWYVDGVEVHNAKTKPREE |
| | | QFNSTYRVVSVLTVLHQDWLNGKEYKCKVSNKGLPSSIEKT |
| | | ISKAKGQPREPQVYTLPPSQEEMTKNQVSLTCLVKGFYPSD |
| | | IAVEWESNGQPENNYKTTPPVLDSDGSFFLYSRLTVDKSRW |
| | | QEGNVFSCSVMHEALHNHYTQKSLSLSLGK |
| 86 | Nivolumab LC (as used | EIVLTQSPATLSLSPGERATLSCRASQSVSSYLAWYQQKPG |
| | in examples) | QAPRLLIYDASNRATGIPARFSGSGSGTDFTLTISSLEPED |
| | | FAVYYCQQSSNWPRTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 87 | OC04-hIgG4v1-HC | CAGGTGCAGCTGGTTGAATCTGGTGGCGGAGTGGTGCAGCC |
| | (nucleotide) | TGGCAGATCTCTGAGACTGTCTTGTGCCGCCTCCGGCTTCA |
| | | CCTTCTCCTCTTACGGAATCCACTGGGTCCGACAGGCCCCT |
| | | GGCAAAGGATTGGAATGGGTCGCCGTGATTTGGTACGACGG |
| | | CTCCAACAAGTACTACGCCGACTCCGTGAAGGGCAGATTCA |
| | | CCATCTCTCGGGACAACTCCAAGAACACCCTGTACCTGCAG |
| | | ATGAACTCCCTGAGAGCCGAGGACACCGCCGTGTACTACTG |
| | | TGCCAGAAGAGGCGACTCCTCCTCCTGGCACTTTGACTATT |
| | | GGGGCCAGGGCACCCTGGTCACCGTTAGTTCTGCTAGCACC |
| | | AAAGGGCCGTCCGTCTTCCCCCTGGCCCCCTGCAGCCGGTC |
| | | GACGTCCGAGTCCACCGCCGCCCTCGGGTGCCTGGTCAAGG |
| | | ACTACTTCCCGGAGCCGGTAACCGTGAGCTGGAACTCCGGC |
| | | GCGCTGACCTCCGGCGTGCACACGTTCCCCGCCGTCCTGCA |
| | | GTCCTCCGGCCTCTACTCCCTCTCGTCCGTCGTCACCGTCC |
| | | CGAGCAGTTCCCTGGGCACCAAGACCTACACGTGCAACGTC |
| | | GACCACAAGCCCTCCAACACCAAGGTAGACAAGCGCGTCGA |
| | | GTCCAAATACGGCCCCCCGTGCCCGCCCTGTCCGGCCCCCG |
| | | AGGCCGCGGGCGGCCCCTCAGTGTTCCTGTTCCCGCCGAAG |
| | | CCCAAGGACACCCTGATGATCTCGCGCACGCCCGAGGTCAC |
| | | GTGCGTGGTCGTCGACGTCTCACAGGAAGACCCCGAGGTGC |
| | | AGTTCAACTGGTATGTCGACGGCGTGGAGGTTCACAACGCG |
| | | AAGACCAAGCCCCGGGAGGAGCAGTTCAACAGCACATACCG |
| | | GGTGGTGTCGGTCCTCACCGTGCTGCATCAGGACTGGCTGA |
| | | ACGGTAAGGAGTACAAGTGCAAGGTGTCCAACAAGGGCCTC |
| | | CCGAGCAGCATAGAGAAGACCATCTCCAAGGCGAAGGGTCA |
| | | GCCCCGCGAACCGCAGGTGTACACCCTGCCGCCGAGCCAGG |
| | | AGGAGATGACGAAGAACCAGGTCTCCCTGACGTGCCTGGTG |
| | | AAGGGTTTCTACCCCTCGGACATCGCGGTCGAATGGGAATC |
| | | GAACGGGCAGCCGGAGAACAACTACAAGACCACGCCGCCGG |
| | | TCCTGGACTCCGACGGGTCCTTCTTCCTGTACTCCCGGCTG |
| | | ACCGTAGACAAGTCGCGCTGGCAGGAGGGTAACGTGTTCAG |
| | | CTGCAGCGTGATGCACGAGGCCCTCCACAACCACTACACGC |
| | | AAAAGTCGCTCTCCCTGTCCCTGGGC |
| 88 | OC04-LC-lambda | CAGTCTGCTCTGACCCAGCCTGCTTCTGTGTCTGGCTCTCC |
| | (nucleotide) | CGGCCAGTCCATCACCATCTCTTGTACCGGCACCTCCTCCG |
| | | ACGTGGGCAGACACAATCTGGTGTCCTGGTATCAGCAGCAT |
| | | CCCGGCAAGGTGCCCAAAGTGATGATCTACGAAGTGACCAA |
| | | GCGGCCTGCCGGCGTGTCCAACAGATTCTCCGGATCCAAGT |
| | | CCGGCAACACCGCCAGCCTGACAATTAGCGGACTGCAGGCT |
| | | GAGGACGAGGCCGACTACTACTGCTGTTCTTACGCCGGCTC |
| | | CTCCACCTACGTGTTCGGCACTGGAACAAGAGTGACAGTGC |
| | | TGGGCCAGCCCAAGGCCGCCCCTTCTGTTACTCTGTTCCCT |
| | | CCATCCTCCGAGGAACTGCAGGCCAACAAGGCTACCCTCGT |
| | | GTGCCTGATCTCCGACTTTTACCCTGGCGCTGTGACCGTGG |
| | | CCTGGAAGGCTGATAGTTCTCCTGTGAAGGCCGGCGTGGAA |
| | | ACCACCACACCTTCCAAGCAGTCCAACAACAAATACGCCGC |
| | | CTCCTCCTACCTGTCTCTGACCCCTGAACAGTGGAAGTCCC |
| | | ACCGGTCCTACAGCTGCCAAGTGACCCATGAGGGCTCCACC |
| | | GTGGAAAAGACCGTGGCTCCTACCGAGTGCTCT |
| 89 | OB02-hIgG4v1-HC | CAGGTTCAGCTGCAGCAATCTGGACCTGGCCTGGTCAAGCC |
| | (nucleotide) | CTCTCAGACCCTGTCTCTGACCTGTGCCATCTCCGGCGACT |
| | | CCGTGTCCTCTAATTCTGCCGCCTGGAACTGGATCCGGCAG |
| | | TCTCCATCTTCTGGCCTGGAATGGCTGGGCAGAACCTACTA |
| | | CCGGTCCAAGTGGTTCGACGACTACGCCGTGTCTGTGAAGG |
| | | GCAGAATCACCATCAATCCCGACACCTCCAAGAACCAGTTC |
| | | TCCCTGCAGCTCAAGTTCGTGATCCCTGAGGACACCGCCGT |
| | | GTACTACTGCGCCAGAAGAGTGCATTGGGGCGGACCCTTTG |
| | | ATTACTGGGGCCAGGGAACACTGGTCACCGTGTCATCTGCT |
| | | AGCACCAAAGGGCCGTCCGTCTTCCCCCTGGCCCCCTGCAG |
| | | CCGGTCGACGTCCGAGTCCACCGCCGCCCTCGGGTGCCTGG |
| | | TCAAGGACTACTTCCCGGAGCCGGTAACCGTGAGCTGGAAC |
| | | TCCGGCGCGCTGACCTCCGGCGTGCACACGTTCCCCGCCGT |
| | | CCTGCAGTCCTCCGGCCTCTACTCCCTCTCGTCCGTCGTCA |
| | | CCGTCCCGAGCAGTTCCCTGGGCACCAAGACCTACACGTGC |
| | | AACGTCGACCACAAGCCCTCCAACACCAAGGTAGACAAGCG |
| | | CGTCGAGTCCAAATACGGCCCCCCGTGCCCGCCCTGTCCGG |
| | | CCCCCGAGGCCGCGGGCGGCCCCTCAGTGTTCCTGTTCCCG |
| | | CCGAAGCCCAAGGACACCCTGATGATCTCGCGCACGCCCGA |
| | | GGTCACGTGCGTGGTCGTCGACGTCTCACAGGAAGACCCCG |
| | | AGGTGCAGTTCAACTGGTATGTCGACGGCGTGGAGGTTCAC |
| | | AACGCGAAGACCAAGCCCCGGGAGGAGCAGTTCAACAGCAC |
| | | ATACCGGGTGGTGTCGGTCCTCACCGTGCTGCATCAGGACT |
| | | GGCTGAACGGTAAGGAGTACAAGTGCAAGGTGTCCAACAAG |
| | | GGCCTCCCGAGCAGCATAGAGAAGACCATCTCCAAGGCGAA |
| | | GGGTCAGCCCCGCGAACCGCAGGTGTACACCCTGCCGCCGA |
| | | GCCAGGAGGAGATGACGAAGAACCAGGTCTCCCTGACGTGC |
| | | CTGGTGAAGGGTTTCTACCCCTCGGACATCGCGGTCGAATG |
| | | GGAATCGAACGGGCAGCCGGAGAACAACTACAAGACCACGC |
| | | CGCCGGTCCTGGACTCCGACGGGTCCTTCTTCCTGTACTCC |
| | | CGGCTGACCGTAGACAAGTCGCGCTGGCAGGAGGGTAACGT |
| | | GTTCAGCTGCAGCGTGATGCACGAGGCCCTCCACAACCACT |
| | | ACACGCAAAAGTCGCTCTCCCTGTCCCTGGGC |
| 90 | OB02-LC-kappa | GACATCCAGCTGACCCAGTCTCCATCTTTCCTGTCCGCCTC |
| | (nucleotide) | TGTGGGCGACAGAGTGACCATCACCTGTAGAGCCTCTCAGG |
| | | GCATCTCCTCCTACCTGGCCTGGTATCAGCAGAGGCCTGGC |
| | | AAGGCTCCTAAGGTGCTGATCTCCGTGGCTAGCACACTGCA |
| | | GTCTGGCGTGCCCTCTAGATTCTCCGGCTCTGGCTCTGGCA |
| | | CCGACTTTACCCTGACAATCTCCAGCCTGCAGCCTGAGGAC |
| | | TTCGCCACCTACTACTGCCAGCAGCTGACCGGCTATCCCTT |
| | | CACCTTTGGACCTGGCACCAAGGTGGACATCAAGCGGACAG |
| | | TGGCCGCTCCTTCCGTGTTCATCTTCCCACCTTCCGACGAG |
| | | CAGCTGAAGTCCGGCACAGCTTCTGTCGTGTGCCTGCTGAA |
| | | CAACTTCTACCCTCGGGAAGCCAAGGTGCAGTGGAAGGTGG |
| | | ACAATGCCCTGCAGTCCGGCAACTCCCAAGAGTCTGTGACC |
| | | GAGCAGGACTCCAAGGACAGCACCTACAGCCTGTCCTCCAC |
| | | ACTGACCCTGTCCAAGGCCGACTACGAGAAGCACAAGGTGT |
| | | ACGCCTGCGAAGTGACCCATCAGGGCCTGTCTAGCCCTGTG |
| | | ACCAAGTCTTTCAACCGGGGCGAGTGT |
| 96 | EB03-hIgG4v1-HC-L1- | QVQLVQSGSELKKPGASVKVSCKASGYTFTTYVMNWVRQAP |
| | Lipocalin Mutein I | GQGLEWMGWINTNTGNPTYAQGFTGRFVFSVDTSVSTAYLQ |
| | | ISSLKAEDTAVYYCARDRVLLWFGAGYYHYGMDVWGQGTTV |
| | | TVSSASTKGPSVFPLAPCSRSTSESTAALGCLVKDYFPEPV |
| | | TVSWNSGALTSGVHTFPAVLQSSGLYSLSSVVTVPSSSLGT |
| | | KTYTCNVDHKPSNTKVDKRVESKYGPPCPPCPAPEAAGGPS |
| | | VFLFPPKPKDTLMISRTPEVTCVVVDVSQEDPEVQFNWYVD |
| | | GVEVHNAKTKPREEQFNSTYRVVSVLTVLHQDWLNGKEYKC |
| | | KVSNKGLPSSIEKTISKAKGQPREPQVYTLPPSQEEMTKNQ |
| | | VSLTCLVKGFYPSDIAVEWESNGQPENNYKTTPPVLDSDGS |
| | | FFLYSRLTVDKSRWQEGNVESCSVMHEALHNHYTQKSLSLS |
| | | LGGGGGSGGGGSGGGGSQDSTSDLIPAPPLSKVPLQQNFQD |
| | | NQFHGKWYVVGQAGNIRLREDKDPIKMMATIYELKEDKSYD |
| | | VTMVKFDDKKCMYDIWTFVPGSQPGEFTLGKIKSFPGHTSS |
| | | LVRVVSTNYNQHAMVFFKFVFQNREEFYITLYGRTKELTSE |
| | | LKENFIRFSKSLGLPENHIVFPVPIDQCIDG |
| 97 | EB03-LC-lambda | SSELTQDPAVSVALGQTVRITCQGDSLRSYYTSWYQQKPGQ |
| | | APVFVIYGKNNRPSGIPDRFSGSSSGNTASLTITGAQAEDE |
| | | ADYYCNSRDSSGNHVVFGGGTKLTVLGQPKAAPSVTLEPPS |
| | | SEELQANKATLVCLISDFYPGAVTVAWKADSSPVKAGVETT |
| | | TPSKQSNNKYAASSYLSLTPEQWKSHRSYSCQVTHEGSTVE |
| | | KTVAPTECS |
| 98 | EB03-hIgG4v1-HC | QVQLVQSGSELKKPGASVKVSCKASGYTFTTYVMNWVRQAP |
| | | GQGLEWMGWINTNTGNPTYAQGFTGRFVFSVDTSVSTAYLQ |
| | | ISSLKAEDTAVYYCARDRVLLWFGAGYYHYGMDVWGQGTTV |
| | | TVSSASTKGPSVFPLAPCSRSTSESTAALGCLVKDYFPEPV |
| | | TVSWNSGALTSGVHTFPAVLQSSGLYSLSSVVTVPSSSLGT |
| | | KTYTCNVDHKPSNTKVDKRVESKYGPPCPPCPAPEAAGGPS |
| | | VFLFPPKPKDTLMISRTPEVTCVVVDVSQEDPEVQFNWYVD |
| | | GVEVHNAKTKPREEQFNSTYRVVSVLTVLHQDWLNGKEYKC |
| | | KVSNKGLPSSIEKTISKAKGQPREPQVYTLPPSQEEMTKNQ |
| | | VSLTCLVKGFYPSDIAVEWESNGQPENNYKTTPPVLDSDGS |
| | | FFLYSRLTVDKSRWQEGNVFSCSVMHEALHNHYTQKSLSLS |
| | | LG |
| 99 | EB03-LC-lambda-L1- | SSELTQDPAVSVALGQTVRITCQGDSLRSYYTSWYQQKPGQ |
| | Lipocalin Mutein I | APVFVIYGKNNRPSGIPDRFSGSSSGNTASLTITGAQAEDE |
| | | ADYYCNSRDSSGNHVVFGGGTKLTVLGQPKAAPSVTLEPPS |
| | | SEELQANKATLVCLISDFYPGAVTVAWKADSSPVKAGVETT |
| | | TPSKQSNNKYAASSYLSLTPEQWKSHRSYSCQVTHEGSTVE |
| | | KTVAPTECSGGGGSGGGGSGGGGSQDSTSDLIPAPPLSKVP |
| | | LQQNFQDNQFHGKWYVVGQAGNIRLREDKDPIKMMATIYEL |
| | | KEDKSYDVTMVKFDDKKCMYDIWTFVPGSQPGEFTLGKIKS |
| | | FPGHTSSLVRVVSTNYNQHAMVFFKFVFQNREEFYITLYGR |
| | | TKELTSELKENFIRFSKSLGLPENHIVEPVPIDQCIDG |
| 100 | EE03-hIgG4v1-HC-L1- | QVQLQESGPGLVKPSQTLSLTCTVSGGSISSGGYYWSWIRQ |
| | Lipocalin Mutein I | HPGKGLEWIGYIHYIGSTYQNPSLKSRVTMSVDTSKNQFSL |
| | | RLTSVTAADTAVYYCARESGDTAMVFDYWGQGTQVTVSSAS |
| | | TKGPSVFPLAPCSRSTSESTAALGCLVKDYFPEPVTVSWNS |
| | | GALTSGVHTFPAVLQSSGLYSLSSVVTVPSSSLGTKTYTCN |
| | | VDHKPSNTKVDKRVESKYGPPCPPCPAPEAAGGPSVFLFPP |
| | | KPKDTLMISRTPEVTCVVVDVSQEDPEVQFNWYVDGVEVHN |
| | | AKTKPREEQFNSTYRVVSVLTVLHQDWLNGKEYKCKVSNKG |
| | | LPSSIEKTISKAKGQPREPQVYTLPPSQEEMTKNQVSLTCL |
| | | VKGFYPSDIAVEWESNGQPENNYKTTPPVLDSDGSFFLYSR |
| | | LTVDKSRWQEGNVFSCSVMHEALHNHYTQKSLSLSLGGGGG |
| | | SGGGGSGGGGSQDSTSDLIPAPPLSKVPLQQNFQDNQFHGK |
| | | WYVVGQAGNIRLREDKDPIKMMATIYELKEDKSYDVTMVKE |
| | | DDKKCMYDIWTFVPGSQPGEFTLGKIKSFPGHTSSLVRVVS |
| | | TNYNQHAMVFFKFVFQNREEFYITLYGRTKELTSELKENFI |
| | | RFSKSLGLPENHIVEPVPIDQCIDG |
| 101 | EE03-LC-lambda | QSALTQPPSASGSPGQSVTISCTGTSSDVGGYNYVSWYQQH |
| | | PGKAPKVMIYEVSTRPSGVPDRFSGSKSGNTASLTVSGLQA |
| | | EDEADYYCSSYAGNNNFVFGTGTKVTVLGQPKAAPSVTLFP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS |
| 102 | EE03-hIgG4v1-HC | QVQLQESGPGLVKPSQTLSLTCTVSGGSISSGGYYWSWIRQ |
| | | HPGKGLEWIGYIHYIGSTYQNPSLKSRVTMSVDTSKNQFSL |
| | | RLTSVTAADTAVYYCARESGDTAMVFDYWGQGTQVTVSSAS |
| | | TKGPSVFPLAPCSRSTSESTAALGCLVKDYFPEPVTVSWNS |
| | | GALTSGVHTFPAVLQSSGLYSLSSVVTVPSSSLGTKTYTCN |
| | | VDHKPSNTKVDKRVESKYGPPCPPCPAPEAAGGPSVFLFPP |
| | | KPKDTLMISRTPEVTCVVVDVSQEDPEVQFNWYVDGVEVHN |
| | | AKTKPREEQFNSTYRVVSVLTVLHQDWLNGKEYKCKVSNKG |
| | | LPSSIEKTISKAKGQPREPQVYTLPPSQEEMTKNQVSLTCL |
| | | VKGFYPSDIAVEWESNGQPENNYKTTPPVLDSDGSFFLYSR |
| | | LTVDKSRWQEGNVESCSVMHEALHNHYTQKSLSLSLG |
| 103 | EE03-LC-lambda-L1- | QSALTQPPSASGSPGQSVTISCTGTSSDVGGYNYVSWYQQH |
| | Lipocalin Mutein I | PGKAPKVMIYEVSTRPSGVPDRFSGSKSGNTASLTVSGLQA |
| | | EDEADYYCSSYAGNNNFVFGTGTKVTVLGQPKAAPSVTLEP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECSGGGGSGGGGSGGGGSQDSTSDLIPAPPLSK |
| | | VPLQQNFQDNQFHGKWYVVGQAGNIRLREDKDPIKMMATIY |
| | | ELKEDKSYDVTMVKFDDKKCMYDIWTFVPGSQPGEFTLGKI |
| | | KSFPGHTSSLVRVVSTNYNQHAMVFFKFVFQNREEFYITLY |
| | | GRTKELTSELKENFIRESKSLGLPENHIVEPVPIDQCIDG |
| 104 | OE12-hIgG4v1-HC-L1- | QVQLVQSGAEVKKPGASVKVSCKASGYTFTNYDINWVRQAT |
| | Lipocalin Mutein I | GQGLEWMGWMNPNSGNTDYVQKFQGRVAMTRITSISTAYLE |
| | | LSSLRSDDTAVYYCARGLHSGDYYSYGMDVWGQGTTVTVSS |
| | | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEAAGGPSVFLF |
| | | PPKPKDTLMISRTPEVTCVVVDVSQEDPEVQFNWYVDGVEV |
| | | HNAKTKPREEQFNSTYRVVSVLTVLHQDWLNGKEYKCKVSN |
| | | KGLPSSIEKTISKAKGQPREPQVYTLPPSQEEMTKNQVSLT |
| | | CLVKGFYPSDIAVEWESNGQPENNYKTTPPVLDSDGSFFLY |
| | | SRLTVDKSRWQEGNVFSCSVMHEALHNHYTQKSLSLSLGGG |
| | | GGSGGGGSGGGGSQDSTSDLIPAPPLSKVPLQQNFQDNQFH |
| | | GKWYVVGQAGNIRLREDKDPIKMMATIYELKEDKSYDVTMV |
| | | KFDDKKCMYDIWTFVPGSQPGEFTLGKIKSFPGHTSSLVRV |
| | | VSTNYNQHAMVFFKFVFQNREEFYITLYGRTKELTSELKEN |
| | | FIRFSKSLGLPENHIVEPVPIDQCIDG |
| 105 | OE12-LC-lambda | SYELTQPPSVSVSPGQTARITCSGDALPNQYAYWYQQKPGQ |
| | | APVLVIYKDSERPSGIPERFSGSSSGTTVTLTISGVQAEDE |
| | | ADYYCQSADSSGTVVFGGGTKLSVLGQPKAAPSVTLFPPSS |
| | | EELQANKATLVCLISDFYPGAVTVAWKADSSPVKAGVETTT |
| | | PSKQSNNKYAASSYLSLTPEQWKSHRSYSCQVTHEGSTVEK |
| | | TVAPTECS |
| 106 | OE12-hIgG4v1-HC | QVQLVQSGAEVKKPGASVKVSCKASGYTFTNYDINWVRQAT |
| | | GQGLEWMGWMNPNSGNTDYVQKFQGRVAMTRITSISTAYLE |
| | | LSSLRSDDTAVYYCARGLHSGDYYSYGMDVWGQGTTVTVSS |
| | | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEAAGGPSVFLF |
| | | PPKPKDTLMISRTPEVTCVVVDVSQEDPEVQFNWYVDGVEV |
| | | HNAKTKPREEQFNSTYRVVSVLTVLHQDWLNGKEYKCKVSN |
| | | KGLPSSIEKTISKAKGQPREPQVYTLPPSQEEMTKNQVSLT |
| | | CLVKGFYPSDIAVEWESNGQPENNYKTTPPVLDSDGSFFLY |
| | | SRLTVDKSRWQEGNVFSCSVMHEALHNHYTQKSLSLSLG |
| 107 | OE12-LC-lambda-L1- | SYELTQPPSVSVSPGQTARITCSGDALPNQYAYWYQQKPGQ |
| | Lipocalin Mutein I | APVLVIYKDSERPSGIPERFSGSSSGTTVTLTISGVQAEDE |
| | | ADYYCQSADSSGTVVFGGGTKLSVLGQPKAAPSVTLEPPSS |
| | | EELQANKATLVCLISDFYPGAVTVAWKADSSPVKAGVETTT |
| | | PSKQSNNKYAASSYLSLTPEQWKSHRSYSCQVTHEGSTVEK |
| | | TVAPTECSGGGGSGGGGSGGGGSQDSTSDLIPAPPLSKVPL |
| | | QQNFQDNQFHGKWYVVGQAGNIRLREDKDPIKMMATIYELK |
| | | EDKSYDVTMVKEDDKKCMYDIWTFVPGSQPGEFTLGKIKSF |
| | | PGHTSSLVRVVSTNYNQHAMVFFKFVFQNREEFYITLYGRT |
| | | KELTSELKENFIRFSKSLGLPENHIVFPVPIDQCIDG |
| 108 | Human CD228 protein | MRGPSGALWLLLALRTVLGGMEVRWCATSDPEQHKCGNMSE |
| | sequence- | AFREAGIQPSLLCVRGTSADHCVQLIAAQEADAITLDGGAI |
| | NP_005920.2 | YEAGKEHGLKPVVGEVYDQEVGTSYYAVAVVRRSSHVTIDT |
| | | LKGVKSCHTGINRTVGWNVPVGYLVESGRLSVMGCDVLKAV |
| | | SDYFGGSCVPGAGETSYSESLCRLCRGDSSGEGVCDKSPLE |
| | | RYYDYSGAFRCLAEGAGDVAFVKHSTVLENTDGKTLPSWGQ |
| | | ALLSQDFELLCRDGSRADVTEWRQCHLARVPAHAVVVRADT |
| | | DGGLIFRLLNEGQRLFSHEGSSFQMESSEAYGQKDLLFKDS |
| | | TSELVPIATQTYEAWLGHEYLHAMKGLLCDPNRLPPYLRWC |
| | | VLSTPEIQKCGDMAVAFRRQRLKPEIQCVSAKSPQHCMERI |
| | | QAEQVDAVTLSGEDIYTAGKTYGLVPAAGEHYAPEDSSNSY |
| | | YVVAVVRRDSSHAFTLDELRGKRSCHAGFGSPAGWDVPVGA |
| | | LIQRGFIRPKDCDVLTAVSEFFNASCVPVNNPKNYPSSLCA |
| | | LCVGDEQGRNKCVGNSQERYYGYRGAFRCLVENAGDVAFVR |
| | | HTTVFDNTNGHNSEPWAAELRSEDYELLCPNGARAEVSQFA |
| | | ACNLAQIPPHAVMVRPDTNIFTVYGLLDKAQDLFGDDHNKN |
| | | GFKMFDSSNYHGQDLLFKDATVRAVPVGEKTTYRGWLGLDY |
| | | VAALEGMSSQQCSGAAAPAPGAPLLPLLLPALAARLLPPAL |
| 109 | Cyno CD228 protein | MRGPSRALWLLLALRTVLGGMEVRWCVTSDPEQQKCSNMST |
| | sequence | AFREAGIQPSLLCVQGTSPDHCIQLIAAQEADAITLDGGAI |
| | | YEAGKEHGLKPVVGEVYDQEVGTSYYAVAVVKRSSQVTINT |
| | | LKGVKSCHTGINRTVGWNVPVGYLVESGRLSVMGCDVLKAV |
| | | SDYFGGSCVPGAGETRYSESLCRLCRGDSSGEGVCDKSPLE |
| | | RYYDYSGAFRCLAEGAGDVAFVKHSTVLENTDGKTLPSWGQ |
| | | ALLSQDFELLCRDGSRADVTEWRQCHLARVPAHAVVVRADT |
| | | DGGLIFRLLNEGQRLFSHEGSSFQMFSSKAYGQKDLLFKDS |
| | | TSELVPIATQTYEAWLGQEYLHAMKGLLCDPNRLPPYLRWC |
| | | VLSTPEIQKCGDMAVAFGRQQLKPEIQCVSAKSPQHCMEQI |
| | | QAGQIDTVTLSGEDIYTAGKTYGLAPAAGEGYASEDSSNSY |
| | | FVVAVVRRDSSHAFTLDELRGKRSCHAGFGSPAGWDIPVGA |
| | | LIRRGFIRPKDCDVLTAVSEFFNASCVPVNNPKNYPSSLCA |
| | | LCVGDEQGRNKCVGNSQERYYGNSGAFRCLVENAGDVAFVR |
| | | HTTVEDNTNGHNSEPWAAELRSEDYELLCPNGARAEVSQFA |
| | | ACNLAQMPPHAVMVRPDTNIFTVYGLLDKAQDLFGDDHNKN |
| | | GFKMFDSSNYHGQDLLFKDATVRAVPVGEKTTYRDWLGLDY |
| | | VAALEGMLSEQCSGAAAPAPGAPLLPLLLPALAARLLPPAL |
| 110 | Anti-CD228 antibody | SGGYYWS |
| | clone 8 CDR-H1 | |
| | (Kabat) | |
| 111 | Anti-CD228 antibody | YIHYIGSTYQNPSLKS |
| | clone 8 CDR-H2 | |
| | (Kabat) | |
| 112 | Anti-CD228 antibody | ESGDTAMVEDY |
| | clone 8 CDR-H3 | |
| | (Kabat) | |
| 113 | Anti-CD228 antibody | GGSISSGGYY |
| | clone 8 CDR-H1 | |
| | (IMGT) | |
| 114 | Anti-CD228 antibody | IHYIGST |
| | clone 8 CDR-H2 | |
| | (IMGT) | |
| 115 | Anti-CD228 antibody | ARESGDTAMVEDY |
| | clone 8 CDR-H3 | |
| | (IMGT) | |
| 116 | Anti-CD228 antibody | TGTSSDVGGYNYVS |
| | clone 8 CDR-L1 | |
| | (Kabat) | |
| 117 | Anti-CD228 antibody | EVSTRPS |
| | clone 8 CDR-L2 | |
| | (Kabat) | |
| 118 | Anti-CD228 antibody | SSYAGNNNFV |
| | clone 8 CDR-L3 | |
| | (Kabat) | |
| 119 | Anti-CD228 antibody | SSDVGGYNY |
| | clone 8 CDR-L1 | |
| | (IMGT) | |
| 120 | Anti-CD228 antibody | EVS |
| | clone 8 CDR-L2 | |
| | (IMGT) | |
| 121 | Anti-CD228 antibody | SSYAGNNNFV |
| | clone 8 CDR-L3 | |
| | (IMGT) | |
| 122 | Anti-CD228 antibody | QVQLQESGPGLVKPSQTLSLTCTVSGGSISSGGYYWSWIRQ |
| | clone 8 VH amino acid | HPGKGLEWIGYIHYIGSTYQNPSLKSRVTMSVDTSKNQFSL |
| | | RLTSVTAADTAVYYCARESGDTAMVFDYWGQGTQVTVSS |
| 123 | Anti-CD228 antibody | CAGGTGCAGCTGCAGGAGTCGGGCCCAGGACTGGTGAAGCC |
| | clone 8 VH nucleic acid | TTCACAGACCCTGTCCCTCACCTGCACTGTCTCTGGTGGCT |
| | | CCATCAGCAGTGGTGGTTACTACTGGAGCTGGATCCGCCAG |
| | | CACCCAGGGAAGGGCCTGGAGTGGATTGGGTACATCCATTA |
| | | CATTGGGAGCACCTACCAGAACCCGTCCCTCAAGAGTCGAG |
| | | TTACCATGTCAGTAGACACGTCTAAGAACCAGTTCTCCCTG |
| | | AGGCTGACCTCTGTGACTGCCGCGGACACGGCCGTGTATTA |
| | | CTGTGCGAGAGAAAGTGGGGATACAGCTATGGTCTTTGACT |
| | | ACTGGGGCCAGGGAACCCAGGTCACCGTCTCCTCA |
| 124 | Anti-CD228 antibody | QSALTQPPSASGSPGQSVTISCTGTSSDVGGYNYVSWYQQH |
| | clone 8 VL amino acid | PGKAPKVMIYEVSTRPSGVPDRFSGSKSGNTASLTVSGLQA |
| | | EDEADYYCSSYAGNNNFVFGTGTKVTVL |
| 125 | Anti-CD228 antibody | CAGTCTGCCCTGACTCAGCCTCCCTCCGCGTCCGGGTCTCC |
| | clone 8 VL nucleic acid | TGGACAGTCAGTCACCATCTCCTGCACTGGAACCAGCAGTG |
| | | ACGTTGGTGGTTATAACTATGTCTCCTGGTACCAACAGCAC |
| | | CCAGGCAAAGCCCCCAAAGTCATGATTTATGAGGTCAGTAC |
| | | GCGGCCCTCAGGGGTCCCTGATCGCTTCTCTGGCTCCAAGT |
| | | CTGGCAACACGGCCTCCCTGACCGTCTCTGGGCTCCAGGCT |
| | | GAGGATGAGGCTGATTATTACTGCAGCTCATATGCAGGCAA |
| | | CAACAATTTTGTCTTCGGAACTGGGACCAAGGTCACCGTCC |
| | | TA |
| 126 | Anti-CD228 antibody | QVQLQESGPGLVKPSQTLSLTCTVSGGSISSGGYYWSWIRQ |
| | clone 8 HC amino acid | HPGKGLEWIGYIHYIGSTYQNPSLKSRVTMSVDTSKNQFSL |
| | | RLTSVTAADTAVYYCARESGDTAMVFDYWGQGTQVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| 127 | Anti-CD228 antibody | CAGGTGCAGCTGCAGGAGTCGGGCCCAGGACTGGTGAAGCC |
| | clone 8 HC nucleic acid | TTCACAGACCCTGTCCCTCACCTGCACTGTCTCTGGTGGCT |
| | | CCATCAGCAGTGGTGGTTACTACTGGAGCTGGATCCGCCAG |
| | | CACCCAGGGAAGGGCCTGGAGTGGATTGGGTACATCCATTA |
| | | CATTGGGAGCACCTACCAGAACCCGTCCCTCAAGAGTCGAG |
| | | TTACCATGTCAGTAGACACGTCTAAGAACCAGTTCTCCCTG |
| | | AGGCTGACCTCTGTGACTGCCGCGGACACGGCCGTGTATTA |
| | | CTGTGCGAGAGAAAGTGGGGATACAGCTATGGTCTTTGACT |
| | | ACTGGGGCCAGGGAACCCAGGTCACCGTCTCCTCAGCTAGC |
| | | ACCAAGGGCCCATCTGTCTTCCCCCTGGCACCCTCCTCCAA |
| | | GAGCACCTCTGGGGGCACAGCTGCCCTGGGCTGCCTGGTCA |
| | | AGGACTACTTCCCTGAACCTGTGACAGTGTCCTGGAACTCA |
| | | GGAGCCCTGACCAGCGGCGTGCACACCTTCCCGGCTGTCCT |
| | | ACAGTCCTCAGGACTCTACTCCCTCAGCAGCGTGGTGACCG |
| | | TGCCCTCCAGCAGCTTGGGCACCCAGACCTACATCTGCAAC |
| | | GTGAATCACAAGCCCAGCAACACCAAGGTGGACAAGAAAGT |
| | | TGAGCCCAAATCTTGTGACAAAACTCACACATGCCCACCGT |
| | | GCCCAGCACCTGAACTCCTGGGGGGACCGTCAGTCTTCCTC |
| | | TTCCCCCCAAAACCCAAGGACACCCTCATGATCTCCCGGAC |
| | | CCCTGAGGTCACATGCGTGGTGGTGGACGTGAGCCACGAAG |
| | | ACCCTGAGGTCAAGTTCAACTGGTACGTGGACGGCGTGGAG |
| | | GTGCATAATGCCAAGACAAAGCCGCGGGAGGAGCAGTACAA |
| | | CAGCACGTACCGTGTGGTCAGCGTCCTCACCGTCCTGCACC |
| | | AGGACTGGCTGAATGGCAAGGAGTACAAGTGCAAGGTCTCC |
| | | AACAAAGCCCTCCCAGCCCCCATCGAGAAAACCATCTCCAA |
| | | AGCCAAAGGGCAGCCCCGAGAACCACAGGTGTACACCCTGC |
| | | CCCCATCCCGGGATGAGCTGACCAAGAACCAGGTCAGCCTG |
| | | ACCTGCCTGGTCAAAGGCTTCTATCCCAGCGACATCGCCGT |
| | | GGAGTGGGAGAGCAATGGGCAGCCGGAGAACAACTACAAGA |
| | | CCACGCCTCCCGTGCTGGACTCCGACGGCTCCTTCTTCCTC |
| | | TACAGCAAGCTCACCGTGGACAAGAGCAGATGGCAGCAGGG |
| | | GAACGTCTTCTCATGCTCCGTGATGCATGAGGCTCTGCACA |
| | | ACCACTACACACAGAAGAGCCTCTCCCTGTCTCCGGGCAAA |
| | | TGA |
| 128 | Anti-CD228 antibody | QSALTQPPSASGSPGQSVTISCTGTSSDVGGYNYVSWYQQH |
| | clone 8 LC amino acid | PGKAPKVMIYEVSTRPSGVPDRFSGSKSGNTASLTVSGLQA |
| | | EDEADYYCSSYAGNNNFVFGTGTKVTVLGQPKAAPSVTLEP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS |
| 129 | Anti-CD228 antibody | CAGTCTGCCCTGACTCAGCCTCCCTCCGCGTCCGGGTCTCC |
| | clone 8 LC nucleic acid | TGGACAGTCAGTCACCATCTCCTGCACTGGAACCAGCAGTG |
| | | ACGTTGGTGGTTATAACTATGTCTCCTGGTACCAACAGCAC |
| | | CCAGGCAAAGCCCCCAAAGTCATGATTTATGAGGTCAGTAC |
| | | GCGGCCCTCAGGGGTCCCTGATCGCTTCTCTGGCTCCAAGT |
| | | CTGGCAACACGGCCTCCCTGACCGTCTCTGGGCTCCAGGCT |
| | | GAGGATGAGGCTGATTATTACTGCAGCTCATATGCAGGCAA |
| | | CAACAATTTTGTCTTCGGAACTGGGACCAAGGTCACCGTCC |
| | | TAGGTCAGCCCAAGGCGGCGCCCTCGGTCACTCTGTTCCCG |
| | | CCCTCCTCTGAGGAGCTTCAAGCCAACAAGGCCACACTGGT |
| | | GTGTCTCATAAGTGACTTCTACCCGGGAGCCGTGACAGTGG |
| | | CCTGGAAGGCAGATAGCAGCCCCGTCAAGGCGGGAGTGGAG |
| | | ACCACCACACCCTCCAAACAAAGCAACAACAAGTACGCGGC |
| | | CAGCAGCTATCTGAGCCTGACGCCTGAGCAGTGGAAGTCCC |
| | | ACAGAAGCTACAGCTGCCAGGTCACGCATGAAGGGAGCACC |
| | | GTGGAGAAGACAGTGGCCCCTACAGAATGTTCATAG |
| 130 | Anti-CD228 antibody | SGNYYWS |
| | clone 11 CDR-H1 | |
| | (Kabat) | |
| 131 | Anti-CD228 antibody | YIYYSGSTYYNPSLKS |
| | clone 11 CDR-H2 | |
| | (Kabat) | |
| 132 | Anti-CD228 antibody | ADWNDQGDAFDI |
| | clone 11 CDR-H3 | |
| | (Kabat) | |
| 133 | Anti-CD228 antibody | GGSINSGNYY |
| | clone 11 CDR-H1 | |
| | (IMGT) | |
| 134 | Anti-CD228 antibody | IYYSGST |
| | clone 11 CDR-H2 | |
| | (IMGT) | |
| 135 | Anti-CD228 antibody | ARADWNDQGDAFDI |
| | clone 11 CDR-H3 | |
| | (IMGT) | |
| 136 | Anti-CD228 antibody | RASQDIRNDLG |
| | clone 11 CDR-L1 | |
| | (Kabat) | |
| 137 | Anti-CD228 antibody | AASSLQS |
| | clone 11 CDR-L2 | |
| | (Kabat) | |
| 138 | Anti-CD228 antibody | LQDYNYPFT |
| | clone 11 CDR-L3 | |
| | (Kabat) | |
| 139 | Anti-CD228 antibody | QDIRND |
| | clone 11 CDR-L1 | |
| | (IMGT) | |
| 140 | Anti-CD228 antibody | AAS |
| | clone 11 CDR-L2 | |
| | (IMGT) | |
| 141 | Anti-CD228 antibody | LQDYNYPFT |
| | clone 11 CDR-L3 | |
| | (IMGT) | |
| 142 | Anti-CD228 antibody | QVQLQESGPGLVKPSETLSLTCTVSGGSINSGNYYWSWIRQ |
| | clone 11 VH amino | HPGKGLEWIGYIYYSGSTYYNPSLKSRVTISADTSKNQFSL |
| | acid | KLSSVTAADTAVYYCARADWNDQGDAFDIWGQGTMATVSS |
| 143 | Anti-CD228 antibody | CAGGTGCAGCTGCAGGAGTCGGGCCCAGGACTGGTGAAGCC |
| | clone 11 VH nucleic | TTCAGAGACCCTGTCCCTCACCTGCACTGTCTCTGGTGGCT |
| | acid | CCATCAACAGTGGTAATTACTACTGGAGCTGGATCCGCCAG |
| | | CACCCAGGGAAGGGCCTGGAGTGGATTGGGTACATCTATTA |
| | | CAGTGGGAGCACCTACTACAACCCGTCCCTCAAGAGTCGAG |
| | | TTACCATATCAGCAGACACGTCTAAGAACCAGTTCTCCCTG |
| | | AAGCTGAGCTCTGTGACTGCCGCGGACACGGCCGTGTATTA |
| | | CTGTGCGAGAGCAGACTGGAACGACCAGGGCGATGCTTTTG |
| | | ATATCTGGGGCCAAGGGACAATGGCCACCGTCTCTTCA |
| 144 | Anti-CD228 antibody | AIQMTQSPSSLSASVGDRVTITCRASQDIRNDLGWYQQKPG |
| | clone 11 VL amino acid | KAPKLLIYAASSLQSGVPSRFSGSGSGTDFTLTISSLQPED |
| | | FVTYYCLQDYNYPFTFGPGTKVDIK |
| 145 | Anti-CD228 antibody | GCCATCCAGATGACCCAGTCTCCATCCTCCCTGTCTGCATC |
| | clone 11 VL nucleic | TGTAGGAGACAGAGTCACCATCACTTGCCGGGCAAGTCAGG |
| | acid | ACATTAGAAATGATTTAGGCTGGTATCAGCAGAAACCAGGG |
| | | AAAGCCCCTAAGCTCCTGATCTATGCTGCATCCAGTTTACA |
| | | AAGTGGGGTCCCATCAAGGTTCAGCGGCAGTGGATCTGGCA |
| | | CAGATTTCACTCTCACCATCAGCAGCCTGCAGCCTGAAGAT |
| | | TTTGTAACTTATTACTGTCTACAAGATTACAATTACCCATT |
| | | CACTTTCGGCCCTGGGACCAAAGTGGATATCAAA |
| 146 | Anti-CD228 antibody | QVQLQESGPGLVKPSETLSLTCTVSGGSINSGNYYWSWIRQ |
| | clone 11 HC amino | HPGKGLEWIGYIYYSGSTYYNPSLKSRVTISADTSKNQFSL |
| | acid | KLSSVTAADTAVYYCARADWNDQGDAFDIWGQGTMATVSSA |
| | | STKGPSVFPLAPSSKSTSGGTAALGCLVKDYFPEPVTVSWN |
| | | SGALTSGVHTFPAVLQSSGLYSLSSVVTVPSSSLGTQTYIC |
| | | NVNHKPSNTKVDKKVEPKSCDKTHTCPPCPAPELLGGPSVF |
| | | LFPPKPKDTLMISRTPEVTCVVVDVSHEDPEVKFNWYVDGV |
| | | EVHNAKTKPREEQYNSTYRVVSVLTVLHQDWLNGKEYKCKV |
| | | SNKALPAPIEKTISKAKGQPREPQVYTLPPSRDELTKNQVS |
| | | LTCLVKGFYPSDIAVEWESNGQPENNYKTTPPVLDSDGSFF |
| | | LYSKLTVDKSRWQQGNVFSCSVMHEALHNHYTQKSLSLSPG |
| | | K |
| 147 | Anti-CD228 antibody | CAGGTGCAGCTGCAGGAGTCGGGCCCAGGACTGGTGAAGCC |
| | clone 11 HC nucleic | TTCAGAGACCCTGTCCCTCACCTGCACTGTCTCTGGTGGCT |
| | acid | CCATCAACAGTGGTAATTACTACTGGAGCTGGATCCGCCAG |
| | | CACCCAGGGAAGGGCCTGGAGTGGATTGGGTACATCTATTA |
| | | CAGTGGGAGCACCTACTACAACCCGTCCCTCAAGAGTCGAG |
| | | TTACCATATCAGCAGACACGTCTAAGAACCAGTTCTCCCTG |
| | | AAGCTGAGCTCTGTGACTGCCGCGGACACGGCCGTGTATTA |
| | | CTGTGCGAGAGCAGACTGGAACGACCAGGGCGATGCTTTTG |
| | | ATATCTGGGGCCAAGGGACAATGGCCACCGTCTCTTCAGCT |
| | | AGCACCAAGGGCCCATCTGTCTTCCCCCTGGCACCCTCCTC |
| | | CAAGAGCACCTCTGGGGGCACAGCTGCCCTGGGCTGCCTGG |
| | | TCAAGGACTACTTCCCTGAACCTGTGACAGTGTCCTGGAAC |
| | | TCAGGAGCCCTGACCAGCGGCGTGCACACCTTCCCGGCTGT |
| | | CCTACAGTCCTCAGGACTCTACTCCCTCAGCAGCGTGGTGA |
| | | CCGTGCCCTCCAGCAGCTTGGGCACCCAGACCTACATCTGC |
| | | AACGTGAATCACAAGCCCAGCAACACCAAGGTGGACAAGAA |
| | | AGTTGAGCCCAAATCTTGTGACAAAACTCACACATGCCCAC |
| | | CGTGCCCAGCACCTGAACTCCTGGGGGGACCGTCAGTCTTC |
| | | CTCTTCCCCCCAAAACCCAAGGACACCCTCATGATCTCCCG |
| | | GACCCCTGAGGTCACATGCGTGGTGGTGGACGTGAGCCACG |
| | | AAGACCCTGAGGTCAAGTTCAACTGGTACGTGGACGGCGTG |
| | | GAGGTGCATAATGCCAAGACAAAGCCGCGGGAGGAGCAGTA |
| | | CAACAGCACGTACCGTGTGGTCAGCGTCCTCACCGTCCTGC |
| | | ACCAGGACTGGCTGAATGGCAAGGAGTACAAGTGCAAGGTC |
| | | TCCAACAAAGCCCTCCCAGCCCCCATCGAGAAAACCATCTC |
| | | CAAAGCCAAAGGGCAGCCCCGAGAACCACAGGTGTACACCC |
| | | TGCCCCCATCCCGGGATGAGCTGACCAAGAACCAGGTCAGC |
| | | CTGACCTGCCTGGTCAAAGGCTTCTATCCCAGCGACATCGC |
| | | CGTGGAGTGGGAGAGCAATGGGCAGCCGGAGAACAACTACA |
| | | AGACCACGCCTCCCGTGCTGGACTCCGACGGCTCCTTCTTC |
| | | CTCTACAGCAAGCTCACCGTGGACAAGAGCAGATGGCAGCA |
| | | GGGGAACGTCTTCTCATGCTCCGTGATGCATGAGGCTCTGC |
| | | ACAACCACTACACACAGAAGAGCCTCTCCCTGTCTCCGGGC |
| | | AAATGA |
| 148 | Anti-CD228 antibody | AIQMTQSPSSLSASVGDRVTITCRASQDIRNDLGWYQQKPG |
| | clone 11 LC amino acid | KAPKLLIYAASSLQSGVPSRFSGSGSGTDFTLTISSLQPED |
| | | FVTYYCLQDYNYPFTFGPGTKVDIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 149 | Anti-CD228 antibody | GCCATCCAGATGACCCAGTCTCCATCCTCCCTGTCTGCATC |
| | clone 11 LC nucleic | TGTAGGAGACAGAGTCACCATCACTTGCCGGGCAAGTCAGG |
| | acid | ACATTAGAAATGATTTAGGCTGGTATCAGCAGAAACCAGGG |
| | | AAAGCCCCTAAGCTCCTGATCTATGCTGCATCCAGTTTACA |
| | | AAGTGGGGTCCCATCAAGGTTCAGCGGCAGTGGATCTGGCA |
| | | CAGATTTCACTCTCACCATCAGCAGCCTGCAGCCTGAAGAT |
| | | TTTGTAACTTATTACTGTCTACAAGATTACAATTACCCATT |
| | | CACTTTCGGCCCTGGGACCAAAGTGGATATCAAACGTACGG |
| | | TGGCTGCACCATCTGTCTTCATCTTCCCGCCATCTGATGAG |
| | | CAGTTGAAATCTGGAACTGCCTCTGTTGTGTGCCTGCTGAA |
| | | TAACTTCTATCCCAGAGAGGCCAAAGTACAGTGGAAGGTGG |
| | | ATAACGCCCTCCAATCGGGTAACTCCCAGGAGAGTGTCACA |
| | | GAGCAGGACAGCAAGGACAGCACCTACAGCCTCAGCAGCAC |
| | | CCTGACGCTGAGCAAAGCAGACTACGAGAAACACAAAGTCT |
| | | ACGCCTGCGAAGTCACCCATCAGGGCCTGAGCTCGCCCGTC |
| | | ACAAAGAGCTTCAACAGGGGAGAGTGTTAG |
| 150 | Anti-CD228 antibody | SGGYYWS |
| | clone 14 CDR-H1 | |
| | (Kabat) | |
| 151 | Anti-CD228 antibody | YIHYIGSTYQNPSLKS |
| | clone 14 CDR-H2 | |
| | (Kabat) | |
| 152 | Anti-CD228 antibody | ESGDTAMVEDY |
| | clone 14 CDR-H3 | |
| | (Kabat) | |
| 153 | Anti-CD228 antibody | GGSISSGGYY |
| | clone 14 CDR-H1 | |
| | (IMGT) | |
| 154 | Anti-CD228 antibody | IHYIGST |
| | clone 14 CDR-H2 | |
| | (IMGT) | |
| 155 | Anti-CD228 antibody | ARESGDTAMVEDY |
| | clone 14 CDR-H3 | |
| | (IMGT) | |
| 156 | Anti-CD228 antibody | TGTSSDVGGYNYVS |
| | clone 14 CDR-L1 | |
| | (Kabat) | |
| 157 | Anti-CD228 antibody | EVSTRPS |
| | clone 14 CDR-L2 | |
| | (Kabat) | |
| 158 | Anti-CD228 antibody | SSYAGNNNFV |
| | clone 14 CDR-L3 | |
| | (Kabat) | |
| 159 | Anti-CD228 antibody | SSDVGGYNY |
| | clone 14 CDR-L1 | |
| | (IMGT) | |
| 160 | Anti-CD228 antibody | EVS |
| | clone 14 CDR-L2 | |
| | (IMGT) | |
| 161 | Anti-CD228 antibody | SSYAGNNNFV |
| | clone 14 CDR-L3 | |
| | (IMGT) | |
| 162 | Anti-CD228 antibody | QVQLQESGPGLVKPSQTLSLTCTVSGGSISSGGYYWSWIRQ |
| | clone 14 VH amino | HPGKGLEWIGYIHYIGSTYQNPSLKSRVTMSVDTSKNQFSL |
| | acid | RLTSVTAADTAVYYCARESGDTAMVFDYWGQGTQVTVSS |
| 163 | Anti-CD228 antibody | CAGGTGCAGCTGCAGGAGTCGGGCCCAGGACTGGTGAAGCC |
| | clone 14 VH nucleic | TTCACAGACCCTGTCCCTCACCTGCACTGTCTCTGGTGGCT |
| | acid | CCATCAGCAGTGGTGGTTACTACTGGAGCTGGATCCGCCAG |
| | | CACCCAGGGAAGGGCCTGGAGTGGATTGGATACATCCATTA |
| | | CATTGGGAGCACCTACCAGAACCCGTCCCTCAAGAGTCGAG |
| | | TTACCATGTCAGTAGACACGTCTAAGAACCAGTTCTCCCTG |
| | | AGGCTGACCTCTGTGACTGCCGCGGACACGGCCGTTTATTA |
| | | CTGTGCGAGAGAAAGTGGGGATACAGCTATGGTCTTTGACT |
| | | ACTGGGGCCAGGGAACCCAGGTCACCGTCTCCTCA |
| 164 | Anti-CD228 antibody | QSALTQPPSASGSPGQSVTISCTGTSSDVGGYNYVSWYQQH |
| | clone 14 VL amino acid | PGKAPKVMIYEVSTRPSGVPDRFSGSKSGNTASLTVSGLQA |
| | | EDEADYYCSSYAGNNNFVFGTGTKVTVL |
| 165 | Anti-CD228 antibody | CAGTCTGCCCTGACTCAGCCTCCCTCCGCGTCCGGGTCTCC |
| | clone 14 VL nucleic | TGGACAGTCAGTCACCATCTCCTGCACTGGAACCAGCAGTG |
| | acid | ACGTTGGTGGTTATAACTATGTCTCCTGGTACCAACAGCAC |
| | | CCAGGCAAAGCCCCCAAAGTCATGATTTATGAGGTCAGTAC |
| | | GCGGCCCTCAGGGGTCCCTGATCGCTTCTCTGGCTCCAAGT |
| | | CTGGCAACACGGCCTCCCTGACCGTCTCTGGGCTCCAGGCT |
| | | GAGGATGAGGCTGATTATTACTGCAGCTCATATGCAGGCAA |
| | | CAACAATTTTGTCTTCGGAACTGGGACCAAGGTCACCGTCC |
| | | TA |
| 166 | Anti-CD228 antibody | QVQLQESGPGLVKPSQTLSLTCTVSGGSISSGGYYWSWIRQ |
| | clone 14 HC amino | HPGKGLEWIGYIHYIGSTYQNPSLKSRVTMSVDTSKNQFSL |
| | acid | RLTSVTAADTAVYYCARESGDTAMVFDYWGQGTQVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| 167 | Anti-CD228 antibody | CAGGTGCAGCTGCAGGAGTCGGGCCCAGGACTGGTGAAGCC |
| | clone 14 HC nucleic | TTCACAGACCCTGTCCCTCACCTGCACTGTCTCTGGTGGCT |
| | acid | CCATCAGCAGTGGTGGTTACTACTGGAGCTGGATCCGCCAG |
| | | CACCCAGGGAAGGGCCTGGAGTGGATTGGATACATCCATTA |
| | | CATTGGGAGCACCTACCAGAACCCGTCCCTCAAGAGTCGAG |
| | | TTACCATGTCAGTAGACACGTCTAAGAACCAGTTCTCCCTG |
| | | AGGCTGACCTCTGTGACTGCCGCGGACACGGCCGTTTATTA |
| | | CTGTGCGAGAGAAAGTGGGGATACAGCTATGGTCTTTGACT |
| | | ACTGGGGCCAGGGAACCCAGGTCACCGTCTCCTCAGCTAGC |
| | | ACCAAGGGCCCATCTGTCTTCCCCCTGGCACCCTCCTCCAA |
| | | GAGCACCTCTGGGGGCACAGCTGCCCTGGGCTGCCTGGTCA |
| | | AGGACTACTTCCCTGAACCTGTGACAGTGTCCTGGAACTCA |
| | | GGAGCCCTGACCAGCGGCGTGCACACCTTCCCGGCTGTCCT |
| | | ACAGTCCTCAGGACTCTACTCCCTCAGCAGCGTGGTGACCG |
| | | TGCCCTCCAGCAGCTTGGGCACCCAGACCTACATCTGCAAC |
| | | GTGAATCACAAGCCCAGCAACACCAAGGTGGACAAGAAAGT |
| | | TGAGCCCAAATCTTGTGACAAAACTCACACATGCCCACCGT |
| | | GCCCAGCACCTGAACTCCTGGGGGGACCGTCAGTCTTCCTC |
| | | TTCCCCCCAAAACCCAAGGACACCCTCATGATCTCCCGGAC |
| | | CCCTGAGGTCACATGCGTGGTGGTGGACGTGAGCCACGAAG |
| | | ACCCTGAGGTCAAGTTCAACTGGTACGTGGACGGCGTGGAG |
| | | GTGCATAATGCCAAGACAAAGCCGCGGGAGGAGCAGTACAA |
| | | CAGCACGTACCGTGTGGTCAGCGTCCTCACCGTCCTGCACC |
| | | AGGACTGGCTGAATGGCAAGGAGTACAAGTGCAAGGTCTCC |
| | | AACAAAGCCCTCCCAGCCCCCATCGAGAAAACCATCTCCAA |
| | | AGCCAAAGGGCAGCCCCGAGAACCACAGGTGTACACCCTGC |
| | | CCCCATCCCGGGATGAGCTGACCAAGAACCAGGTCAGCCTG |
| | | ACCTGCCTGGTCAAAGGCTTCTATCCCAGCGACATCGCCGT |
| | | GGAGTGGGAGAGCAATGGGCAGCCGGAGAACAACTACAAGA |
| | | CCACGCCTCCCGTGCTGGACTCCGACGGCTCCTTCTTCCTC |
| | | TACAGCAAGCTCACCGTGGACAAGAGCAGATGGCAGCAGGG |
| | | GAACGTCTTCTCATGCTCCGTGATGCATGAGGCTCTGCACA |
| | | ACCACTACACACAGAAGAGCCTCTCCCTGTCTCCGGGCAAA |
| 168 | Anti-CD228 antibody | QSALTQPPSASGSPGQSVTISCTGTSSDVGGYNYVSWYQQH |
| | clone 14 LC amino acid | PGKAPKVMIYEVSTRPSGVPDRFSGSKSGNTASLTVSGLQA |
| | | EDEADYYCSSYAGNNNFVFGTGTKVTVLGQPKAAPSVTLEP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS |
| 169 | Anti-CD228 antibody | CAGTCTGCCCTGACTCAGCCTCCCTCCGCGTCCGGGTCTCC |
| | clone 14 LC nucleic | TGGACAGTCAGTCACCATCTCCTGCACTGGAACCAGCAGTG |
| | acid | ACGTTGGTGGTTATAACTATGTCTCCTGGTACCAACAGCAC |
| | | CCAGGCAAAGCCCCCAAAGTCATGATTTATGAGGTCAGTAC |
| | | GCGGCCCTCAGGGGTCCCTGATCGCTTCTCTGGCTCCAAGT |
| | | CTGGCAACACGGCCTCCCTGACCGTCTCTGGGCTCCAGGCT |
| | | GAGGATGAGGCTGATTATTACTGCAGCTCATATGCAGGCAA |
| | | CAACAATTTTGTCTTCGGAACTGGGACCAAGGTCACCGTCC |
| | | TAGGTCAGCCCAAGGCGGCGCCCTCGGTCACTCTGTTCCCG |
| | | CCCTCCTCTGAGGAGCTTCAAGCCAACAAGGCCACACTGGT |
| | | GTGTCTCATAAGTGACTTCTACCCGGGAGCCGTGACAGTGG |
| | | CCTGGAAGGCAGATAGCAGCCCCGTCAAGGCGGGAGTGGAG |
| | | ACCACCACACCCTCCAAACAAAGCAACAACAAGTACGCGGC |
| | | CAGCAGCTATCTGAGCCTGACGCCTGAGCAGTGGAAGTCCC |
| | | ACAGAAGCTACAGCTGCCAGGTCACGCATGAAGGGAGCACC |
| | | GTGGAGAAGACAGTGGCCCCTACAGAATGTTCATAG |
| 170 | Anti-CD228 antibody | TYVMN |
| | clone 24 CDR-H1 | |
| | (Kabat) | |
| 171 | Anti-CD228 antibody | WINTNTGNPTYAQGFTG |
| | clone 24 CDR-H2 | |
| | (Kabat) | |
| 172 | Anti-CD228 antibody | DRVLLWFGAGYYHYGMDV |
| | clone 24 CDR-H3 | |
| | (Kabat) | |
| 173 | Anti-CD228 antibody | GYTFTTYV |
| | clone 24 CDR-H1 | |
| | (IMGT) | |
| 174 | Anti-CD228 antibody | INTNTGNP |
| | clone 24 CDR-H2 | |
| | (IMGT) | |
| 175 | Anti-CD228 antibody | ARDRVLLWFGAGYYHYGMDV |
| | clone 24 CDR-H3 | |
| | (IMGT) | |
| 176 | Anti-CD228 antibody | QGDSLRSYYTS |
| | clone 24 CDR-L1 | |
| | (Kabat) | |
| 177 | Anti-CD228 antibody | GKNNRPS |
| | clone 24 CDR-L2 | |
| | (Kabat) | |
| 178 | Anti-CD228 antibody | NSRDSSGNHVV |
| | clone 24 CDR-L3 | |
| | (Kabat) | |
| 179 | Anti-CD228 antibody | SLRSYY |
| | clone 24 CDR-L1 | |
| | (IMGT) | |
| 180 | Anti-CD228 antibody | GKN |
| | clone 24 CDR-L2 | |
| | (IMGT) | |
| 181 | Anti-CD228 antibody | NSRDSSGNHVV |
| | clone 24 CDR-L3 | |
| | (IMGT) | |
| 182 | Anti-CD228 antibody | QVQLVQSGSELKKPGASVKVSCKASGYTFTTYVMNWVRQAP |
| | clone 24 VH amino | GQGLEWMGWINTNTGNPTYAQGFTGRFVFSVDTSVSTAYLQ |
| | acid | ISSLKAEDTAVYYCARDRVLLWFGAGYYHYGMDVWGQGTTV |
| | | TVSS |
| 183 | Anti-CD228 antibody | CAGGTGCAGCTGGTGCAATCTGGGTCTGAGTTGAAGAAGCC |
| | clone 24 VH nucleic | TGGGGCCTCAGTGAAGGTTTCCTGCAAGGCTTCTGGATACA |
| | acid | CCTTCACTACCTATGTTATGAATTGGGTGCGACAGGCCCCT |
| | | GGACAAGGGCTCGAGTGGATGGGATGGATCAACACCAACAC |
| | | TGGGAACCCAACGTATGCCCAGGGCTTCACAGGACGGTTTG |
| | | TCTTCTCCGTGGACACCTCTGTCAGCACGGCATATCTGCAG |
| | | ATCAGCAGCCTAAAGGCTGAGGACACTGCCGTGTATTACTG |
| | | TGCGAGAGATCGAGTATTACTATGGTTCGGGGCGGGGTACT |
| | | ACCACTACGGTATGGACGTCTGGGGCCAAGGGACCACGGTC |
| | | ACCGTCTCCTCA |
| 184 | Anti-CD228 antibody | SSELTQDPAVSVALGQTVRITCQGDSLRSYYTSWYQQKPGQ |
| | clone 24 VL amino acid | APVFVIYGKNNRPSGIPDRFSGSSSGNTASLTITGAQAEDE |
| | | ADYYCNSRDSSGNHVVFGGGTKLTVL |
| 185 | Anti-CD228 antibody | TCTTCTGAGCTGACTCAGGACCCTGCTGTGTCTGTGGCCTT |
| | clone 24 VL nucleic | GGGACAGACAGTCAGGATCACATGCCAAGGAGACAGCCTCA |
| | acid | GATCCTATTATACAAGCTGGTACCAGCAGAAGCCAGGACAG |
| | | GCCCCTGTATTTGTCATCTATGGTAAAAACAACCGGCCCTC |
| | | AGGGATCCCAGACCGATTCTCTGGCTCCAGCTCAGGAAACA |
| | | CAGCTTCCTTGACCATCACTGGGGCTCAGGCGGAAGATGAG |
| | | GCTGACTATTACTGTAATTCCCGGGACAGCAGTGGTAACCA |
| | | TGTGGTTTTCGGCGGAGGGACCAAGCTGACCGTCCTA |
| 186 | Anti-CD228 antibody | QVQLVQSGSELKKPGASVKVSCKASGYTFTTYVMNWVRQAP |
| | clone 24 HC amino | GQGLEWMGWINTNTGNPTYAQGFTGRFVFSVDTSVSTAYLQ |
| | acid | ISSLKAEDTAVYYCARDRVLLWFGAGYYHYGMDVWGQGTTV |
| | | TVSSASTKGPSVFPLAPSSKSTSGGTAALGCLVKDYFPEPV |
| | | TVSWNSGALTSGVHTFPAVLQSSGLYSLSSVVTVPSSSLGT |
| | | QTYICNVNHKPSNTKVDKKVEPKSCDKTHTCPPCPAPELLG |
| | | GPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSRDELT |
| | | KNQVSLTCLVKGFYPSDIAVEWESNGQPENNYKTTPPVLDS |
| | | DGSFFLYSKLTVDKSRWQQGNVFSCSVMHEALHNHYTQKSL |
| | | SLSPGK |
| 187 | Anti-CD228 antibody | CAGGTGCAGCTGGTGCAATCTGGGTCTGAGTTGAAGAAGCC |
| | clone 24 HC nucleic | TGGGGCCTCAGTGAAGGTTTCCTGCAAGGCTTCTGGATACA |
| | acid | CCTTCACTACCTATGTTATGAATTGGGTGCGACAGGCCCCT |
| | | GGACAAGGGCTCGAGTGGATGGGATGGATCAACACCAACAC |
| | | TGGGAACCCAACGTATGCCCAGGGCTTCACAGGACGGTTTG |
| | | TCTTCTCCGTGGACACCTCTGTCAGCACGGCATATCTGCAG |
| | | ATCAGCAGCCTAAAGGCTGAGGACACTGCCGTGTATTACTG |
| | | TGCGAGAGATCGAGTATTACTATGGTTCGGGGCGGGGTACT |
| | | ACCACTACGGTATGGACGTCTGGGGCCAAGGGACCACGGTC |
| | | ACCGTCTCCTCAGCTAGCACCAAGGGCCCATCTGTCTTCCC |
| | | CCTGGCACCCTCCTCCAAGAGCACCTCTGGGGGCACAGCTG |
| | | CCCTGGGCTGCCTGGTCAAGGACTACTTCCCTGAACCTGTG |
| | | ACAGTGTCCTGGAACTCAGGAGCCCTGACCAGCGGCGTGCA |
| | | CACCTTCCCGGCTGTCCTACAGTCCTCAGGACTCTACTCCC |
| | | TCAGCAGCGTGGTGACCGTGCCCTCCAGCAGCTTGGGCACC |
| | | CAGACCTACATCTGCAACGTGAATCACAAGCCCAGCAACAC |
| | | CAAGGTGGACAAGAAAGTTGAGCCCAAATCTTGTGACAAAA |
| | | CTCACACATGCCCACCGTGCCCAGCACCTGAACTCCTGGGG |
| | | GGACCGTCAGTCTTCCTCTTCCCCCCAAAACCCAAGGACAC |
| | | CCTCATGATCTCCCGGACCCCTGAGGTCACATGCGTGGTGG |
| | | TGGACGTGAGCCACGAAGACCCTGAGGTCAAGTTCAACTGG |
| | | TACGTGGACGGCGTGGAGGTGCATAATGCCAAGACAAAGCC |
| | | GCGGGAGGAGCAGTACAACAGCACGTACCGTGTGGTCAGCG |
| | | TCCTCACCGTCCTGCACCAGGACTGGCTGAATGGCAAGGAG |
| | | TACAAGTGCAAGGTCTCCAACAAAGCCCTCCCAGCCCCCAT |
| | | CGAGAAAACCATCTCCAAAGCCAAAGGGCAGCCCCGAGAAC |
| | | CACAGGTGTACACCCTGCCCCCATCCCGGGATGAGCTGACC |
| | | AAGAACCAGGTCAGCCTGACCTGCCTGGTCAAAGGCTTCTA |
| | | TCCCAGCGACATCGCCGTGGAGTGGGAGAGCAATGGGCAGC |
| | | CGGAGAACAACTACAAGACCACGCCTCCCGTGCTGGACTCC |
| | | GACGGCTCCTTCTTCCTCTACAGCAAGCTCACCGTGGACAA |
| | | GAGCAGATGGCAGCAGGGGAACGTCTTCTCATGCTCCGTGA |
| | | TGCATGAGGCTCTGCACAACCACTACACACAGAAGAGCCTC |
| | | TCCCTGTCTCCGGGCAAA |
| 188 | Anti-CD228 antibody | SSELTQDPAVSVALGQTVRITCQGDSLRSYYTSWYQQKPGQ |
| | clone 24 LC amino acid | APVFVIYGKNNRPSGIPDRFSGSSSGNTASLTITGAQAEDE |
| | | ADYYCNSRDSSGNHVVFGGGTKLTVLRTVAAPSVFIFPPSD |
| | | EQLKSGTASVVCLLNNFYPREAKVQWKVDNALQSGNSQESV |
| | | TEQDSKDSTYSLSSTLTLSKADYEKHKVYACEVTHQGLSSP |
| | | VTKSENRGEC |
| 189 | Anti-CD228 antibody | TCTTCTGAGCTGACTCAGGACCCTGCTGTGTCTGTGGCCTT |
| | clone 24 LC nucleic | GGGACAGACAGTCAGGATCACATGCCAAGGAGACAGCCTCA |
| | acid | GATCCTATTATACAAGCTGGTACCAGCAGAAGCCAGGACAG |
| | | GCCCCTGTATTTGTCATCTATGGTAAAAACAACCGGCCCTC |
| | | AGGGATCCCAGACCGATTCTCTGGCTCCAGCTCAGGAAACA |
| | | CAGCTTCCTTGACCATCACTGGGGCTCAGGCGGAAGATGAG |
| | | GCTGACTATTACTGTAATTCCCGGGACAGCAGTGGTAACCA |
| | | TGTGGTTTTCGGCGGAGGGACCAAGCTGACCGTCCTACGTA |
| | | CGGTGGCTGCACCATCTGTCTTCATCTTCCCGCCATCTGAT |
| | | GAGCAGTTGAAATCTGGAACTGCCTCTGTTGTGTGCCTGCT |
| | | GAATAACTTCTATCCCAGAGAGGCCAAAGTACAGTGGAAGG |
| | | TGGATAACGCCCTCCAATCGGGTAACTCCCAGGAGAGTGTC |
| | | ACAGAGCAGGACAGCAAGGACAGCACCTACAGCCTCAGCAG |
| | | CACCCTGACGCTGAGCAAAGCAGACTACGAGAAACACAAAG |
| | | TCTACGCCTGCGAAGTCACCCATCAGGGCCTGAGCTCGCCC |
| | | GTCACAAAGAGCTTCAACAGGGGAGAGTGT |
| 190 | Anti-CD228 antibody | DYYMS |
| | clone 28 CDR-H1 | |
| | (Kabat) | |
| 191 | Anti-CD228 antibody | YISSSGGAIFYADSVKG |
| | clone 28 CDR-H2 | |
| | (Kabat) | |
| 192 | Anti-CD228 antibody | RYSSGWFEYFKH |
| | clone 28 CDR-H3 | |
| | (Kabat) | |
| 193 | Anti-CD228 antibody | GFTFSDYY |
| | clone 28 CDR-H1 | |
| | (IMGT) | |
| 194 | Anti-CD228 antibody | ISSSGGAI |
| | clone 28 CDR-H2 | |
| | (IMGT) | |
| 195 | Anti-CD228 antibody | ARRYSSGWFEYFKH |
| | clone 28 CDR-H3 | |
| | (IMGT) | |
| 196 | Anti-CD228 antibody | KSSQSVENSSNKKNYLA |
| | clone 28 CDR-L1 | |
| | (Kabat) | |
| 197 | Anti-CD228 antibody | WASTRES |
| | clone 28 CDR-L2 | |
| | (Kabat) | |
| 198 | Anti-CD228 antibody | QQYYLAPYT |
| | clone 28 CDR-L3 | |
| | (Kabat) | |
| 199 | Anti-CD228 antibody | QSVENSSNKKNY |
| | clone 28 CDR-L1 | |
| | (IMGT) | |
| 200 | Anti-CD228 antibody | WAS |
| | clone 28 CDR-L2 | |
| | (IMGT) | |
| 201 | Anti-CD228 antibody | QQYYLAPYT |
| | clone 28 CDR-L3 | |
| | (IMGT) | |
| 202 | Anti-CD228 antibody | QVQLVESGGGLVKPGGSLRLSCAASGFTFSDYYMSWIRQAP |
| | clone 28 VH amino | GKGLEWISYISSSGGAIFYADSVKGRFTISRDNAKNSLYLQ |
| | acid | MNSLRTEDTAVYYCARRYSSGWFEYFKHWGQGTLVTVSS |
| 203 | Anti-CD228 antibody | CAGGTGCAGCTGGTGGAGTCTGGGGGAGGCTTGGTCAAGCC |
| | clone 28 VH nucleic | TGGAGGGTCCCTGAGACTCTCCTGTGCAGCCTCTGGATTCA |
| | acid | CCTTCAGTGACTACTACATGAGCTGGATCCGCCAGGCTCCA |
| | | GGGAAGGGGCTGGAGTGGATTTCATACATTAGTAGTAGTGG |
| | | TGGTGCCATATTCTACGCAGACTCTGTGAAGGGCCGATTCA |
| | | CCATCTCCAGGGACAACGCCAAGAACTCACTGTATCTGCAA |
| | | ATGAACAGCCTGAGAACCGAGGACACGGCCGTGTATTACTG |
| | | TGCGAGAAGGTATAGCAGTGGCTGGTTCGAATACTTCAAAC |
| | | ACTGGGGCCAGGGCACCCTGGTCACCGTCTCCTCA |
| 204 | Anti-CD228 antibody | DIVMTQSPDSLAVSLGERATINCKSSQSVFNSSNKKNYLAW |
| | clone 28 VL amino acid | YQQKPGQPPKLLIYWASTRESGVPDRFSGSGSGTDFTLTIS |
| | | SLQAEDVAVYYCQQYYLAPYTFGQGTKLEIK |
| 205 | Anti-CD228 antibody | GACATCGTGATGACCCAGTCTCCAGACTCCCTGGCTGTGTC |
| | clone 28 VL nucleic | TCTGGGCGAGAGGGCCACCATCAACTGCAAGTCCAGCCAGA |
| | acid | GTGTTTTCAACAGTTCCAACAAAAAGAACTACTTAGCTTGG |
| | | TACCAGCAGAAACCAGGACAGCCTCCTAAGCTGCTCATTTA |
| | | CTGGGCATCTACCCGGGAATCCGGGGTCCCTGACCGATTCA |
| | | GTGGCAGCGGGTCTGGGACAGATTTCACTCTCACCATCAGC |
| | | AGCCTGCAGGCTGAAGATGTGGCAGTTTATTACTGTCAGCA |
| | | ATATTATCTTGCTCCGTACACTTTTGGCCAGGGGACCAAGC |
| | | TGGAGATCAAACGT |
| 206 | Anti-CD228 antibody | QVQLVESGGGLVKPGGSLRLSCAASGFTFSDYYMSWIRQAP |
| | clone 28 HC amino | GKGLEWISYISSSGGAIFYADSVKGRFTISRDNAKNSLYLQ |
| | acid | MNSLRTEDTAVYYCARRYSSGWFEYFKHWGQGTLVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| 207 | Anti-CD228 antibody | CAGGTGCAGCTGGTGGAGTCTGGGGGAGGCTTGGTCAAGCC |
| | clone 28 HC nucleic | TGGAGGGTCCCTGAGACTCTCCTGTGCAGCCTCTGGATTCA |
| | acid | CCTTCAGTGACTACTACATGAGCTGGATCCGCCAGGCTCCA |
| | | GGGAAGGGGCTGGAGTGGATTTCATACATTAGTAGTAGTGG |
| | | TGGTGCCATATTCTACGCAGACTCTGTGAAGGGCCGATTCA |
| | | CCATCTCCAGGGACAACGCCAAGAACTCACTGTATCTGCAA |
| | | ATGAACAGCCTGAGAACCGAGGACACGGCCGTGTATTACTG |
| | | TGCGAGAAGGTATAGCAGTGGCTGGTTCGAATACTTCAAAC |
| | | ACTGGGGCCAGGGCACCCTGGTCACCGTCTCCTCAGCTAGC |
| | | ACCAAGGGCCCATCTGTCTTCCCCCTGGCACCCTCCTCCAA |
| | | GAGCACCTCTGGGGGCACAGCTGCCCTGGGCTGCCTGGTCA |
| | | AGGACTACTTCCCTGAACCTGTGACAGTGTCCTGGAACTCA |
| | | GGAGCCCTGACCAGCGGCGTGCACACCTTCCCGGCTGTCCT |
| | | ACAGTCCTCAGGACTCTACTCCCTCAGCAGCGTGGTGACCG |
| | | TGCCCTCCAGCAGCTTGGGCACCCAGACCTACATCTGCAAC |
| | | GTGAATCACAAGCCCAGCAACACCAAGGTGGACAAGAAAGT |
| | | TGAGCCCAAATCTTGTGACAAAACTCACACATGCCCACCGT |
| | | GCCCAGCACCTGAACTCCTGGGGGGACCGTCAGTCTTCCTC |
| | | TTCCCCCCAAAACCCAAGGACACCCTCATGATCTCCCGGAC |
| | | CCCTGAGGTCACATGCGTGGTGGTGGACGTGAGCCACGAAG |
| | | ACCCTGAGGTCAAGTTCAACTGGTACGTGGACGGCGTGGAG |
| | | GTGCATAATGCCAAGACAAAGCCGCGGGAGGAGCAGTACAA |
| | | CAGCACGTACCGTGTGGTCAGCGTCCTCACCGTCCTGCACC |
| | | AGGACTGGCTGAATGGCAAGGAGTACAAGTGCAAGGTCTCC |
| | | AACAAAGCCCTCCCAGCCCCCATCGAGAAAACCATCTCCAA |
| | | AGCCAAAGGGCAGCCCCGAGAACCACAGGTGTACACCCTGC |
| | | CCCCATCCCGGGATGAGCTGACCAAGAACCAGGTCAGCCTG |
| | | ACCTGCCTGGTCAAAGGCTTCTATCCCAGCGACATCGCCGT |
| | | GGAGTGGGAGAGCAATGGGCAGCCGGAGAACAACTACAAGA |
| | | CCACGCCTCCCGTGCTGGACTCCGACGGCTCCTTCTTCCTC |
| | | TACAGCAAGCTCACCGTGGACAAGAGCAGATGGCAGCAGGG |
| | | GAACGTCTTCTCATGCTCCGTGATGCATGAGGCTCTGCACA |
| | | ACCACTACACACAGAAGAGCCTCTCCCTGTCTCCGGGCAAA |
| 208 | Anti-CD228 antibody | DIVMTQSPDSLAVSLGERATINCKSSQSVENSSNKKNYLAW |
| | clone 28 LC amino acid | YQQKPGQPPKLLIYWASTRESGVPDRFSGSGSGTDFTLTIS |
| | | SLQAEDVAVYYCQQYYLAPYTFGQGTKLEIKRTVAAPSVFI |
| | | FPPSDEQLKSGTASVVCLLNNFYPREAKVQWKVDNALQSGN |
| | | SQESVTEQDSKDSTYSLSSTLTLSKADYEKHKVYACEVTHQ |
| | | GLSSPVTKSFNRGEC |
| 209 | Anti-CD228 antibody | GACATCGTGATGACCCAGTCTCCAGACTCCCTGGCTGTGTC |
| | clone 28 LC nucleic | TCTGGGCGAGAGGGCCACCATCAACTGCAAGTCCAGCCAGA |
| | acid | GTGTTTTCAACAGTTCCAACAAAAAGAACTACTTAGCTTGG |
| | | TACCAGCAGAAACCAGGACAGCCTCCTAAGCTGCTCATTTA |
| | | CTGGGCATCTACCCGGGAATCCGGGGTCCCTGACCGATTCA |
| | | GTGGCAGCGGGTCTGGGACAGATTTCACTCTCACCATCAGC |
| | | AGCCTGCAGGCTGAAGATGTGGCAGTTTATTACTGTCAGCA |
| | | ATATTATCTTGCTCCGTACACTTTTGGCCAGGGGACCAAGC |
| | | TGGAGATCAAACGTACGGTGGCTGCACCATCTGTCTTCATC |
| | | TTCCCGCCATCTGATGAGCAGTTGAAATCTGGAACTGCCTC |
| | | TGTTGTGTGCCTGCTGAATAACTTCTATCCCAGAGAGGCCA |
| | | AAGTACAGTGGAAGGTGGATAACGCCCTCCAATCGGGTAAC |
| | | TCCCAGGAGAGTGTCACAGAGCAGGACAGCAAGGACAGCAC |
| | | CTACAGCCTCAGCAGCACCCTGACGCTGAGCAAAGCAGACT |
| | | ACGAGAAACACAAAGTCTACGCCTGCGAAGTCACCCATCAG |
| | | GGCCTGAGCTCGCCCGTCACAAAGAGCTTCAACAGGGGAGA |
| | | GTGT |
| 210 | Anti-CD228 antibody | SYGIH |
| | clone 30 CDR-H1 | |
| | (Kabat) | |
| 211 | Anti-CD228 antibody | VIWYDGSNKYYADSVKG |
| | clone 30 CDR-H2 | |
| | (Kabat) | |
| 212 | Anti-CD228 antibody | RGDSSSWHEDY |
| | clone 30 CDR-H3 | |
| | (Kabat) | |
| 213 | Anti-CD228 antibody | GFTFSSYG |
| | clone 30 CDR-H1 | |
| | (IMGT) | |
| 214 | Anti-CD228 antibody | IWYDGSNK |
| | clone 30 CDR-H2 | |
| | (IMGT) | |
| 215 | Anti-CD228 antibody | ARRGDSSSWHEDY |
| | clone 30 CDR-H3 | |
| | (IMGT) | |
| 216 | Anti-CD228 antibody | TGTSSDVGRHNLVS |
| | clone 30 CDR-L1 | |
| | (Kabat) | |
| 217 | Anti-CD228 antibody | EVTKRPA |
| | clone 30 CDR-L2 | |
| | (Kabat) | |
| 218 | Anti-CD228 antibody | CSYAGSSTYV |
| | clone 30 CDR-L3 | |
| | (Kabat) | |
| 219 | Anti-CD228 antibody | SSDVGRHNL |
| | clone 30 CDR-L1 | |
| | (IMGT) | |
| 220 | Anti-CD228 antibody | EVT |
| | clone 30 CDR-L2 | |
| | (IMGT) | |
| 221 | Anti-CD228 antibody | CSYAGSSTYV |
| | clone 30 CDR-L3 | |
| | (IMGT) | |
| 222 | Anti-CD228 antibody | QVQLVESGGGVVQPGRSLRLSCAASGFTFSSYGIHWVRQAP |
| | clone 30 VH amino | GKGLEWVAVIWYDGSNKYYADSVKGRFTISRDNSKNTLYLQ |
| | acid | MNSLRAEDTAVYYCARRGDSSSWHFDYWGQGTLVTVSS |
| 223 | Anti-CD228 antibody | CAGGTGCAGCTGGTTGAATCTGGTGGCGGAGTGGTGCAGCC |
| | clone 30 VH nucleic | TGGCAGATCTCTGAGACTGTCTTGTGCCGCCTCCGGCTTCA |
| | acid | CCTTCTCCTCTTACGGAATCCACTGGGTCCGACAGGCCCCT |
| | | GGCAAAGGATTGGAATGGGTCGCCGTGATTTGGTACGACGG |
| | | CTCCAACAAGTACTACGCCGACTCCGTGAAGGGCAGATTCA |
| | | CCATCTCTCGGGACAACTCCAAGAACACCCTGTACCTGCAG |
| | | ATGAACTCCCTGAGAGCCGAGGACACCGCCGTGTACTACTG |
| | | TGCCAGAAGAGGCGACTCCTCCTCCTGGCACTTTGACTATT |
| | | GGGGCCAGGGCACCCTGGTCACCGTTAGTTCT |
| 224 | Anti-CD228 antibody | QSALTQPASVSGSPGQSITISCTGTSSDVGRHNLVSWYQQH |
| | clone 30 VL amino acid | PGKVPKVMIYEVTKRPAGVSNRFSGSKSGNTASLTISGLQA |
| | | EDEADYYCCSYAGSSTYVFGTGTRVTVL |
| 225 | Anti-CD228 antibody | CAGTCTGCTCTGACCCAGCCTGCTTCTGTGTCTGGCTCTCC |
| | clone 30 VL nucleic | CGGCCAGTCCATCACCATCTCTTGTACCGGCACCTCCTCCG |
| | acid | ACGTGGGCAGACACAATCTGGTGTCCTGGTATCAGCAGCAT |
| | | CCCGGCAAGGTGCCCAAAGTGATGATCTACGAAGTGACCAA |
| | | GCGGCCTGCCGGCGTGTCCAACAGATTCTCCGGATCCAAGT |
| | | CCGGCAACACCGCCAGCCTGACAATTAGCGGACTGCAGGCT |
| | | GAGGACGAGGCCGACTACTACTGCTGTTCTTACGCCGGCTC |
| | | CTCCACCTACGTGTTCGGCACTGGAACAAGAGTGACAGTGC |
| | | TG |
| 226 | Anti-CD228 antibody | QVQLVESGGGVVQPGRSLRLSCAASGFTFSSYGIHWVRQAP |
| | clone 30 HC amino | GKGLEWVAVIWYDGSNKYYADSVKGRFTISRDNSKNTLYLQ |
| | acid | MNSLRAEDTAVYYCARRGDSSSWHFDYWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| 227 | Anti-CD228 antibody | CAGGTGCAGCTGGTGGAGTCTGGGGGAGGCGTGGTCCAGCC |
| | clone 30 HC nucleic | TGGGAGGTCCCTGAGACTCTCCTGTGCAGCGTCTGGATTCA |
| | acid | CCTTCAGTAGCTATGGCATTCACTGGGTCCGCCAGGCTCCA |
| | | GGCAAGGGGCTGGAGTGGGTGGCAGTTATATGGTATGATGG |
| | | AAGTAATAAATACTATGCAGACTCCGTGAAGGGCCGATTCA |
| | | CCATCTCCAGAGACAATTCCAAGAACACGCTGTATCTGCAA |
| | | ATGAACAGCCTGAGAGCCGAGGACACGGCTGTGTATTACTG |
| | | TGCGAGGAGAGGCGATAGCAGCAGCTGGCACTTTGACTACT |
| | | GGGGCCAGGGAACCCTGGTCACCGTCTCCTCAGCTAGCACC |
| | | AAGGGCCCATCTGTCTTCCCCCTGGCACCCTCCTCCAAGAG |
| | | CACCTCTGGGGGCACAGCTGCCCTGGGCTGCCTGGTCAAGG |
| | | ACTACTTCCCTGAACCTGTGACAGTGTCCTGGAACTCAGGA |
| | | GCCCTGACCAGCGGCGTGCACACCTTCCCGGCTGTCCTACA |
| | | GTCCTCAGGACTCTACTCCCTCAGCAGCGTGGTGACCGTGC |
| | | CCTCCAGCAGCTTGGGCACCCAGACCTACATCTGCAACGTG |
| | | AATCACAAGCCCAGCAACACCAAGGTGGACAAGAAAGTTGA |
| | | GCCCAAATCTTGTGACAAAACTCACACATGCCCACCGTGCC |
| | | CAGCACCTGAACTCCTGGGGGGACCGTCAGTCTTCCTCTTC |
| | | CCCCCAAAACCCAAGGACACCCTCATGATCTCCCGGACCCC |
| | | TGAGGTCACATGCGTGGTGGTGGACGTGAGCCACGAAGACC |
| | | CTGAGGTCAAGTTCAACTGGTACGTGGACGGCGTGGAGGTG |
| | | CATAATGCCAAGACAAAGCCGCGGGAGGAGCAGTACAACAG |
| | | CACGTACCGTGTGGTCAGCGTCCTCACCGTCCTGCACCAGG |
| | | ACTGGCTGAATGGCAAGGAGTACAAGTGCAAGGTCTCCAAC |
| | | AAAGCCCTCCCAGCCCCCATCGAGAAAACCATCTCCAAAGC |
| | | CAAAGGGCAGCCCCGAGAACCACAGGTGTACACCCTGCCCC |
| | | CATCCCGGGATGAGCTGACCAAGAACCAGGTCAGCCTGACC |
| | | TGCCTGGTCAAAGGCTTCTATCCCAGCGACATCGCCGTGGA |
| | | GTGGGAGAGCAATGGGCAGCCGGAGAACAACTACAAGACCA |
| | | CGCCTCCCGTGCTGGACTCCGACGGCTCCTTCTTCCTCTAC |
| | | AGCAAGCTCACCGTGGACAAGAGCAGATGGCAGCAGGGGAA |
| | | CGTCTTCTCATGCTCCGTGATGCATGAGGCTCTGCACAACC |
| | | ACTACACACAGAAGAGCCTCTCCCTGTCTCCGGGCAAA |
| 228 | Anti-CD228 antibody | QSALTQPASVSGSPGQSITISCTGTSSDVGRHNLVSWYQQH |
| | clone 30 LC amino acid | PGKVPKVMIYEVTKRPAGVSNRFSGSKSGNTASLTISGLQA |
| | | EDEADYYCCSYAGSSTYVFGTGTRVTVLGQPKAAPSVTLEP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS |
| 229 | Anti-CD228 antibody | CAGTCTGCCCTGACTCAGCCTGCCTCCGTGTCTGGGTCTCC |
| | clone 30 LC nucleic | TGGACAGTCGATCACCATCTCCTGCACTGGAACCAGCAGTG |
| | acid | ATGTTGGGAGGCATAACCTTGTCTCCTGGTACCAACAGCAC |
| | | CCAGGCAAAGTCCCCAAAGTCATGATTTATGAGGTCACTAA |
| | | GCGGCCCGCAGGGGTGTCTAATCGCTTCTCTGGCTCCAAGT |
| | | CTGGCAACACGGCCTCCCTGACAATCTCTGGGCTCCAGGCT |
| | | GAGGACGAGGCTGATTATTACTGCTGCTCATATGCAGGTAG |
| | | TAGCACTTATGTCTTCGGAACTGGGACCAGGGTCACCGTCC |
| | | TTGGTCAGCCCAAGGCGGCGCCCTCGGTCACTCTGTTCCCG |
| | | CCCTCCTCTGAGGAGCTTCAAGCCAACAAGGCCACACTGGT |
| | | GTGTCTCATAAGTGACTTCTACCCGGGAGCCGTGACAGTGG |
| | | CCTGGAAGGCAGATAGCAGCCCCGTCAAGGCGGGAGTGGAG |
| | | ACCACCACACCCTCCAAACAAAGCAACAACAAGTACGCGGC |
| | | CAGCAGCTATCTGAGCCTGACGCCTGAGCAGTGGAAGTCCC |
| | | ACAGAAGCTACAGCTGCCAGGTCACGCATGAAGGGAGCACC |
| | | GTGGAGAAGACAGTGGCCCCTACAGAATGTTCA |
| 230 | Anti-CD228 antibody | DYYMS |
| | clone 32 CDR-H1 | |
| | (Kabat) | |
| 231 | Anti-CD228 antibody | YISSSGGAIFYADSVKG |
| | clone 32 CDR-H2 | |
| | (Kabat) | |
| 232 | Anti-CD228 antibody | RYSSGWFEYFKH |
| | clone 32 CDR-H3 | |
| | (Kabat) | |
| 233 | Anti-CD228 antibody | GFTFSDYY |
| | clone 32 CDR-H1 | |
| | (IMGT) | |
| 234 | Anti-CD228 antibody | ISSSGGAI |
| | clone 32 CDR-H2 | |
| | (IMGT) | |
| 235 | Anti-CD228 antibody | ARRYSSGWFEYFKH |
| | clone 32 CDR-H3 | |
| | (IMGT) | |
| 236 | Anti-CD228 antibody | KSSQSVLNSSNKKNYLA |
| | clone 32 CDR-L1 | |
| | (Kabat) | |
| 237 | Anti-CD228 antibody | WASTRES |
| | clone 32 CDR-L2 | |
| | (Kabat) | |
| 238 | Anti-CD228 antibody | QQYYLAPYT |
| | clone 32 CDR-L3 | |
| | (Kabat) | |
| 239 | Anti-CD228 antibody | QSVLNSSNKKNY |
| | clone 32 CDR-L1 | |
| | (IMGT) | |
| 240 | Anti-CD228 antibody | WAS |
| | clone 32 CDR-L2 | |
| | (IMGT) | |
| 241 | Anti-CD228 antibody | QQYYLAPYT |
| | clone 32 CDR-L3 | |
| | (IMGT) | |
| 242 | Anti-CD228 antibody | QVQLVESGGGLVKPGGSLRLSCAASGFTFSDYYMSWIRQAP |
| | clone 32 VH amino | GKGLEWISYISSSGGAIFYADSVKGRFTISRDNAKNSLYLQ |
| | acid | MNSLRTEDTAVYYCARRYSSGWFEYFKHWGQGTLVTVSS |
| 243 | Anti-CD228 antibody | CAGGTGCAGCTGGTGGAGTCTGGGGGAGGCTTGGTCAAGCC |
| | clone 32 VH nucleic | TGGAGGGTCCCTGAGACTCTCCTGTGCAGCCTCTGGATTCA |
| | acid | CCTTCAGTGACTACTACATGAGCTGGATCCGCCAGGCTCCA |
| | | GGGAAGGGGCTGGAGTGGATTTCATACATTAGTAGTAGTGG |
| | | TGGTGCCATATTCTACGCAGACTCTGTGAAGGGCCGATTCA |
| | | CCATCTCCAGGGACAACGCCAAGAACTCACTGTATCTGCAA |
| | | ATGAACAGCCTGAGAACCGAGGACACGGCCGTGTATTACTG |
| | | TGCGAGAAGGTATAGCAGTGGCTGGTTCGAGTACTTCAAAC |
| | | ACTGGGGCCAGGGCACCCTGGTCACCGTCTCCTCA |
| 244 | Anti-CD228 antibody | DIVMTQSPDSLAVSLGERATINCKSSQSVLNSSNKKNYLAW |
| | clone 32 VL amino acid | YQQKPGQPPKLLIYWASTRESGVPDRFSGSGSGTDFTLTIS |
| | | SLQAEDVAVYYCQQYYLAPYTFGQGTKLEIK |
| 245 | Anti-CD228 antibody | GACATCGTGATGACCCAGTCTCCAGACTCCCTGGCTGTGTC |
| | clone 32 VL nucleic | TCTGGGCGAGAGGGCCACCATCAACTGCAAGTCCAGCCAGA |
| | acid | GTGTTTTAAACAGTTCCAACAAAAAGAACTACTTAGCTTGG |
| | | TACCAGCAGAAACCAGGACAGCCTCCTAAGCTGCTCATTTA |
| | | CTGGGCATCTACCCGGGAATCCGGGGTCCCTGACCGATTCA |
| | | GTGGCAGCGGGTCTGGGACAGATTTCACTCTCACCATCAGC |
| | | AGCCTGCAGGCTGAAGATGTGGCAGTTTATTACTGTCAGCA |
| | | ATATTATCTTGCTCCGTACACTTTTGGCCAGGGGACCAAGC |
| | | TGGAGATCAAACGT |
| 246 | Anti-CD228 antibody | QVQLVESGGGLVKPGGSLRLSCAASGFTFSDYYMSWIRQAP |
| | clone 32 HC amino | GKGLEWISYISSSGGAIFYADSVKGRFTISRDNAKNSLYLQ |
| | acid | MNSLRTEDTAVYYCARRYSSGWFEYFKHWGQGTLVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| 247 | Anti-CD228 antibody | CAGGTGCAGCTGGTGGAGTCTGGGGGAGGCTTGGTCAAGCC |
| | clone 32 HC nucleic | TGGAGGGTCCCTGAGACTCTCCTGTGCAGCCTCTGGATTCA |
| | acid | CCTTCAGTGACTACTACATGAGCTGGATCCGCCAGGCTCCA |
| | | GGGAAGGGGCTGGAGTGGATTTCATACATTAGTAGTAGTGG |
| | | TGGTGCCATATTCTACGCAGACTCTGTGAAGGGCCGATTCA |
| | | CCATCTCCAGGGACAACGCCAAGAACTCACTGTATCTGCAA |
| | | ATGAACAGCCTGAGAACCGAGGACACGGCCGTGTATTACTG |
| | | TGCGAGAAGGTATAGCAGTGGCTGGTTCGAGTACTTCAAAC |
| | | ACTGGGGCCAGGGCACCCTGGTCACCGTCTCCTCAGCTAGC |
| | | ACCAAGGGCCCATCTGTCTTCCCCCTGGCACCCTCCTCCAA |
| | | GAGCACCTCTGGGGGCACAGCTGCCCTGGGCTGCCTGGTCA |
| | | AGGACTACTTCCCTGAACCTGTGACAGTGTCCTGGAACTCA |
| | | GGAGCCCTGACCAGCGGCGTGCACACCTTCCCGGCTGTCCT |
| | | ACAGTCCTCAGGACTCTACTCCCTCAGCAGCGTGGTGACCG |
| | | TGCCCTCCAGCAGCTTGGGCACCCAGACCTACATCTGCAAC |
| | | GTGAATCACAAGCCCAGCAACACCAAGGTGGACAAGAAAGT |
| | | TGAGCCCAAATCTTGTGACAAAACTCACACATGCCCACCGT |
| | | GCCCAGCACCTGAACTCCTGGGGGGACCGTCAGTCTTCCTC |
| | | TTCCCCCCAAAACCCAAGGACACCCTCATGATCTCCCGGAC |
| | | CCCTGAGGTCACATGCGTGGTGGTGGACGTGAGCCACGAAG |
| | | ACCCTGAGGTCAAGTTCAACTGGTACGTGGACGGCGTGGAG |
| | | GTGCATAATGCCAAGACAAAGCCGCGGGAGGAGCAGTACAA |
| | | CAGCACGTACCGTGTGGTCAGCGTCCTCACCGTCCTGCACC |
| | | AGGACTGGCTGAATGGCAAGGAGTACAAGTGCAAGGTCTCC |
| | | AACAAAGCCCTCCCAGCCCCCATCGAGAAAACCATCTCCAA |
| | | AGCCAAAGGGCAGCCCCGAGAACCACAGGTGTACACCCTGC |
| | | CCCCATCCCGGGATGAGCTGACCAAGAACCAGGTCAGCCTG |
| | | ACCTGCCTGGTCAAAGGCTTCTATCCCAGCGACATCGCCGT |
| | | GGAGTGGGAGAGCAATGGGCAGCCGGAGAACAACTACAAGA |
| | | CCACGCCTCCCGTGCTGGACTCCGACGGCTCCTTCTTCCTC |
| | | TACAGCAAGCTCACCGTGGACAAGAGCAGATGGCAGCAGGG |
| | | GAACGTCTTCTCATGCTCCGTGATGCATGAGGCTCTGCACA |
| | | ACCACTACACACAGAAGAGCCTCTCCCTGTCTCCGGGCAAA |
| 248 | Anti-CD228 antibody | DIVMTQSPDSLAVSLGERATINCKSSQSVLNSSNKKNYLAW |
| | clone 32 LC amino acid | YQQKPGQPPKLLIYWASTRESGVPDRFSGSGSGTDFTLTIS |
| | | SLQAEDVAVYYCQQYYLAPYTFGQGTKLEIKRTVAAPSVFI |
| | | FPPSDEQLKSGTASVVCLLNNFYPREAKVQWKVDNALQSGN |
| | | SQESVTEQDSKDSTYSLSSTLTLSKADYEKHKVYACEVTHQ |
| | | GLSSPVTKSFNRGEC |
| 249 | Anti-CD228 antibody | GACATCGTGATGACCCAGTCTCCAGACTCCCTGGCTGTGTC |
| | clone 32 LC nucleic | TCTGGGCGAGAGGGCCACCATCAACTGCAAGTCCAGCCAGA |
| | acid | GTGTTTTAAACAGTTCCAACAAAAAGAACTACTTAGCTTGG |
| | | TACCAGCAGAAACCAGGACAGCCTCCTAAGCTGCTCATTTA |
| | | CTGGGCATCTACCCGGGAATCCGGGGTCCCTGACCGATTCA |
| | | GTGGCAGCGGGTCTGGGACAGATTTCACTCTCACCATCAGC |
| | | AGCCTGCAGGCTGAAGATGTGGCAGTTTATTACTGTCAGCA |
| | | ATATTATCTTGCTCCGTACACTTTTGGCCAGGGGACCAAGC |
| | | TGGAGATCAAACGTACGGTGGCTGCACCATCTGTCTTCATC |
| | | TTCCCGCCATCTGATGAGCAGTTGAAATCTGGAACTGCCTC |
| | | TGTTGTGTGCCTGCTGAATAACTTCTATCCCAGAGAGGCCA |
| | | AAGTACAGTGGAAGGTGGATAACGCCCTCCAATCGGGTAAC |
| | | TCCCAGGAGAGTGTCACAGAGCAGGACAGCAAGGACAGCAC |
| | | CTACAGCCTCAGCAGCACCCTGACGCTGAGCAAAGCAGACT |
| | | ACGAGAAACACAAAGTCTACGCCTGCGAAGTCACCCATCAG |
| | | GGCCTGAGCTCGCCCGTCACAAAGAGCTTCAACAGGGGAGA |
| | | GTGT |
| 250 | Anti-CD228 antibody | SNSAAWN |
| | clone 35 CDR-H1 | |
| | (Kabat) | |
| 251 | Anti-CD228 antibody | RTYYRSKWEDDYAVSVKG |
| | clone 35 CDR-H2 | |
| | (Kabat) | |
| 252 | Anti-CD228 antibody | RVHWGGPEDY |
| | clone 35 CDR-H3 | |
| | (Kabat) | |
| 253 | Anti-CD228 antibody | GDSVSSNSAA |
| | clone 35 CDR-H1 | |
| | (IMGT) | |
| 254 | Anti-CD228 antibody | TYYRSKWED |
| | clone 35 CDR-H2 | |
| | (IMGT) | |
| 255 | Anti-CD228 antibody | ARRVHWGGPFDY |
| | clone 35 CDR-H3 | |
| | (IMGT) | |
| 256 | Anti-CD228 antibody | RASQGISSYLA |
| | clone 35 CDR-L1 | |
| | (Kabat) | |
| 257 | Anti-CD228 antibody | VASTLQS |
| | clone 35 CDR-L2 | |
| | (Kabat) | |
| 258 | Anti-CD228 antibody | QQLTGYPFT |
| | clone 35 CDR-L3 | |
| | (Kabat) | |
| 259 | Anti-CD228 antibody | QGISSY |
| | clone 35 CDR-L1 | |
| | (IMGT) | |
| 260 | Anti-CD228 antibody | VAS |
| | clone 35 CDR-L2 | |
| | (IMGT) | |
| 261 | Anti-CD228 antibody | QQLTGYPFT |
| | clone 35 CDR-L3 | |
| | (IMGT) | |
| 262 | Anti-CD228 antibody | QVQLQQSGPGLVKPSQTLSLTCAISGDSVSSNSAAWNWIRQ |
| | clone 35 VH amino | SPSSGLEWLGRTYYRSKWFDDYAVSVKGRITINPDTSKNQF |
| | acid | SLQLKFVIPEDTAVYYCARRVHWGGPFDYWGQGTLVTVSS |
| 263 | Anti-CD228 antibody | CAGGTACAGCTGCAGCAGTCAGGTCCAGGACTGGTGAAGCC |
| | clone 35 VH nucleic | CTCGCAGACCCTCTCACTCACCTGTGCCATCTCCGGGGACA |
| | acid | GTGTCTCTAGCAACAGTGCTGCTTGGAACTGGATCAGGCAG |
| | | TCCCCCTCGAGTGGCCTTGAGTGGCTGGGAAGGACTTACTA |
| | | CAGGTCCAAGTGGTTTGATGATTATGCAGTCTCTGTGAAAG |
| | | GTCGAATAACCATCAACCCAGACACATCCAAGAACCAGTTC |
| | | TCCCTTCAGTTAAAGTTTGTGATTCCCGAGGACACGGCTGT |
| | | TTATTATTGTGCAAGAAGAGTCCACTGGGGGGGTCCCTTTG |
| | | ACTATTGGGGCCAGGGAACCCTGGTCACCGTCTCCTCA |
| 264 | Anti-CD228 antibody | DIQLTQSPSFLSASVGDRVTITCRASQGISSYLAWYQQRPG |
| | clone 35 VL amino acid | KAPKVLISVASTLQSGVPSRFSGSGSGTDFTLTISSLQPED |
| | | FATYYCQQLTGYPFTFGPGTKVDIK |
| 265 | Anti-CD228 antibody | GACATCCAGTTGACCCAGTCTCCATCCTTCCTGTCTGCCTC |
| | clone 35 VL nucleic | TGTAGGAGACAGAGTCACCATCACTTGCCGGGCCAGTCAGG |
| | acid | GCATTAGCAGTTATTTAGCCTGGTATCAGCAAAGACCAGGG |
| | | AAAGCCCCTAAGGTCTTGATCTCTGTTGCATCCACTTTGCA |
| | | AAGTGGGGTCCCATCAAGGTTCAGCGGCAGTGGATCTGGGA |
| | | CAGATTTCACTCTCACAATCAGCAGCCTGCAGCCTGAGGAT |
| | | TTTGCGACTTATTACTGTCAACAGCTTACTGGTTACCCATT |
| | | CACTTTCGGCCCTGGGACCAAAGTGGATATCAAACGT |
| 266 | Anti-CD228 antibody | QVQLQQSGPGLVKPSQTLSLTCAISGDSVSSNSAAWNWIRQ |
| | clone 35 HC amino | SPSSGLEWLGRTYYRSKWEDDYAVSVKGRITINPDTSKNQF |
| | acid | SLQLKFVIPEDTAVYYCARRVHWGGPFDYWGQGTLVTVSSA |
| | | STKGPSVFPLAPSSKSTSGGTAALGCLVKDYFPEPVTVSWN |
| | | SGALTSGVHTFPAVLQSSGLYSLSSVVTVPSSSLGTQTYIC |
| | | NVNHKPSNTKVDKKVEPKSCDKTHTCPPCPAPELLGGPSVF |
| | | LFPPKPKDTLMISRTPEVTCVVVDVSHEDPEVKFNWYVDGV |
| | | EVHNAKTKPREEQYNSTYRVVSVLTVLHQDWLNGKEYKCKV |
| | | SNKALPAPIEKTISKAKGQPREPQVYTLPPSRDELTKNQVS |
| | | LTCLVKGFYPSDIAVEWESNGQPENNYKTTPPVLDSDGSFF |
| | | LYSKLTVDKSRWQQGNVFSCSVMHEALHNHYTQKSLSLSPG |
| | | K |
| 267 | Anti-CD228 antibody | CAGGTACAGCTGCAGCAGTCAGGTCCAGGACTGGTGAAGCC |
| | clone 35 HC nucleic | CTCGCAGACCCTCTCACTCACCTGTGCCATCTCCGGGGACA |
| | acid | GTGTCTCTAGCAACAGTGCTGCTTGGAACTGGATCAGGCAG |
| | | TCCCCCTCGAGTGGCCTTGAGTGGCTGGGAAGGACTTACTA |
| | | CAGGTCCAAGTGGTTTGATGATTATGCAGTCTCTGTGAAAG |
| | | GTCGAATAACCATCAACCCAGACACATCCAAGAACCAGTTC |
| | | TCCCTTCAGTTAAAGTTTGTGATTCCCGAGGACACGGCTGT |
| | | TTATTATTGTGCAAGAAGAGTCCACTGGGGGGGTCCCTTTG |
| | | ACTATTGGGGCCAGGGAACCCTGGTCACCGTCTCCTCAGCT |
| | | AGCACCAAGGGCCCATCTGTCTTCCCCCTGGCACCCTCCTC |
| | | CAAGAGCACCTCTGGGGGCACAGCTGCCCTGGGCTGCCTGG |
| | | TCAAGGACTACTTCCCTGAACCTGTGACAGTGTCCTGGAAC |
| | | TCAGGAGCCCTGACCAGCGGCGTGCACACCTTCCCGGCTGT |
| | | CCTACAGTCCTCAGGACTCTACTCCCTCAGCAGCGTGGTGA |
| | | CCGTGCCCTCCAGCAGCTTGGGCACCCAGACCTACATCTGC |
| | | AACGTGAATCACAAGCCCAGCAACACCAAGGTGGACAAGAA |
| | | AGTTGAGCCCAAATCTTGTGACAAAACTCACACATGCCCAC |
| | | CGTGCCCAGCACCTGAACTCCTGGGGGGACCGTCAGTCTTC |
| | | CTCTTCCCCCCAAAACCCAAGGACACCCTCATGATCTCCCG |
| | | GACCCCTGAGGTCACATGCGTGGTGGTGGACGTGAGCCACG |
| | | AAGACCCTGAGGTCAAGTTCAACTGGTACGTGGACGGCGTG |
| | | GAGGTGCATAATGCCAAGACAAAGCCGCGGGAGGAGCAGTA |
| | | CAACAGCACGTACCGTGTGGTCAGCGTCCTCACCGTCCTGC |
| | | ACCAGGACTGGCTGAATGGCAAGGAGTACAAGTGCAAGGTC |
| | | TCCAACAAAGCCCTCCCAGCCCCCATCGAGAAAACCATCTC |
| | | CAAAGCCAAAGGGCAGCCCCGAGAACCACAGGTGTACACCC |
| | | TGCCCCCATCCCGGGATGAGCTGACCAAGAACCAGGTCAGC |
| | | CTGACCTGCCTGGTCAAAGGCTTCTATCCCAGCGACATCGC |
| | | CGTGGAGTGGGAGAGCAATGGGCAGCCGGAGAACAACTACA |
| | | AGACCACGCCTCCCGTGCTGGACTCCGACGGCTCCTTCTTC |
| | | CTCTACAGCAAGCTCACCGTGGACAAGAGCAGATGGCAGCA |
| | | GGGGAACGTCTTCTCATGCTCCGTGATGCATGAGGCTCTGC |
| | | ACAACCACTACACACAGAAGAGCCTCTCCCTGTCTCCGGGC |
| | | AAA |
| 268 | Anti-CD228 antibody | DIQLTQSPSFLSASVGDRVTITCRASQGISSYLAWYQQRPG |
| | clone 35 LC amino acid | KAPKVLISVASTLQSGVPSRFSGSGSGTDFTLTISSLQPED |
| | | FATYYCQQLTGYPFTFGPGTKVDIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 269 | Anti-CD228 antibody | GACATCCAGTTGACCCAGTCTCCATCCTTCCTGTCTGCCTC |
| | clone 35 LC nucleic | TGTAGGAGACAGAGTCACCATCACTTGCCGGGCCAGTCAGG |
| | acid | GCATTAGCAGTTATTTAGCCTGGTATCAGCAAAGACCAGGG |
| | | AAAGCCCCTAAGGTCTTGATCTCTGTTGCATCCACTTTGCA |
| | | AAGTGGGGTCCCATCAAGGTTCAGCGGCAGTGGATCTGGGA |
| | | CAGATTTCACTCTCACAATCAGCAGCCTGCAGCCTGAGGAT |
| | | TTTGCGACTTATTACTGTCAACAGCTTACTGGTTACCCATT |
| | | CACTTTCGGCCCTGGGACCAAAGTGGATATCAAACGTACGG |
| | | TGGCTGCACCATCTGTCTTCATCTTCCCGCCATCTGATGAG |
| | | CAGTTGAAATCTGGAACTGCCTCTGTTGTGTGCCTGCTGAA |
| | | TAACTTCTATCCCAGAGAGGCCAAAGTACAGTGGAAGGTGG |
| | | ATAACGCCCTCCAATCGGGTAACTCCCAGGAGAGTGTCACA |
| | | GAGCAGGACAGCAAGGACAGCACCTACAGCCTCAGCAGCAC |
| | | CCTGACGCTGAGCAAAGCAGACTACGAGAAACACAAAGTCT |
| | | ACGCCTGCGAAGTCACCCATCAGGGCCTGAGCTCGCCCGTC |
| | | ACAAAGAGCTTCAACAGGGGAGAGTGT |
| 270 | Anti-CD228 antibody | NYDIN |
| | clone 36 CDR-H1 | |
| | (Kabat) | |
| 271 | Anti-CD228 antibody | WMNPNSGNTDYVQKFQG |
| | clone 36 CDR-H2 | |
| | (Kabat) | |
| 272 | Anti-CD228 antibody | GLHSGDYYSYGMDV |
| | clone 36 CDR-H3 | |
| | (Kabat) | |
| 273 | Anti-CD228 antibody | GYTFTNYD |
| | clone 36 CDR-H1 | |
| | (IMGT) | |
| 274 | Anti-CD228 antibody | MNPNSGNT |
| | clone 36 CDR-H2 | |
| | (IMGT) | |
| 275 | Anti-CD228 antibody | ARGLHSGDYYSYGMDV |
| | clone 36 CDR-H3 | |
| | (IMGT) | |
| 276 | Anti-CD228 antibody | SGDALPNQYAY |
| | clone 36 CDR-L1 | |
| | (Kabat) | |
| 277 | Anti-CD228 antibody | KDSERPS |
| | clone 36 CDR-L2 | |
| | (Kabat) | |
| 278 | Anti-CD228 antibody | QSADSSGTVV |
| | clone 36 CDR-L3 | |
| | (Kabat) | |
| 279 | Anti-CD228 antibody | ALPNQY |
| | clone 36 CDR-L1 | |
| | (IMGT) | |
| 280 | Anti-CD228 antibody | KDS |
| | clone 36 CDR-L2 | |
| | (IMGT) | |
| 281 | Anti-CD228 antibody | QSADSSGTVV |
| | clone 36 CDR-L3 | |
| | (IMGT) | |
| 282 | Anti-CD228 antibody | QVQLVQSGAEVKKPGASVKVSCKASGYTFTNYDINWVRQAT |
| | clone 36 VH amino | GQGLEWMGWMNPNSGNTDYVQKFQGRVAMTRITSISTAYLE |
| | acid | LSSLRSDDTAVYYCARGLHSGDYYSYGMDVWGQGTTVTVSS |
| 283 | Anti-CD228 antibody | CAGGTGCAGCTGGTGCAGTCTGGGGCTGAGGTGAAGAAGCC |
| | clone 36 VH nucleic | TGGGGCCTCAGTGAAGGTCTCCTGCAAGGCTTCTGGATACA |
| | acid | CCTTCACCAATTATGATATCAACTGGGTGCGACAGGCCACT |
| | | GGACAAGGGCTTGAGTGGATGGGATGGATGAACCCTAACAG |
| | | TGGTAACACAGACTATGTACAGAAGTTCCAGGGCAGAGTCG |
| | | CCATGACCAGGATCACCTCCATAAGCACAGCCTACTTGGAG |
| | | CTGAGCAGCCTGAGATCTGACGACACGGCCGTGTATTATTG |
| | | TGCGAGAGGCCTCCATAGTGGGGATTACTACTCCTACGGTA |
| | | TGGACGTCTGGGGCCAAGGGACCACGGTCACCGTCTCCTCA |
| 284 | Anti-CD228 antibody | SYELTQPPSVSVSPGQTARITCSGDALPNQYAYWYQQKPGQ |
| | clone 36 VL amino acid | APVLVIYKDSERPSGIPERFSGSSSGTTVTLTISGVQAEDE |
| | | ADYYCQSADSSGTVVFGGGTKLSVL |
| 285 | Anti-CD228 antibody | TCCTATGAGCTGACACAGCCACCCTCGGTGTCAGTGTCCCC |
| | clone 36 VL nucleic | AGGACAGACGGCCAGGATCACCTGCTCTGGAGATGCATTGC |
| | acid | CAAACCAATATGCTTATTGGTACCAACAGAAGCCAGGCCAG |
| | | GCCCCTGTCCTAGTGATATATAAAGACAGTGAGAGGCCCTC |
| | | AGGGATCCCTGAGCGATTCTCTGGCTCCAGCTCAGGGACAA |
| | | CAGTCACGTTGACCATCAGTGGAGTCCAGGCAGAAGACGAG |
| | | GCTGACTATTACTGTCAATCAGCAGACAGCAGTGGTACTGT |
| | | GGTTTTCGGCGGAGGGACCAAGCTGTCCGTCTTA |
| 286 | Anti-CD228 antibody | QVQLVQSGAEVKKPGASVKVSCKASGYTFTNYDINWVRQAT |
| | clone 36 HC amino | GQGLEWMGWMNPNSGNTDYVQKFQGRVAMTRITSISTAYLE |
| | acid | LSSLRSDDTAVYYCARGLHSGDYYSYGMDVWGQGTTVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYI |
| | | CNVNHKPSNTKVDKKVEPKSCDKTHTCPPCPAPELLGGPSV |
| | | FLFPPKPKDTLMISRTPEVTCVVVDVSHEDPEVKFNWYVDG |
| | | VEVHNAKTKPREEQYNSTYRVVSVLTVLHQDWLNGKEYKCK |
| | | VSNKALPAPIEKTISKAKGQPREPQVYTLPPSRDELTKNQV |
| | | SLTCLVKGFYPSDIAVEWESNGQPENNYKTTPPVLDSDGSE |
| | | FLYSKLTVDKSRWQQGNVFSCSVMHEALHNHYTQKSLSLSP |
| | | GK* |
| 287 | Anti-CD228 antibody | CAGGTGCAGCTGGTGCAGTCTGGGGCTGAGGTGAAGAAGCC |
| | clone 36 HC nucleic | TGGGGCCTCAGTGAAGGTCTCCTGCAAGGCTTCTGGATACA |
| | acid | CCTTCACCAATTATGATATCAACTGGGTGCGACAGGCCACT |
| | | GGACAAGGGCTTGAGTGGATGGGATGGATGAACCCTAACAG |
| | | TGGTAACACAGACTATGTACAGAAGTTCCAGGGCAGAGTCG |
| | | CCATGACCAGGATCACCTCCATAAGCACAGCCTACTTGGAG |
| | | CTGAGCAGCCTGAGATCTGACGACACGGCCGTGTATTATTG |
| | | TGCGAGAGGCCTCCATAGTGGGGATTACTACTCCTACGGTA |
| | | TGGACGTCTGGGGCCAAGGGACCACGGTCACCGTCTCCTCA |
| | | GCTAGCACCAAGGGCCCATCTGTCTTCCCCCTGGCACCCTC |
| | | CTCCAAGAGCACCTCTGGGGGCACAGCTGCCCTGGGCTGCC |
| | | TGGTCAAGGACTACTTCCCTGAACCTGTGACAGTGTCCTGG |
| | | AACTCAGGAGCCCTGACCAGCGGCGTGCACACCTTCCCGGC |
| | | TGTCCTACAGTCCTCAGGACTCTACTCCCTCAGCAGCGTGG |
| | | TGACCGTGCCCTCCAGCAGCTTGGGCACCCAGACCTACATC |
| | | TGCAACGTGAATCACAAGCCCAGCAACACCAAGGTGGACAA |
| | | GAAAGTTGAGCCCAAATCTTGTGACAAAACTCACACATGCC |
| | | CACCGTGCCCAGCACCTGAACTCCTGGGGGGACCGTCAGTC |
| | | TTCCTCTTCCCCCCAAAACCCAAGGACACCCTCATGATCTC |
| | | CCGGACCCCTGAGGTCACATGCGTGGTGGTGGACGTGAGCC |
| | | ACGAAGACCCTGAGGTCAAGTTCAACTGGTACGTGGACGGC |
| | | GTGGAGGTGCATAATGCCAAGACAAAGCCGCGGGAGGAGCA |
| | | GTACAACAGCACGTACCGTGTGGTCAGCGTCCTCACCGTCC |
| | | TGCACCAGGACTGGCTGAATGGCAAGGAGTACAAGTGCAAG |
| | | GTCTCCAACAAAGCCCTCCCAGCCCCCATCGAGAAAACCAT |
| | | CTCCAAAGCCAAAGGGCAGCCCCGAGAACCACAGGTGTACA |
| | | CCCTGCCCCCATCCCGGGATGAGCTGACCAAGAACCAGGTC |
| | | AGCCTGACCTGCCTGGTCAAAGGCTTCTATCCCAGCGACAT |
| | | CGCCGTGGAGTGGGAGAGCAATGGGCAGCCGGAGAACAACT |
| | | ACAAGACCACGCCTCCCGTGCTGGACTCCGACGGCTCCTTC |
| | | TTCCTCTACAGCAAGCTCACCGTGGACAAGAGCAGATGGCA |
| | | GCAGGGGAACGTCTTCTCATGCTCCGTGATGCATGAGGCTC |
| | | TGCACAACCACTACACACAGAAGAGCCTCTCCCTGTCTCCG |
| | | GGCAAATGA |
| 288 | Anti-CD228 antibody | SYELTQPPSVSVSPGQTARITCSGDALPNQYAYWYQQKPGQ |
| | clone 36 LC amino acid | APVLVIYKDSERPSGIPERFSGSSSGTTVTLTISGVQAEDE |
| | | ADYYCQSADSSGTVVFGGGTKLSVLGQPKAAPSVTLFPPSS |
| | | EELQANKATLVCLISDFYPGAVTVAWKADSSPVKAGVETTT |
| | | PSKQSNNKYAASSYLSLTPEQWKSHRSYSCQVTHEGSTVEK |
| | | TVAPTECS* |
| 289 | Anti-CD228 antibody | TCCTATGAGCTGACACAGCCACCCTCGGTGTCAGTGTCCCC |
| | clone 36 LC nucleic | AGGACAGACGGCCAGGATCACCTGCTCTGGAGATGCATTGC |
| | acid | CAAACCAATATGCTTATTGGTACCAACAGAAGCCAGGCCAG |
| | | GCCCCTGTCCTAGTGATATATAAAGACAGTGAGAGGCCCTC |
| | | AGGGATCCCTGAGCGATTCTCTGGCTCCAGCTCAGGGACAA |
| | | CAGTCACGTTGACCATCAGTGGAGTCCAGGCAGAAGACGAG |
| | | CCAAGGCGGCGCCCTCGGTCACTCTGTTCCCGCCCTCCTCT |
| | | GCTGACTATTACTGTCAATCAGCAGACAGCAGTGGTACTGT |
| | | GGTTTTCGGCGGAGGGACCAAGCTGTCCGTCCTAGGTCAGC |
| | | GAGGAGCTTCAAGCCAACAAGGCCACACTGGTGTGTCTCAT |
| | | AAGTGACTTCTACCCGGGAGCCGTGACAGTGGCCTGGAAGG |
| | | CAGATAGCAGCCCCGTCAAGGCGGGAGTGGAGACCACCACA |
| | | CCCTCCAAACAAAGCAACAACAAGTACGCGGCCAGCAGCTA |
| | | TCTGAGCCTGACGCCTGAGCAGTGGAAGTCCCACAGAAGCT |
| | | ACAGCTGCCAGGTCACGCATGAAGGGAGCACCGTGGAGAAG |
| | | ACAGTGGCCCCTACAGAATGTTCATAG |

1. An antibody or an antigen-binding fragment thereof that binds CD228, wherein the antibody or the antigen-binding fragment thereof comprises:
a heavy chain variable domain (VH) comprising (a) CDR-H1 comprising the amino acid sequence of SEQ ID NO: 110, (b) CDR-H2 comprising the amino acid sequence of SEQ ID NO: 111, and (c) CDR-H3 comprising the amino acid sequence of SEQ ID NO: 112, and a light chain variable domain (VL) comprising (d) CDR-L1 comprising the amino acid sequence of SEQ ID NO: 116, (e) CDR-L2 comprising the amino acid sequence of SEQ ID NO: 117, and (f) CDR-L3 comprising the amino acid sequence of SEQ ID NO: 118;
a VH comprising (a) CDR-H1 comprising the amino acid sequence of SEQ ID NO: 113, (b) CDR-H2 comprising the amino acid sequence of SEQ ID NO: 114, and (c) CDR-H3 comprising the amino acid sequence of SEQ ID NO: 115, and a VL comprising (d) CDR-L1 comprising the amino acid sequence of SEQ ID NO: 119, (e) CDR-L2 comprising the amino acid sequence of SEQ ID NO: 120, and (f) CDR-L3 comprising the amino acid sequence of SEQ ID NO: 121;
a VH comprising (a) CDR-H1 comprising the amino acid sequence of SEQ ID NO: 130, (b) CDR-H2 comprising the amino acid sequence of SEQ ID NO: 131, and (c) CDR-H3 comprising the amino acid sequence of SEQ ID NO: 132, and a VL comprising (d) CDR-L1 comprising the amino acid sequence of SEQ ID NO: 136, (e) CDR-L2 comprising the amino acid sequence of SEQ ID NO: 137, and (f) CDR-L3 comprising the amino acid sequence of SEQ ID NO: 138;
a VH comprising (a) CDR-H1 comprising the amino acid sequence of SEQ ID NO: 133, (b) CDR-H2 comprising the amino acid sequence of SEQ ID NO: 134, and (c) CDR-H3 comprising the amino acid sequence of SEQ ID NO: 135, and a VL comprising (d) CDR-L1 comprising the amino acid sequence of SEQ ID NO: 139, (e) CDR-L2 comprising the amino acid sequence of SEQ ID NO: 140, and (f) CDR-L3 comprising the amino acid sequence of SEQ ID NO: 141;
a VH comprising (a) CDR-H1 comprising the amino acid sequence of SEQ ID NO: 150, (b) CDR-H2 comprising the amino acid sequence of SEQ ID NO: 151, and (c) CDR-H3 comprising the amino acid sequence of SEQ ID NO: 152, and a VL comprising (d) CDR-L1 comprising the amino acid sequence of SEQ ID NO: 156, (e) CDR-L2 comprising the amino acid sequence of SEQ ID NO: 157, and (f) CDR-L3 comprising the amino acid sequence of SEQ ID NO: 158;
a VH comprising (a) CDR-H1 comprising the amino acid sequence of SEQ ID NO: 153, (b) CDR-H2 comprising the amino acid sequence of SEQ ID NO: 154, and (c) CDR-H3 comprising the amino acid sequence of SEQ ID NO: 155, and a VL comprising (d) CDR-L1 comprising the amino acid sequence of SEQ ID NO: 159, (e) CDR-L2 comprising the amino acid sequence of SEQ ID NO: 160, and (f) CDR-L3 comprising the amino acid sequence of SEQ ID NO: 161;
a VH comprising (a) CDR-H1 comprising the amino acid sequence of SEQ ID NO: 170, (b) CDR-H2 comprising the amino acid sequence of SEQ ID NO: 171, and (c) CDR-H3 comprising the amino acid sequence of SEQ ID NO: 172, and a VL comprising (d) CDR-L1 comprising the amino acid sequence of SEQ ID NO: 176, (e) CDR-L2 comprising the amino acid sequence of SEQ ID NO: 177, and (f) CDR-L3 comprising the amino acid sequence of SEQ ID NO: 178;
a VH comprising (a) CDR-H1 comprising the amino acid sequence of SEQ ID NO: 173, (b) CDR-H2 comprising the amino acid sequence of SEQ ID NO: 174, and (c) CDR-H3 comprising the amino acid sequence of SEQ ID NO: 175, and a VL comprising (d) CDR-L1 comprising the amino acid sequence of SEQ ID NO: 179, (e) CDR-L2 comprising the amino acid sequence of SEQ ID NO: 180, and (f) CDR-L3 comprising the amino acid sequence of SEQ ID NO: 181;
a VH comprising (a) CDR-H1 comprising the amino acid sequence of SEQ ID NO: 190, (b) CDR-H2 comprising the amino acid sequence of SEQ ID NO: 191, and (c) CDR-H3 comprising the amino acid sequence of SEQ ID NO: 192, and a VL comprising (d) CDR-L1 comprising the amino acid sequence of SEQ ID NO: 196, (e) CDR-L2 comprising the amino acid sequence of SEQ ID NO: 197, and (f) CDR-L3 comprising the amino acid sequence of SEQ ID NO: 198;
a VH comprising (a) CDR-H1 comprising the amino acid sequence of SEQ ID NO: 193, (b) CDR-H2 comprising the amino acid sequence of SEQ ID NO: 194, and (c) CDR-H3 comprising the amino acid sequence of SEQ ID NO: 195, and a VL comprising (d) CDR-L1 comprising the amino acid sequence of SEQ ID NO: 199, (e) CDR-L2 comprising the amino acid sequence of SEQ ID NO: 200, and (f) CDR-L3 comprising the amino acid sequence of SEQ ID NO: 201;
a VH comprising (a) CDR-H1 comprising the amino acid sequence of SEQ ID NO: 210, (b) CDR-H2 comprising the amino acid sequence of SEQ ID NO: 211, and (c) CDR-H3 comprising the amino acid sequence of SEQ ID NO: 212, and a VL comprising (d) CDR-L1 comprising the amino acid sequence of SEQ ID NO: 216, (e) CDR-L2 comprising the amino acid sequence of SEQ ID NO: 217, and (f) CDR-L3 comprising the amino acid sequence of SEQ ID NO: 218;
a VH comprising (a) CDR-H1 comprising the amino acid sequence of SEQ ID NO: 213, (b) CDR-H2 comprising the amino acid sequence of SEQ ID NO: 214, and (c) CDR-H3 comprising the amino acid sequence of SEQ ID NO: 215, and a VL comprising (d) CDR-L1 comprising the amino acid sequence of SEQ ID NO: 219, (e) CDR-L2 comprising the amino acid sequence of SEQ ID NO: 220, and (f) CDR-L3 comprising the amino acid sequence of SEQ ID NO: 221;
a VH comprising (a) CDR-H1 comprising the amino acid sequence of SEQ ID NO: 230, (b) CDR-H2 comprising the amino acid sequence of SEQ ID NO: 231, and (c) CDR-H3 comprising the amino acid sequence of SEQ ID NO: 232, and a VL comprising (d) CDR-L1 comprising the amino acid sequence of SEQ ID NO: 236, (e) CDR-L2 comprising the amino acid sequence of SEQ ID NO: 237, and (f) CDR-L3 comprising the amino acid sequence of SEQ ID NO: 238;
a VH comprising (a) CDR-H1 comprising the amino acid sequence of SEQ ID NO: 233, (b) CDR-H2 comprising the amino acid sequence of SEQ ID NO: 234, and (c) CDR-H3 comprising the amino acid sequence of SEQ ID NO: 235, and a VL comprising (d) CDR-L1 comprising the amino acid sequence of SEQ ID NO: 239, (e) CDR-L2 comprising the amino acid sequence of SEQ ID NO: 240, and (f) CDR-L3 comprising the amino acid sequence of SEQ ID NO: 241;
a VH comprising (a) CDR-H1 comprising the amino acid sequence of SEQ ID NO: 250, (b) CDR-H2 comprising the amino acid sequence of SEQ ID NO: 251, and (c) CDR-H3 comprising the amino acid sequence of SEQ ID NO: 252, and a VL comprising (d) CDR-L1 comprising the amino acid sequence of SEQ ID NO: 256, (e) CDR-L2 comprising the amino acid sequence of SEQ ID NO: 257, and (f) CDR-L3 comprising the amino acid sequence of SEQ ID NO: 258;
a VH comprising (a) CDR-H1 comprising the amino acid sequence of SEQ ID NO: 253, (b) CDR-H2 comprising the amino acid sequence of SEQ ID NO: 254, and (c) CDR-H3 comprising the amino acid sequence of SEQ ID NO: 255, and a VL comprising (d) CDR-L1 comprising the amino acid sequence of SEQ ID NO: 259, (e) CDR-L2 comprising the amino acid sequence of SEQ ID NO: 260, and (f) CDR-L3 comprising the amino acid sequence of SEQ ID NO: 261;
a VH comprising (a) CDR-H1 comprising the amino acid sequence of SEQ ID NO: 270, (b) CDR-H2 comprising the amino acid sequence of SEQ ID NO: 271, and (c) CDR-H3 comprising the amino acid sequence of SEQ ID NO: 272, and a VL comprising (d) CDR-L1 comprising the amino acid sequence of SEQ ID NO: 276, (e) CDR-L2 comprising the amino acid sequence of SEQ ID NO: 277, and (f) CDR-L3 comprising the amino acid sequence of SEQ ID NO: 278; or
a VH comprising (a) CDR-H1 comprising the amino acid sequence of SEQ ID NO: 273, (b) CDR-H2 comprising the amino acid sequence of SEQ ID NO: 274, and (c) CDR-H3 comprising the amino acid sequence of SEQ ID NO: 275, and a VL comprising (d) CDR-L1 comprising the amino acid sequence of SEQ ID NO: 279, (e) CDR-L2 comprising the amino acid sequence of SEQ ID NO: 280, and (f) CDR-L3 comprising the amino acid sequence of SEQ ID NO: 281.

2. The antibody or the antigen-binding fragment thereof of claim 1, wherein the antibody or the antigen-binding fragment thereof comprises:
a VH comprising (a) CDR-H1 comprising the amino acid sequence of SEQ ID NO: 210, (b) CDR-H2 comprising the amino acid sequence of SEQ ID NO: 211, and (c) CDR-H3 comprising the amino acid sequence of SEQ ID NO: 212, and a VL comprising (d) CDR-L1 comprising the amino acid sequence of SEQ ID NO: 216, (e) CDR-L2 comprising the amino acid sequence of SEQ ID NO: 217, and (f) CDR-L3 comprising the amino acid sequence of SEQ ID NO: 218;
a VH comprising (a) CDR-H1 comprising the amino acid sequence of SEQ ID NO: 213, (b) CDR-H2 comprising the amino acid sequence of SEQ ID NO: 214, and (c) CDR-H3 comprising the amino acid sequence of SEQ ID NO: 215, and a VL comprising (d) CDR-L1 comprising the amino acid sequence of SEQ ID NO: 219, (e) CDR-L2 comprising the amino acid sequence of SEQ ID NO: 220, and (f) CDR-L3 comprising the amino acid sequence of SEQ ID NO: 221;
a VH comprising (a) CDR-H1 comprising the amino acid sequence of SEQ ID NO: 250, (b) CDR-H2 comprising the amino acid sequence of SEQ ID NO: 251, and (c) CDR-H3 comprising the amino acid sequence of SEQ ID NO: 252, and a VL comprising (d) CDR-L1 comprising the amino acid sequence of SEQ ID NO: 256, (e) CDR-L2 comprising the amino acid sequence of SEQ ID NO: 257, and (f) CDR-L3 comprising the amino acid sequence of SEQ ID NO: 258; or
a VH comprising (a) CDR-H1 comprising the amino acid sequence of SEQ ID NO: 253, (b) CDR-H2 comprising the amino acid sequence of SEQ ID NO: 254, and (c) CDR-H3 comprising the amino acid sequence of SEQ ID NO: 255, and a VL comprising (d) CDR-L1 comprising the amino acid sequence of SEQ ID NO: 259, (e) CDR-L2 comprising the amino acid sequence of SEQ ID NO: 260, and (f) CDR-L3 comprising the amino acid sequence of SEQ ID NO: 261.

3. The antibody or the antigen-binding fragment thereof of claim 1 or 2, wherein the antibody or the antigen-binding fragment thereof comprises:
a VH comprising (a) CDR-H1 comprising the amino acid sequence of SEQ ID NO: 210, (b) CDR-H2 comprising the amino acid sequence of SEQ ID NO: 211, and (c) CDR-H3 comprising the amino acid sequence of SEQ ID NO: 212, and a VL comprising (d) CDR-L1 comprising the amino acid sequence of SEQ ID NO: 216, (e) CDR-L2 comprising the amino acid sequence of SEQ ID NO: 217, and (f) CDR-L3 comprising the amino acid sequence of SEQ ID NO: 218; or
a VH comprising (a) CDR-H1 comprising the amino acid sequence of SEQ ID NO: 213, (b) CDR-H2 comprising the amino acid sequence of SEQ ID NO: 214, and (c) CDR-H3 comprising the amino acid sequence of SEQ ID NO: 215, and a VL comprising (d) CDR-L1 comprising the amino acid sequence of SEQ ID NO: 219, (e) CDR-L2 comprising the amino acid sequence of SEQ ID NO: 220, and (f) CDR-L3 comprising the amino acid sequence of SEQ ID NO: 221.

4. The antibody or the antigen-binding fragment thereof of claim 1, wherein the antibody or the antigen-binding fragment thereof comprises:
a VH comprising an amino acid sequence having at least 95% sequence identity to SEQ ID NO: 122, and a VL comprising an amino acid sequence having at least 95% sequence identity to SEQ ID NO: 124;
a VH comprising an amino acid sequence having at least 95% sequence identity to SEQ ID NO: 142, and a VL comprising an amino acid sequence having at least 95% sequence identity to SEQ ID NO: 144;
a VH comprising an amino acid sequence having at least 95% sequence identity to SEQ ID NO: 162, and a VL comprising an amino acid sequence having at least 95% sequence identity to SEQ ID NO: 164;
a VH comprising an amino acid sequence having at least 95% sequence identity to SEQ ID NO: 182, and a VL comprising an amino acid sequence having at least 95% sequence identity to SEQ ID NO: 184;
a VH comprising an amino acid sequence having at least 95% sequence identity to SEQ ID NO: 202, and a VL comprising an amino acid sequence having at least 95% sequence identity to SEQ ID NO: 204;
a VH comprising an amino acid sequence having at least 95% sequence identity to SEQ ID NO: 222, and a VL comprising an amino acid sequence having at least 95% sequence identity to SEQ ID NO: 224;
a VH comprising an amino acid sequence having at least 95% sequence identity to SEQ ID NO: 242, and a VL comprising an amino acid sequence having at least 95% sequence identity to SEQ ID NO: 244;
a VH comprising an amino acid sequence having at least 95% sequence identity to SEQ ID NO: 262, and a VL comprising an amino acid sequence having at least 95% sequence identity to SEQ ID NO: 264; or
a VH comprising an amino acid sequence having at least 95% sequence identity to SEQ ID NO: 282, and a VL comprising an amino acid sequence having at least 95% sequence identity to SEQ ID NO: 284.

5. The antibody or the antigen-binding fragment thereof of any one of claims 1, 2, and 4, wherein the antibody or the antigen-binding fragment thereof comprises:
a VH comprising an amino acid sequence having at least 95% sequence identity to SEQ ID NO: 222, and a VL comprising an amino acid sequence having at least 95% sequence identity to SEQ ID NO: 224; or
a VH comprising an amino acid sequence having at least 95% sequence identity to SEQ ID NO: 262, and a VL comprising an amino acid sequence having at least 95% sequence identity to SEQ ID NO: 264.

6. The antibody or the antigen-binding fragment thereof of any one of claims 1 to 5, wherein the antibody or the antigen-binding fragment thereof comprises:
a VH comprising an amino acid sequence having at least 95% sequence identity to SEQ ID NO: 222, and a VL comprising an amino acid sequence having at least 95% sequence identity to SEQ ID NO: 224.

7. The antibody or the antigen-binding fragment thereof of claim 1 or 4, wherein the antibody or the antigen-binding fragment thereof comprises:
a VH comprising the amino acid sequence of SEQ ID NO: 122, and a VL comprising the amino acid sequence of SEQ ID NO: 124;
a VH comprising the amino acid sequence of SEQ ID NO: 142, and a VL comprising the amino acid sequence of SEQ ID NO: 144;
a VH comprising the amino acid sequence of SEQ ID NO: 162, and a VL comprising the amino acid sequence of SEQ ID NO: 164;
a VH comprising the amino acid sequence of SEQ ID NO: 182, and a VL comprising the amino acid sequence of SEQ ID NO: 184;
a VH comprising the amino acid sequence of SEQ ID NO: 202, and a VL comprising the amino acid sequence of SEQ ID NO: 204;
a VH comprising the amino acid sequence of SEQ ID NO: 222, and a VL comprising the amino acid sequence of SEQ ID NO: 224;
a VH comprising the amino acid sequence of SEQ ID NO: 242, and a VL comprising the amino acid sequence of SEQ ID NO: 244;
a VH comprising the amino acid sequence of SEQ ID NO: 262, and a VL comprising the amino acid sequence of SEQ ID NO: 264; or
a VH comprising the amino acid sequence of SEQ ID NO: 282, and a VL comprising the amino acid sequence of SEQ ID NO: 284.

8. The antibody or the antigen-binding fragment thereof of any one of claims 1, 2, 4, 5, and 7, wherein the antibody or the antigen-binding fragment thereof comprises:
a VH comprising the amino acid sequence of SEQ ID NO: 222, and a VL comprising the amino acid sequence of SEQ ID NO: 224; or
a VH comprising the amino acid sequence of SEQ ID NO: 262, and a VL comprising the amino acid sequence of SEQ ID NO: 264.

9. The antibody or the antigen-binding fragment thereof of any one of claims 1 to 8, wherein the antibody or the antigen-binding fragment thereof comprises:
a VH comprising the amino acid sequence of SEQ ID NO: 222, and a VL comprising the amino acid sequence of SEQ ID NO: 224.

10. The antibody or the antigen-binding fragment thereof of any one of claims 1, 4, and 7, wherein the antibody or the antigen-binding fragment thereof comprises:
a heavy chain (HC) comprising an amino acid sequence having at least 95% sequence identity to SEQ ID NO: 126, and a light chain (LC) comprising an amino acid sequence having at least 95% sequence identity to SEQ ID NO: 128;
a HC comprising an amino acid sequence having at least 95% sequence identity to SEQ ID NO: 146, and a LC comprising an amino acid sequence having at least 95% sequence identity to SEQ ID NO: 148;
a HC comprising an amino acid sequence having at least 95% sequence identity to SEQ ID NO: 166, and a LC comprising an amino acid sequence having at least 95% sequence identity to SEQ ID NO: 168;
a HC comprising an amino acid sequence having at least 95% sequence identity to SEQ ID NO: 186, and a LC comprising an amino acid sequence having at least 95% sequence identity to SEQ ID NO: 188;
a HC comprising an amino acid sequence having at least 95% sequence identity to SEQ ID NO: 206, and a LC comprising an amino acid sequence having at least 95% sequence identity to SEQ ID NO: 208;
a HC comprising an amino acid sequence having at least 95% sequence identity to SEQ ID NO: 226, and a LC comprising an amino acid sequence having at least 95% sequence identity to SEQ ID NO: 228;
a HC comprising an amino acid sequence having at least 95% sequence identity to SEQ ID NO: 246, and a LC comprising an amino acid sequence having at least 95% sequence identity to SEQ ID NO: 248;
a HC comprising an amino acid sequence having at least 95% sequence identity to SEQ ID NO: 266, and a LC comprising an amino acid sequence having at least 95% sequence identity to SEQ ID NO: 268; or
a HC comprising an amino acid sequence having at least 95% sequence identity to SEQ ID NO: 286, and a LC comprising an amino acid sequence having at least 95% sequence identity to SEQ ID NO: 288.

11. The antibody or the antigen-binding fragment thereof of any one of claims 1, 2, 4, 5, 7, 8, and 10, wherein the antibody or the antigen-binding fragment thereof comprises:
a HC comprising an amino acid sequence having at least 95% sequence identity to SEQ ID NO: 226, and a LC comprising an amino acid sequence having at least 95% sequence identity to SEQ ID NO: 228; or
a HC comprising an amino acid sequence having at least 95% sequence identity to SEQ ID NO: 266, and a LC comprising an amino acid sequence having at least 95% sequence identity to SEQ ID NO: 268.

12. The antibody or the antigen-binding fragment thereof of any one of claims 1 to 11, wherein the antibody or the antigen-binding fragment thereof comprises:
a HC comprising an amino acid sequence having at least 95% sequence identity to SEQ ID NO: 226, and a LC comprising an amino acid sequence having at least 95% sequence identity to SEQ ID NO: 228.

13. The antibody or the antigen-binding fragment thereof of any one of claims 1, 4, 7, and 10, wherein the antibody or the antigen-binding fragment thereof comprises:
a HC comprising the amino acid sequence of SEQ ID NO: 126, and a LC comprising the amino acid sequence of SEQ ID NO: 128;
a HC comprising the amino acid sequence of SEQ ID NO: 146, and a LC comprising the amino acid sequence of SEQ ID NO: 148;
a HC comprising the amino acid sequence of SEQ ID NO: 166, and a LC comprising the amino acid sequence of SEQ ID NO: 168;
a HC comprising the amino acid sequence of SEQ ID NO: 186, and a LC comprising the amino acid sequence of SEQ ID NO: 188;
a HC comprising the amino acid sequence of SEQ ID NO: 206, and a LC comprising the amino acid sequence of SEQ ID NO: 208;
a HC comprising the amino acid sequence of SEQ ID NO: 226, and a LC comprising the amino acid sequence of SEQ ID NO: 228;
a HC comprising the amino acid sequence of SEQ ID NO: 246, and a LC comprising the amino acid sequence of SEQ ID NO: 248;
a HC comprising the amino acid sequence of SEQ ID NO: 266, and a LC comprising the amino acid sequence of SEQ ID NO: 268; or
a HC comprising the amino acid sequence of SEQ ID NO: 286, and a LC comprising the amino acid sequence of SEQ ID NO: 288.

14. The antibody or the antigen-binding fragment thereof of any one of claims 1, 2, 4, 5, 7, 8, 10, 11, and 13, wherein the antibody or the antigen-binding fragment thereof comprises:
a HC comprising the amino acid sequence of SEQ ID NO: 226, and a LC comprising the amino acid sequence of SEQ ID NO: 228; or
a HC comprising the amino acid sequence of SEQ ID NO: 266, and a LC comprising the amino acid sequence of SEQ ID NO: 268.

15. The antibody or the antigen-binding fragment thereof of any one of claims 1 to 14, wherein the antibody or the antigen-binding fragment thereof comprises:
a HC comprising the amino acid sequence of SEQ ID NO: 226, and a LC comprising the amino acid sequence of SEQ ID NO: 228.

16. The antibody or the antigen-binding fragment thereof of any one of claims 1 to 15, wherein the antibody is a monoclonal antibody.

17. The antibody or the antigen-binding fragment thereof of any one of claims 1 to 16, wherein the antibody is a humanized or chimeric antibody.

18. The antibody or the antigen-binding fragment thereof of any one of claims 1 to 17, wherein the antibody is an IgG1, IgG2, IgG3, or IgG4 antibody.

19. The antibody or the antigen-binding fragment thereof of any one of claims 1 to 18, wherein the antibody or the antigen-binding fragment thereof binds CD228 with a K_{D} value of 175 nM or less.

20. The antibody or the antigen-binding fragment thereof of any one of claims 1 to 19, wherein the antibody or the antigen-binding fragment thereof binds cynomolgus monkey CD228.

21. The antibody or the antigen-binding fragment thereof of any one of claims 1 to 20, wherein the antibody or the antigen-binding fragment thereof does not bind murine CD228, or binds murine CD228 with at least 100-fold reduced affinity compared to human CD228.

22. The antibody or the antigen-binding fragment thereof of any one of claims 1 to 21, wherein the antibody or the antigen-binding fragment thereof does not bind transferrin or lactotransferrin.

23. An antibody or an antigen-binding fragment thereof that binds CD228, wherein the antibody or the antigen-binding fragment thereof competes for binding with CD228 with the antibody or the antigen-binding fragment thereof of any one of claims 1 to 22.

24. The antibody or the antigen-binding fragment of claim 23, wherein the antibody is a monoclonal antibody.

25. The antibody or the antigen-binding fragment thereof of claim 23 or 24, wherein the antibody is a humanized or chimeric antibody.

26. The antibody or the antigen-binding fragment thereof of any one of claims 23 to 25, wherein the antibody is an IgG1, IgG2, IgG3, or IgG4 antibody.

27. The antibody or the antigen-binding fragment thereof of any one of claims 23 to 26, wherein the antibody or the antigen-binding fragment thereof binds CD228 with a K_{D} value of 175 nM or less.

28. The antibody or the antigen-binding fragment thereof of any one of claims 23 to 27, wherein the antibody or the antigen-binding fragment thereof binds cynomolgus monkey CD228.

29. The antibody or the antigen-binding fragment thereof of any one of claims 23 to 28, wherein the antibody or the antigen-binding fragment thereof does not bind murine CD228, or binds murine CD228 with at least 100-fold reduced affinity compared to human CD228.

30. The antibody or the antigen-binding fragment thereof of any one of claims 23 to 29, wherein the antibody or the antigen-binding fragment thereof does not bind transferrin or lactotransferrin.

31. The antibody or the antigen-binding fragment thereof of any one of claims 23 to 30, wherein the antibody or antigen-binding fragment thereof competes for binding with CD228 with an antibody or antigen-binding fragment thereof comprising:
a VH comprising the amino acid sequence of SEQ ID NO: 202, and a VL comprising the amino acid sequence of SEQ ID NO: 204;
a VH comprising the amino acid sequence of SEQ ID NO: 242, and a VL comprising the amino acid sequence of SEQ ID NO: 244; or
a VH comprising the amino acid sequence of SEQ ID NO: 262, and a VL comprising the amino acid sequence of SEQ ID NO: 264.

32. The antibody or the antigen-binding fragment thereof of claim 31, wherein the antibody or antigen-binding fragment thereof competes for binding with CD228 with an antibody or antigen-binding fragment thereof comprising:
a VH comprising the amino acid sequence of SEQ ID NO: 202, and a VL comprising the amino acid sequence of SEQ ID NO: 204; or
a VH comprising the amino acid sequence of SEQ ID NO: 242, and a VL comprising the amino acid sequence of SEQ ID NO: 244.

33. The antibody or the antigen-binding fragment thereof of any one of claims 23 to 32, wherein competition for binding with CD228 is measured by flow cytometry using at least one labeled antibody or antigen-binding fragment thereof.

34. An isolated nucleic acid molecule comprising a nucleotide sequence encoding the antibody or the antigen-binding fragment thereof of any one of claims 1-33.

35. The isolated nucleic acid molecule of claim 34, wherein the nucleic acid molecule is operably linked to a regulatory sequence to allow expression of said nucleic acid molecule.

36. A vector comprising the nucleic acid molecule of claim 34 or claim 35.

37. A host cell comprising the nucleic acid molecule of claim 34 or 35 or the vector of claim 36.

38. A host cell that expresses the antibody or antigen-binding fragment thereof of any one of claims 1-33.

39. The host cell of claim 37 or claim 38, wherein the host cell is a prokaryotic cell or a eukaryotic cell.

40. A method of producing the antibody or the antigen-binding fragment thereof of any one of claims 1-33 comprising incubating the host cell of any one of claims 37-39 under conditions suitable to produce the antibody or antigen-binding fragment thereof.

41. The method of claim 40, further comprising isolating the antibody or the antigen-binding fragment thereof.

42. A pharmaceutical composition comprising the antibody or antigen-binding fragment thereof of any one of claims 1-33 and a pharmaceutically acceptable excipient.

43. An immunoconjugate comprising an antibody or antigen-binding fragment thereof of any one of claims 1-33 and a cytotoxic agent.

44. The immunoconjugate of claim 43, wherein the cytotoxic agent is an auristatin.

45. The immunoconjugate of claim 44, wherein the cytotoxic agent is monomethyl auristatin E (MMAE).

46. The immunoconjugate of claim 44, wherein the cytotoxic agent is monomethyl auristatin F (MMAF).

47. A method of treating cancer comprising administering to a subject with cancer the antibody or antigen-binding fragment thereof of any one of claims 1-33, the pharmaceutical composition of claim 42, or the immunoconjugate of any one of claims 43-46.


SEQUENCE LISTING (289 sequences)

SEQ ID NO: 1 - protein, 158 aa, Homo sapiens
HHLLASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYCEGELHGKPVRGVKLVGRDPKNNLEALEDFEKAAGARGLSTESILIPRQSETCSPGSD

SEQ ID NO: 2 - protein, 178 aa, Homo sapiens
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 3-5 - skipped (3 entries)

SEQ ID NO: 6 - protein, 698 aa, Homo sapiens
GMEVRWCATSDPEQHKCGNMSEAFREAGIQPSLLCVRGTSADHCVQLIAAQEADAITLDGGAIYEAGKEHGLKPVVGEVYDQEVGTSYYAVAVVRRSSHVTIDTLKGVKSCHTGINRTVGWNVPVGYLVESGRLSVMGCDVLKAVSDYFGGSCVPGAGETSYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSEAYGQKDLLFKDSTSELVPIATQTYEAWLGHEYLHAMKGLLCDPNRLPPYLRWCVLSTPEIQKCGDMAVAFRRQRLKPEIQCVSAKSPQHCMERIQAEQVDAVTLSGEDIYTAGKTYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVMVRPDTNIFTVYGLLDKAQDLFGDDHNKNGFKMFDSSNYHGQDLLFKDATVRAVPVGEKTTYRGWLGLDYVAALEGMSSQQCSGHHHHHH

SEQ ID NO: 7 - protein, 232 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 8 - protein, 703 aa, Macaca fascicularis
GMEVRWCVTSDPEQQKCSNMSTAFREAGIQPSLLCVQGTSPDHCIQLIAAQEADAITLDGGAIYEAGKEHGLKPVVGEVYDQEVGTSYYAVAVVKRSSQVTINTLKGVKSCHTGINRTVGWNVPVGYLVESGRLSVMGCDVLKAVSDYFGGSCVPGAGETRYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSKAYGQKDLLFKDSTSELVPIATQTYEAWLGQEYLHAMKGLLCDPNRLPPYLRWCVLSTPEIQKCGDMAVAFGRQQLKPEIQCVSAKSPQHCMEQIQAGQIDTVTLSGEDIYTAGKTYGLAPAAGEGYASEDSSNSYFVVAVVRRDSSHAFTLDELRGKRSCHAGFGSPAGWDIPVGALIRRGFIRPKDCDVLTAVSEFFNASCVPVNNPKNYPSSLCALCVGDEQGRNKCVGNSQERYYGNSGAFRCLVENAGDVAFVRHTTVFDNTNGHNSEPWAAELRSEDYELLCPNGARAEVSQFAACNLAQMPPHAVMVRPDTNIFTVYGLLDKAQDLFGDDHNKNGFKMFDSSNYHGQDLLFKDATVRAVPVGEKTTYRDWLGLDYVAALEGMLSEQCSGGHHHHHHHHHH

SEQ ID NO: 9 - protein, 231 aa, Macaca fascicularis
LQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

SEQ ID NO: 10-23 - skipped (14 entries)

SEQ ID NO: 24 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 448 aa, synthetic construct
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 216 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 31 - protein, 228 aa, synthetic construct
ESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 32-39 - skipped (8 entries)

SEQ ID NO: 40 - protein, 178 aa, synthetic construct
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 41-57 - skipped (17 entries)

SEQ ID NO: 58 - protein, 8 aa, synthetic construct
GFTFSSYG

SEQ ID NO: 59 - protein, 8 aa, synthetic construct
IWYDGSNK

SEQ ID NO: 60 - protein, 13 aa, synthetic construct
ARRGDSSSWHFDY

SEQ ID NO: 61 - protein, 9 aa, synthetic construct
SSDVGRHNL

SEQ ID NO: 62 - skipped

SEQ ID NO: 63 - protein, 10 aa, synthetic construct
CSYAGSSTYV

SEQ ID NO: 64 - protein, 10 aa, synthetic construct
GDSVSSNSAA

SEQ ID NO: 65 - protein, 9 aa, synthetic construct
TYYRSKWFD

SEQ ID NO: 66 - protein, 12 aa, synthetic construct
ARRVHWGGPFDY

SEQ ID NO: 67 - protein, 6 aa, synthetic construct
QGISSY

SEQ ID NO: 68 - skipped

SEQ ID NO: 69 - protein, 9 aa, synthetic construct
QQLTGYPFT

SEQ ID NO: 70 - protein, 120 aa, synthetic construct
QVQLVESGGGVVQPGRSLRLSCAASGFTFSSYGIHWVRQAPGKGLEWVAVIWYDGSNKYYADSVKGRFTISRDNSKNTLYLQMNSLRAEDTAVYYCARRGDSSSWHFDYWGQGTLVTVSS

SEQ ID NO: 71 - protein, 110 aa, synthetic construct
QSALTQPASVSGSPGQSITISCTGTSSDVGRHNLVSWYQQHPGKVPKVMIYEVTKRPAGVSNRFSGSKSGNTASLTISGLQAEDEADYYCCSYAGSSTYVFGTGTRVTVL

SEQ ID NO: 72 - protein, 122 aa, synthetic construct
QVQLQQSGPGLVKPSQTLSLTCAISGDSVSSNSAAWNWIRQSPSSGLEWLGRTYYRSKWFDDYAVSVKGRITINPDTSKNQFSLQLKFVIPEDTAVYYCARRVHWGGPFDYWGQGTLVTVSS

SEQ ID NO: 73 - protein, 107 aa, synthetic construct
DIQLTQSPSFLSASVGDRVTITCRASQGISSYLAWYQQRPGKAPKVLISVASTLQSGVPSRFSGSGSGTDFTLTISSLQPEDFATYYCQQLTGYPFTFGPGTKVDIK

SEQ ID NO: 74 - protein, 450 aa, synthetic construct
QVQLVESGGGVVQPGRSLRLSCAASGFTFSSYGIHWVRQAPGKGLEWVAVIWYDGSNKYYADSVKGRFTISRDNSKNTLYLQMNSLRAEDTAVYYCARRGDSSSWHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 446 aa, synthetic construct
QVQLVESGGGVVQPGRSLRLSCAASGFTFSSYGIHWVRQAPGKGLEWVAVIWYDGSNKYYADSVKGRFTISRDNSKNTLYLQMNSLRAEDTAVYYCARRGDSSSWHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 76 - protein, 216 aa, synthetic construct
QSALTQPASVSGSPGQSITISCTGTSSDVGRHNLVSWYQQHPGKVPKVMIYEVTKRPAGVSNRFSGSKSGNTASLTISGLQAEDEADYYCCSYAGSSTYVFGTGTRVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 77 - protein, 452 aa, synthetic construct
QVQLQQSGPGLVKPSQTLSLTCAISGDSVSSNSAAWNWIRQSPSSGLEWLGRTYYRSKWFDDYAVSVKGRITINPDTSKNQFSLQLKFVIPEDTAVYYCARRVHWGGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 448 aa, synthetic construct
QVQLQQSGPGLVKPSQTLSLTCAISGDSVSSNSAAWNWIRQSPSSGLEWLGRTYYRSKWFDDYAVSVKGRITINPDTSKNQFSLQLKFVIPEDTAVYYCARRVHWGGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 79 - protein, 214 aa, synthetic construct
DIQLTQSPSFLSASVGDRVTITCRASQGISSYLAWYQQRPGKAPKVLISVASTLQSGVPSRFSGSGSGTDFTLTISSLQPEDFATYYCQQLTGYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 639 aa, synthetic construct
QVQLVESGGGVVQPGRSLRLSCAASGFTFSSYGIHWVRQAPGKGLEWVAVIWYDGSNKYYADSVKGRFTISRDNSKNTLYLQMNSLRAEDTAVYYCARRGDSSSWHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 81 - protein, 409 aa, synthetic construct
QSALTQPASVSGSPGQSITISCTGTSSDVGRHNLVSWYQQHPGKVPKVMIYEVTKRPAGVSNRFSGSKSGNTASLTISGLQAEDEADYYCCSYAGSSTYVFGTGTRVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 82 - protein, 641 aa, synthetic construct
QVQLQQSGPGLVKPSQTLSLTCAISGDSVSSNSAAWNWIRQSPSSGLEWLGRTYYRSKWFDDYAVSVKGRITINPDTSKNQFSLQLKFVIPEDTAVYYCARRVHWGGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 83 - protein, 407 aa, synthetic construct
DIQLTQSPSFLSASVGDRVTITCRASQGISSYLAWYQQRPGKAPKVLISVASTLQSGVPSRFSGSGSGTDFTLTISSLQPEDFATYYCQQLTGYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 84 - protein, 422 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 85 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 214 aa, synthetic construct; identical to SEQ ID NO: 25

SEQ ID NO: 87 - DNA, 1338 bp, synthetic construct
  source 1..1338: mol_type = other DNA
caggtgcagctggttgaatctggtggcggagtggtgcagcctggcagatctctgagactgtcttgtgccgcctccggcttcaccttctcctcttacggaatccactgggtccgacaggcccctggcaaaggattggaatgggtcgccgtgatttggtacgacggctccaacaagtactacgccgactccgtgaagggcagattcaccatctctcgggacaactccaagaacaccctgtacctgcagatgaactccctgagagccgaggacaccgccgtgtactactgtgccagaagaggcgactcctcctcctggcactttgactattggggccagggcaccctggtcaccgttagttctgctagcaccaaagggccgtccgtcttccccctggccccctgcagccggtcgacgtccgagtccaccgccgccctcgggtgcctggtcaaggactacttcccggagccggtaaccgtgagctggaactccggcgcgctgacctccggcgtgcacacgttccccgccgtcctgcagtcctccggcctctactccctctcgtccgtcgtcaccgtcccgagcagttccctgggcaccaagacctacacgtgcaacgtcgaccacaagccctccaacaccaaggtagacaagcgcgtcgagtccaaatacggccccccgtgcccgccctgtccggcccccgaggccgcgggcggcccctcagtgttcctgttcccgccgaagcccaaggacaccctgatgatctcgcgcacgcccgaggtcacgtgcgtggtcgtcgacgtctcacaggaagaccccgaggtgcagttcaactggtatgtcgacggcgtggaggttcacaacgcgaagaccaagccccgggaggagcagttcaacagcacataccgggtggtgtcggtcctcaccgtgctgcatcaggactggctgaacggtaaggagtacaagtgcaaggtgtccaacaagggcctcccgagcagcatagagaagaccatctccaaggcgaagggtcagccccgcgaaccgcaggtgtacaccctgccgccgagccaggaggagatgacgaagaaccaggtctccctgacgtgcctggtgaagggtttctacccctcggacatcgcggtcgaatgggaatcgaacgggcagccggagaacaactacaagaccacgccgccggtcctggactccgacgggtccttcttcctgtactcccggctgaccgtagacaagtcgcgctggcaggagggtaacgtgttcagctgcagcgtgatgcacgaggccctccacaaccactacacgcaaaagtcgctctccctgtccctgggc

SEQ ID NO: 88 - DNA, 648 bp, synthetic construct
  source 1..648: mol_type = other DNA
cagtctgctctgacccagcctgcttctgtgtctggctctcccggccagtccatcaccatctcttgtaccggcacctcctccgacgtgggcagacacaatctggtgtcctggtatcagcagcatcccggcaaggtgcccaaagtgatgatctacgaagtgaccaagcggcctgccggcgtgtccaacagattctccggatccaagtccggcaacaccgccagcctgacaattagcggactgcaggctgaggacgaggccgactactactgctgttcttacgccggctcctccacctacgtgttcggcactggaacaagagtgacagtgctgggccagcccaaggccgccccttctgttactctgttccctccatcctccgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctct

SEQ ID NO: 89 - DNA, 1344 bp, synthetic construct
  source 1..1344: mol_type = other DNA
caggttcagctgcagcaatctggacctggcctggtcaagccctctcagaccctgtctctgacctgtgccatctccggcgactccgtgtcctctaattctgccgcctggaactggatccggcagtctccatcttctggcctggaatggctgggcagaacctactaccggtccaagtggttcgacgactacgccgtgtctgtgaagggcagaatcaccatcaatcccgacacctccaagaaccagttctccctgcagctcaagttcgtgatccctgaggacaccgccgtgtactactgcgccagaagagtgcattggggcggaccctttgattactggggccagggaacactggtcaccgtgtcatctgctagcaccaaagggccgtccgtcttccccctggccccctgcagccggtcgacgtccgagtccaccgccgccctcgggtgcctggtcaaggactacttcccggagccggtaaccgtgagctggaactccggcgcgctgacctccggcgtgcacacgttccccgccgtcctgcagtcctccggcctctactccctctcgtccgtcgtcaccgtcccgagcagttccctgggcaccaagacctacacgtgcaacgtcgaccacaagccctccaacaccaaggtagacaagcgcgtcgagtccaaatacggccccccgtgcccgccctgtccggcccccgaggccgcgggcggcccctcagtgttcctgttcccgccgaagcccaaggacaccctgatgatctcgcgcacgcccgaggtcacgtgcgtggtcgtcgacgtctcacaggaagaccccgaggtgcagttcaactggtatgtcgacggcgtggaggttcacaacgcgaagaccaagccccgggaggagcagttcaacagcacataccgggtggtgtcggtcctcaccgtgctgcatcaggactggctgaacggtaaggagtacaagtgcaaggtgtccaacaagggcctcccgagcagcatagagaagaccatctccaaggcgaagggtcagccccgcgaaccgcaggtgtacaccctgccgccgagccaggaggagatgacgaagaaccaggtctccctgacgtgcctggtgaagggtttctacccctcggacatcgcggtcgaatgggaatcgaacgggcagccggagaacaactacaagaccacgccgccggtcctggactccgacgggtccttcttcctgtactcccggctgaccgtagacaagtcgcgctggcaggagggtaacgtgttcagctgcagcgtgatgcacgaggccctccacaaccactacacgcaaaagtcgctctccctgtccctgggc

SEQ ID NO: 90 - DNA, 642 bp, synthetic construct
  source 1..642: mol_type = other DNA
gacatccagctgacccagtctccatctttcctgtccgcctctgtgggcgacagagtgaccatcacctgtagagcctctcagggcatctcctcctacctggcctggtatcagcagaggcctggcaaggctcctaaggtgctgatctccgtggctagcacactgcagtctggcgtgccctctagattctccggctctggctctggcaccgactttaccctgacaatctccagcctgcagcctgaggacttcgccacctactactgccagcagctgaccggctatcccttcacctttggacctggcaccaaggtggacatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 91-95 - skipped (5 entries)

SEQ ID NO: 96 - protein, 646 aa, synthetic construct
QVQLVQSGSELKKPGASVKVSCKASGYTFTTYVMNWVRQAPGQGLEWMGWINTNTGNPTYAQGFTGRFVFSVDTSVSTAYLQISSLKAEDTAVYYCARDRVLLWFGAGYYHYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 97 - protein, 214 aa, synthetic construct
SSELTQDPAVSVALGQTVRITCQGDSLRSYYTSWYQQKPGQAPVFVIYGKNNRPSGIPDRFSGSSSGNTASLTITGAQAEDEADYYCNSRDSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 98 - protein, 453 aa, synthetic construct
QVQLVQSGSELKKPGASVKVSCKASGYTFTTYVMNWVRQAPGQGLEWMGWINTNTGNPTYAQGFTGRFVFSVDTSVSTAYLQISSLKAEDTAVYYCARDRVLLWFGAGYYHYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 99 - protein, 407 aa, synthetic construct
SSELTQDPAVSVALGQTVRITCQGDSLRSYYTSWYQQKPGQAPVFVIYGKNNRPSGIPDRFSGSSSGNTASLTITGAQAEDEADYYCNSRDSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 100 - protein, 640 aa, synthetic construct
QVQLQESGPGLVKPSQTLSLTCTVSGGSISSGGYYWSWIRQHPGKGLEWIGYIHYIGSTYQNPSLKSRVTMSVDTSKNQFSLRLTSVTAADTAVYYCARESGDTAMVFDYWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 101 - protein, 216 aa, synthetic construct
QSALTQPPSASGSPGQSVTISCTGTSSDVGGYNYVSWYQQHPGKAPKVMIYEVSTRPSGVPDRFSGSKSGNTASLTVSGLQAEDEADYYCSSYAGNNNFVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 102 - protein, 447 aa, synthetic construct
QVQLQESGPGLVKPSQTLSLTCTVSGGSISSGGYYWSWIRQHPGKGLEWIGYIHYIGSTYQNPSLKSRVTMSVDTSKNQFSLRLTSVTAADTAVYYCARESGDTAMVFDYWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 103 - protein, 409 aa, synthetic construct
QSALTQPPSASGSPGQSVTISCTGTSSDVGGYNYVSWYQQHPGKAPKVMIYEVSTRPSGVPDRFSGSKSGNTASLTVSGLQAEDEADYYCSSYAGNNNFVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 104 - protein, 642 aa, synthetic construct
QVQLVQSGAEVKKPGASVKVSCKASGYTFTNYDINWVRQATGQGLEWMGWMNPNSGNTDYVQKFQGRVAMTRITSISTAYLELSSLRSDDTAVYYCARGLHSGDYYSYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 105 - protein, 213 aa, synthetic construct
SYELTQPPSVSVSPGQTARITCSGDALPNQYAYWYQQKPGQAPVLVIYKDSERPSGIPERFSGSSSGTTVTLTISGVQAEDEADYYCQSADSSGTVVFGGGTKLSVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 106 - protein, 449 aa, synthetic construct
QVQLVQSGAEVKKPGASVKVSCKASGYTFTNYDINWVRQATGQGLEWMGWMNPNSGNTDYVQKFQGRVAMTRITSISTAYLELSSLRSDDTAVYYCARGLHSGDYYSYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 107 - protein, 406 aa, synthetic construct
SYELTQPPSVSVSPGQTARITCSGDALPNQYAYWYQQKPGQAPVLVIYKDSERPSGIPERFSGSSSGTTVTLTISGVQAEDEADYYCQSADSSGTVVFGGGTKLSVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 108 - protein, 738 aa, Homo sapiens
MRGPSGALWLLLALRTVLGGMEVRWCATSDPEQHKCGNMSEAFREAGIQPSLLCVRGTSADHCVQLIAAQEADAITLDGGAIYEAGKEHGLKPVVGEVYDQEVGTSYYAVAVVRRSSHVTIDTLKGVKSCHTGINRTVGWNVPVGYLVESGRLSVMGCDVLKAVSDYFGGSCVPGAGETSYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSEAYGQKDLLFKDSTSELVPIATQTYEAWLGHEYLHAMKGLLCDPNRLPPYLRWCVLSTPEIQKCGDMAVAFRRQRLKPEIQCVSAKSPQHCMERIQAEQVDAVTLSGEDIYTAGKTYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVMVRPDTNIFTVYGLLDKAQDLFGDDHNKNGFKMFDSSNYHGQDLLFKDATVRAVPVGEKTTYRGWLGLDYVAALEGMSSQQCSGAAAPAPGAPLLPLLLPALAARLLPPAL

SEQ ID NO: 109 - protein, 738 aa, Macaca fascicularis
MRGPSRALWLLLALRTVLGGMEVRWCVTSDPEQQKCSNMSTAFREAGIQPSLLCVQGTSPDHCIQLIAAQEADAITLDGGAIYEAGKEHGLKPVVGEVYDQEVGTSYYAVAVVKRSSQVTINTLKGVKSCHTGINRTVGWNVPVGYLVESGRLSVMGCDVLKAVSDYFGGSCVPGAGETRYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSKAYGQKDLLFKDSTSELVPIATQTYEAWLGQEYLHAMKGLLCDPNRLPPYLRWCVLSTPEIQKCGDMAVAFGRQQLKPEIQCVSAKSPQHCMEQIQAGQIDTVTLSGEDIYTAGKTYGLAPAAGEGYASEDSSNSYFVVAVVRRDSSHAFTLDELRGKRSCHAGFGSPAGWDIPVGALIRRGFIRPKDCDVLTAVSEFFNASCVPVNNPKNYPSSLCALCVGDEQGRNKCVGNSQERYYGNSGAFRCLVENAGDVAFVRHTTVFDNTNGHNSEPWAAELRSEDYELLCPNGARAEVSQFAACNLAQMPPHAVMVRPDTNIFTVYGLLDKAQDLFGDDHNKNGFKMFDSSNYHGQDLLFKDATVRAVPVGEKTTYRDWLGLDYVAALEGMLSEQCSGAAAPAPGAPLLPLLLPALAARLLPPAL

SEQ ID NO: 110 - protein, 7 aa, synthetic construct
SGGYYWS

SEQ ID NO: 111 - protein, 16 aa, synthetic construct
YIHYIGSTYQNPSLKS

SEQ ID NO: 112 - protein, 11 aa, synthetic construct
ESGDTAMVFDY

SEQ ID NO: 113 - protein, 10 aa, synthetic construct
GGSISSGGYY

SEQ ID NO: 114 - protein, 7 aa, synthetic construct
IHYIGST

SEQ ID NO: 115 - protein, 13 aa, synthetic construct
ARESGDTAMVFDY

SEQ ID NO: 116 - protein, 14 aa, synthetic construct
TGTSSDVGGYNYVS

SEQ ID NO: 117 - protein, 7 aa, synthetic construct
EVSTRPS

SEQ ID NO: 118 - protein, 10 aa, synthetic construct
SSYAGNNNFV

SEQ ID NO: 119 - protein, 9 aa, synthetic construct
SSDVGGYNY

SEQ ID NO: 120 - skipped

SEQ ID NO: 121 - protein, 10 aa, synthetic construct
SSYAGNNNFV

SEQ ID NO: 122 - protein, 121 aa, synthetic construct
QVQLQESGPGLVKPSQTLSLTCTVSGGSISSGGYYWSWIRQHPGKGLEWIGYIHYIGSTYQNPSLKSRVTMSVDTSKNQFSLRLTSVTAADTAVYYCARESGDTAMVFDYWGQGTQVTVSS

SEQ ID NO: 123 - DNA, 363 bp, synthetic construct
  source 1..363: mol_type = other DNA
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatccattacattgggagcacctaccagaacccgtccctcaagagtcgagttaccatgtcagtagacacgtctaagaaccagttctccctgaggctgacctctgtgactgccgcggacacggccgtgtattactgtgcgagagaaagtggggatacagctatggtctttgactactggggccagggaacccaggtcaccgtctcctca

SEQ ID NO: 124 - protein, 110 aa, synthetic construct
QSALTQPPSASGSPGQSVTISCTGTSSDVGGYNYVSWYQQHPGKAPKVMIYEVSTRPSGVPDRFSGSKSGNTASLTVSGLQAEDEADYYCSSYAGNNNFVFGTGTKVTVL

SEQ ID NO: 125 - DNA, 330 bp, synthetic construct
  source 1..330: mol_type = other DNA
cagtctgccctgactcagcctccctccgcgtccgggtctcctggacagtcagtcaccatctcctgcactggaaccagcagtgacgttggtggttataactatgtctcctggtaccaacagcacccaggcaaagcccccaaagtcatgatttatgaggtcagtacgcggccctcaggggtccctgatcgcttctctggctccaagtctggcaacacggcctccctgaccgtctctgggctccaggctgaggatgaggctgattattactgcagctcatatgcaggcaacaacaattttgtcttcggaactgggaccaaggtcaccgtccta

SEQ ID NO: 126 - protein, 451 aa, synthetic construct
QVQLQESGPGLVKPSQTLSLTCTVSGGSISSGGYYWSWIRQHPGKGLEWIGYIHYIGSTYQNPSLKSRVTMSVDTSKNQFSLRLTSVTAADTAVYYCARESGDTAMVFDYWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - DNA, 1356 bp, synthetic construct
  source 1..1356: mol_type = other DNA
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatccattacattgggagcacctaccagaacccgtccctcaagagtcgagttaccatgtcagtagacacgtctaagaaccagttctccctgaggctgacctctgtgactgccgcggacacggccgtgtattactgtgcgagagaaagtggggatacagctatggtctttgactactggggccagggaacccaggtcaccgtctcctcagctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacacagaagagcctctccctgtctccgggcaaatga

SEQ ID NO: 128 - protein, 216 aa, synthetic construct; identical to SEQ ID NO: 101

SEQ ID NO: 129 - DNA, 651 bp, synthetic construct
  source 1..651: mol_type = other DNA
cagtctgccctgactcagcctccctccgcgtccgggtctcctggacagtcagtcaccatctcctgcactggaaccagcagtgacgttggtggttataactatgtctcctggtaccaacagcacccaggcaaagcccccaaagtcatgatttatgaggtcagtacgcggccctcaggggtccctgatcgcttctctggctccaagtctggcaacacggcctccctgaccgtctctgggctccaggctgaggatgaggctgattattactgcagctcatatgcaggcaacaacaattttgtcttcggaactgggaccaaggtcaccgtcctaggtcagcccaaggcggcgccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 130 - protein, 7 aa, synthetic construct
SGNYYWS

SEQ ID NO: 131 - protein, 16 aa, synthetic construct
YIYYSGSTYYNPSLKS

SEQ ID NO: 132 - protein, 12 aa, synthetic construct
ADWNDQGDAFDI

SEQ ID NO: 133 - protein, 10 aa, synthetic construct
GGSINSGNYY

SEQ ID NO: 134 - protein, 7 aa, synthetic construct
IYYSGST

SEQ ID NO: 135 - protein, 14 aa, synthetic construct
ARADWNDQGDAFDI

SEQ ID NO: 136 - protein, 11 aa, synthetic construct
RASQDIRNDLG

SEQ ID NO: 137 - protein, 7 aa, synthetic construct
AASSLQS

SEQ ID NO: 138 - protein, 9 aa, synthetic construct
LQDYNYPFT

SEQ ID NO: 139 - protein, 6 aa, synthetic construct
QDIRND

SEQ ID NO: 140 - skipped

SEQ ID NO: 141 - protein, 9 aa, synthetic construct
LQDYNYPFT

SEQ ID NO: 142 - protein, 122 aa, synthetic construct
QVQLQESGPGLVKPSETLSLTCTVSGGSINSGNYYWSWIRQHPGKGLEWIGYIYYSGSTYYNPSLKSRVTISADTSKNQFSLKLSSVTAADTAVYYCARADWNDQGDAFDIWGQGTMATVSS

SEQ ID NO: 143 - DNA, 366 bp, synthetic construct
  source 1..366: mol_type = other DNA
caggtgcagctgcaggagtcgggcccaggactggtgaagccttcagagaccctgtccctcacctgcactgtctctggtggctccatcaacagtggtaattactactggagctggatccgccagcacccagggaagggcctggagtggattgggtacatctattacagtgggagcacctactacaacccgtccctcaagagtcgagttaccatatcagcagacacgtctaagaaccagttctccctgaagctgagctctgtgactgccgcggacacggccgtgtattactgtgcgagagcagactggaacgaccagggcgatgcttttgatatctggggccaagggacaatggccaccgtctcttca

SEQ ID NO: 144 - protein, 107 aa, synthetic construct
AIQMTQSPSSLSASVGDRVTITCRASQDIRNDLGWYQQKPGKAPKLLIYAASSLQSGVPSRFSGSGSGTDFTLTISSLQPEDFVTYYCLQDYNYPFTFGPGTKVDIK

SEQ ID NO: 145 - DNA, 321 bp, synthetic construct
  source 1..321: mol_type = other DNA
gccatccagatgacccagtctccatcctccctgtctgcatctgtaggagacagagtcaccatcacttgccgggcaagtcaggacattagaaatgatttaggctggtatcagcagaaaccagggaaagcccctaagctcctgatctatgctgcatccagtttacaaagtggggtcccatcaaggttcagcggcagtggatctggcacagatttcactctcaccatcagcagcctgcagcctgaagattttgtaacttattactgtctacaagattacaattacccattcactttcggccctgggaccaaagtggatatcaaa

SEQ ID NO: 146 - protein, 452 aa, synthetic construct
QVQLQESGPGLVKPSETLSLTCTVSGGSINSGNYYWSWIRQHPGKGLEWIGYIYYSGSTYYNPSLKSRVTISADTSKNQFSLKLSSVTAADTAVYYCARADWNDQGDAFDIWGQGTMATVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - DNA, 1359 bp, synthetic construct
  source 1..1359: mol_type = other DNA
caggtgcagctgcaggagtcgggcccaggactggtgaagccttcagagaccctgtccctcacctgcactgtctctggtggctccatcaacagtggtaattactactggagctggatccgccagcacccagggaagggcctggagtggattgggtacatctattacagtgggagcacctactacaacccgtccctcaagagtcgagttaccatatcagcagacacgtctaagaaccagttctccctgaagctgagctctgtgactgccgcggacacggccgtgtattactgtgcgagagcagactggaacgaccagggcgatgcttttgatatctggggccaagggacaatggccaccgtctcttcagctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacacagaagagcctctccctgtctccgggcaaatga

SEQ ID NO: 148 - protein, 214 aa, synthetic construct
AIQMTQSPSSLSASVGDRVTITCRASQDIRNDLGWYQQKPGKAPKLLIYAASSLQSGVPSRFSGSGSGTDFTLTISSLQPEDFVTYYCLQDYNYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - DNA, 645 bp, synthetic construct
  source 1..645: mol_type = other DNA
gccatccagatgacccagtctccatcctccctgtctgcatctgtaggagacagagtcaccatcacttgccgggcaagtcaggacattagaaatgatttaggctggtatcagcagaaaccagggaaagcccctaagctcctgatctatgctgcatccagtttacaaagtggggtcccatcaaggttcagcggcagtggatctggcacagatttcactctcaccatcagcagcctgcagcctgaagattttgtaacttattactgtctacaagattacaattacccattcactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 150 - protein, 7 aa, synthetic construct
SGGYYWS

SEQ ID NO: 151 - protein, 16 aa, synthetic construct
YIHYIGSTYQNPSLKS

SEQ ID NO: 152 - protein, 11 aa, synthetic construct
ESGDTAMVFDY

SEQ ID NO: 153 - protein, 10 aa, synthetic construct
GGSISSGGYY

SEQ ID NO: 154 - protein, 7 aa, synthetic construct
IHYIGST

SEQ ID NO: 155 - protein, 13 aa, synthetic construct
ARESGDTAMVFDY

SEQ ID NO: 156 - protein, 14 aa, synthetic construct
TGTSSDVGGYNYVS

SEQ ID NO: 157 - protein, 7 aa, synthetic construct
EVSTRPS

SEQ ID NO: 158 - protein, 10 aa, synthetic construct
SSYAGNNNFV

SEQ ID NO: 159 - protein, 9 aa, synthetic construct
SSDVGGYNY

SEQ ID NO: 160 - skipped

SEQ ID NO: 161 - protein, 10 aa, synthetic construct
SSYAGNNNFV

SEQ ID NO: 162 - protein, 121 aa, synthetic construct; identical to SEQ ID NO: 122

SEQ ID NO: 163 - DNA, 363 bp, synthetic construct
  source 1..363: mol_type = other DNA
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattggatacatccattacattgggagcacctaccagaacccgtccctcaagagtcgagttaccatgtcagtagacacgtctaagaaccagttctccctgaggctgacctctgtgactgccgcggacacggccgtttattactgtgcgagagaaagtggggatacagctatggtctttgactactggggccagggaacccaggtcaccgtctcctca

SEQ ID NO: 164 - protein, 110 aa, synthetic construct; identical to SEQ ID NO: 124

SEQ ID NO: 165 - DNA, 330 bp, synthetic construct; identical to SEQ ID NO: 125
  source 1..330: mol_type = other DNA

SEQ ID NO: 166 - protein, 451 aa, synthetic construct; identical to SEQ ID NO: 126

SEQ ID NO: 167 - DNA, 1353 bp, synthetic construct
  source 1..1353: mol_type = other DNA
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattggatacatccattacattgggagcacctaccagaacccgtccctcaagagtcgagttaccatgtcagtagacacgtctaagaaccagttctccctgaggctgacctctgtgactgccgcggacacggccgtttattactgtgcgagagaaagtggggatacagctatggtctttgactactggggccagggaacccaggtcaccgtctcctcagctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacacagaagagcctctccctgtctccgggcaaa

SEQ ID NO: 168 - protein, 216 aa, synthetic construct; identical to SEQ ID NO: 101

SEQ ID NO: 169 - DNA, 651 bp, synthetic construct; identical to SEQ ID NO: 129
  source 1..651: mol_type = other DNA

SEQ ID NO: 170 - protein, 5 aa, synthetic construct
TYVMN

SEQ ID NO: 171 - protein, 17 aa, synthetic construct
WINTNTGNPTYAQGFTG

SEQ ID NO: 172 - protein, 18 aa, synthetic construct
DRVLLWFGAGYYHYGMDV

SEQ ID NO: 173 - protein, 8 aa, synthetic construct
GYTFTTYV

SEQ ID NO: 174 - protein, 8 aa, synthetic construct
INTNTGNP

SEQ ID NO: 175 - protein, 20 aa, synthetic construct
ARDRVLLWFGAGYYHYGMDV

SEQ ID NO: 176 - protein, 11 aa, synthetic construct
QGDSLRSYYTS

SEQ ID NO: 177 - protein, 7 aa, synthetic construct
GKNNRPS

SEQ ID NO: 178 - protein, 11 aa, synthetic construct
NSRDSSGNHVV

SEQ ID NO: 179 - protein, 6 aa, synthetic construct
SLRSYY

SEQ ID NO: 180 - skipped

SEQ ID NO: 181 - protein, 11 aa, synthetic construct
NSRDSSGNHVV

SEQ ID NO: 182 - protein, 127 aa, synthetic construct
QVQLVQSGSELKKPGASVKVSCKASGYTFTTYVMNWVRQAPGQGLEWMGWINTNTGNPTYAQGFTGRFVFSVDTSVSTAYLQISSLKAEDTAVYYCARDRVLLWFGAGYYHYGMDVWGQGTTVTVSS

SEQ ID NO: 183 - DNA, 381 bp, synthetic construct
  source 1..381: mol_type = other DNA
caggtgcagctggtgcaatctgggtctgagttgaagaagcctggggcctcagtgaaggtttcctgcaaggcttctggatacaccttcactacctatgttatgaattgggtgcgacaggcccctggacaagggctcgagtggatgggatggatcaacaccaacactgggaacccaacgtatgcccagggcttcacaggacggtttgtcttctccgtggacacctctgtcagcacggcatatctgcagatcagcagcctaaaggctgaggacactgccgtgtattactgtgcgagagatcgagtattactatggttcggggcggggtactaccactacggtatggacgtctggggccaagggaccacggtcaccgtctcctca

SEQ ID NO: 184 - protein, 108 aa, synthetic construct
SSELTQDPAVSVALGQTVRITCQGDSLRSYYTSWYQQKPGQAPVFVIYGKNNRPSGIPDRFSGSSSGNTASLTITGAQAEDEADYYCNSRDSSGNHVVFGGGTKLTVL

SEQ ID NO: 185 - DNA, 324 bp, synthetic construct
  source 1..324: mol_type = other DNA
tcttctgagctgactcaggaccctgctgtgtctgtggccttgggacagacagtcaggatcacatgccaaggagacagcctcagatcctattatacaagctggtaccagcagaagccaggacaggcccctgtatttgtcatctatggtaaaaacaaccggccctcagggatcccagaccgattctctggctccagctcaggaaacacagcttccttgaccatcactggggctcaggcggaagatgaggctgactattactgtaattcccgggacagcagtggtaaccatgtggttttcggcggagggaccaagctgaccgtccta

SEQ ID NO: 186 - protein, 457 aa, synthetic construct
QVQLVQSGSELKKPGASVKVSCKASGYTFTTYVMNWVRQAPGQGLEWMGWINTNTGNPTYAQGFTGRFVFSVDTSVSTAYLQISSLKAEDTAVYYCARDRVLLWFGAGYYHYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - DNA, 1371 bp, synthetic construct
  source 1..1371: mol_type = other DNA
caggtgcagctggtgcaatctgggtctgagttgaagaagcctggggcctcagtgaaggtttcctgcaaggcttctggatacaccttcactacctatgttatgaattgggtgcgacaggcccctggacaagggctcgagtggatgggatggatcaacaccaacactgggaacccaacgtatgcccagggcttcacaggacggtttgtcttctccgtggacacctctgtcagcacggcatatctgcagatcagcagcctaaaggctgaggacactgccgtgtattactgtgcgagagatcgagtattactatggttcggggcggggtactaccactacggtatggacgtctggggccaagggaccacggtcaccgtctcctcagctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacacagaagagcctctccctgtctccgggcaaa

SEQ ID NO: 188 - protein, 215 aa, synthetic construct
SSELTQDPAVSVALGQTVRITCQGDSLRSYYTSWYQQKPGQAPVFVIYGKNNRPSGIPDRFSGSSSGNTASLTITGAQAEDEADYYCNSRDSSGNHVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - DNA, 645 bp, synthetic construct
  source 1..645: mol_type = other DNA
tcttctgagctgactcaggaccctgctgtgtctgtggccttgggacagacagtcaggatcacatgccaaggagacagcctcagatcctattatacaagctggtaccagcagaagccaggacaggcccctgtatttgtcatctatggtaaaaacaaccggccctcagggatcccagaccgattctctggctccagctcaggaaacacagcttccttgaccatcactggggctcaggcggaagatgaggctgactattactgtaattcccgggacagcagtggtaaccatgtggttttcggcggagggaccaagctgaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 190 - protein, 5 aa, synthetic construct
DYYMS

SEQ ID NO: 191 - protein, 17 aa, synthetic construct
YISSSGGAIFYADSVKG

SEQ ID NO: 192 - protein, 12 aa, synthetic construct
RYSSGWFEYFKH

SEQ ID NO: 193 - protein, 8 aa, synthetic construct
GFTFSDYY

SEQ ID NO: 194 - protein, 8 aa, synthetic construct
ISSSGGAI

SEQ ID NO: 195 - protein, 14 aa, synthetic construct
ARRYSSGWFEYFKH

SEQ ID NO: 196 - protein, 17 aa, synthetic construct
KSSQSVFNSSNKKNYLA

SEQ ID NO: 197 - protein, 7 aa, synthetic construct
WASTRES

SEQ ID NO: 198 - protein, 9 aa, synthetic construct
QQYYLAPYT

SEQ ID NO: 199 - protein, 12 aa, synthetic construct
QSVFNSSNKKNY

SEQ ID NO: 200 - skipped

SEQ ID NO: 201 - protein, 9 aa, synthetic construct
QQYYLAPYT

SEQ ID NO: 202 - protein, 121 aa, synthetic construct
QVQLVESGGGLVKPGGSLRLSCAASGFTFSDYYMSWIRQAPGKGLEWISYISSSGGAIFYADSVKGRFTISRDNAKNSLYLQMNSLRTEDTAVYYCARRYSSGWFEYFKHWGQGTLVTVSS

SEQ ID NO: 203 - DNA, 363 bp, synthetic construct
  source 1..363: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtggatttcatacattagtagtagtggtggtgccatattctacgcagactctgtgaagggccgattcaccatctccagggacaacgccaagaactcactgtatctgcaaatgaacagcctgagaaccgaggacacggccgtgtattactgtgcgagaaggtatagcagtggctggttcgaatacttcaaacactggggccagggcaccctggtcaccgtctcctca

SEQ ID NO: 204 - protein, 113 aa, synthetic construct
DIVMTQSPDSLAVSLGERATINCKSSQSVFNSSNKKNYLAWYQQKPGQPPKLLIYWASTRESGVPDRFSGSGSGTDFTLTISSLQAEDVAVYYCQQYYLAPYTFGQGTKLEIK

SEQ ID NO: 205 - DNA, 342 bp, synthetic construct
  source 1..342: mol_type = other DNA
gacatcgtgatgacccagtctccagactccctggctgtgtctctgggcgagagggccaccatcaactgcaagtccagccagagtgttttcaacagttccaacaaaaagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatcttgctccgtacacttttggccaggggaccaagctggagatcaaacgt

SEQ ID NO: 206 - protein, 451 aa, synthetic construct
QVQLVESGGGLVKPGGSLRLSCAASGFTFSDYYMSWIRQAPGKGLEWISYISSSGGAIFYADSVKGRFTISRDNAKNSLYLQMNSLRTEDTAVYYCARRYSSGWFEYFKHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - DNA, 1353 bp, synthetic construct
  source 1..1353: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtggatttcatacattagtagtagtggtggtgccatattctacgcagactctgtgaagggccgattcaccatctccagggacaacgccaagaactcactgtatctgcaaatgaacagcctgagaaccgaggacacggccgtgtattactgtgcgagaaggtatagcagtggctggttcgaatacttcaaacactggggccagggcaccctggtcaccgtctcctcagctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacacagaagagcctctccctgtctccgggcaaa

SEQ ID NO: 208 - protein, 220 aa, synthetic construct
DIVMTQSPDSLAVSLGERATINCKSSQSVFNSSNKKNYLAWYQQKPGQPPKLLIYWASTRESGVPDRFSGSGSGTDFTLTISSLQAEDVAVYYCQQYYLAPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - DNA, 660 bp, synthetic construct
  source 1..660: mol_type = other DNA
gacatcgtgatgacccagtctccagactccctggctgtgtctctgggcgagagggccaccatcaactgcaagtccagccagagtgttttcaacagttccaacaaaaagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatcttgctccgtacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 210 - protein, 5 aa, synthetic construct
SYGIH

SEQ ID NO: 211 - protein, 17 aa, synthetic construct
VIWYDGSNKYYADSVKG

SEQ ID NO: 212 - protein, 11 aa, synthetic construct
RGDSSSWHFDY

SEQ ID NO: 213 - protein, 8 aa, synthetic construct
GFTFSSYG

SEQ ID NO: 214 - protein, 8 aa, synthetic construct
IWYDGSNK

SEQ ID NO: 215 - protein, 13 aa, synthetic construct
ARRGDSSSWHFDY

SEQ ID NO: 216 - protein, 14 aa, synthetic construct
TGTSSDVGRHNLVS

SEQ ID NO: 217 - protein, 7 aa, synthetic construct
EVTKRPA

SEQ ID NO: 218 - protein, 10 aa, synthetic construct
CSYAGSSTYV

SEQ ID NO: 219 - protein, 9 aa, synthetic construct
SSDVGRHNL

SEQ ID NO: 220 - skipped

SEQ ID NO: 221 - protein, 10 aa, synthetic construct
CSYAGSSTYV

SEQ ID NO: 222 - protein, 120 aa, synthetic construct; identical to SEQ ID NO: 70

SEQ ID NO: 223 - DNA, 360 bp, synthetic construct
  source 1..360: mol_type = other DNA
caggtgcagctggttgaatctggtggcggagtggtgcagcctggcagatctctgagactgtcttgtgccgcctccggcttcaccttctcctcttacggaatccactgggtccgacaggcccctggcaaaggattggaatgggtcgccgtgatttggtacgacggctccaacaagtactacgccgactccgtgaagggcagattcaccatctctcgggacaactccaagaacaccctgtacctgcagatgaactccctgagagccgaggacaccgccgtgtactactgtgccagaagaggcgactcctcctcctggcactttgactattggggccagggcaccctggtcaccgttagttct

SEQ ID NO: 224 - protein, 110 aa, synthetic construct; identical to SEQ ID NO: 71

SEQ ID NO: 225 - DNA, 330 bp, synthetic construct
  source 1..330: mol_type = other DNA
cagtctgctctgacccagcctgcttctgtgtctggctctcccggccagtccatcaccatctcttgtaccggcacctcctccgacgtgggcagacacaatctggtgtcctggtatcagcagcatcccggcaaggtgcccaaagtgatgatctacgaagtgaccaagcggcctgccggcgtgtccaacagattctccggatccaagtccggcaacaccgccagcctgacaattagcggactgcaggctgaggacgaggccgactactactgctgttcttacgccggctcctccacctacgtgttcggcactggaacaagagtgacagtgctg

SEQ ID NO: 226 - protein, 450 aa, synthetic construct; identical to SEQ ID NO: 74

SEQ ID NO: 227 - DNA, 1350 bp, synthetic construct
  source 1..1350: mol_type = other DNA
caggtgcagctggtggagtctgggggaggcgtggtccagcctgggaggtccctgagactctcctgtgcagcgtctggattcaccttcagtagctatggcattcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgaggagaggcgatagcagcagctggcactttgactactggggccagggaaccctggtcaccgtctcctcagctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacacagaagagcctctccctgtctccgggcaaa

SEQ ID NO: 228 - protein, 216 aa, synthetic construct; identical to SEQ ID NO: 76

SEQ ID NO: 229 - DNA, 648 bp, synthetic construct
  source 1..648: mol_type = other DNA
cagtctgccctgactcagcctgcctccgtgtctgggtctcctggacagtcgatcaccatctcctgcactggaaccagcagtgatgttgggaggcataaccttgtctcctggtaccaacagcacccaggcaaagtccccaaagtcatgatttatgaggtcactaagcggcccgcaggggtgtctaatcgcttctctggctccaagtctggcaacacggcctccctgacaatctctgggctccaggctgaggacgaggctgattattactgctgctcatatgcaggtagtagcacttatgtcttcggaactgggaccagggtcaccgtccttggtcagcccaaggcggcgccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 230 - protein, 5 aa, synthetic construct
DYYMS

SEQ ID NO: 231 - protein, 17 aa, synthetic construct
YISSSGGAIFYADSVKG

SEQ ID NO: 232 - protein, 12 aa, synthetic construct
RYSSGWFEYFKH

SEQ ID NO: 233 - protein, 8 aa, synthetic construct
GFTFSDYY

SEQ ID NO: 234 - protein, 8 aa, synthetic construct
ISSSGGAI

SEQ ID NO: 235 - protein, 14 aa, synthetic construct
ARRYSSGWFEYFKH

SEQ ID NO: 236 - protein, 17 aa, synthetic construct
KSSQSVLNSSNKKNYLA

SEQ ID NO: 237 - protein, 7 aa, synthetic construct
WASTRES

SEQ ID NO: 238 - protein, 9 aa, synthetic construct
QQYYLAPYT

SEQ ID NO: 239 - protein, 12 aa, synthetic construct
QSVLNSSNKKNY

SEQ ID NO: 240 - skipped

SEQ ID NO: 241 - protein, 9 aa, synthetic construct
QQYYLAPYT

SEQ ID NO: 242 - protein, 121 aa, synthetic construct; identical to SEQ ID NO: 202

SEQ ID NO: 243 - DNA, 363 bp, synthetic construct
  source 1..363: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtggatttcatacattagtagtagtggtggtgccatattctacgcagactctgtgaagggccgattcaccatctccagggacaacgccaagaactcactgtatctgcaaatgaacagcctgagaaccgaggacacggccgtgtattactgtgcgagaaggtatagcagtggctggttcgagtacttcaaacactggggccagggcaccctggtcaccgtctcctca

SEQ ID NO: 244 - protein, 113 aa, synthetic construct
DIVMTQSPDSLAVSLGERATINCKSSQSVLNSSNKKNYLAWYQQKPGQPPKLLIYWASTRESGVPDRFSGSGSGTDFTLTISSLQAEDVAVYYCQQYYLAPYTFGQGTKLEIK

SEQ ID NO: 245 - DNA, 342 bp, synthetic construct
  source 1..342: mol_type = other DNA
gacatcgtgatgacccagtctccagactccctggctgtgtctctgggcgagagggccaccatcaactgcaagtccagccagagtgttttaaacagttccaacaaaaagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatcttgctccgtacacttttggccaggggaccaagctggagatcaaacgt

SEQ ID NO: 246 - protein, 451 aa, synthetic construct; identical to SEQ ID NO: 206

SEQ ID NO: 247 - DNA, 1353 bp, synthetic construct
  source 1..1353: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtggatttcatacattagtagtagtggtggtgccatattctacgcagactctgtgaagggccgattcaccatctccagggacaacgccaagaactcactgtatctgcaaatgaacagcctgagaaccgaggacacggccgtgtattactgtgcgagaaggtatagcagtggctggttcgagtacttcaaacactggggccagggcaccctggtcaccgtctcctcagctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacacagaagagcctctccctgtctccgggcaaa

SEQ ID NO: 248 - protein, 220 aa, synthetic construct
DIVMTQSPDSLAVSLGERATINCKSSQSVLNSSNKKNYLAWYQQKPGQPPKLLIYWASTRESGVPDRFSGSGSGTDFTLTISSLQAEDVAVYYCQQYYLAPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 249 - DNA, 660 bp, synthetic construct
  source 1..660: mol_type = other DNA
gacatcgtgatgacccagtctccagactccctggctgtgtctctgggcgagagggccaccatcaactgcaagtccagccagagtgttttaaacagttccaacaaaaagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatcttgctccgtacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 250 - protein, 7 aa, synthetic construct
SNSAAWN

SEQ ID NO: 251 - protein, 18 aa, synthetic construct
RTYYRSKWFDDYAVSVKG

SEQ ID NO: 252 - protein, 10 aa, synthetic construct
RVHWGGPFDY

SEQ ID NO: 253 - protein, 10 aa, synthetic construct
GDSVSSNSAA

SEQ ID NO: 254 - protein, 9 aa, synthetic construct
TYYRSKWFD

SEQ ID NO: 255 - protein, 12 aa, synthetic construct
ARRVHWGGPFDY

SEQ ID NO: 256 - protein, 11 aa, synthetic construct
RASQGISSYLA

SEQ ID NO: 257 - protein, 7 aa, synthetic construct
VASTLQS

SEQ ID NO: 258 - protein, 9 aa, synthetic construct
QQLTGYPFT

SEQ ID NO: 259 - protein, 6 aa, synthetic construct
QGISSY

SEQ ID NO: 260 - skipped

SEQ ID NO: 261 - protein, 9 aa, synthetic construct
QQLTGYPFT

SEQ ID NO: 262 - protein, 122 aa, synthetic construct; identical to SEQ ID NO: 72

SEQ ID NO: 263 - DNA, 366 bp, synthetic construct
  source 1..366: mol_type = other DNA
caggtacagctgcagcagtcaggtccaggactggtgaagccctcgcagaccctctcactcacctgtgccatctccggggacagtgtctctagcaacagtgctgcttggaactggatcaggcagtccccctcgagtggccttgagtggctgggaaggacttactacaggtccaagtggtttgatgattatgcagtctctgtgaaaggtcgaataaccatcaacccagacacatccaagaaccagttctcccttcagttaaagtttgtgattcccgaggacacggctgtttattattgtgcaagaagagtccactgggggggtccctttgactattggggccagggaaccctggtcaccgtctcctca

SEQ ID NO: 264 - protein, 107 aa, synthetic construct; identical to SEQ ID NO: 73

SEQ ID NO: 265 - DNA, 324 bp, synthetic construct
  source 1..324: mol_type = other DNA
gacatccagttgacccagtctccatccttcctgtctgcctctgtaggagacagagtcaccatcacttgccgggccagtcagggcattagcagttatttagcctggtatcagcaaagaccagggaaagcccctaaggtcttgatctctgttgcatccactttgcaaagtggggtcccatcaaggttcagcggcagtggatctgggacagatttcactctcacaatcagcagcctgcagcctgaggattttgcgacttattactgtcaacagcttactggttacccattcactttcggccctgggaccaaagtggatatcaaacgt

SEQ ID NO: 266 - protein, 452 aa, synthetic construct; identical to SEQ ID NO: 77

SEQ ID NO: 267 - DNA, 1356 bp, synthetic construct
  source 1..1356: mol_type = other DNA
caggtacagctgcagcagtcaggtccaggactggtgaagccctcgcagaccctctcactcacctgtgccatctccggggacagtgtctctagcaacagtgctgcttggaactggatcaggcagtccccctcgagtggccttgagtggctgggaaggacttactacaggtccaagtggtttgatgattatgcagtctctgtgaaaggtcgaataaccatcaacccagacacatccaagaaccagttctcccttcagttaaagtttgtgattcccgaggacacggctgtttattattgtgcaagaagagtccactgggggggtccctttgactattggggccagggaaccctggtcaccgtctcctcagctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacacagaagagcctctccctgtctccgggcaaa

SEQ ID NO: 268 - protein, 214 aa, synthetic construct; identical to SEQ ID NO: 79

SEQ ID NO: 269 - DNA, 642 bp, synthetic construct
  source 1..642: mol_type = other DNA
gacatccagttgacccagtctccatccttcctgtctgcctctgtaggagacagagtcaccatcacttgccgggccagtcagggcattagcagttatttagcctggtatcagcaaagaccagggaaagcccctaaggtcttgatctctgttgcatccactttgcaaagtggggtcccatcaaggttcagcggcagtggatctgggacagatttcactctcacaatcagcagcctgcagcctgaggattttgcgacttattactgtcaacagcttactggttacccattcactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 270 - protein, 5 aa, synthetic construct
NYDIN

SEQ ID NO: 271 - protein, 17 aa, synthetic construct
WMNPNSGNTDYVQKFQG

SEQ ID NO: 272 - protein, 14 aa, synthetic construct
GLHSGDYYSYGMDV

SEQ ID NO: 273 - protein, 8 aa, synthetic construct
GYTFTNYD

SEQ ID NO: 274 - protein, 8 aa, synthetic construct
MNPNSGNT

SEQ ID NO: 275 - protein, 16 aa, synthetic construct
ARGLHSGDYYSYGMDV

SEQ ID NO: 276 - protein, 11 aa, synthetic construct
SGDALPNQYAY

SEQ ID NO: 277 - protein, 7 aa, synthetic construct
KDSERPS

SEQ ID NO: 278 - protein, 10 aa, synthetic construct
QSADSSGTVV

SEQ ID NO: 279 - protein, 6 aa, synthetic construct
ALPNQY

SEQ ID NO: 280 - skipped

SEQ ID NO: 281 - protein, 10 aa, synthetic construct
QSADSSGTVV

SEQ ID NO: 282 - protein, 123 aa, synthetic construct
QVQLVQSGAEVKKPGASVKVSCKASGYTFTNYDINWVRQATGQGLEWMGWMNPNSGNTDYVQKFQGRVAMTRITSISTAYLELSSLRSDDTAVYYCARGLHSGDYYSYGMDVWGQGTTVTVSS

SEQ ID NO: 283 - DNA, 369 bp, synthetic construct
  source 1..369: mol_type = other DNA
caggtgcagctggtgcagtctggggctgaggtgaagaagcctggggcctcagtgaaggtctcctgcaaggcttctggatacaccttcaccaattatgatatcaactgggtgcgacaggccactggacaagggcttgagtggatgggatggatgaaccctaacagtggtaacacagactatgtacagaagttccagggcagagtcgccatgaccaggatcacctccataagcacagcctacttggagctgagcagcctgagatctgacgacacggccgtgtattattgtgcgagaggcctccatagtggggattactactcctacggtatggacgtctggggccaagggaccacggtcaccgtctcctca

SEQ ID NO: 284 - protein, 107 aa, synthetic construct
SYELTQPPSVSVSPGQTARITCSGDALPNQYAYWYQQKPGQAPVLVIYKDSERPSGIPERFSGSSSGTTVTLTISGVQAEDEADYYCQSADSSGTVVFGGGTKLSVL

SEQ ID NO: 285 - DNA, 321 bp, synthetic construct
  source 1..321: mol_type = other DNA
tcctatgagctgacacagccaccctcggtgtcagtgtccccaggacagacggccaggatcacctgctctggagatgcattgccaaaccaatatgcttattggtaccaacagaagccaggccaggcccctgtcctagtgatatataaagacagtgagaggccctcagggatccctgagcgattctctggctccagctcagggacaacagtcacgttgaccatcagtggagtccaggcagaagacgaggctgactattactgtcaatcagcagacagcagtggtactgtggttttcggcggagggaccaagctgtccgtctta

SEQ ID NO: 286 - protein, 453 aa, synthetic construct
QVQLVQSGAEVKKPGASVKVSCKASGYTFTNYDINWVRQATGQGLEWMGWMNPNSGNTDYVQKFQGRVAMTRITSISTAYLELSSLRSDDTAVYYCARGLHSGDYYSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - DNA, 1362 bp, synthetic construct
  source 1..1362: mol_type = other DNA
caggtgcagctggtgcagtctggggctgaggtgaagaagcctggggcctcagtgaaggtctcctgcaaggcttctggatacaccttcaccaattatgatatcaactgggtgcgacaggccactggacaagggcttgagtggatgggatggatgaaccctaacagtggtaacacagactatgtacagaagttccagggcagagtcgccatgaccaggatcacctccataagcacagcctacttggagctgagcagcctgagatctgacgacacggccgtgtattattgtgcgagaggcctccatagtggggattactactcctacggtatggacgtctggggccaagggaccacggtcaccgtctcctcagctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacacagaagagcctctccctgtctccgggcaaatga

SEQ ID NO: 288 - protein, 213 aa, synthetic construct; identical to SEQ ID NO: 105

SEQ ID NO: 289 - DNA, 642 bp, synthetic construct
  source 1..642: mol_type = other DNA
tcctatgagctgacacagccaccctcggtgtcagtgtccccaggacagacggccaggatcacctgctctggagatgcattgccaaaccaatatgcttattggtaccaacagaagccaggccaggcccctgtcctagtgatatataaagacagtgagaggccctcagggatccctgagcgattctctggctccagctcagggacaacagtcacgttgaccatcagtggagtccaggcagaagacgaggctgactattactgtcaatcagcagacagcagtggtactgtggttttcggcggagggaccaagctgtccgtcctaggtcagcccaaggcggcgccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag